(19) United States
(12) Patent Application Publication
(10) Pub. No.: US 2022/0401335 A1
(43) Pub. Date: Dec. 22, 2022

(54) SILK HAIR CARE COMPOSITIONS

(71) Applicant: Evolved by Nature, Inc., Medford, MA (US)

(72) Inventors: Sara A. JOHNSON, Medford, MA (US); Carlos J. BOSQUES, Arlington, MA (US); Gregory H. ALTMAN, Providence, RI (US); Abigail GIARROSSO, Methuen, MA (US); Laura R. MUOLLO, Dracut, MA (US); Ilan GOLDBERG, Somerville, MA (US); Marius COSTACHE, Lexington, MA (US)

(73) Assignee: Evolved by Nature, Inc., Medford, MA (US)

(21) Appl. No.: 17/604,192

(22) PCT Filed: Apr. 16, 2020
(86) PCT No.: PCT/US2020/028580

§ 371 Date: Oct. 15, 2021

Related U.S. Application Data
(60) Provisional application No. 62/834,520, filed on Apr. 16, 2019.
(60) Provisional application No. 63/009,587, filed on Apr. 14, 2020.

(51) Int. Cl. A61K 8/64; A61K 8/20; A61K 8/92; A61K 8/06; A61K 8/41; A61Q 5/00

(52) U.S. Cl. CPC A61K 8/64; A61K 8/20; A61K 8/92; A61K 8/062; A61K 8/416; A61Q 5/002; A61K 2800/596

(57) ABSTRACT

The disclosure describes silk hair care compositions and methods of using and making thereof.

### FIELD

This disclosure is in the field of novel hair care compositions including SPF, e.g., without limitation silk fibroin protein fragments. The silk hair care compositions can provide hair conditioning, nourishing, improved feel in use, cleaning, hair styling, hair repairing, hair strengthening, heat protection, preventing color loss from dyed hair, enhancing color delivery, UV protection, and the like.

### BACKGROUND

Silk is a natural polymer produced by a variety of insects and spiders. Silk comprises a filament core protein, silk fibroin, and a glue-like coating consisting of a nonfilamentous protein, sericin.

Undamaged virgin hair is smooth and shiny. Its cuticles on the surface of the hair lie down smoothly making the combing easy. The hair surface is also hydrophobic in nature preventing excessive water absorption during washing. When the hair is either mechanically or chemically damaged through bleaching, sun or other sources of UV radiation exposure, perming, coloring, or washing with harsh surfactants, the hair surface becomes rough and frizzy, and difficult to detangle and comb. As the hair surface becomes more hydrophilic, the resulting hair fibers swell during washing making the hair increasingly more difficult to comb.

There exists a need for stable silk fibroin peptide solution suitable for hair care products. Additionally, there is a need for novel hair care products that enhance the beneficial effects of the self-assembly and coating properties of the silk fibroin peptides.

### SUMMARY

The disclosure provides a hair care composition including silk fibroin fragments having an average weight average molecular weight selected from between about 1 kDa and about 5 kDa, between about 5 kDa and about 10 kDa, between about 6 kDa and about 17 kDa, between about 10 kDa and about 15 kDa, between about 15 kDa and about 20 kDa, between about 17 kDa and about 39 kDa, between about 20 kDa and about 25 kDa, between about 25 kDa and about 30 kDa, between about 30 kDa and about 35 kDa, between about 35 kDa and about 40 kDa, between about 39 kDa and about 80 kDa, between about 40 kDa and about 45 kDa, between about 45 kDa and about 50 kDa, between about 60 kDa and about 100 kDa, and between about 80 kDa and about 144 kDa, and a polydispersity between 1 and about 5; 0 to 500 ppm lithium bromide; 0 to 500 ppm sodium carbonate; and a dermatologically acceptable carrier. In some embodiments, the silk fibroin fragments have a polydispersity between 1 and about 1.5. In some embodiments, the silk fibroin fragments have a polydispersity between about 1.5 and about 2.0. In some embodiments, the silk fibroin fragments have a polydispersity between about 1.5 and about 3.0. In some embodiments, the silk fibroin fragments have a polydispersity between about 2.0 and about 2.5. In some embodiments, the silk fibroin fragments have a polydispersity between about 2.5 and about 3.0. In some embodiments, the silk fibroin fragments are present in the hair care composition at about 0.01 wt. % to about 10.0 wt. % relative to the total weight of the hair care composition. In some embodiments, the silk fibroin fragments are present in the hair care composition at about 0.01 wt. % to about 1.0 wt. % relative to the total weight of the hair care composition. In some embodiments, the silk fibroin fragments are present in the hair care composition at about 1.0 wt. % to about 2.0 wt. % relative to the total weight of the hair care composition. In some embodiments, the silk fibroin fragments are present in the hair care composition at about 2.0 wt. % to about 3.0 wt. % relative to the total weight of the hair care composition. In some embodiments, the silk fibroin fragments are present in the hair care composition at about 3.0 wt. % to about 4.0 wt. % relative to the total weight of the hair care composition. In some embodiments, the silk fibroin fragments are present in the hair care composition at about 4.0 wt. % to about 5.0 wt. % relative to the total weight of the hair care composition. In some embodiments, the silk fibroin fragments are present in the hair care composition at about 5.0 wt. % to about 6.0 wt. % relative to the total weight of the hair care composition. In some embodiments, the silk fibroin fragments include hydrophilic domains. In some embodiments, the silk fibroin fragments include hydrophobic domains. In some embodiments, the silk fibroin fragments include both hydrophilic and hydrophobic domains.

The disclosure also provides a hair care composition including silk fibroin fragments as described herein, and further including about 0.01% (w/w) to about 10% (w/w) sericin relative to the total weight of the hair care composition. In some embodiments, the hair care composition further includes about 0.01% (w/w) to about 10% (w/w) sericin relative to the silk fibroin fragments.

The disclosure also provides a hair care composition including silk fibroin fragments as described herein, wherein the silk fibroin fragments do not spontaneously or gradually gel and do not visibly change in color or turbidity when in an aqueous solution for at least 10 days prior to formulation into the hair care composition. In some embodiments, the silk fibroin fragments do not spontaneously or gradually gel and do not visibly change in color or turbidity when in an aqueous solution for at least 1 day, at least 2 days, at least 3 days, at least 4 days, at least 5 days, at least 6 days, at least 7 days, at least 8 days, at least 9 days, at least 11 days, at least 12 days, at least 13 days, at least 2 weeks, at least 3 weeks, at least 4 weeks, or at least one month prior to formulation into the hair care composition.

The disclosure also provides a hair care composition including silk fibroin fragments as described herein, and a dermatologically acceptable carrier. In some embodiments, the dermatologically acceptable carrier includes an oil phase. In some embodiments, the dermatologically acceptable carrier includes an aqueous phase. In some embodiments, the dermatologically acceptable carrier includes suspended solids. In some embodiments, the hair care composition further includes an emulsifier. In some embodiments, the dermatologically acceptable carrier includes an oil-in-water emulsion or a water-in-oil emulsion.

The disclosure also provides a hair care composition including silk fibroin fragments as described herein, and a dermatologically acceptable carrier as described herein, and further including a hydrocarbon oil, a fatty acid, a fatty oil, a fatty acid ester, a cationic quaternary ammonium salt, or any combination thereof. In some embodiments, the hair care composition further includes a detersive, a detergent, or a combination thereof. In some embodiments, the hair care composition further includes a hair styling polymer. In some embodiments, the hair care composition further comprises a coloring agent, a pigment, a dye molecule, a color protecting agent, a UV filter, a scalp protecting agent, and combinations thereof.

The disclosure also provides a hair care composition including silk fibroin fragments as described herein, and a dermatologically acceptable carrier as described herein, wherein the hair care composition is transparent, translucent, or opaque.

The disclosure also provides a hair pre-shampoo product including a hair care composition including silk fibroin fragments as described herein, and a dermatologically acceptable carrier as described herein. In some embodiments, the disclosure provides a hair shampoo product including a hair care composition including silk fibroin fragments as described herein, and a dermatologically acceptable carrier as described herein. In some embodiments, the disclosure provides a hair rinsing product including a hair care composition including silk fibroin fragments as described herein, and a dermatologically acceptable carrier as described herein. In some embodiments, the disclosure provides a hair conditioner product including a hair care composition including silk fibroin fragments as described herein, and a dermatologically acceptable carrier as described herein. In some embodiments, the disclosure provides a hair treatment product including a hair care composition including silk fibroin fragments as described herein, and a dermatologically acceptable carrier as described herein. In some embodiments, the disclosure provides a hair setting lotion product including a hair care composition including silk fibroin fragments as described herein, and a dermatologically acceptable carrier as described herein. In some embodiments, the disclosure provides a blow-styling lotion product including a hair care composition including silk fibroin fragments as described herein, and a dermatologically acceptable carrier as described herein. In some embodiments, the disclosure provides a hair spray product including a hair care composition including silk fibroin fragments as described herein, and a dermatologically acceptable carrier as described herein. In some embodiments, the disclosure provides a hair-styling foam product including a hair care composition including silk fibroin fragments as described herein, and a dermatologically acceptable carrier as described herein. In some embodiments, the disclosure provides a hair-styling gel product including a hair care composition including silk fibroin fragments as described herein, and a dermatologically acceptable carrier as described herein. In some embodiments, the disclosure provides a hair oil product including a hair care composition including silk fibroin fragments as described herein, and a dermatologically acceptable carrier as described herein. In some embodiments, the disclosure provides a hair liquid product including a hair care composition including silk fibroin fragments as described herein, and a dermatologically acceptable carrier as described herein. In some embodiments, the disclosure provides a hair tonic product including a hair care composition including silk fibroin fragments as described herein, and a dermatologically acceptable carrier as described herein. In some embodiments, the disclosure provides a hair cream product including a hair care composition including silk fibroin fragments as described herein, and a dermatologically acceptable carrier as described herein. In some embodiments, the disclosure provides a hair cosmetic product including a hair care composition including silk fibroin fragments as described herein, and a dermatologically acceptable carrier as described herein. In some embodiments, the disclosure provides a permanent waving agent product including a hair care composition including silk fibroin fragments as described herein, and a dermatologically acceptable carrier as described herein. In some embodiments, the disclosure provides a hair mousse product including a hair care composition including silk fibroin fragments as described herein, and a dermatologically acceptable carrier as described herein. In some embodiments, the disclosure provides a hair gel product including a hair care composition including silk fibroin fragments as described herein, and a dermatologically acceptable carrier as described herein. In some embodiments, the disclosure provides a hair tonic product including a hair care composition including silk fibroin fragments as described herein, and a dermatologically acceptable carrier as described herein. In some embodiments, the disclosure provides a hair foam product including a hair care composition including silk fibroin fragments as described herein, and a dermatologically acceptable carrier as described herein. In some embodiments, the disclosure provides a hair paste product including a hair care composition including silk fibroin fragments as described herein, and a dermatologically acceptable carrier as described herein. In some embodiments, the disclosure provides a hair lotion product including a hair care composition including silk fibroin fragments as described herein, and a dermatologically acceptable carrier as described herein. In some embodiments, the disclosure provides a hair wax product including a hair care composition including silk fibroin fragments as described herein, and a dermatologically acceptable carrier as described herein.

SPF, e.g., without limitation silk fibroin protein fragment solutions and hair care products manufactured therefrom are disclosed herein. In an embodiment, this disclosure provides a hair care composition comprising (i) silk fibroin-based protein fragments that are substantially devoid of sericin at weight percent ranging from about 0.0001 wt. % to about 10.0 wt. % by the total weight of the hair care composition, (ii) at least one hair care active agent, and (iii) a dermatologically acceptable carrier, wherein the silk fibroin-based protein fragments have a weight average molecular weight ranging from about 5 kDa to about 80 kDa, wherein the silk fibroin-based protein fragments have a polydispersity ranging from about 1.5 to about 3.0.

In an embodiment, this disclosure provides a hair care composition comprising (i) silk fibroin-based protein fragments that are substantially devoid of sericin at weight percent ranging from about 0.0001 wt. % to about 10.0 wt. % by the total weight of the hair care composition, (ii) at least one hair care active agent, and (iii) a dermatologically acceptable carrier, wherein the silk fibroin-based protein fragments have a weight average molecular weight ranging from about 1 kDa to about 5 kDa, from about 5 kDa to about 10 kDa, from about 6 kDa to about 17 kDa, from about 10 kDa to about 15 kDa, from about 15 kDa to about 20 kDa, from about 17 kDa to about 39 kDa, from about 20 kDa to about 25 kDa, from about 25 kDa to about 30 kDa, from about 30 kDa to about 35 kDa, from about 35 kDa to about 40 kDa, from about 39 kDa to about 80 kDa, from about 40 kDa to about 45 kDa, from about 45 kDa to about 50 kDa, from about 60 kDa to about 100 kDa, or from about 80 kDa to about 144 kDa, wherein the silk fibroin-based protein fragments have a polydispersity ranging from 1 to about 5.0.

In an embodiment, this disclosure provides a tunable hair care composition comprising (i) silk fibroin-based protein fragments that are substantially devoid of sericin at weight percent ranging from about 0.0001 wt. % to about 10.0 wt. % by the total weight of the hair care composition, (ii) at least one hair care active agent, and (iii) a dermatologically acceptable carrier, wherein the silk fibroin-based protein fragments have a weight average molecular weight ranging from about 1 kDa to about 5 kDa, from about 5 kDa to about 10 kDa, from about 6 kDa to about 17 kDa, from about 10 kDa to about 15 kDa, from about 15 kDa to about 20 kDa, from about 17 kDa to about 39 kDa, from about 20 kDa to about 25 kDa, from about 25 kDa to about 30 kDa, from about 30 kDa to about 35 kDa, from about 35 kDa to about 40 kDa, from about 39 kDa to about 80 kDa, from about 40 kDa to about 45 kDa, from about 45 kDa to about 50 kDa, from about 60 kDa to about 100 kDa, or from about 80 kDa to about 144 kDa, wherein the silk fibroin-based protein fragments have a polydispersity ranging from 1 to about 5.0.

In an embodiment, this disclosure provides a thermoresponsive hair care composition comprising (i) silk fibroin-based protein fragments that are substantially devoid of sericin at weight percent ranging from about 0.0001 wt. % to about 10.0 wt. % by the total weight of the hair care composition, (ii) at least one hair care active agent, and (iii) a dermatologically acceptable carrier, wherein the silk fibroin-based protein fragments have a weight average molecular weight ranging from about 1 kDa to about 5 kDa, from about 5 kDa to about 10 kDa, from about 6 kDa to about 17 kDa, from about 10 kDa to about 15 kDa, from about 15 kDa to about 20 kDa, from about 17 kDa to about 39 kDa, from about 20 kDa to about 25 kDa, from about 25 kDa to about 30 kDa, from about 30 kDa to about 35 kDa, from about 35 kDa to about 40 kDa, from about 39 kDa to about 80 kDa, from about 40 kDa to about 45 kDa, from about 45 kDa to about 50 kDa, from about 60 kDa to about 100 kDa, or from about 80 kDa to about 144 kDa, wherein the silk fibroin-based protein fragments have a polydispersity ranging from 1 to about 5.0.

In some embodiments, the hair care composition comprising the silk fibroin-based protein fragments at a weight percent ranging from about 0.01 wt. % to about 6.0 wt. % by the total weight of the hair care composition. In some embodiments, the hair care composition comprising the silk fibroin-based protein fragments at a weight percent ranging from about 3.0 wt. % to about 6.0 wt. % by the total weight of the hair care composition. In some embodiments, the hair care composition comprising the silk fibroin-based protein fragments at a weight percent ranging from about 1.0 wt. % to about 3.0 wt. % by the total weight of the hair care composition. In some embodiments, the hair care composition comprising the silk fibroin-based protein fragments at a weight percent of about 5.0 wt. % by the total weight of the hair care composition.

In some embodiments, the silk fibroin-based protein fragments have a weight average molecular weight ranging from about 5 kDa to about 18 kDa. In some embodiments, the silk fibroin-based protein fragments have a weight average molecular weight ranging from about 17 kDa to about 39 kDa. In some embodiments, the silk fibroin-based protein fragments have a weight average molecular weight ranging from about 39 kDa to about 80 kDa (hair styling product).

In some embodiments, the dermatologically acceptable carrier comprising an oil-in-water emulsion or a water-in-oil emulsion. In some embodiments, the dermatologically acceptable carrier comprising an oil phase and an aqueous phase. In some embodiments, the dermatologically acceptable carrier comprising emulsifying system.

In some embodiments, the hair care active agent is selected from the group consisting of a hair conditioning agent selected from hydrocarbon oil, fatty acid ester, cationic quaternary ammonium salt, and combination thereof. In some embodiments, the hair care active agent is a detersive detergent. In some embodiments, the hair care active agent comprises a hair styling polymer.

In some embodiments, the hair care composition is in a form selected from the group consisting of aqueous solution, ethanolic solution, oil, gel, emulsion, suspension, mousses, liquid crystal, solid, gels, lotions, creams, aerosol sprays, paste, foam and tonics.

In some embodiments, the hair care composition is a hair care product selected from the group consisting of pre-shampoo products, shampoo products, hair rinses, hair conditioners, hair treatments, setting lotions, blow-styling lotions, hair sprays, hair-styling foams, hair-styling gels, hair oil, hair liquids, hair tonics, hair creams, hair cosmetics and permanent waving agents.

In some embodiments, the hair care composition is transparent, or translucent.

In some embodiments, the hair care composition is for a rinse-off use, application, or product. In some embodiments, the hair care composition is for a leave-on use, application, or product.

In some embodiments, the disclosure provides a hair wax product including a hair care composition including silk fibroin fragments as described herein, and a dermatologically acceptable carrier as described herein. In some embodiments, this disclosure provides a hair care composition capable of foaming comprising SPF, e.g., without limitation silk fibroin protein fragments as disclosed herein, and less than 20.0 wt. % by the total weight of the hair care composition of a surfactant system containing (a) a sulfate-based surfactant, (b) a silicon-based surfactant, (c) synthetic surfactant.

In some embodiments, this disclosure provides a hair care composition comprising silk fragments, capable of coating hair cuticles and managing hair frizz.

In some embodiments, this disclosure provides a hair care composition capable of increasing hair sheen comprising SPF, e.g., without limitation silk fibroin protein fragments as disclosed herein.

In some embodiments, this disclosure provides a hair care composition capable of enhancing hair color comprising SPF, e.g., without limitation silk fibroin protein fragments as disclosed herein.

In some embodiments, this disclosure provides functionalized silk fibroin-based fragments comprise of a compound selected from Formulae (1)-(8):

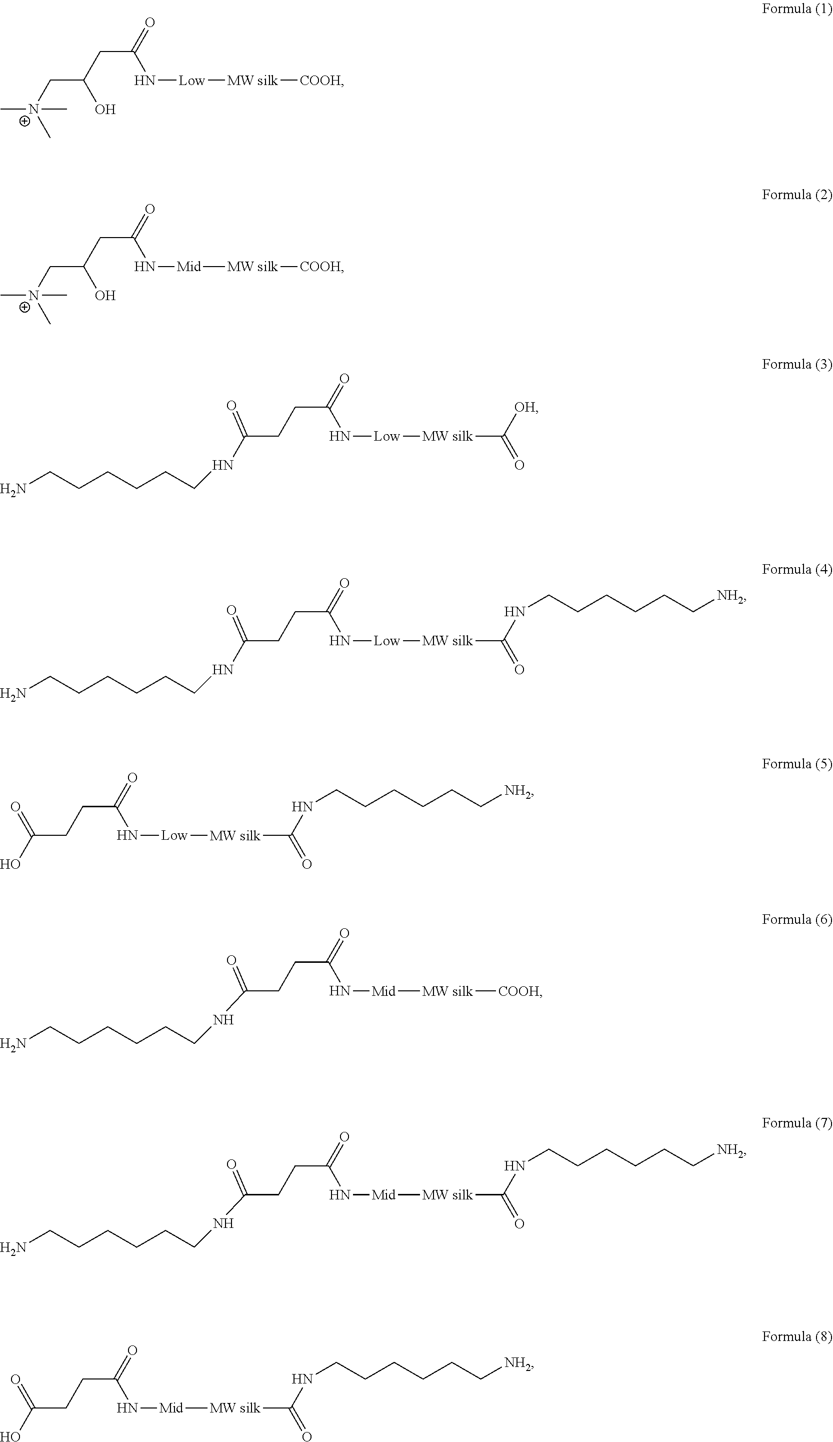

wherein, the Low-MW silk has a weight average molecular weight ranging from about 5 kDa to about 30 kDa, and a polydispersity ranging from 1.0 to about 5.0,

and the Mid-MW silk has a weight average molecular weight ranging from about 25 kDa to about 60 kDa, and a polydispersity ranging from 1.0 to about 5.0.

In some embodiments, the functionalized silk comprises a compound of Formula (1)

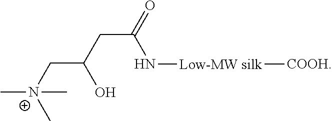

In some embodiments, the Low-MW silk has a weight average molecular weight ranging from about 5 kDa to about 30 kDa and a polydispersity ranging from about 1.5 to about 3.0. In some embodiments, the Low-MW silk has a weight average molecular weight ranging from about 5 kDa to about 17 kDa and a polydispersity ranging from about 1.5 to about 3.0.

In some embodiments, the functionalized silk comprises a compound of Formula (2)

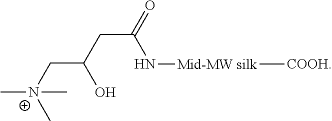

In some embodiments, the Mid-MW silk has a weight average molecular weight ranging from about 25 kDa to 60 kDa, and a polydispersity ranging from about 1.5 to about 3.0. In some embodiments, the Mid-MW silk has a weight average molecular weight ranging from about 39 kDa to about 54 kDa, and a polydispersity ranging from about 1.5 to about 3.0.

In some embodiments, the functionalized silk comprises a compound of Formula (3)

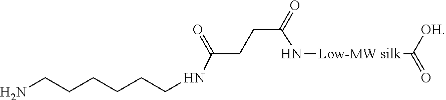

In some embodiments, the Low-MW silk has a weight average molecular weight ranging from about 5 kDa to about 30 kDa and a polydispersity ranging from about 1.5 to about 3.0. In some embodiments, the Low-MW silk has a weight average molecular weight ranging from about 5 kDa to about 17 kDa and a polydispersity ranging from about 1.5 to about 3.0.

In some embodiments, the functionalized silk comprises a compound of Formula (4)

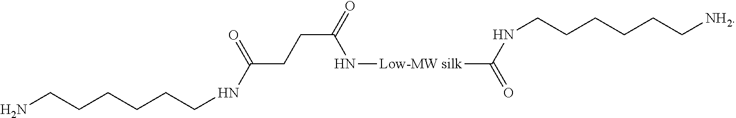

In some embodiments, the Low-MW silk has a weight average molecular weight ranging from about 5 kDa to about 30 kDa and a polydispersity ranging from about 1.5 to about 3.0. In some embodiments, the Low-MW silk has a weight average molecular weight ranging from about 5 kDa to about 17 kDa and a polydispersity ranging from about 1.5 to about 3.0.

In some embodiments, the functionalized silk comprises a compound of Formula (5)

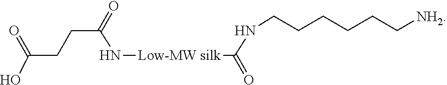

In some embodiments, the Low-MW silk has a weight average molecular weight ranging from about 5 kDa to about 30 kDa and a polydispersity ranging from about 1.5 to about 3.0. In some embodiments, the Low-MW silk has a weight average molecular weight ranging from about 5 kDa to about 17 kDa and a polydispersity ranging from about 1.5 to about 3.0.

In some embodiments, the functionalized silk comprises a compound of Formula (6)

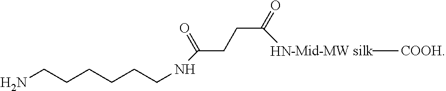

In some embodiments, the Mid-MW silk has a weight average molecular weight ranging from about 25 kDa to 60 kDa, and a polydispersity ranging from about 1.5 to about 3.0. In some embodiments, the Mid-MW silk has a weight average molecular weight ranging from about 39 kDa to about 54 kDa, and a polydispersity ranging from about 1.5 to about 3.0.

In some embodiments, the functionalized silk comprises a compound of Formula (7)

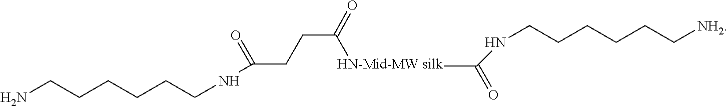

In some embodiments, the Mid-MW silk has a weight average molecular weight ranging from about 25 kDa to 60 kDa, and a polydispersity ranging from about 1.5 to about 3.0. In some embodiments, the Mid-MW silk has a weight average molecular weight ranging from about 39 kDa to about 54 kDa, and a polydispersity ranging from about 1.5 to about 3.0.

In some embodiments, the functionalized silk comprises a compound of Formula (8)

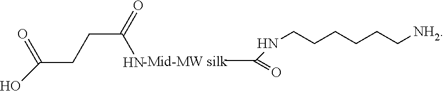

In some embodiments, the Mid-MW silk has a weight average molecular weight ranging from about 25 kDa to 60 kDa, and a polydispersity ranging from about 1.5 to about 3.0. In some embodiments, the Mid-MW silk has a weight average molecular weight ranging from about 39 kDa to about 54 kDa, and a polydispersity ranging from about 1.5 to about 3.0.

In some embodiments, this disclosure provides a hair care composition comprising the functionalized silk selected from Fomulae (1)-(8). In some embodiments, the hair care composition is formulated as a hair care product selected from pre-shampoo products, shampoo products, hair rinses, hair conditioners, hair treatments, setting lotions, blow-styling lotions, hair sprays, hair-styling foams, hair-styling gels, hair oil, hair liquids, hair tonics, hair creams, hair cosmetics, or permanent waving agents

### BRIEF DESCRIPTION OF THE DRAWINGS

FIGS. 1A-1C illustrate silk solutions as hair styling resin for straightening and curling hairs, provide style and curl retention. FIG. 1A: untreated brown tress; FIG. 1B: Medium molecular weight Silk Fibroin Solution (6 wt. %, 0.8 mL) was applied to an untreated brown tress; the tress was combed 10 times with a fine-tooth comb, and blow-dried on high heat for 120 seconds. A straightened style is achieved; FIG. 1C: Medium molecular weight Silk Fibroin Solution (6 wt %, 0.8 mL) was applied to a brown tress, the tress was combed 10 times with a fine-tooth comb and blow-dried on high heat for 120 seconds while wrapped around a conical tube (30 mm diameter). A curled style is achieved; typical resin concentration in high-hold styling products: 3-6 wt %; typical resin concentration in relaxed-hold styling products: 1-3 wt %.

FIGS. 2A-2D illustrate silk solution as a surfactant/shampoo active; silk provides a lather when massaged into hair, washes out well, and provides a clean, clarifying feeling during application. FIG. 2A: untreated brown tress; FIG. 2B water (0.3 mL) was applied to a brown tress to dampen it, and then Tween 20 (5 wt %, 0.6 mL) was applied to the tress; the tress was combed 10 times with a fine-tooth comb and then blow-dried on high heat for 120 seconds; FIG. 2C: water (0.3 mL) was applied to a brown tress to dampen it, and then medium molecular weight Silk Fibroin Solution (5% solids, 0.6 mL) was applied to the tress; the tress was combed 10 times with a fine-tooth comb and then blow-dried on high heat for 120 seconds; FIG. 2D: water (0.3 mL) was applied to a brown tress to dampen it, and low molecular weight Silk Fibroin Solution (5% solids, 0.6 mL) was applied to the tress; the tress was combed 10 times with a fine-tooth comb and then blow-dried on high heat for 120 seconds. All surfactants tested provided some lather while washing. Low molecular weight Silk Fibroin Solution felt especially clarifying during massage, indicative of good surfactant performance. All samples blow-dried well, with no discernible residue left behind.

FIGS. 3A-B show the damaged hair (FIG. 3A, untreated with silk) compared to silk treated hair (FIG. 3B).

FIGS. 4A-J show the pictures of the tresses used for visual ratings of nourishing parameters. FIG. 4A shows an untreated curly hair tress. The FIGS. 4B-J show the impacts of curl definition, frizz and smoothness on the curly hair tresses in pairs of two treated with solutions T001 to T009. The hair tresses in FIG. 4B were treated with T001 solution (water). The hair tresses in FIG. 4C were treated with T002 solution (hydrolyzed wheat protein (2%)). The hair tresses in FIG. 4D were treated with T003 solution (Low-MW silk (2%)). The hair tresses in FIG. 4E were treated with T004 solution (Mid-MW silk (2%)). The hair tress in FIG. 4F were treated with T005 solution (Low Permacharge (2%)). The hair tress in FIG. 4G were treated with T006 solution (Mid-098-02-02 (2%)). The hair tress in FIG. 4H were treated with T007 solution (Mid 098-08-02 (2%)). The hair tress in FIG. 4I were treated with T008 solution (Hexamine (2%)). The hair tress in FIG. 4J were treated with T009 solution (New Silicone (2%)).

FIG. 5 illustrates a summary graph of the visual ratings for each nourishing parameter. The performance of all of the analyzed silk (Low, Mid, Low-098-02-01, Mid-098-02-02, Mid 098-08-02, Hexamine, and New Silicone) are compared against the performance of the industry standard (hydrolyzed wheat protein).

FIGS. 6A-D illustrate treated hair on a mannequin head demonstrating curl retention capability of silk. FIG. 6A shows the curls of water treated hair versus FIG. 6C shows that the curls fell out after finger combed. FIG. 6B shows the tight and high curls of hair treated with Low Silk versus FIG. 6D shows that the curls retained after finger combed.

FIGS. 7A-B illustrate the treated hair on a mannequin head demonstrating the volume retention capability of silk. The FIG. 7A shows the initial visual inspection of the hair volume versus 24 hours later shown in FIG. 7B. The silk coated side retained its volume and the water treated side has significantly fallen.

FIGS. 8A-D illustrate hair samples treated with solutions 002-A (FIG. 8A), 002-B (FIG. 8B), 002-C (FIG. 8C) and 002-D (FIG. 8D). Hair sample in FIG. 8C, treated with a solution containing low molecular weight silk, retained the tightest curl among the four samples tested.

FIGS. 9A-D illustrate the data analysis by PEAKS software for the mass spectrum obtained for functionalized silk samples: 077-027-1 (FIG. 9A), 077-024-2 (FIG. 9B), 077-028-2 (FIG. 9C) and 077-030-1 (FIG. 9D).

FIGS. 10A-B show the electrophoresis gel for silk fibroin-based protein fragments (FIG. 10A), and functionalized silk fibroin-based protein fragments samples 077-024-2 (Lane 3), 077-027-1 (Lane 4), 077-027-2 (Lane 5), 077-028-2 (Lane 6), and 077-030-1 (Lane 7) (FIG. 10B). Lane 1 of FIG. 10B shows BioRad IEF Standards of molecular weight bands. Lane 2 of FIG. 10B shows IEF Sample buffer. Lane 8 of FIG. 10B shows MC-1. Lane 9 of FIG. 10B shows 5700-SP. Lane 10 of FIG. 10B shows DBr-7. Lane 11 of FIG. 10B shows Ser-1. FIG. 10A shows the electrophoresis gel from several Activated Silks™, and FIG. 10B shows the electrophoresis gel for chemically modified Activated Silks™.

FIG. 11 shows the SEC-RI chromatograms of two modified Mid-MW silks (098-29-02, and 098-30-02) compared to an unmodified Mid-MW weight silk. FIG. 12A-B show the m/z and ms2 fragmentation patterns for two subunits: heavy chain (FIG. 12A), light chain (FIG. 12B) in the mass spectra for the modified Low-MW silk (077-027-1).

FIG. 13A-C show the m/z and ms2 fragmentation patterns for all three subunits: heavy chain (FIG. 13A), light chain (FIG. 13B), and fibrohexamerin (FIG. 13C) in the mass spectra for the modified Low-MW silk (077-024-2).

FIG. 14 shows the m/z and ms2 fragmentation patterns light chain in the mass spectrum for the modified Low-MW silk (077-028-2).

FIG. 15 shows the m/z and ms2 fragmentation patterns light chain in the mass spectrum for the modified Low-MW silk (077-030-1).

FIG. 16 is a flow chart showing various embodiments for producing pure silk fibroin protein fragments (SPFs) of the present disclosure.

FIG. 17 is a flow chart showing various parameters that can be modified during the process of producing a silk protein fragment solution of the present disclosure during the extraction and the dissolution steps.

### DETAILED DESCRIPTION

Most conditioning agents in the hair care products are synthetic polymers used to improve the feel of hair. Common ingredients include polyethylene glycols (PEGs), propylene glycol, siloxanes, and polyquaterniums. PEGs and propylene glycol are used as emollients and lubricants for hair but are derived from petroleum which can contain harmful impurities such as ethylene oxide and 1,4-dioxane. Silioxanes, such as cyclomethicone and dimethicone are used for improving feel of the formulation and hair. But silioxanes are derived from petroleum, are known to bioaccumulate, and have limited biodegradability. Polyquaterniums are cationic polymers used in a variety of hair care products to impart various use effects such as improving hair feel, water repellency, hair hold and texture. However, polyquaterniums can be derived from petroleum.

Traditional semi-permanent and permanent hair shaping technologies are typically based on harsh chemistries which both damage the hair and pose a threat to the health of consumers and hair care professionals. Hair curling (“perms”) involves the reduction of disulfide bonds of the keratin in hair, most often by thioglycolates, which are toxic and carcinogenic. After re-shaping with curlers and heat, the disulfide bonds are re-formed by oxidation with reagents that are often irritating, such as hydrogen peroxide. Relaxers often use the same chemistry, and/or or use caustic chemicals such as sodium hydroxide in high concentrations. Straightening and/or smoothing treatments such as the Brazilian Blowout also involve reduction of disulfide bonds with harsh chemistries. In the re-shaping step of Brazilian Blowout, keratin is bound to the hair by a crosslinking mechanism with formaldehyde, a known carcinogen and respiratory irritant. “Formaldehyde-free” variations include methylene glycol in the formulation, which becomes dehydrating upon heating to form formaldehyde as the active component. There is a need in the industry for a safe, effective replacement for these chemistries.

Silk proteins have found applications in hair care products. Silk can be used to replace the conventional hair conditioning ingredients with a sustainable, safe, and biodegradable alternative that still improves the look and feel of hair. In these disclosures, silk solutions were used because of the low solubility of the raw SPF, e.g., without limitation silk fibroin proteins. While providing some beneficial coating effect, the silk protein peptides are not as effective as the intact proteins. The colloidal silk fibroin protein was reported as an additive in hair care products (DE 3139438). However, the colloidal silk fibroin protein is not as effective in film-forming and coating for hair treatment as a soluble silk protein. The natural or recombinant spider silk proteins were reported as active ingredient for incorporation into cosmetic and dermatological compositions such as hair care, skin care, make-up, and sunscreen products (U.S. Pat. No. 6,280,747). However, the spider silk is not water soluble. Therefore, the beneficial effects of the self-assembly and coating properties of the spider silk proteins are not realized.

Methods of making silk fibroin or silk fibroin fragments are known and are described for example in U.S. Pat. Nos. 9,187,538, 9,511,012, 9,517,191, 9,522,107, 9,522,108, 9,545,369, and 10,166,177, all of which are incorporated herein in their entireties. Methods of using silk fibroin or silk fibroin fragments in coating applications, including coating applications of animal hair, are known and are described for example in U.S. Patent Application Publications Nos. 20160222579, and 20160281294.

SPFs, e.g., without limitation silk fibroin protein fragments have useful applications in varieties of hair care products. In an embodiment, this disclosure provides a hair care composition comprises (i) silk fibroin-based protein fragments that are substantially devoid of sericin at weight percent ranging from about 0.0001 wt. % to about 10.0 wt. % by the total weight of the hair care composition, (ii) at least one hair care active agent, and (iii) a dermatologically acceptable carrier, wherein the silk fibroin-based protein fragments have a weight average molecular weight ranging from about 5 kDa to about 80 kDa, wherein the silk fibroin-based protein fragments have a polydispersity of between about 1.5 and about 3.0.

In some embodiments, the hair care composition is in a form selected from the group consisting of an aqueous solution, an ethanolic solution, an oil, a gel, an emulsion, a suspension, a mousses, a liquid crystal, a solid, a lotion, a cream, an aerosol spray, a paste, a foam, and a tonic.

In some embodiments, the hair care composition is a hair care product selected from the group consisting of pre-shampoo products, shampoo products, hair rinses, hair conditioners, hair treatments, setting lotions, blow-styling lotions, hair sprays, hair-styling foams, hair-styling gels, hair oil, hair liquids, hair tonics, hair creams, hair coloring cosmetics, and permanent waving agents. In some embodiments, the hair care composition is transparent, or translucent. In some embodiments, the hair care composition is for a leave in use.

### Definitions

As used in the preceding sections and throughout the rest of this specification, unless defined otherwise, all technical and scientific terms used herein have the same meaning as is commonly understood by one skilled in the art to which this disclosure belongs. All patents and publications referred to herein are incorporated by reference in their entireties.

All percentages, parts and ratios are based upon the total weight of the hair care compositions of the present disclosure, unless otherwise specified. All such weights as they pertain to listed ingredients are based on the active level and, therefore, do not include solvents or by-products that may be included in commercially available materials, unless otherwise specified. The term “weight percent” may be denoted as “wt. %” or % w/w herein.

As used herein, the term “a”, “an”, or “the” generally is construed to cover both the singular and the plural forms.

The term “about” as used herein, generally refers to a particular numeric value that include variation and an acceptable error range as determined by one of ordinary skill in the art, which will depend in part on how the numeric value is measured or determined, i.e., the limitations of the measurement system. For example, “about” can mean zero variation, and a range of ±20%, ±10%, or ±5% of a given numeric value.

As used herein, “average weight average molecular weight” refers to an average of two or more values of weight average molecular weight of silk fibroin or fragments thereof of the same compositions, the two or more values determined by two or more separate experimental readings.

As used herein, the term “hair” refers to hair on the human head and scalp. “Hair shaft” means an individual hair, and may be used interchangeably with the term “hair”.

As used herein, the term “hair care product” involves all toiletry products to be applied to the hair. Examples thereof include pre-shampoo products, shampoo products, hair rinses, hair conditioners, hair treatments, setting lotions, blow-styling lotions, hair sprays, hair-styling foams, hair-styling gels, hair liquids, hair tonics, hair creams, permanent waving agents, etc. Further, the hair care product of the present disclosure may be processed into various forms (aqueous solution, ethanolic solution, emulsion, suspension, gel, liquid crystal, solid, aerosol, etc.) depending on the purpose of the use.

As used herein, the term “dermatologically acceptable carrier” means a carrier suitable for use in contact with mammalian keratinous tissue without causing any adverse effects such as undue toxicity, incompatibility, instability, allergic response, for example. A dermatologically acceptable carrier may include, without limitations, water, liquid or solid emollients, humectants, solvents, and the like.

As used herein, the term “hydrophilic-lipophilic balance” (HLB) of a surfactant is a measure of the degree to which it is hydrophilic or hydrophobic, as determined by calculating values for the different regions of the molecule, as described by Griffin's method HLB=20*Mₕ/M, where Mₕ is the molecular mass of the hydrophilic portion of the surfactant, and M is the molecular mass of the entire surfactant molecule, giving a result on a scale of 0 to 20. A HLB value of 0 corresponds to a completely lipophilic molecule, and a value of 20 corresponds to a completely hydrophilic molecule. The HLB value can be used to predict the surfactant properties of a molecule: HLB<10: Lipid-soluble (water-insoluble), HLB>10: Water-soluble (lipid-insoluble), HLB=1-3: anti-foaming agent, 3-6: W/O (water-in-oil) emulsifier, 7-9: wetting and spreading agent, 8-16: O/W (oil-in-water) emulsifier, 13-16: detergent. 16-18: solubilizer or hydrotrope.

As used herein, the terms “peptide” or “protein” refers to a chain of amino acids that are held together by peptide bonds (also called amide bonds). The basic distinguishing factors for proteins and peptides are size and structure. Peptides are smaller than proteins. Traditionally, peptides are defined as molecules that consist of between 2 and 50 amino acids, whereas proteins are made up of 50 or more amino acids. In addition, peptides tend to be less well defined in structure than proteins, which can adopt complex conformations known as secondary, tertiary, and quaternary structures.

As used herein, the term polymer “polydispersity (PD)” is generally used as a measure of the broadness of a molecular weight distribution of a polymer, and is defined by the formula polydispersity

$$P D = \frac{M w}{M n} .$$

As used herein, the term “substantially homogeneous” may refer to silk fibroin-based protein fragments that are distributed in a normal distribution about an identified molecular weight. As used herein, the term “substantially homogeneous” may refer to an even distribution of a component or an additive, for example, silk fibroin fragments, dermatologically acceptable carrier, etc., throughout a composition of the present disclosure.

SPF Definitions and Properties

As used herein, “silk protein fragments” (SPF) include one or more of: “silk fibroin fragments” as defined herein; “recombinant silk fragments” as defined herein; “spider silk fragments” as defined herein; “silk fibroin-like protein fragments” as defined herein; and/or “chemically modified silk fragments” as defined herein. SPF may have any molecular weight values or ranges described herein, and any polydispersity values or ranges described herein. As used herein, in some embodiments the term “silk protein fragment” also refers to a silk protein that comprises or consists of at least two identical repetitive units which each independently selected from naturally-occurring silk polypeptides or of variations thereof, amino acid sequences of naturally-occurring silk polypeptides, or of combinations of both.

SPF Molecular Weight and Polydispersity

In an embodiment, a composition of the present disclosure includes SPF having an average weight average molecular weight ranging from 6 kDa to 17 kDa. In an embodiment, a composition of the present disclosure includes SPF having a weight average molecular weight ranging from 17 kDa to 39 kDa. In an embodiment, a composition of the present disclosure includes SPF having an average weight average molecular weight ranging from 39 kDa to 80 kDa. In an embodiment, a composition of the present disclosure includes SPF having an average weight average molecular weight ranging from about 1 to about 5 kDa. In an embodiment, a composition of the present disclosure includes SPF having an average weight average molecular weight ranging from about 5 to about 10 kDa. In an embodiment, a composition of the present disclosure includes SPF having an average weight average molecular weight ranging from about 10 to about 15 kDa. In an embodiment, a composition of the present disclosure includes SPF having an average weight average molecular weight ranging from about 15 to about 20 kDa. In an embodiment, a composition of the present disclosure includes SPF having an average weight average molecular weight ranging from about 20 to about 25 kDa. In an embodiment, a composition of the present disclosure includes SPF having an average weight average molecular weight ranging from about 25 to about 30 kDa. In an embodiment, a composition of the present disclosure includes SPF having an average weight average molecular weight ranging from about 30 to about 35 kDa. In an embodiment, a composition of the present disclosure includes SPF having an average weight average molecular weight ranging from about 35 to about 40 kDa. In an embodiment, a composition of the present disclosure includes SPF having an average weight average molecular weight ranging from about 40 to about 45 kDa. In an embodiment, a composition of the present disclosure includes SPF having an average weight average molecular weight ranging from about 45 to about 50 kDa. In an embodiment, a composition of the present disclosure includes SPF having an average weight average molecular weight ranging from about 50 to about 55 kDa. In an embodiment, a composition of the present disclosure includes SPF having an average weight average molecular weight ranging from about 55 to about 60 kDa. In an embodiment, a composition of the present disclosure includes SPF having an average weight average molecular weight ranging from about 60 to about 65 kDa. In an embodiment, a composition of the present disclosure includes SPF having an average weight average molecular weight ranging from about 65 to about 70 kDa. In an embodiment, a composition of the present disclosure includes SPF having an average weight average molecular weight ranging from about 70 to about 75 kDa. In an embodiment, a composition of the present disclosure includes SPF having an average weight average molecular weight ranging from about 75 to about 80 kDa. In an embodiment, a composition of the present disclosure includes SPF having an average weight average molecular weight ranging from about 80 to about 85 kDa. In an embodiment, a composition of the present disclosure includes SPF having an average weight average molecular weight ranging from about 85 to about 90 kDa. In an embodiment, a composition of the present disclosure includes SPF having an average weight average molecular weight ranging from about 90 to about 95 kDa. In an embodiment, a composition of the present disclosure includes SPF having an average weight average molecular weight ranging from about 95 to about 100 kDa. In an embodiment, a composition of the present disclosure includes SPF having an average weight average molecular weight ranging from about 100 to about 105 kDa. In an embodiment, a composition of the present disclosure includes SPF having an average weight average molecular weight ranging from about 105 to about 110 kDa. In an embodiment, a composition of the present disclosure includes SPF having an average weight average molecular weight ranging from about 110 to about 115 kDa. In an embodiment, a composition of the present disclosure includes SPF having an average weight average molecular weight ranging from about 115 to about 120 kDa. In an embodiment, a composition of the present disclosure includes SPF having an average weight average molecular weight ranging from about 120 to about 125 kDa. In an embodiment, a composition of the present disclosure includes SPF having an average weight average molecular weight ranging from about 125 to about 130 kDa. In an embodiment, a composition of the present disclosure includes SPF having an average weight average molecular weight ranging from about 130 to about 135 kDa. In an embodiment, a composition of the present disclosure includes SPF having an average weight average molecular weight ranging from about 135 to about 140 kDa. In an embodiment, a composition of the present disclosure includes SPF having an average weight average molecular weight ranging from about 140 to about 145 kDa. In an embodiment, a composition of the present disclosure includes SPF having an average weight average molecular weight ranging from about 145 to about 150 kDa. In an embodiment, a composition of the present disclosure includes SPF having an average weight average molecular weight ranging from about 150 to about 155 kDa. In an embodiment, a composition of the present disclosure includes SPF having an average weight average molecular weight ranging from about 155 to about 160 kDa. In an embodiment, a composition of the present disclosure includes SPF having an average weight average molecular weight ranging from about 160 to about 165 kDa. I In an embodiment, a composition of the present disclosure includes SPF having an average weight average molecular weight ranging from about 165 to about 170 kDa. In an embodiment, a composition of the present disclosure includes SPF having an average weight average molecular weight ranging from about 170 to about 175 kDa. In an embodiment, a composition of the present disclosure includes SPF having an average weight average molecular weight ranging from about 175 to about 180 kDa. In an embodiment, a composition of the present disclosure includes SPF having an average weight average molecular weight ranging from about 180 to about 185 kDa. In an embodiment, a composition of the present disclosure includes SPF having an average weight average molecular weight ranging from about 185 to about 190 kDa. In an embodiment, a composition of the present disclosure includes SPF having an average weight average molecular weight ranging from about 190 to about 195 kDa. In an embodiment, a composition of the present disclosure includes SPF having an average weight average molecular weight ranging from about 195 to about 200 kDa. In an embodiment, a composition of the present disclosure includes SPF having an average weight average molecular weight ranging from about 200 to about 205 kDa. In an embodiment, a composition of the present disclosure includes SPF having an average weight average molecular weight ranging from about 205 to about 210 kDa. In an embodiment, a composition of the present disclosure includes SPF having an average weight average molecular weight ranging from about 210 to about 215 kDa. In an embodiment, a composition of the present disclosure includes SPF having an average weight average molecular weight ranging from about 215 to about 220 kDa. In an embodiment, a composition of the present disclosure includes SPF having an average weight average molecular weight ranging from about 220 to about 225 kDa. In an embodiment, a composition of the present disclosure includes SPF having an average weight average molecular weight ranging from about 225 to about 230 kDa. In an embodiment, a composition of the present disclosure includes SPF having an average weight average molecular weight ranging from about 230 to about 235 kDa. In an embodiment, a composition of the present disclosure includes SPF having an average weight average molecular weight ranging from about 235 to about 240 kDa. In an embodiment, a composition of the present disclosure includes SPF having an average weight average molecular weight ranging from about 240 to about 245 kDa. In an embodiment, a composition of the present disclosure includes SPF having an average weight average molecular weight ranging from about 245 to about 250 kDa. In an embodiment, a composition of the present disclosure includes SPF having an average weight average molecular weight ranging from about 250 to about 255 kDa. In an embodiment, a composition of the present disclosure includes SPF having an average weight average molecular weight ranging from about 255 to about 260 kDa. In an embodiment, a composition of the present disclosure includes SPF having an average weight average molecular weight ranging from about 260 to about 265 kDa. In an embodiment, a composition of the present disclosure includes SPF having an average weight average molecular weight ranging from about 265 to about 270 kDa. In an embodiment, a composition of the present disclosure includes SPF having an average weight average molecular weight ranging from about 270 to about 275 kDa. In an embodiment, a composition of the present disclosure includes SPF having an average weight average molecular weight ranging from about 275 to about 280 kDa. In an embodiment, a composition of the present disclosure includes SPF having an average weight average molecular weight ranging from about 280 to about 285 kDa. In an embodiment, a composition of the present disclosure includes SPF having an average weight average molecular weight ranging from about 285 to about 290 kDa. In an embodiment, a composition of the present disclosure includes SPF having an average weight average molecular weight ranging from about 290 to about 295 kDa. In an embodiment, a composition of the present disclosure includes SPF having an average weight average molecular weight ranging from about 295 to about 300 kDa. In an embodiment, a composition of the present disclosure includes SPF having an average weight average molecular weight ranging from about 300 to about 305 kDa. In an embodiment, a composition of the present disclosure includes SPF having an average weight average molecular weight ranging from about 305 to about 310 kDa. In an embodiment, a composition of the present disclosure includes SPF having an average weight average molecular weight ranging from about 310 to about 315 kDa. In an embodiment, a composition of the present disclosure includes SPF having an average weight average molecular weight ranging from about 315 to about 320 kDa. In an embodiment, a composition of the present disclosure includes SPF having an average weight average molecular weight ranging from about 320 to about 325 kDa. In an embodiment, a composition of the present disclosure includes SPF having an average weight average molecular weight ranging from about 325 to about 330 kDa. In an embodiment, a composition of the present disclosure includes SPF having an average weight average molecular weight ranging from about 330 to about 335 kDa. In an embodiment, a composition of the present disclosure includes SPF having an average weight average molecular weight ranging from about 335 to about 340 kDa. In an embodiment, a composition of the present disclosure includes SPF having an average weight average molecular weight ranging from about 340 to about 345 kDa. In an embodiment, a composition of the present disclosure includes SPF having an average weight average molecular weight ranging from about 345 to about 350 kDa.

In some embodiments, compositions of the present disclosure include SPF compositions selected from compositions #1001 to #2450, having weight average molecular weights selected from about 1 kDa to about 145 kDa, and a polydispersity range selected from between 1 and about 9 (including, without limitation, a polydispersity of 1), between 1 and about 1.5 (including, without limitation, a polydispersity of 1), between about 1.5 and about 2, between about 1.5 and about 3, between about 2 and about 2.5, between about 2.5 and about 3, between about 3 and about 3.5, between about 3.5 and about 4, between about 4 and about 4.5, and between about 4.5 and about 5:

| | | PDI (about) | | | | | | | | | |
|---|---|---|---|---|---|---|---|---|---|---|---|
| MW (about) | | 1-5 | 1-1.5 | 1.5-2 | 1.5-3 | 2-2.5 | 2.5-3 | 3-3.5 | 3.5-4 | 4-4.5 | 4.5-5 |
| 1 | kDa | 1001 | 1002 | 1003 | 1004 | 1005 | 1006 | 1007 | 1008 | 1009 | 1010 |
| 2 | kDa | 1011 | 1012 | 1013 | 1014 | 1015 | 1016 | 1017 | 1018 | 1019 | 1020 |
| 3 | kDa | 1021 | 1022 | 1023 | 1024 | 1025 | 1026 | 1027 | 1028 | 1029 | 1030 |
| 4 | kDa | 1031 | 1032 | 1033 | 1034 | 1035 | 1036 | 1037 | 1038 | 1039 | 1040 |
| 5 | kDa | 1041 | 1042 | 1043 | 1044 | 1045 | 1046 | 1047 | 1048 | 1049 | 1050 |
| 6 | kDa | 1051 | 1052 | 1053 | 1054 | 1055 | 1056 | 1057 | 1058 | 1059 | 1060 |
| 7 | kDa | 1061 | 1062 | 1063 | 1064 | 1065 | 1066 | 1067 | 1068 | 1069 | 1070 |
| 8 | kDa | 1071 | 1072 | 1073 | 1074 | 1075 | 1076 | 1077 | 1078 | 1079 | 1080 |
| 9 | kDa | 1081 | 1082 | 1083 | 1084 | 1085 | 1086 | 1087 | 1088 | 1089 | 1090 |
| 10 | kDa | 1091 | 1092 | 1093 | 1094 | 1095 | 1096 | 1097 | 1098 | 1099 | 1100 |
| 11 | kDa | 1101 | 1102 | 1103 | 1104 | 1105 | 1106 | 1107 | 1108 | 1109 | 1110 |
| 12 | kDa | 1111 | 1112 | 1113 | 1114 | 1115 | 1116 | 1117 | 1118 | 1119 | 1120 |
| 13 | kDa | 1121 | 1122 | 1123 | 1124 | 1125 | 1126 | 1127 | 1128 | 1129 | 1130 |
| 14 | kDa | 1131 | 1132 | 1133 | 1134 | 1135 | 1136 | 1137 | 1138 | 1139 | 1140 |
| 15 | kDa | 1141 | 1142 | 1143 | 1144 | 1145 | 1146 | 1147 | 1148 | 1149 | 1150 |
| 16 | kDa | 1151 | 1152 | 1153 | 1154 | 1155 | 1156 | 1157 | 1158 | 1159 | 1160 |
| 17 | kDa | 1161 | 1162 | 1163 | 1164 | 1165 | 1166 | 1167 | 1168 | 1169 | 1170 |
| 18 | kDa | 1171 | 1172 | 1173 | 1174 | 1175 | 1176 | 1177 | 1178 | 1179 | 1180 |
| 19 | kDa | 1181 | 1182 | 1183 | 1184 | 1185 | 1186 | 1187 | 1188 | 1189 | 1190 |
| 20 | kDa | 1191 | 1192 | 1193 | 1194 | 1195 | 1196 | 1197 | 1198 | 1199 | 1200 |
| 21 | kDa | 1201 | 1202 | 1203 | 1204 | 1205 | 1206 | 1207 | 1208 | 1209 | 1210 |
| 22 | kDa | 1211 | 1212 | 1213 | 1214 | 1215 | 1216 | 1217 | 1218 | 1219 | 1220 |
| 23 | kDa | 1221 | 1222 | 1223 | 1224 | 1225 | 1226 | 1227 | 1228 | 1229 | 1230 |
| 24 | kDa | 1231 | 1232 | 1233 | 1234 | 1235 | 1236 | 1237 | 1238 | 1239 | 1240 |
| 25 | kDa | 1241 | 1242 | 1243 | 1244 | 1245 | 1246 | 1247 | 1248 | 1249 | 1250 |
| 26 | kDa | 1251 | 1252 | 1253 | 1254 | 1255 | 1256 | 1257 | 1258 | 1259 | 1260 |
| 27 | kDa | 1261 | 1262 | 1263 | 1264 | 1265 | 1266 | 1267 | 1268 | 1269 | 1270 |
| 28 | kDa | 1271 | 1272 | 1273 | 1274 | 1275 | 1276 | 1277 | 1278 | 1279 | 1280 |
| 29 | kDa | 1281 | 1282 | 1283 | 1284 | 1285 | 1286 | 1287 | 1288 | 1289 | 1290 |
| 30 | kDa | 1291 | 1292 | 1293 | 1294 | 1295 | 1296 | 1297 | 1298 | 1299 | 1300 |
| 31 | kDa | 1301 | 1302 | 1303 | 1304 | 1305 | 1306 | 1307 | 1308 | 1309 | 1310 |
| 32 | kDa | 1311 | 1312 | 1313 | 1314 | 1315 | 1316 | 1317 | 1318 | 1319 | 1320 |
| 33 | kDa | 1321 | 1322 | 1323 | 1324 | 1325 | 1326 | 1327 | 1328 | 1329 | 1330 |
| 34 | kDa | 1331 | 1332 | 1333 | 1334 | 1335 | 1336 | 1337 | 1338 | 1339 | 1340 |
| 35 | kDa | 1341 | 1342 | 1343 | 1344 | 1345 | 1346 | 1347 | 1348 | 1349 | 1350 |
| 36 | kDa | 1351 | 1352 | 1353 | 1354 | 1355 | 1356 | 1357 | 1358 | 1359 | 1360 |
| 37 | kDa | 1361 | 1362 | 1363 | 1364 | 1365 | 1366 | 1367 | 1368 | 1369 | 1370 |
| 38 | kDa | 1371 | 1372 | 1373 | 1374 | 1375 | 1376 | 1377 | 1378 | 1379 | 1380 |
| 39 | kDa | 1381 | 1382 | 1383 | 1384 | 1385 | 1386 | 1387 | 1388 | 1389 | 1390 |
| 40 | kDa | 1391 | 1392 | 1393 | 1394 | 1395 | 1396 | 1397 | 1398 | 1399 | 1400 |
| 41 | kDa | 1401 | 1402 | 1403 | 1404 | 1405 | 1406 | 1407 | 1408 | 1409 | 1410 |
| 42 | kDa | 1411 | 1412 | 1413 | 1414 | 1415 | 1416 | 1417 | 1418 | 1419 | 1420 |
| 43 | kDa | 1421 | 1422 | 1423 | 1424 | 1425 | 1426 | 1427 | 1428 | 1429 | 1430 |
| 44 | kDa | 1431 | 1432 | 1433 | 1434 | 1435 | 1436 | 1437 | 1438 | 1439 | 1440 |
| 45 | kDa | 1441 | 1442 | 1443 | 1444 | 1445 | 1446 | 1447 | 1448 | 1449 | 1450 |
| 46 | kDa | 1451 | 1452 | 1453 | 1454 | 1455 | 1456 | 1457 | 1458 | 1459 | 1460 |
| 47 | kDa | 1461 | 1462 | 1463 | 1464 | 1465 | 1466 | 1467 | 1468 | 1469 | 1470 |
| 48 | kDa | 1471 | 1472 | 1473 | 1474 | 1475 | 1476 | 1477 | 1478 | 1479 | 1480 |
| 49 | kDa | 1481 | 1482 | 1483 | 1484 | 1485 | 1486 | 1487 | 1488 | 1489 | 1490 |
| 50 | kDa | 1491 | 1492 | 1493 | 1494 | 1495 | 1496 | 1497 | 1498 | 1499 | 1500 |
| 51 | kDa | 1501 | 1502 | 1503 | 1504 | 1505 | 1506 | 1507 | 1508 | 1509 | 1510 |
| 52 | kDa | 1511 | 1512 | 1513 | 1514 | 1515 | 1516 | 1517 | 1518 | 1519 | 1520 |
| 53 | kDa | 1521 | 1522 | 1523 | 1524 | 1525 | 1526 | 1527 | 1528 | 1529 | 1530 |
| 54 | kDa | 1531 | 1532 | 1533 | 1534 | 1535 | 1536 | 1537 | 1538 | 1539 | 1540 |
| 55 | kDa | 1541 | 1542 | 1543 | 1544 | 1545 | 1546 | 1547 | 1548 | 1549 | 1550 |
| 56 | kDa | 1551 | 1552 | 1553 | 1554 | 1555 | 1556 | 1557 | 1558 | 1559 | 1560 |
| 57 | kDa | 1561 | 1562 | 1563 | 1564 | 1565 | 1566 | 1567 | 1568 | 1569 | 1570 |
| 58 | kDa | 1571 | 1572 | 1573 | 1574 | 1575 | 1576 | 1577 | 1578 | 1579 | 1580 |
| 59 | kDa | 1581 | 1582 | 1583 | 1584 | 1585 | 1586 | 1587 | 1588 | 1589 | 1590 |
| 60 | kDa | 1591 | 1592 | 1593 | 1594 | 1595 | 1596 | 1597 | 1598 | 1599 | 1600 |
| 61 | kDa | 1601 | 1602 | 1603 | 1604 | 1605 | 1606 | 1607 | 1608 | 1609 | 1610 |
| 62 | kDa | 1611 | 1612 | 1613 | 1614 | 1615 | 1616 | 1617 | 1618 | 1619 | 1620 |
| 63 | kDa | 1621 | 1622 | 1623 | 1624 | 1625 | 1626 | 1627 | 1628 | 1629 | 1630 |
| 64 | kDa | 1631 | 1632 | 1633 | 1634 | 1635 | 1636 | 1637 | 1638 | 1639 | 1640 |
| 65 | kDa | 1641 | 1642 | 1643 | 1644 | 1645 | 1646 | 1647 | 1648 | 1649 | 1650 |
| 66 | kDa | 1651 | 1652 | 1653 | 1654 | 1655 | 1656 | 1657 | 1658 | 1659 | 1660 |
| 67 | kDa | 1661 | 1662 | 1663 | 1664 | 1665 | 1666 | 1667 | 1668 | 1669 | 1670 |
| 68 | kDa | 1671 | 1672 | 1673 | 1674 | 1675 | 1676 | 1677 | 1678 | 1679 | 1680 |
| 69 | kDa | 1681 | 1682 | 1683 | 1684 | 1685 | 1686 | 1687 | 1688 | 1689 | 1690 |
| 70 | kDa | 1691 | 1692 | 1693 | 1694 | 1695 | 1696 | 1697 | 1698 | 1699 | 1700 |
| 71 | kDa | 1701 | 1702 | 1703 | 1704 | 1705 | 1706 | 1707 | 1708 | 1709 | 1710 |
| 72 | kDa | 1711 | 1712 | 1713 | 1714 | 1715 | 1716 | 1717 | 1718 | 1719 | 1720 |
| 73 | kDa | 1721 | 1722 | 1723 | 1724 | 1725 | 1726 | 1727 | 1728 | 1729 | 1730 |
| 74 | kDa | 1731 | 1732 | 1733 | 1734 | 1735 | 1736 | 1737 | 1738 | 1739 | 1740 |
| 75 | kDa | 1741 | 1742 | 1743 | 1744 | 1745 | 1746 | 1747 | 1748 | 1749 | 1750 |
| 76 | kDa | 1751 | 1752 | 1753 | 1754 | 1755 | 1756 | 1757 | 1758 | 1759 | 1760 |
| 77 | kDa | 1761 | 1762 | 1763 | 1764 | 1765 | 1766 | 1767 | 1768 | 1769 | 1770 |
| 78 | kDa | 1771 | 1772 | 1773 | 1774 | 1775 | 1776 | 1777 | 1778 | 1779 | 1780 |
| 79 | kDa | 1781 | 1782 | 1783 | 1784 | 1785 | 1786 | 1787 | 1788 | 1789 | 1790 |
| 80 | kDa | 1791 | 1792 | 1793 | 1794 | 1795 | 1796 | 1797 | 1798 | 1799 | 1800 |
| 81 | kDa | 1801 | 1802 | 1803 | 1804 | 1805 | 1806 | 1807 | 1808 | 1809 | 1810 |
| 82 | kDa | 1811 | 1812 | 1813 | 1814 | 1815 | 1816 | 1817 | 1818 | 1819 | 1820 |
| 83 | kDa | 1821 | 1822 | 1823 | 1824 | 1825 | 1826 | 1827 | 1828 | 1829 | 1830 |
| 84 | kDa | 1831 | 1832 | 1833 | 1834 | 1835 | 1836 | 1837 | 1838 | 1839 | 1840 |
| 85 | kDa | 1841 | 1842 | 1843 | 1844 | 1845 | 1846 | 1847 | 1848 | 1849 | 1850 |
| 86 | kDa | 1851 | 1852 | 1853 | 1854 | 1855 | 1856 | 1857 | 1858 | 1859 | 1860 |
| 87 | kDa | 1861 | 1862 | 1863 | 1864 | 1865 | 1866 | 1867 | 1868 | 1869 | 1870 |
| 88 | kDa | 1871 | 1872 | 1873 | 1874 | 1875 | 1876 | 1877 | 1878 | 1879 | 1880 |
| 89 | kDa | 1881 | 1882 | 1883 | 1884 | 1885 | 1886 | 1887 | 1888 | 1889 | 1890 |
| 90 | kDa | 1891 | 1892 | 1893 | 1894 | 1895 | 1896 | 1897 | 1898 | 1899 | 1900 |
| 91 | kDa | 1901 | 1902 | 1903 | 1904 | 1905 | 1906 | 1907 | 1908 | 1909 | 1910 |
| 92 | kDa | 1911 | 1912 | 1913 | 1914 | 1915 | 1916 | 1917 | 1918 | 1919 | 1920 |
| 93 | kDa | 1921 | 1922 | 1923 | 1924 | 1925 | 1926 | 1927 | 1928 | 1929 | 1930 |
| 94 | kDa | 1931 | 1932 | 1933 | 1934 | 1935 | 1936 | 1937 | 1938 | 1939 | 1940 |
| 95 | kDa | 1941 | 1942 | 1943 | 1944 | 1945 | 1946 | 1947 | 1948 | 1949 | 1950 |
| 96 | kDa | 1951 | 1952 | 1953 | 1954 | 1955 | 1956 | 1957 | 1958 | 1959 | 1960 |
| 97 | kDa | 1961 | 1962 | 1963 | 1964 | 1965 | 1966 | 1967 | 1968 | 1969 | 1970 |
| 98 | kDa | 1971 | 1972 | 1973 | 1974 | 1975 | 1976 | 1977 | 1978 | 1979 | 1980 |
| 99 | kDa | 1981 | 1982 | 1983 | 1984 | 1985 | 1986 | 1987 | 1988 | 1989 | 1990 |
| 100 | kDa | 1991 | 1992 | 1993 | 1994 | 1995 | 1996 | 1997 | 1998 | 1999 | 2000 |
| 101 | kDa | 2001 | 2002 | 2003 | 2004 | 2005 | 2006 | 2007 | 2008 | 2009 | 2010 |
| 102 | kDa | 2011 | 2012 | 2013 | 2014 | 2015 | 2016 | 2017 | 2018 | 2019 | 2020 |
| 103 | kDa | 2021 | 2022 | 2023 | 2024 | 2025 | 2026 | 2027 | 2028 | 2029 | 2030 |
| 104 | kDa | 2031 | 2032 | 2033 | 2034 | 2035 | 2036 | 2037 | 2038 | 2039 | 2040 |
| 105 | kDa | 2041 | 2042 | 2043 | 2044 | 2045 | 2046 | 2047 | 2048 | 2049 | 2050 |
| 106 | kDa | 2051 | 2052 | 2053 | 2054 | 2055 | 2056 | 2057 | 2058 | 2059 | 2060 |
| 107 | kDa | 2061 | 2062 | 2063 | 2064 | 2065 | 2066 | 2067 | 2068 | 2069 | 2070 |
| 108 | kDa | 2071 | 2072 | 2073 | 2074 | 2075 | 2076 | 2077 | 2078 | 2079 | 2080 |
| 109 | kDa | 2081 | 2082 | 2083 | 2084 | 2085 | 2086 | 2087 | 2088 | 2089 | 2090 |
| 110 | kDa | 2091 | 2092 | 2093 | 2094 | 2095 | 2096 | 2097 | 2098 | 2099 | 2100 |
| 111 | kDa | 2101 | 2102 | 2103 | 2104 | 2105 | 2106 | 2107 | 2108 | 2109 | 2110 |
| 112 | kDa | 2111 | 2112 | 2113 | 2114 | 2115 | 2116 | 2117 | 2118 | 2119 | 2120 |
| 113 | kDa | 2121 | 2122 | 2123 | 2124 | 2125 | 2126 | 2127 | 2128 | 2129 | 2130 |
| 114 | kDa | 2131 | 2132 | 2133 | 2134 | 2135 | 2136 | 2137 | 2138 | 2139 | 2140 |
| 115 | kDa | 2141 | 2142 | 2143 | 2144 | 2145 | 2146 | 2147 | 2148 | 2149 | 2150 |
| 116 | kDa | 2151 | 2152 | 2153 | 2154 | 2155 | 2156 | 2157 | 2158 | 2159 | 2160 |
| 117 | kDa | 2161 | 2162 | 2163 | 2164 | 2165 | 2166 | 2167 | 2168 | 2169 | 2170 |
| 118 | kDa | 2171 | 2172 | 2173 | 2174 | 2175 | 2176 | 2177 | 2178 | 2179 | 2180 |
| 119 | kDa | 2181 | 2182 | 2183 | 2184 | 2185 | 2186 | 2187 | 2188 | 2189 | 2190 |
| 120 | kDa | 2191 | 2192 | 2193 | 2194 | 2195 | 2196 | 2197 | 2198 | 2199 | 2200 |
| 121 | kDa | 2201 | 2202 | 2203 | 2204 | 2205 | 2206 | 2207 | 2208 | 2209 | 2210 |
| 122 | kDa | 2211 | 2212 | 2213 | 2214 | 2215 | 2216 | 2217 | 2218 | 2219 | 2220 |
| 123 | kDa | 2221 | 2222 | 2223 | 2224 | 2225 | 2226 | 2227 | 2228 | 2229 | 2230 |
| 124 | kDa | 2231 | 2232 | 2233 | 2234 | 2235 | 2236 | 2237 | 2238 | 2239 | 2240 |
| 125 | kDa | 2241 | 2242 | 2243 | 2244 | 2245 | 2246 | 2247 | 2248 | 2249 | 2250 |
| 126 | kDa | 2251 | 2252 | 2253 | 2254 | 2255 | 2256 | 2257 | 2258 | 2259 | 2260 |
| 127 | kDa | 2261 | 2262 | 2263 | 2264 | 2265 | 2266 | 2267 | 2268 | 2269 | 2270 |
| 128 | kDa | 2271 | 2272 | 2273 | 2274 | 2275 | 2276 | 2277 | 2278 | 2279 | 2280 |
| 129 | kDa | 2281 | 2282 | 2283 | 2284 | 2285 | 2286 | 2287 | 2288 | 2289 | 2290 |
| 130 | kDa | 2291 | 2292 | 2293 | 2294 | 2295 | 2296 | 2297 | 2298 | 2299 | 2300 |
| 131 | kDa | 2301 | 2302 | 2303 | 2304 | 2305 | 2306 | 2307 | 2308 | 2309 | 2310 |
| 132 | kDa | 2311 | 2312 | 2313 | 2314 | 2315 | 2316 | 2317 | 2318 | 2319 | 2320 |
| 133 | kDa | 2321 | 2322 | 2323 | 2324 | 2325 | 2326 | 2327 | 2328 | 2329 | 2330 |
| 134 | kDa | 2331 | 2332 | 2333 | 2334 | 2335 | 2336 | 2337 | 2338 | 2339 | 2340 |
| 135 | kDa | 2341 | 2342 | 2343 | 2344 | 2345 | 2346 | 2347 | 2348 | 2349 | 2350 |
| 136 | kDa | 2351 | 2352 | 2353 | 2354 | 2355 | 2356 | 2357 | 2358 | 2359 | 2360 |
| 137 | kDa | 2361 | 2362 | 2363 | 2364 | 2365 | 2366 | 2367 | 2368 | 2369 | 2370 |
| 138 | kDa | 2371 | 2372 | 2373 | 2374 | 2375 | 2376 | 2377 | 2378 | 2379 | 2380 |
| 139 | kDa | 2381 | 2382 | 2383 | 2384 | 2385 | 2386 | 2387 | 2388 | 2389 | 2390 |
| 140 | kDa | 2391 | 2392 | 2393 | 2394 | 2395 | 2396 | 2397 | 2398 | 2399 | 2400 |
| 141 | kDa | 2401 | 2402 | 2403 | 2404 | 2405 | 2406 | 2407 | 2408 | 2409 | 2410 |
| 142 | kDa | 2411 | 2412 | 2413 | 2414 | 2415 | 2416 | 2417 | 2418 | 2419 | 2420 |
| 143 | kDa | 2421 | 2422 | 2423 | 2424 | 2425 | 2426 | 2427 | 2428 | 2429 | 2430 |
| 144 | kDa | 2431 | 2432 | 2433 | 2434 | 2435 | 2436 | 2437 | 2438 | 2439 | 2440 |
| 145 | kDa | 2441 | 2442 | 2443 | 2444 | 2445 | 2446 | 2447 | 2448 | 2449 | 2450 |

As used herein, “low molecular weight,” “low MW,” or “low-MW” SPF may include SPF having a weight average molecular weight, or average weight average molecular weight in a range of about 5 kDa to about 30 kDa, about 14 kDa to about 30 kDa, or about 6 kDa to about 17 kDa. In some embodiments, a target low molecular weight for certain SPF may be weight average molecular weight of about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, about 20 kDa, about 21 kDa, about 22 kDa, about 23 kDa, about 24 kDa, about 25 kDa, about 26 kDa, about 27 kDa, about 28 kDa, about 29 kDa, or about 30 kDa.

As used herein, “medium molecular weight,” “medium MW,” or “mid-MW” SPF may include SPF having a weight average molecular weight, or average weight average molecular weight in a range of about 20 kDa to about 55 kDa, about 39 kDa to about 54 kDa, or about 17 kDa to about 39 kDa. In some embodiments, a target medium molecular weight for certain SPF may be weight average molecular weight of about 17 kDa, about 18 kDa, about 19 kDa, about 20 kDa, about 21 kDa, about 22 kDa, about 23 kDa, about 24 kDa, about 25 kDa, about 26 kDa, about 27 kDa, about 28 kDa, about 29 kDa, about 30 kDa, about 31 kDa, about 32 kDa, about 33 kDa, about 34 kDa, about 35 kDa, about 36 kDa, about 37 kDa, about 38 kDa, about 39 kDa, about 40 kDa, about 41 kDa, about 42 kDa, about 43 kDa, about 44 kDa, about 45 kDa, about 46 kDa, about 47 kDa, about 48 kDa, about 49 kDa, about 50 kDa, about 51 kDa, about 52 kDa, about 53 kDa, about 54 kDa, or about 55 kDa.

As used herein, “high molecular weight,” “high MW,” or “high-MW” SPF may include SPF having a weight average molecular weight, or average weight average molecular weight that is in a range of about 55 kDa to about 150 kDa, or about 39 kDa to about 80 kDa. In some embodiments, a target high molecular weight for certain SPF may be about 39 kDa, about 40 kDa, about 41 kDa, about 42 kDa, about 43 kDa, about 44 kDa, about 45 kDa, about 46 kDa, about 47 kDa, about 48 kDa, about 49 kDa, about 50 kDa, about 51 kDa, about 52 kDa, about 53 kDa, about 54 kDa, about 55 kDa, about 56 kDa, about 57 kDa, about 58 kDa, about 59 kDa, about 60 kDa, about 61 kDa, about 62 kDa, about 63 kDa, about 64 kDa, about 65 kDa, about 66 kDa, about 67 kDa, about 68 kDa, about 69 kDa, about 70 kDa, about 71 kDa, about 72 kDa, about 73 kDa, about 74 kDa, about 75 kDa, about 76 kDa, about 77 kDa, about 78 kDa, about 79 kDa, or about 80 kDa.

In some embodiments, the molecular weights described herein (e.g., low molecular weight silk, medium molecular weight silk, high molecular weight silk) may be converted to the approximate number of amino acids contained within the respective SPF, as would be understood by a person having ordinary skill in the art. For example, the average weight of an amino acid may be about 110 daltons (i.e., 110 g/mol). Therefore, in some embodiments, dividing the molecular weight of a linear protein by 110 daltons may be used to approximate the number of amino acid residues contained therein.

In an embodiment, SPF in a composition of the present disclosure have a polydispersity ranging from 1 to about 5.0, including, without limitation, a polydispersity of 1. In an embodiment, SPF in a composition of the present disclosure have a polydispersity ranging from about 1.5 to about 3.0. In an embodiment, SPF in a composition of the present disclosure have a polydispersity ranging from 1 to about 1.5, including, without limitation, a polydispersity of 1. In an embodiment, SPF in a composition of the present disclosure have a polydispersity ranging from about 1.5 to about 2.0. In an embodiment, SPF in a composition of the present disclosure have a polydispersity ranging from about 2.0 to about 2.5. In an embodiment, SPF in a composition of the present disclosure have a polydispersity ranging from about 2.5 to about 3.0. In an embodiment, SPF in a composition of the present disclosure have a polydispersity ranging from about 3.0 to about 3.5. In an embodiment, SPF in a composition of the present disclosure have a polydispersity ranging from about 3.5 to about 4.0. In an embodiment, SPF in a composition of the present disclosure have a polydispersity ranging from about 4.0 to about 4.5. In an embodiment, SPF in a composition of the present disclosure have a polydispersity ranging from about 4.5 to about 5.0.

In an embodiment, SPF in a composition of the present disclosure have a polydispersity of 1. In an embodiment, SPF in a composition of the present disclosure have a polydispersity of about 1.1. In an embodiment, SPF in a composition of the present disclosure have a polydispersity of about 1.2. In an embodiment, SPF in a composition of the present disclosure have a polydispersity of about 1.3. In an embodiment, SPF in a composition of the present disclosure have a polydispersity of about 1.4. In an embodiment, SPF in a composition of the present disclosure have a polydispersity of about 1.5. In an embodiment, SPF in a composition of the present disclosure have a polydispersity of about 1.6. In an embodiment, SPF in a composition of the present disclosure have a polydispersity of about 1.7. In an embodiment, SPF in a composition of the present disclosure have a polydispersity of about 1.8. In an embodiment, SPF in a composition of the present disclosure have a polydispersity of about 1.9. In an embodiment, SPF in a composition of the present disclosure have a polydispersity of about 2.0. In an embodiment, SPF in a composition of the present disclosure have a polydispersity of about 2.1. In an embodiment, SPF in a composition of the present disclosure have a polydispersity of about 2.2. In an embodiment, SPF in a composition of the present disclosure have a polydispersity of about 2.3. In an embodiment, SPF in a composition of the present disclosure have a polydispersity of about 2.4. In an embodiment, SPF in a composition of the present disclosure have a polydispersity of about 2.5. In an embodiment, SPF in a composition of the present disclosure have a polydispersity of about 2.6. In an embodiment, SPF in a composition of the present disclosure have a polydispersity of about 2.7. In an embodiment, SPF in a composition of the present disclosure have a polydispersity of about 2.8. In an embodiment, SPF in a composition of the present disclosure have a polydispersity of about 2.9. In an embodiment, SPF in a composition of the present disclosure have a polydispersity of about 3.0. In an embodiment, SPF in a composition of the present disclosure have a polydispersity of about 3.1. In an embodiment, SPF in a composition of the present disclosure have a polydispersity of about 3.2. In an embodiment, SPF in a composition of the present disclosure have a polydispersity of about 3.3. In an embodiment, SPF in a composition of the present disclosure have a polydispersity of about 3.4. In an embodiment, SPF in a composition of the present disclosure have a polydispersity of about 3.5. In an embodiment, SPF in a composition of the present disclosure have a polydispersity of about 3.6. In an embodiment, SPF in a composition of the present disclosure have a polydispersity of about 3.7. In an embodiment, SPF in a composition of the present disclosure have a polydispersity of about 3.8. In an embodiment, SPF in a composition of the present disclosure have a polydispersity of about 3.9. In an embodiment, SPF in a composition of the present disclosure have a polydispersity of about 4.0. In an embodiment, SPF in a composition of the present disclosure have a polydispersity of about 4.1. In an embodiment, SPF in a composition of the present disclosure have a polydispersity of about 4.2. In an embodiment, SPF in a composition of the present disclosure have a polydispersity of about 4.3. In an embodiment, SPF in a composition of the present disclosure have a polydispersity of about 4.4. In an embodiment, SPF in a composition of the present disclosure have a polydispersity of about 4.5. In an embodiment, SPF in a composition of the present disclosure have a polydispersity of about 4.6. In an embodiment, SPF in a composition of the present disclosure have a polydispersity of about 4.7. In an embodiment, SPF in a composition of the present disclosure have a polydispersity of about 4.8. In an embodiment, SPF in a composition of the present disclosure have a polydispersity of about 4.9. In an embodiment, SPF in a composition of the present disclosure have a polydispersity of about 5.0.

In some embodiments, in compositions described herein having combinations of low, medium, and/or high molecular weight SPF, such low, medium, and/or high molecular weight SPF may have the same or different polydispersities.

Silk Fibroin Fragments

Methods of making silk fibroin or silk fibroin protein fragments and their applications in various fields are known and are described for example in U.S. Pat. Nos. 9,187,538, 9,511,012, 9,517,191, 9,522,107, 9,522,108, 9,545,369, and 10,166,177, 10,287,728 and 10,301,768, all of which are incorporated herein in their entireties. Raw silk from silkworm Bombyx mori is composed of two primary proteins: silk fibroin (approximately 75%) and sericin (approximately 25%). Silk fibroin is a fibrous protein with a semi-crystalline structure that provides stiffness and strength. As used herein, the term “silk fibroin” means the fibers of the cocoon of Bombyx mori having a weight average molecular weight of about 370,000 Da. The crude silkworm fiber consists of a double thread of fibroin. The adhesive substance holding these double fibers together is sericin. The silk fibroin is composed of a heavy chain having a weight average molecular weight of about 350,000 Da (H chain), and a light chain having a weight average molecular weight about 25,000 Da (L chain). Silk fibroin is an amphiphilic polymer with large hydrophobic domains occupying the major component of the polymer, which has a high molecular weight. The hydrophobic regions are interrupted by small hydrophilic spacers, and the N- and C-termini of the chains are also highly hydrophilic. The hydrophobic domains of the H-chain contain a repetitive hexapeptide sequence of Gly-Ala-Gly-Ala-Gly-Ser and repeats of Gly-Ala/Ser/Tyr dipeptides, which can form stable anti-parallel-sheet crystallites. The amino acid sequence of the L-chain is non-repetitive, so the L-chain is more hydrophilic and relatively elastic. The hydrophilic (Tyr, Ser) and hydrophobic (Gly, Ala) chain segments in silk fibroin molecules are arranged alternatively such that allows self-assembling of silk fibroin molecules.

Provided herein are methods for producing pure and highly scalable silk fibroin-protein fragment mixture solutions that may be used across multiple industries for a variety of applications. Without wishing to be bound by any particular theory, it is believed that these methods are equally applicable to fragmentation of any SPF described herein, including without limitation recombinant silk proteins, and fragmentation of silk-like or fibroin-like proteins.

As used herein, the term “fibroin” includes silk worm fibroin and insect or spider silk protein. In an embodiment, fibroin is obtained from Bombyx mori. Raw silk from Bombyx mori is composed of two primary proteins: silk fibroin (approximately 75%) and sericin (approximately 25%). Silk fibroin is a fibrous protein with a semi-crystalline structure that provides stiffness and strength. As used herein, the term “silk fibroin” means the fibers of the cocoon of Bombyx mori having a weight average molecular weight of about 370,000 Da. Conversion of these insoluble silk fibroin fibrils into water-soluble silk fibroin protein fragments requires the addition of a concentrated neutral salt (e.g., 8-10 M lithium bromide), which interferes with inter- and intramolecular ionic and hydrogen bonding that would otherwise render the fibroin protein insoluble in water. Methods of making silk fibroin protein fragments, and/or compositions thereof, are known and are described for example in U.S. Pat. Nos. 9,187,538, 9,511,012, 9,517,191, 9,522,107, 9,522,108, 9,545,369, and 10,166,177.

The raw silk cocoons from the silkworm Bombyx mori was cut into pieces. The pieces silk cocoons were processed in an aqueous solution of Na₂CO₃ at about 100° C. for about 60 minutes to remove sericin (degumming). The volume of the water used equals about 0.4×raw silk weight and the amount of Na₂CO₃ is about 0.848×the weight of the raw silk cocoon pieces. The resulting degummed silk cocoon pieces were rinsed with deionized water three times at about 60° C. (20 minutes per rinse). The volume of rinse water for each cycle was 0.2 L× the weight of the raw silk cocoon pieces. The excess water from the degummed silk cocoon pieces was removed. After the DI water washing step, the wet degummed silk cocoon pieces were dried at room temperature. The degummed silk cocoon pieces were mixed with a LiBr solution, and the mixture was heated to about 100° C. The warmed mixture was placed in a dry oven and was heated at about 100° C. for about 60 minutes to achieve complete dissolution of the native silk protein. The resulting silk fibroin solution was filtered and dialyzed using Tangential Flow Filtration (TFF) and a 10 kDa membrane against deionized water for 72 hours. The resulting silk fibroin aqueous solution has a concentration of about 8.5 wt. %. Then, 8.5% silk solution was diluted with water to result in a 1.0% w/v silk solution. TFF can then be used to further concentrate the pure silk solution to a concentration of 20.0% w/w silk to water.

Dialyzing the silk through a series of water changes is a manual and time intensive process, which could be accelerated by changing certain parameters, for example diluting the silk solution prior to dialysis. The dialysis process could be scaled for manufacturing by using semi-automated equipment, for example a tangential flow filtration system.

In some embodiments, the silk solutions are prepared under various preparation condition parameters such as: 90° C. 30 min, 90° C. 60 min, 100° C. 30 min, and 100° C. 60 min. Briefly, 9.3 M LiBr was prepared and allowed to sit at room temperature for at least 30 minutes. 5 mL of LiBr solution was added to 1.25 g of silk and placed in the 60° C. oven. Samples from each set were removed at 4, 6, 8, 12, 24, 168 and 192 hours.

In some embodiments, the silk solutions are prepared under various preparation condition parameters such as: 90° C. 30 min, 90° C. 60 min, 100° C. 30 min, and 100° C. 60 min. Briefly, 9.3 M LiBr solution was heated to one of four temperatures: 60° C., 80° C., 100° C. or boiling. 5 mL of hot LiBr solution was added to 1.25 g of silk and placed in the 60° C. oven. Samples from each set were removed at 1, 4 and 6 hours.

In some embodiments, the silk solutions are prepared under various preparation condition parameters such as: Four different silk extraction combinations were used: 90° C. 30 min, 90° C. 60 min, 100° C. 30 min, and 100° C. 60 min. Briefly, 9.3 M LiBr solution was heated to one of four temperatures: 60° C., 80° C., 100° C. or boiling. 5 mL of hot LiBr solution was added to 1.25 g of silk and placed in the oven at the same temperature of the LiBr. Samples from each set were removed at 1, 4 and 6 hours. 1 mL of each sample was added to 7.5 mL of 9.3 M LiBr and refrigerated for viscosity testing.

In some embodiments, SPF are obtained by dissolving raw unscoured, partially scoured, or scoured silkworm fibers with a neutral lithium bromide salt. The raw silkworm silks are processed under selected temperature and other conditions in order to remove any sericin and achieve the desired weight average molecular weight (Mw) and polydispersity (PD) of the fragment mixture. Selection of process parameters may be altered to achieve distinct final silk protein fragment characteristics depending upon the intended use. The resulting final fragment solution is silk fibroin protein fragments and water with parts per million (ppm) to non-detectable levels of process contaminants, levels acceptable in the pharmaceutical, medical and consumer eye care markets. The concentration, size and polydispersity of SPF may further be altered depending upon the desired use and performance requirements.

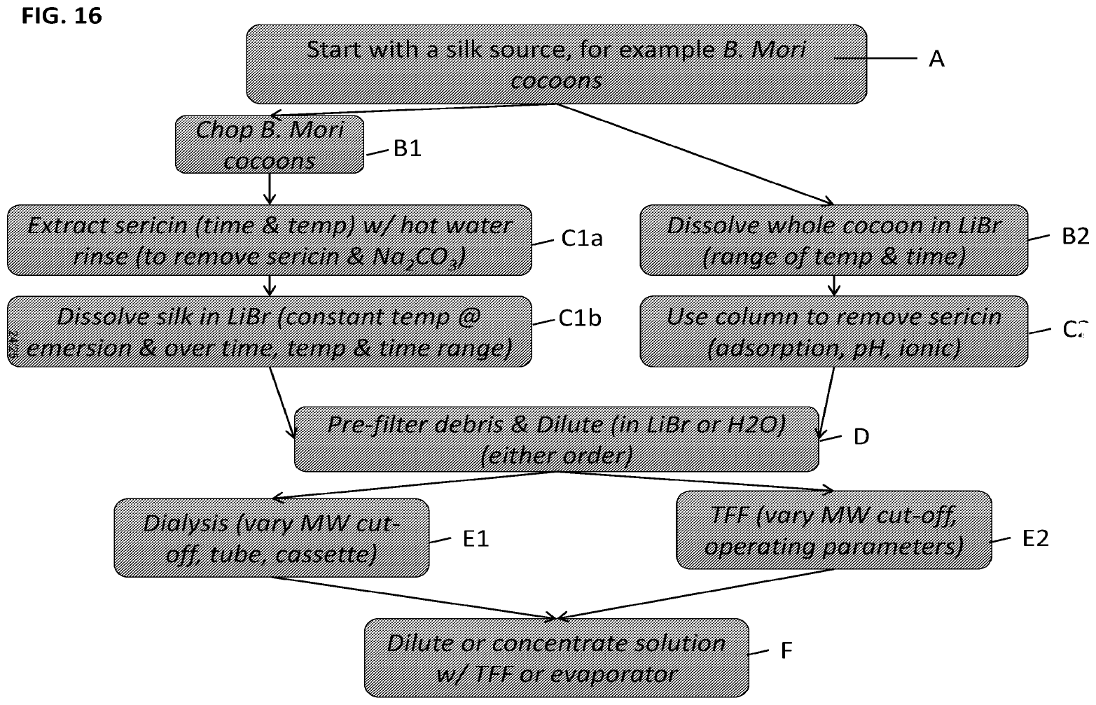

FIG. 16 is a flow chart showing various embodiments for producing pure silk fibroin protein fragments (SPFs) of the present disclosure. It should be understood that not all of the steps illustrated are necessarily required to fabricate all silk solutions of the present disclosure. As illustrated in FIG. 16, step A, cocoons (heat-treated or non-heat-treated), silk fibers, silk powder, spider silk or recombinant spider silk can be used as the silk source. If starting from raw silk cocoons from Bombyx mori, the cocoons can be cut into small pieces, for example pieces of approximately equal size, step B1. The raw silk is then extracted and rinsed to remove any sericin, step C1a. This results in substantially sericin free raw silk. In an embodiment, water is heated to a temperature between 84° C. and 100° C. (ideally boiling) and then Na₂CO₃ (sodium carbonate) is added to the boiling water until the Na₂CO₃ is completely dissolved. The raw silk is added to the boiling water/Na₂CO₃ (100° C.) and submerged for approximately 15-90 minutes, where boiling for a longer time results in smaller silk protein fragments. In an embodiment, the water volume equals about 0.4×raw silk weight and the Na₂CO₃ volume equals about 0.848×raw silk weight. In an embodiment, the water volume equals 0.1×raw silk weight and the Na₂CO₃ volume is maintained at 2.12 g/L.

Subsequently, the water dissolved Na₂CO₃ solution is drained and excess water/Na₂CO₃ is removed from the silk fibroin fibers (e.g., ring out the fibroin extract by hand, spin cycle using a machine, etc.). The resulting silk fibroin extract is rinsed with warm to hot water to remove any remaining adsorbed sericin or contaminate, typically at a temperature range of about 40° C. to about 80° C., changing the volume of water at least once (repeated for as many times as required). The resulting silk fibroin extract is a substantially sericin-depleted silk fibroin. In an embodiment, the resulting silk fibroin extract is rinsed with water at a temperature of about 60° C. In an embodiment, the volume of rinse water for each cycle equals 0.1 L to 0.2 L×raw silk weight. It may be advantageous to agitate, turn or circulate the rinse water to maximize the rinse effect. After rinsing, excess water is removed from the extracted silk fibroin fibers (e.g., ring out fibroin extract by hand or using a machine). Alternatively, methods known to one skilled in the art such as pressure, temperature, or other reagents or combinations thereof may be used for the purpose of sericin extraction. Alternatively, the silk gland (100% sericin free silk protein) can be removed directly from a worm. This would result in liquid silk protein, without any alteration of the protein structure, free of sericin.

The extracted fibroin fibers are then allowed to dry completely. Once dry, the extracted silk fibroin is dissolved using a solvent added to the silk fibroin at a temperature between ambient and boiling, step C1b. In an embodiment, the solvent is a solution of Lithium bromide (LiBr) (boiling for LiBr is 140° C.). Alternatively, the extracted fibroin fibers are not dried but wet and placed in the solvent; solvent concentration can then be varied to achieve similar concentrations as to when adding dried silk to the solvent. The final concentration of LiBr solvent can range from 0.1 M to 9.3 M. Complete dissolution of the extracted fibroin fibers can be achieved by varying the treatment time and temperature along with the concentration of dissolving solvent. Other solvents may be used including, but not limited to, phosphate phosphoric acid, calcium nitrate, calcium chloride solution or other concentrated aqueous solutions of inorganic salts. To ensure complete dissolution, the silk fibers should be fully immersed within the already heated solvent solution and then maintained at a temperature ranging from about 60° C. to about 140° C. for 1-168 hrs. In an embodiment, the silk fibers should be fully immersed within the solvent solution and then placed into a dry oven at a temperature of about 100° C. for about 1 hour.

The temperature at which the silk fibroin extract is added to the LiBr solution (or vice versa) has an effect on the time required to completely dissolve the fibroin and on the resulting molecular weight and polydispersity of the final SPF mixture solution. In an embodiment, silk solvent solution concentration is less than or equal to 20% w/v. In addition, agitation during introduction or dissolution may be used to facilitate dissolution at varying temperatures and concentrations. The temperature of the LiBr solution will provide control over the silk protein fragment mixture molecular weight and polydispersity created. In an embodiment, a higher temperature will more quickly dissolve the silk offering enhanced process scalability and mass production of silk solution. In an embodiment, using a LiBr solution heated to a temperature from 80° C. to 140° C. reduces the time required in an oven in order to achieve full dissolution. Varying time and temperature at or above 60° C. of the dissolution solvent will alter and control the MW and polydispersity of the SPF mixture solutions formed from the original molecular weight of the native silk fibroin protein.

Alternatively, whole cocoons may be placed directly into a solvent, such as LiBr, bypassing extraction, step B2. This requires subsequent filtration of silk worm particles from the silk and solvent solution and sericin removal using methods know in the art for separating hydrophobic and hydrophilic proteins such as a column separation and/or chromatography, ion exchange, chemical precipitation with salt and/or pH, and or enzymatic digestion and filtration or extraction, all methods are common examples and without limitation for standard protein separation methods, step C2. Non-heat treated cocoons with the silkworm removed, may alternatively be placed into a solvent such as LiBr, bypassing extraction. The methods described above may be used for sericin separation, with the advantage that non-heat treated cocoons will contain significantly less worm debris.

Dialysis may be used to remove the dissolution solvent from the resulting dissolved fibroin protein fragment solution by dialyzing the solution against a volume of water, step E1. Pre-filtration prior to dialysis is helpful to remove any debris (i.e., silk worm remnants) from the silk and LiBr solution, step D. In one example, a 3 μm or 5 μm filter is used with a flow-rate of 200-300 mL/min to filter a 0.1% to 1.0% silk-LiBr solution prior to dialysis and potential concentration if desired. A method disclosed herein, as described above, is to use time and/or temperature to decrease the concentration from 9.3 M LiBr to a range from 0.1 M to 9.3 M to facilitate filtration and downstream dialysis, particularly when considering creating a scalable process method. Alternatively, without the use of additional time or temperate, a 9.3 M LiBr-silk protein fragment solution may be diluted with water to facilitate debris filtration and dialysis. The result of dissolution at the desired time and temperate filtration is a translucent particle-free room temperature shelf-stable silk protein fragment-LiBr solution of a known MW and polydispersity. It is advantageous to change the dialysis water regularly until the solvent has been removed (e.g., change water after 1 hour, 4 hours, and then every 12 hours for a total of 6 water changes). The total number of water volume changes may be varied based on the resulting concentration of solvent used for silk protein dissolution and fragmentation. After dialysis, the final silk solution maybe further filtered to remove any remaining debris (i.e., silk worm remnants).

Alternatively, Tangential Flow Filtration (TFF), which is a rapid and efficient method for the separation and purification of biomolecules, may be used to remove the solvent from the resulting dissolved fibroin solution, step E2. TFF offers a highly pure aqueous silk protein fragment solution and enables scalability of the process in order to produce large volumes of the solution in a controlled and repeatable manner. The silk and LiBr solution may be diluted prior to TFF (20% down to 0.1% silk in either water or LiBr). Pre-filtration as described above prior to TFF processing may maintain filter efficiency and potentially avoids the creation of silk gel boundary layers on the filter's surface as the result of the presence of debris particles. Pre-filtration prior to TFF is also helpful to remove any remaining debris (i.e., silk worm remnants) from the silk and LiBr solution that may cause spontaneous or long-term gelation of the resulting water only solution, step D. TFF, recirculating or single pass, may be used for the creation of water-silk protein fragment solutions ranging from 0.1% silk to 30.0% silk (more preferably, 0.1%-6.0% silk). Different cutoff size TFF membranes may be required based upon the desired concentration, molecular weight and polydispersity of the silk protein fragment mixture in solution. Membranes ranging from 1-100 kDa may be necessary for varying molecular weight silk solutions created for example by varying the length of extraction boil time or the time and temperate in dissolution solvent (e.g., LiBr). In an embodiment, a TFF 5 or 10 kDa membrane is used to purify the silk protein fragment mixture solution and to create the final desired silk-to-water ratio. As well, TFF single pass, TFF, and other methods known in the art, such as a falling film evaporator, may be used to concentrate the solution following removal of the dissolution solvent (e.g., LiBr) (with resulting desired concentration ranging from 0.1% to 30% silk). This can be used as an alternative to standard HFIP concentration methods known in the art to create a water-based solution. A larger pore membrane could also be utilized to filter out small silk protein fragments and to create a solution of higher molecular weight silk with and/or without tighter polydispersity values.

An assay for LiBr and Na₂CO₃ detection can be performed using an HPLC system equipped with evaporative light scattering detector (ELSD). The calculation was performed by linear regression of the resulting peak areas for the analyte plotted against concentration. More than one sample of a number of formulations of the present disclosure was used for sample preparation and analysis. Generally, four samples of different formulations were weighed directly in a 10 mL volumetric flask. The samples were suspended in 5 mL of 20 mM ammonium formate (pH 3.0) and kept at 2-8° C. for 2 hours with occasional shaking to extract analytes from the film. After 2 hours the solution was diluted with 20 mM ammonium formate (pH 3.0). The sample solution from the volumetric flask was transferred into HPLC vials and injected into the HPLC-ELSD system for the estimation of sodium carbonate and lithium bromide.

The analytical method developed for the quantitation of Na₂CO₃ and LiBr in silk protein formulations was found to be linear in the range 10-165 μg/mL, with RSD for injection precision as 2% and 1% for area and 0.38% and 0.19% for retention time for sodium carbonate and lithium bromide respectively. The analytical method can be applied for the quantitative determination of sodium carbonate and lithium bromide in silk protein formulations.

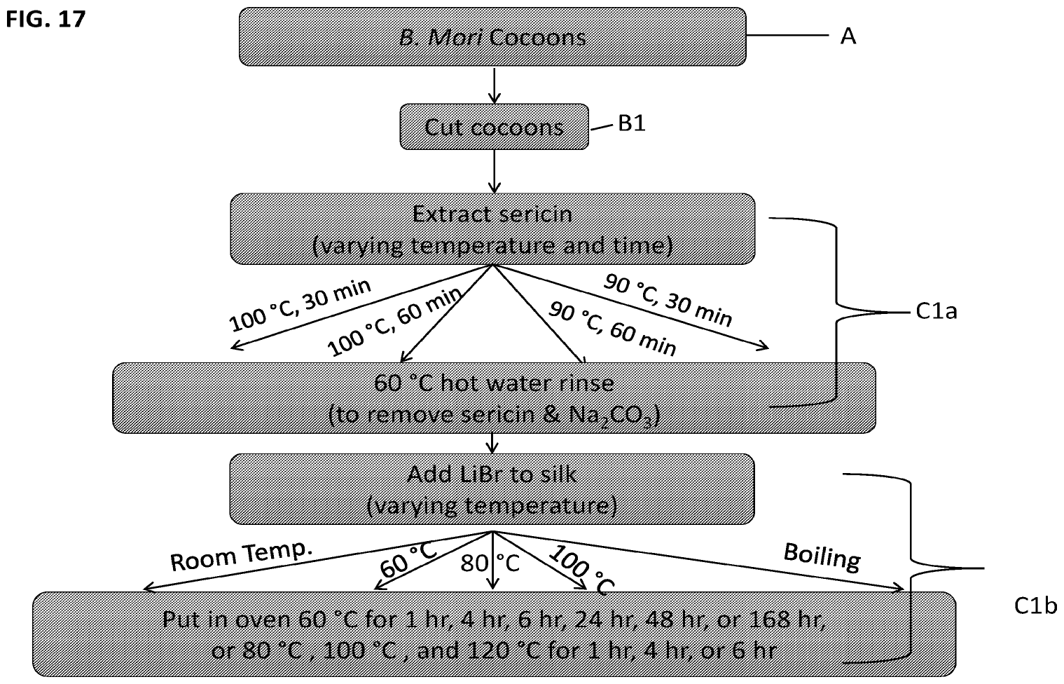

FIG. 17 is a flow chart showing various parameters that can be modified during the process of producing a silk protein fragment solution of the present disclosure during the extraction and the dissolution steps. Select method parameters may be altered to achieve distinct final solution characteristics depending upon the intended use, e.g., molecular weight and polydispersity. It should be understood that not all of the steps illustrated are necessarily required to fabricate all silk solutions of the present disclosure.

In an embodiment, silk protein fragment solutions useful for a wide variety of applications are prepared according to the following steps: forming pieces of silk cocoons from the Bombyx mori silkworm; extracting the pieces at about 100° C. in a Na₂CO₃ water solution for about 60 minutes, wherein a volume of the water equals about 0.4×raw silk weight and the amount of Na₂CO₃ is about 0.848×the weight of the pieces to form a silk fibroin extract; triple rinsing the silk fibroin extract at about 60° C. for about 20 minutes per rinse in a volume of rinse water, wherein the rinse water for each cycle equals about 0.2 L× the weight of the pieces; removing excess water from the silk fibroin extract; drying the silk fibroin extract; dissolving the dry silk fibroin extract in a LiBr solution, wherein the LiBr solution is first heated to about 100° C. to create a silk and LiBr solution and maintained; placing the silk and LiBr solution in a dry oven at about 100° C. for about 60 minutes to achieve complete dissolution and further fragmentation of the native silk protein structure into mixture with desired molecular weight and polydispersity; filtering the solution to remove any remaining debris from the silkworm; diluting the solution with water to result in a 1.0 wt. % silk solution; and removing solvent from the solution using Tangential Flow Filtration (TFF). In an embodiment, a 10 kDa membrane is utilized to purify the silk solution and create the final desired silk-to-water ratio. TFF can then be used to further concentrate the silk solution to a concentration of 2.0 wt. % silk in water.

Without wishing to be bound by any particular theory, varying extraction (i.e., time and temperature), LiBr (i.e., temperature of LiBr solution when added to silk fibroin extract or vice versa) and dissolution (i.e., time and temperature) parameters results in solvent and silk solutions with different viscosities, homogeneities, and colors. Also without wishing to be bound by any particular theory, increasing the temperature for extraction, lengthening the extraction time, using a higher temperature LiBr solution at emersion and over time when dissolving the silk and increasing the time at temperature (e.g., in an oven as shown here, or an alternative heat source) all resulted in less viscous and more homogeneous solvent and silk solutions.

The extraction step could be completed in a larger vessel, for example an industrial washing machine where temperatures at or in between 60° C. to 100° C. can be maintained. The rinsing step could also be completed in the industrial washing machine, eliminating the manual rinse cycles. Dissolution of the silk in LiBr solution could occur in a vessel other than a convection oven, for example a stirred tank reactor. Dialyzing the silk through a series of water changes is a manual and time intensive process, which could be accelerated by changing certain parameters, for example diluting the silk solution prior to dialysis. The dialysis process could be scaled for manufacturing by using semi-automated equipment, for example a tangential flow filtration system.

Varying extraction (i.e., time and temperature), LiBr (i.e., temperature of LiBr solution when added to silk fibroin extract or vice versa) and dissolution (i.e., time and temperature) parameters results in solvent and silk solutions with different viscosities, homogeneities, and colors. Increasing the temperature for extraction, lengthening the extraction time, using a higher temperature LiBr solution at emersion and over time when dissolving the silk and increasing the time at temperature (e.g., in an oven as shown here, or an alternative heat source) all resulted in less viscous and more homogeneous solvent and silk solutions. While almost all parameters resulted in a viable silk solution, methods that allow complete dissolution to be achieved in fewer than 4 to 6 hours are preferred for process scalability.

In an embodiment, solutions of silk fibroin protein fragments having a weight average ranging from about 6 kDa to about 17 kDa are prepared according to following steps: degumming a silk source by adding the silk source to a boiling (100° C.) aqueous solution of sodium carbonate for a treatment time of between about 30 minutes to about 60 minutes; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 60° C. to about 140° C.; maintaining the solution of silk fibroin-lithium bromide in an oven having a temperature of about 140° C. for a period of at most 1 hour; removing the lithium bromide from the silk fibroin extract; and producing an aqueous solution of silk protein fragments, the aqueous solution comprising: fragments having a weight average molecular weight ranging from about 6 kDa to about 17 kDa, and a polydispersity of between 1 and about 5, or between about 1.5 and about 3.0. The method may further comprise drying the silk fibroin extract prior to the dissolving step. The aqueous solution of silk fibroin protein fragments may comprise lithium bromide residuals of less than 300 ppm as measured using a high-performance liquid chromatography lithium bromide assay. The aqueous solution of silk fibroin protein fragments may comprise sodium carbonate residuals of less than 100 ppm as measured using a high-performance liquid chromatography sodium carbonate assay. The aqueous solution of silk fibroin protein fragments may be lyophilized. In some embodiments, the silk fibroin protein fragment solution may be further processed into various forms including gel, powder, and nanofiber.

In an embodiment, solutions of silk fibroin protein fragments having a weight average molecular weight ranging from about 17 kDa to about 39 kDa are prepared according to the following steps: adding a silk source to a boiling (100° C.) aqueous solution of sodium carbonate for a treatment time of between about 30 minutes to about 60 minutes so as to result in degumming; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 80° C. to about 140° C.; maintaining the solution of silk fibroin-lithium bromide in a dry oven having a temperature in the range between about 60° C. to about 100° C. for a period of at most 1 hour; removing the lithium bromide from the silk fibroin extract; and producing an aqueous solution of silk fibroin protein fragments, wherein the aqueous solution of silk fibroin protein fragments comprises lithium bromide residuals of between about 10 ppm and about 300 ppm, wherein the aqueous solution of silk protein fragments comprises sodium carbonate residuals of between about 10 ppm and about 100 ppm, wherein the aqueous solution of silk fibroin protein fragments comprises fragments having a weight average molecular weight ranging from about 17 kDa to about 39 kDa, and a polydispersity of between 1 and about 5, or between about 1.5 and about 3.0. The method may further comprise drying the silk fibroin extract prior to the dissolving step. The aqueous solution of silk fibroin protein fragments may comprise lithium bromide residuals of less than 300 ppm as measured using a high-performance liquid chromatography lithium bromide assay. The aqueous solution of silk fibroin protein fragments may comprise sodium carbonate residuals of less than 100 ppm as measured using a high-performance liquid chromatography sodium carbonate assay.

In some embodiments, a method for preparing an aqueous solution of silk fibroin protein fragments having an average weight average molecular weight ranging from about 6 kDa to about 17 kDa includes the steps of: degumming a silk source by adding the silk source to a boiling (100° C.) aqueous solution of sodium carbonate for a treatment time of between about 30 minutes to about 60 minutes; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 60° C. to about 140° C.; maintaining the solution of silk fibroin-lithium bromide in an oven having a temperature of about 140° C. for a period of at least 1 hour; removing the lithium bromide from the silk fibroin extract; and producing an aqueous solution of silk protein fragments, the aqueous solution comprising: fragments having an average weight average molecular weight ranging from about 6 kDa to about 17 kDa, and a polydispersity of between 1 and about 5, or between about 1.5 and about 3.0. The method may further comprise drying the silk fibroin extract prior to the dissolving step. The aqueous solution of pure silk fibroin protein fragments may comprise lithium bromide residuals of less than 300 ppm as measured using a high-performance liquid chromatography lithium bromide assay. The aqueous solution of pure silk fibroin protein fragments may comprise sodium carbonate residuals of less than 100 ppm as measured using a high-performance liquid chromatography sodium carbonate assay. The method may further comprise adding a therapeutic agent to the aqueous solution of pure silk fibroin protein fragments. The method may further comprise adding a molecule selected from one of an antioxidant or an enzyme to the aqueous solution of pure silk fibroin protein fragments. The method may further comprise adding a vitamin to the aqueous solution of pure silk fibroin protein fragments. The vitamin may be vitamin C or a derivative thereof. The aqueous solution of pure silk fibroin protein fragments may be lyophilized. The method may further comprise adding an alpha hydroxy acid to the aqueous solution of pure silk fibroin protein fragments. The alpha hydroxy acid may be selected from the group consisting of glycolic acid, lactic acid, tartaric acid and citric acid. The method may further comprise adding hyaluronic acid or its salt form at a concentration of about 0.5% to about 10.0% to the aqueous solution of pure silk fibroin protein fragments. The method may further comprise adding at least one of zinc oxide or titanium dioxide. A film may be fabricated from the aqueous solution of pure silk fibroin protein fragments produced by this method. The film may comprise from about 1.0 wt. % to about 50.0 wt. % of vitamin C or a derivative thereof. The film may have a water content ranging from about 2.0 wt. % to about 20.0 wt. %. The film may comprise from about 30.0 wt. % to about 99.5 wt. % of pure silk fibroin protein fragments. A gel may be fabricated from the aqueous solution of pure silk fibroin protein fragments produced by this method. The gel may comprise from about 0.5 wt. % to about 20.0 wt. % of vitamin C or a derivative thereof. The gel may have a silk content of at least 2% and a vitamin content of at least 20%.

In some embodiments, a method for preparing an aqueous solution of silk fibroin protein fragments having an average weight average molecular weight ranging from about 17 kDa to about 39 kDa includes the steps of: adding a silk source to a boiling (100° C.) aqueous solution of sodium carbonate for a treatment time of between about 30 minutes to about 60 minutes so as to result in degumming; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 80° C. to about 140° C.; maintaining the solution of silk fibroin-lithium bromide in a dry oven having a temperature in the range between about 60° C. to about 100° C. for a period of at least 1 hour; removing the lithium bromide from the silk fibroin extract; and producing an aqueous solution of pure silk fibroin protein fragments, wherein the aqueous solution of pure silk fibroin protein fragments comprises lithium bromide residuals of between about 10 ppm and about 300 ppm, wherein the aqueous solution of silk protein fragments comprises sodium carbonate residuals of between about 10 ppm and about 100 ppm, wherein the aqueous solution of pure silk fibroin protein fragments comprises fragments having an average weight average molecular weight ranging from about 17 kDa to about 39 kDa, and a polydispersity of between 1 and about 5, or between about 1.5 and about 3.0. The method may further comprise drying the silk fibroin extract prior to the dissolving step. The aqueous solution of pure silk fibroin protein fragments may comprise lithium bromide residuals of less than 300 ppm as measured using a high-performance liquid chromatography lithium bromide assay. The aqueous solution of pure silk fibroin protein fragments may comprise sodium carbonate residuals of less than 100 ppm as measured using a high-performance liquid chromatography sodium carbonate assay. The method may further comprise adding a therapeutic agent to the aqueous solution of pure silk fibroin protein fragments. The method may further comprise adding a molecule selected from one of an antioxidant or an enzyme to the aqueous solution of pure silk fibroin protein fragments. The method may further comprise adding a vitamin to the aqueous solution of pure silk fibroin protein fragments. The vitamin may be vitamin C or a derivative thereof. The aqueous solution of pure silk fibroin protein fragments may be lyophilized. The method may further comprise adding an alpha hydroxy acid to the aqueous solution of pure silk fibroin protein fragments. The alpha hydroxy acid may be selected from the group consisting of glycolic acid, lactic acid, tartaric acid and citric acid. The method may further comprise adding hyaluronic acid or its salt form at a concentration of about 0.5% to about 10.0% to the aqueous solution of pure silk fibroin protein fragments. The method may further comprise adding at least one of zinc oxide or titanium dioxide. A film may be fabricated from the aqueous solution of pure silk fibroin protein fragments produced by this method. The film may comprise from about 1.0 wt. % to about 50.0 wt. % of vitamin C or a derivative thereof. The film may have a water content ranging from about 2.0 wt. % to about 20.0 wt. %. The film may comprise from about 30.0 wt. % to about 99.5 wt. % of pure silk fibroin protein fragments. A gel may be fabricated from the aqueous solution of pure silk fibroin protein fragments produced by this method. The gel may comprise from about 0.5 wt. % to about 20.0 wt. % of vitamin C or a derivative thereof. The gel may have a silk content of at least 2% and a vitamin content of at least 20%.

In an embodiment, solutions of silk fibroin protein fragments having a weight average molecular weight ranging from about 39 kDa to about 80 kDa are prepared according to the following steps: adding a silk source to a boiling (100° C.) aqueous solution of sodium carbonate for a treatment time of about 30 minutes so as to result in degumming; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 80° C. to about 140° C.; maintaining the solution of silk fibroin-lithium bromide in a dry oven having a temperature in the range between about 60° C. to about 100° C. for a period of at most 1 hour; removing the lithium bromide from the silk fibroin extract; and producing an aqueous solution of silk fibroin protein fragments, wherein the aqueous solution of silk fibroin protein fragments comprises lithium bromide residuals of between about 10 ppm and about 300 ppm, sodium carbonate residuals of between about 10 ppm and about 100 ppm, fragments having a weight average molecular weight ranging from about 39 kDa to about 80 kDa, and a polydispersity of between 1 and about 5, or between about 1.5 and about 3.0. The method may further comprise drying the silk fibroin extract prior to the dissolving step. The aqueous solution of silk fibroin protein fragments may comprise lithium bromide residuals of less than 300 ppm as measured using a high-performance liquid chromatography lithium bromide assay. The aqueous solution of silk fibroin protein fragments may comprise sodium carbonate residuals of less than 100 ppm as measured using a high-performance liquid chromatography sodium carbonate assay. In some embodiments, the method may further comprise adding an active agent (e.g., therapeutic agent) to the aqueous solution of pure silk fibroin protein fragments. The method may further comprise adding an active agent selected from one of an antioxidant or an enzyme to the aqueous solution of pure silk fibroin protein fragments. The method may further comprise adding a vitamin to the aqueous solution of pure silk fibroin protein fragments. The vitamin may be vitamin C or a derivative thereof. The aqueous solution of pure silk fibroin protein fragments may be lyophilized. The method may further comprise adding an alpha-hydroxy acid to the aqueous solution of pure silk fibroin protein fragments. The alpha hydroxy acid may be selected from the group consisting of glycolic acid, lactic acid, tartaric acid and citric acid. The method may further comprise adding hyaluronic acid or its salt form at a concentration of about 0.5% to about 10.0% to the aqueous solution of pure silk fibroin protein fragments. A film may be fabricated from the aqueous solution of pure silk fibroin protein fragments produced by this method. The film may comprise from about 1.0 wt. % to about 50.0 wt. % of vitamin C or a derivative thereof. The film may have a water content ranging from about 2.0 wt. % to about 20.0 wt. %. The film may comprise from about 30.0 wt. % to about 99.5 wt. % of pure silk fibroin protein fragments. A gel may be fabricated from the aqueous solution of pure silk fibroin protein fragments produced by this method. The gel may comprise from about 0.5 wt. % to about 20.0 wt. % of vitamin C or a derivative thereof. The gel may have a silk content of at least 2 wt. % and a vitamin content of at least 20 wt. %.

Molecular weight of the silk protein fragments may be controlled based upon the specific parameters utilized during the extraction step, including extraction time and temperature; specific parameters utilized during the dissolution step, including the LiBr temperature at the time of submersion of the silk in to the lithium bromide and time that the solution is maintained at specific temperatures; and specific parameters utilized during the filtration step. By controlling process parameters using the disclosed methods, it is possible to create silk fibroin protein fragment solutions with polydispersity equal to or lower than 2.5 at a variety of different molecular weight ranging from 5 kDa to 200 kDa, or between 10 kDa and 80 kDa. By altering process parameters to achieve silk solutions with different molecular weights, a range of fragment mixture end products, with desired polydispersity of equal to or less than 2.5 may be targeted based upon the desired performance requirements. For example, a higher molecular weight silk film containing an ophthalmic drug may have a controlled slow release rate compared to a lower molecular weight film making it ideal for a delivery vehicle in eye care products. Additionally, the silk fibroin protein fragment solutions with a polydispersity of greater than 2.5 can be achieved. Further, two solutions with different average molecular weights and polydispersity can be mixed to create combination solutions. Alternatively, a liquid silk gland (100% sericin free silk protein) that has been removed directly from a worm could be used in combination with any of the silk fibroin protein fragment solutions of the present disclosure. Molecular weight of the pure silk fibroin protein fragment composition was determined using High Pressure Liquid Chromatography (HPLC) with a Refractive Index Detector (RID). Polydispersity was calculated using Cirrus GPC Online GPC/SEC Software Version 3.3 (Agilent).

Differences in the processing parameters can result in regenerated silk fibroins that vary in molecular weight, and peptide chain size distribution (polydispersity, PD). This, in turn, influences the regenerated silk fibroin performance, including mechanical strength, water solubility etc.

Parameters were varied during the processing of raw silk cocoons into the silk solution. Varying these parameters affected the MW of the resulting silk solution. Parameters manipulated included (i) time and temperature of extraction, (ii) temperature of LiBr, (iii) temperature of dissolution oven, and (iv) dissolution time. Experiments were carried out to determine the effect of varying the extraction time. Tables 1-7 summarize the results. Below is a summary:
A sericin extraction time of 30 minutes resulted in larger molecular weight than a sericin extraction time of 60 minutes
Molecular weight decreases with time in the oven
140° C. LiBr and oven resulted in the low end of the confidence interval to be below a molecular weight of 29500 Da
30 min extraction at the 1 hour and 4 hour time points have undigested silk
30 mi extraction at the 1 hour time point resulted in a significantly high molecular weight with the low end of the confidence interval being 35,000 Da
The range of molecular weight reached for the high end of the confidence interval was 18000 to 216000 Da (important for offering solutions with specified upper limit).

TABLE 1
The effect of extraction time (30 min vs 60 min) on molecular
weight of silk processed under the conditions of 100°
C. Extraction Temperature, 100° C. Lithium Bromide (LiBr)
and 100° C. Oven Dissolution (Oven/Dissolution Time was varied).

| Boil | Oven | Average | Std | Confidence | | |
|---|---|---|---|---|---|---|
| Time | Time | Mw | dev | Interval | | PD |
| 30 | 1 | 57247 | 12780 | 35093 | 93387 | 1.63 |
| 60 | 1 | 31520 | 1387 | 11633 | 85407 | 2.71 |
| 30 | 4 | 40973 | 2632 | 14268 | 117658 | 2.87 |
| 60 | 4 | 25082 | 1248 | 10520 | 59803 | 2.38 |
| 30 | 6 | 25604 | 1405 | 10252 | 63943 | 2.50 |
| 60 | 6 | 20980 | 1262 | 10073 | 43695 | 2.08 |

TABLE 2
The effect of extraction time (30 min vs 60 min) on molecular
weight of silk processed under the conditions of 100°
C. Extraction Temperature, boiling Lithium Bromide (LiBr)
and 60 ° C. Oven Dissolution for 4 hr.

| | Boil | Average | Std | Confidence | | |
|---|---|---|---|---|---|---|
| Sample | Time | Mw | dev | Interval | | PD |
| 30 min, 4 hr | 30 | 49656 | 4580 | 17306 | 142478 | 2.87 |
| 60 min, 4 hr | 60 | 30042 | 1536 | 11183 | 80705 | 2.69 |

TABLE 3
The effect of extraction time (30 min vs 60 min) on molecular
weight of silk processed under the conditions of 100°
C. Extraction Temperature, 60° C. Lithium Bromide (LiBr)
and 60 ° C. Oven Dissolution (Oven/Dissolution Time was varied).

| | Boil | Oven | Average | Std | Confidence | | |
|---|---|---|---|---|---|---|---|
| Sample | Time | Time | Mw | dev | Interval | | PD |
| 30 min, 1 hr | 30 | 1 | 58436 | | 22201 | 153809 | 2.63 |
| 60 min, 1 hr | 60 | 1 | 31700 | | 11931 | 84224 | 2.66 |
| 30 min, 4 hr | 30 | 4 | 61956.5 | 13337 | 21463 | 178847 | 2.89 |
| 60 min, 4 hr | 60 | 4 | 25578.5 | 2446 | 9979 | 65564 | 2.56 |

TABLE 4
The effect of extraction time (30 min vs 60 min) on
molecular weight of silk processed under the conditions
of 100° C. Extraction Temperature, 80° C.
Lithium Bromide (LiBr) and 80 ° C. Oven Dissolution for 6 hr.

| | Boil | Average | Std | Confidence | | |
|---|---|---|---|---|---|---|
| Sample | Time | Mw | dev | Interval | | PD |
| 30 min, 6 hr | 30 | 63510 | | 18693 | 215775 | 3.40 |
| 60 min, 6 hr | 60 | 25164 | 238 | 9637 | 65706 | 2.61 |

TABLE 5
The effect of extraction time (30 min vs 60 min) on molecular
weight of silk processed under the conditions of 100°
C. Extraction Temperature, 80° C. Lithium Bromide (LiBr)
and 60 ° C. Oven Dissolution (Oven/Dissolution Time was varied).

| | Boil | Oven | Average | Std | Confidence | | |
|---|---|---|---|---|---|---|---|
| Sample | Time | Time | Mw | dev | Interval | | PD |
| 30 min, 4 hr | 30 | 4 | 59202 | 14028 | 19073 | 183760 | 3.10 |
| 60 min, 4 hr | 60 | 4 | 26312.5 | 637 | 10266 | 67442 | 2.56 |
| 30 min, 6 hr | 30 | 6 | 46824 | | 18076 | 121293 | 2.59 |
| 60 min, 6 hr | 60 | 6 | 26353 | | 10168 | 68302 | 2.59 |

TABLE 6
The effect of extraction time (30 min vs 60 min) on molecular
weight of silk processed under the conditions of 100°
C. Extraction Temperature, 140° C. Lithium Bromide (LiBr)
and 140° C. Oven Dissolution (Oven/Dissolution Time was varied).

| | Boil | Oven | Average | Std | Confidence | | |
|---|---|---|---|---|---|---|---|
| Sample | Time | Time | Mw | dev | Interval | | PD |
| 30 min, 4 hr | 30 | 4 | 9024.5 | 1102 | 4493 | 18127 | 2.00865 |
| 60 min, 4 hr | 60 | 4 | 15548 | | 6954 | 34762 | 2.2358 |
| 30 min, 6 hr | 30 | 6 | 13021 | | 5987 | 28319 | 2.1749 |
| 60 min, 6 hr | 60 | 6 | 10888 | | 5364 | 22100 | 2.0298 |

Experiments were carried out to determine the effect of varying the extraction temperature. Table 7 summarizes the results. Below is a summary:
Sericin extraction at 90° C. resulted in higher MW than sericin extraction at 100° C. extraction
Both 90° C. and 100° C. show decreasing MW over time in the oven.

TABLE 7
The effect of extraction temperature (90° C. vs.
100° C.) on molecular weight of silk processed
under the conditions of 60 min. Extraction Temperature,
100° C. Lithium Bromide (LiBr) and 100° C.
Oven Dissolution (Oven/Dissolution Time was varied).

| | Boil | Oven | Average | Std | Confidence | | |
|---|---|---|---|---|---|---|---|
| Sample | Time | Time | Mw | dev | Interval | | PD |
| 90° C., 4 hr | 60 | 4 | 37308 | 4204 | 13368 | 104119 | 2.79 |
| 100° C., 4 hr | 60 | 4 | 25082 | 1248 | 10520 | 59804 | 2.38 |
| 90° C., 6 hr | 60 | 6 | 34224 | 1135 | 12717 | 92100 | 2.69 |
| 100° C., 6 hr | 60 | 6 | 20980 | 1262 | 10073 | 43694 | 2.08 |

Experiments were carried out to determine the effect of varying the Lithium Bromide (LiBr) temperature when added to silk. Tables 8-9 summarize the results. Below is a summary:
No impact on molecular weight or confidence interval (all CI ˜10500-6500 Da)
Studies illustrated that the temperature of LiBr-silk dissolution, as LiBr is added and begins dissolving, rapidly drops below the original LiBr temperature due to the majority of the mass being silk at room temperature

TABLE 8
The effect of Lithium Bromide (LiBr) temperature on molecular
weight of silk processed under the conditions of 60 min.
Extraction Time., 100° C. Extraction Temperature and
60° C. Oven Dissolution (Oven/Dissolution Time was varied).

| | LiBr | | | | | | |
|---|---|---|---|---|---|---|---|
| | Temp | Oven | Average | Std | Confidence | | |
| Sample | (° C.) | Time | Mw | dev | Interval | | PD |
| 60° C. LiBr, | 60 | 1 | 31700 | | 11931 | 84223 | 2.66 |
| 1 hr | | | | | | | |
| 100° C. LiBr, | 100 | 1 | 27907 | 200 | 10735 | 72552 | 2.60 |
| 1 hr | | | | | | | |
| RT LiBr, | RT | 4 | 29217 | 1082 | 10789 | 79119 | 2.71 |
| 4 hr | | | | | | | |
| 60° C. LiBr, | 60 | 4 | 25578 | 2445 | 9978 | 65564 | 2.56 |
| 4 hr | | | | | | | |
| 80° C. LiBr, | 80 | 4 | 26312 | 637 | 10265 | 67441 | 2.56 |
| 4 hr | | | | | | | |
| 100° C. LiBr, | 100 | 4 | 27681 | 1729 | 11279 | 67931 | 2.45 |
| 4 hr | | | | | | | |
| Boil LiBr, | Boil | 4 | 30042 | 1535 | 11183 | 80704 | 2.69 |
| 4 hr | | | | | | | |
| RT LiBr, | RT | 6 | 26543 | 1893 | 10783 | 65332 | 2.46 |
| 6 hr | | | | | | | |
| 80° C. LiBr, | 80 | 6 | 26353 | | 10167 | 68301 | 2.59 |
| 6 hr | | | | | | | |
| 100° C. LiBr, | 100 | 6 | 27150 | 916 | 11020 | 66889 | 2.46 |
| 6 hr | | | | | | | |

TABLE 9
The effect of Lithium Bromide (LiBr) temperature on molecular
weight of silk processed under the conditions of 30 min.
Extraction Time, 100° C. Extraction Temperature and
60° C. Oven Dissolution (Oven/Dissolution Time was varied).

| | LiBr | | | | | | |
|---|---|---|---|---|---|---|---|
| | Temp | Oven | Average | Std | Confidence | | |
| Sample | (° C.) | Time | Mw | dev | Interval | | PD |
| 60° C. LiBr, | 60 | 4 | 61956 | 13336 | 21463 | 178847 | 2.89 |
| 4 hr | | | | | | | |
| 80° C. LiBr, | 80 | 4 | 59202 | 14027 | 19073 | 183760 | 3.10 |
| 4 hr | | | | | | | |
| 100° C. | 100 | 4 | 47853 | | 19757 | 115899 | 2.42 |
| LiBr, 4 hr | | | | | | | |
| 80° C. LiBr, | 80 | 6 | 46824 | | 18075 | 121292 | 2.59 |
| 6 hr | | | | | | | |
| 100° C. | 100 | 6 | 55421 | 8991 | 19152 | 160366 | 2.89 |
| LiBr, 6 hr | | | | | | | |

Experiments were carried out to determine the effect of v oven/dissolution temperature. Tables 10-14 summarize the results. Below is a summary:
Oven temperature has less of an effect on 60 min extracted silk than 30 min extracted silk. Without wishing to be bound by theory, it is believed that the 30 min silk is less degraded during extraction and therefore the oven temperature has more of an effect on the larger MW, less degraded portion of the silk.
For 60° C. vs. 140° C. oven the 30 min extracted silk showed a very significant effect of lower MW at higher oven temp, while 60 min extracted silk had an effect but much less
The 140° C. oven resulted in a low end in the confidence interval at ˜6000 Da.

TABLE 10
The effect of oven/dissolution temperature on molecular weight
of silk processed under the conditions of 100° C. Extraction
Temperature, 30 min. Extraction Time, and 100° C. Lithium
Bromide (LiBr) (Oven/Dissolution Time was varied).

| | Oven | | | | | | |
|---|---|---|---|---|---|---|---|
| Boil | Temp | Oven | Average | Std | Confidence | | |
| Time | (° C.) | Time | Mw | dev | Interval | | PD |
| 30 | 60 | 4 | 47853 | | 19758 | 115900 | 2.42 |
| 30 | 100 | 4 | 40973 | 2632 | 14268 | 117658 | 2.87 |
| 30 | 60 | 6 | 55421 | 8992 | 19153 | 160366 | 2.89 |
| 30 | 100 | 6 | 25604 | 1405 | 10252 | 63943 | 2.50 |

TABLE 11
The effect of oven/dissolution temperature on molecular weight
of silk processed under the conditions of 100° C. Extraction
Temperature, 60 min. Extraction Time, and 100° C. Lithium
Bromide (LiBr) (Oven/Dissolution Time was varied).

| Boil | | | | | | | |
|---|---|---|---|---|---|---|---|
| Time | Oven | Oven | Average | Std | Confidence | | |
| (minutes) | Temp | Time | Mw | dev | Interval | | PD |
| 60 | 60 | 1 | 27908 | 200 | 10735 | 72552 | 2.60 |
| 60 | 100 | 1 | 31520 | 1387 | 11633 | 85407 | 2.71 |
| 60 | 60 | 4 | 27681 | 1730 | 11279 | 72552 | 2.62 |
| 60 | 100 | 4 | 25082 | 1248 | 10520 | 59803 | 2.38 |
| 60 | 60 | 6 | 27150 | 916 | 11020 | 66889 | 2.46 |
| 60 | 100 | 6 | 20980 | 1262 | 10073 | 43695 | 2.08 |

TABLE 12
The effect of oven/dissolution temperature on molecular weight
of silk processed under the conditions of 100° C. Extraction
Temperature, 60 min. Extraction Time, and 140° C. Lithium
Bromide (LiBr) (Oven/Dissolution Time was varied).

| Boil | Oven | | | | | | |
|---|---|---|---|---|---|---|---|
| Time | Temp | Oven | | Std | Confidence | | |
| (minutes) | (° C.) | Time | Average | dev | Interval | | PD |
| 60 | 60 | 4 | 30042 | 1536 | 11183 | 80705 | 2.69 |
| 60 | 140 | 4 | 15548 | | 7255 | 33322 | 2.14 |

TABLE 13
The effect of oven/dissolution temperature on molecular weight
of silk processed under the conditions of 100° C. Extraction
Temperature, 30 min. Extraction Time, and 140° C. Lithium
Bromide (LiBr) (Oven/Dissolution Time was varied).

| Boil | Oven | | | | | | |
|---|---|---|---|---|---|---|---|
| Time | Temp | Oven | Average | Std | Confidence | | |
| (minutes) | (° C.) | Time | Mw | dev | Interval | | PD |
| 30 | 60 | 4 | 49656 | 4580 | 17306 | 142478 | 2.87 |
| 30 | 140 | 4 | 9025 | 1102 | 4493 | 18127 | 2.01 |
| 30 | 60 | 6 | 59383 | 11640 | 17641 | 199889 | 3.37 |
| 30 | 140 | 6 | 13021 | | 5987 | 28319 | 2.17 |

TABLE 14
The effect of oven/dissolution temperature on molecular weight
of silk processed under the conditions of 100° C. Extraction
Temperature, 60 min. Extraction Time, and 80° C. Lithium
Bromide (LiBr) (Oven/Dissolution Time was varied).

| Boil | Oven | | | | | | |
|---|---|---|---|---|---|---|---|
| Time | Temp | Oven | Average | Std | Confidence | | |
| (minutes) | (° C.) | Time | Mw | dev | Interval | | PD |
| 60 | 60 | 4 | 26313 | 637 | 10266 | 67442 | 2.56 |
| 60 | 80 | 4 | 30308 | 4293 | 12279 | 74806 | 2.47 |
| 60 | 60 | 6 | 26353 | | 10168 | 68302 | 2.59 |
| 60 | 80 | 6 | 25164 | 238 | 9637 | 65706 | 2.61 |

The raw silk cocoons from the silkworm Bombyx mori was cut into pieces. The pieces of raw silk cocoons were boiled in an aqueous solution of Na₂CO₃ (about 100° C.) for a period of time between about 30 minutes to about 60 minutes to remove sericin (degumming). The volume of the water used equals about 0.4×raw silk weight and the amount of Na₂CO₃ is about 0.848× the weight of the raw silk cocoon pieces. The resulting degummed silk cocoon pieces were rinsed with deionized water three times at about 60° C. (20 minutes per rinse). The volume of rinse water for each cycle was 0.2 L× the weight of the raw silk cocoon pieces. The excess water from the degummed silk cocoon pieces was removed. After the DI water washing step, the wet degummed silk cocoon pieces were dried at room temperature. The degummed silk cocoon pieces were mixed with a LiBr solution, and the mixture was heated to about 100° C. The warmed mixture was placed in a dry oven and was heated at a temperature ranging from about 60° C. to about 140° C. for about 60 minutes to achieve complete dissolution of the native silk protein. The resulting solution was allowed to cool to room temperature and then was dialyzed to remove LiBr salts using a 3,500 Da MWCO membrane. Multiple exchanges were performed in Di water until Br ions were less than 1 ppm as determined in the hydrolyzed fibroin solution read on an Oakton Bromide (Br⁻) double-junction ion-selective electrode.

The resulting silk fibroin aqueous solution has a concentration of about 8.0% w/v containing pure silk fibroin protein fragments having an average weight average molecular weight ranging from about 6 kDa to about 16 kDa, about 17 kDa to about 39 kDa, and about 39 kDa to about 80 kDa and a polydispersity of between about 1.5 and about 3.0. The 8.0% w/v was diluted with DI water to provide a 1.0% w/v, 2.0% w/v, 3.0% w/v, 4.0% w/v, 5.0% w/v by the coating solution.

A variety of % silk concentrations have been produced through the use of Tangential Flow Filtration (TFF). In all cases a 1% silk solution was used as the input feed. A range of 750-18,000 mL of 1% silk solution was used as the starting volume. Solution is diafiltered in the TFF to remove lithium bromide. Once below a specified level of residual LiBr, solution undergoes ultrafiltration to increase the concentration through removal of water. See examples below.

Six (6) silk solutions were utilized in standard silk structures with the following results:

Solution #1 is a silk concentration of 5.9 wt. %, average MW of 19.8 kDa and 2.2 PDI (made with a 60 min boil extraction, 100° C. LiBr dissolution for 1 hour).

Solution #2 is a silk concentration of 6.4 wt. % (made with a 30 min boil extraction, 60° C. LiBr dissolution for 4 hrs).

Solution #3 is a silk concentration of 6.17 wt. % (made with a 30 min boil extraction 100° C. LiBr dissolution for 1 hour).

Solution #4 is a silk concentration of 7.30 wt. %: A 7.30% silk solution was produced beginning with 30 minute extraction batches of 100 g silk cocoons per batch. Extracted silk fibers were then dissolved using 100° C. 9.3 M LiBr in a 100° C. oven for 1 hour. 100 g of silk fibers were dissolved per batch to create 20% silk in LiBr. Dissolved silk in LiBr was then diluted to 1% silk and filtered through a 5 μm filter to remove large debris. 15,500 mL of 1%, filtered silk solution was used as the starting volume/diafiltration volume for TFF. Once LiBr was removed, the solution was ultrafiltered to a volume around 1300 mL. 1262 mL of 7.30% silk was then collected. Water was added to the feed to help remove the remaining solution and 547 mL of 3.91% silk was then collected.

Solution #5 is a silk concentration of 6.44 wt. %: A 6.44 wt. % silk solution was produced beginning with 60 minute extraction batches of a mix of 25, 33, 50, 75 and 100 g silk cocoons per batch. Extracted silk fibers were then dissolved using 100° C. 9.3 M LiBr in a 100° C. oven for 1 hour. 35, 42, 50 and 71 g per batch of silk fibers were dissolved to create 20% silk in LiBr and combined. Dissolved silk in LiBr was then diluted to 1% silk and filtered through a 5 μm filter to remove large debris. 17,000 mL of 1%, filtered silk solution was used as the starting volume/diafiltration volume for TFF. Once LiBr was removed, the solution was ultrafiltered to a volume around 3000 mL. 1490 mL of 6.44% silk was then collected. Water was added to the feed to help remove the remaining solution and 1454 mL of 4.88% silk was then collected.

Solution #6 is a silk concentration of 2.70 wt. %: A 2.70% silk solution was produced beginning with 60-minute extraction batches of 25 g silk cocoons per batch. Extracted silk fibers were then dissolved using 100° C. 9.3 M LiBr in a 100° C. oven for 1 hour. 35.48 g of silk fibers were dissolved per batch to create 20% silk in LiBr. Dissolved silk in LiBr was then diluted to 1% silk and filtered through a 5 μm filter to remove large debris. 1000 mL of 1%, filtered silk solution was used as the starting volume/diafiltration volume for TFF. Once LiBr was removed, the solution was ultrafiltered to a volume around 300 mL. 312 mL of 2.7% silk was then collected.

The preparation of silk fibroin solutions with higher molecular weights is given in Table 15.

TABLE 15
Preparation and properties of silk fibroin solutions.

| | | | | | | Average | |
|---|---|---|---|---|---|---|---|
| | | | | | | weight | |
| | | | | | | average | |
| | Extrac- | Extrac- | | | | molec- | Average |
| | tion | tion | LiBr | | | ular | poly- |
| Sample | Time | Temp | Temp | Oven/Sol'n | | weight | dispers- |
| Name | (mins) | (° C.) | (° C.) | Temp | | (kDa) | ity |
| Group A | 60 | 100 | 100 | 100° | C. oven | 34.7 | 2.94 |
| TFF | | | | | | | |
| Group A | 60 | 100 | 100 | 100° | C. oven | 44.7 | 3.17 |
| DIS | | | | | | | |
| Group B | 60 | 100 | 100 | 100° | C. sol'n | 41.6 | 3.07 |
| TFF | | | | | | | |
| Group B | 60 | 100 | 100 | 100° | C. sol'n | 44.0 | 3.12 |
| DIS | | | | | | | |
| Group D | 30 | 90 | 60 | 60° | C. sol'n | 129.7 | 2.56 |
| DIS | | | | | | | |
| Group D | 30 | 90 | 60 | 60° | C. sol'n | 144.2 | 2.73 |
| FIL | | | | | | | |
| Group E | 15 | 100 | RT | 60° | C. sol'n | 108.8 | 2.78 |
| DIS | | | | | | | |
| Group E | 15 | 100 | RT | 60° | C. sol'n | 94.8 | 2.62 |
| FIL | | | | | | | |

Silk aqueous coating composition for application to fabrics are given in Tables 16 and 17 below.

TABLE 16
Silk Solution Characteristics

| | Molecular Weight: | 57 | kDa | | | |
|---|---|---|---|---|---|---|
| | Polydispersity: | 1.6 | | | | |
| | % Silk | 5.0% | | 3.0% | 1.0% | 0.5% |
| Process | Extraction | | | | | |
| Parameters | Boil Time: | 30 | minutes | | | |
| | Boil Temperature: | 100° | C. | | | |
| | Rinse Temperature: | 60° | C. | | | |
| | Dissolution | | | | | |
| | LiBr Temperature: | 100 | | | | |
| | Oven Temperature: | 100° | C. | | | |
| | Oven Time: | 60 | minutes | | | |

TABLE 17
Silk Solution Characteristics

| | Molecular Weight: | 25 | kDa | | | |
|---|---|---|---|---|---|---|
| | Polydispersity: | 2.4 | | | | |
| | % Silk | 5.0% | | 3.0% | 1.0% | 0.5% |
| Process | Extraction | | | | | |
| Parameters | Boil Time: | 60 | minutes | | | |
| | Boil Temperature: | 100° | C. | | | |
| | Rinse Temperature: | 60° | C. | | | |
| | Dissolution | | | | | |
| | LiBr Temperature: | 100° | C. | | | |
| | Oven Temperature: | 100° | C. | | | |
| | Oven Time: | 60 | minutes | | | |

Three (3) silk solutions were utilized in film making with the following results:

Solution #1 is a silk concentration of 5.9%, average MW of 19.8 kDa and 2.2 PD (made with a 60 min boil extraction, 100° C. LiBr dissolution for 1 hr).

Solution #2 is a silk concentration of 6.4% (made with a 30 min boil extraction, 60° C. LiBr dissolution for 4 hrs).

Solution #3 is a silk concentration of 6.17% (made with a 30 min boil extraction, 100° C. LiBr dissolution for 1 hour).

Films were made in accordance with Rockwood et al. (Nature Protocols; Vol. 6; No. 10; published on-line Sep. 22, 2011; doi:10.1038/nprot.2011.379). 4 mL of 1% or 2% (wt/vol) aqueous silk solution was added into 100 mm Petri dish (Volume of silk can be varied for thicker or thinner films and is not critical) and allowed to dry overnight uncovered. The bottom of a vacuum desiccator was filled with water. Dry films were placed in the desiccator and vacuum applied, allowing the films to water anneal for 4 hours prior to removal from the dish. Films cast from solution #1 did not result in a structurally continuous film; the film was cracked in several pieces. These pieces of film dissolved in water in spite of the water annealing treatment.

Silk solutions of various molecular weights and/or combinations of molecular weights can be optimized for gel applications. The following provides an example of this process but it not intended to be limiting in application or formulation. Three (3) silk solutions were utilized in gel making with the following results:

Solution #1 is a silk concentration of 5.9%, average MW of 19.8 kDa and 2.2 PD (made with a 60 min boil extraction, 100° C. LiBr dissolution for 1 hr).

Solution #2 is a silk concentration of 6.4% (made with a 30 min boil extraction, 60° C. LiBr dissolution for 4 hrs).

Solution #3 is a silk concentration of 6.17% (made with a 30 min boil extraction, 100° C. LiBr dissolution for 1 hour).

“Egel” is an electrogelation process as described in Rockwood of al. Briefly, 10 ml of aqueous silk solution is added to a 50 ml conical tube and a pair of platinum wire electrodes immersed into the silk solution. A 20 volt potential was applied to the platinum electrodes for 5 minutes, the power supply turned off and the gel collected. Solution #1 did not form an EGEL over the 5 minutes of applied electric current.

Solutions #2 and #3 were gelled in accordance with the published horseradish peroxidase (HRP) protocol. Behavior seemed typical of published solutions.

Materials and Methods: the following equipment and material are used in determination of Silk Molecular weight: Agilent 1100 with chemstation software ver. 10.01; Refractive Index Detector (RID); analytical balance; volumetric flasks (1000 mL, 10 mL and 5 mL); HPLC grade water; ACS grade sodium chloride; ACS grade sodium phosphate dibasic heptahydrate; phosphoric acid; dextran MW Standards-Nominal Molecular Weights of 5 kDa, 11.6 kDa, 23.8 kDa, 48.6 kDa, and 148 kDa; 50 mL PET or polypropylene disposable centrifuge tubes; graduated pipettes; amber glass HPLC vials with Teflon caps; Phenomenex PolySep GFC P-4000 column (size: 7.8 mm×300 mm).

Procedural Steps:

### A) Preparation of 1 L Mobile Phase (0.1 M Sodium Chloride Solution in 0.0125 M Sodium Phosphate Buffer)

Take a 250 mL clean and dry beaker, place it on the balance and tare the weight. Add about 3.3509 g of sodium phosphate dibasic heptahydrate to the beaker. Note down the exact weight of sodium phosphate dibasic weighed. Dissolve the weighed sodium phosphate by adding 100 mL of HPLC water into the beaker. Take care not to spill any of the content of the beaker. Transfer the solution carefully into a clean and dry 1000 mL volumetric flask. Rinse the beaker and transfer the rinse into the volumetric flask. Repeat the rinse 4-5 times. In a separate clean and dry 250 mL beaker weigh exactly about 5.8440 g of sodium chloride. Dissolve the weighed sodium chloride in 50 mL of water and transfer the solution to the sodium phosphate solution in the volumetric flask. Rinse the beaker and transfer the rinse into the volumetric flask. Adjust the pH of the solution to 7.0±0.2 with phosphoric acid. Make up the volume in volumetric flask with HPLC water to 1000 mL and shake it vigorously to homogeneously mix the solution. Filter the solution through 0.45 μm polyamide membrane filter. Transfer the solution to a clean and dry solvent bottle and label the bottle. The volume of the solution can be varied to the requirement by correspondingly varying the amount of sodium phosphate dibasic heptahydrate and sodium chloride.

### B) Preparation of Dextran Molecular Weight Standard Solutions

At least five different molecular weight standards are used for each batch of samples that are run so that the expected value of the sample to be tested is bracketed by the value of the standard used. Label six 20 mL scintillation glass vials respective to the molecular weight standards. Weigh accurately about 5 mg of each of dextran molecular weight standards and record the weights. Dissolve the dextran molecular weight standards in 5 mL of mobile phase to make a 1 mg/mL standard solution.

### C) Preparation of Sample Solutions

When preparing sample solutions, if there are limitations on how much sample is available, the preparations may be scaled as long as the ratios are maintained. Depending on sample type and silk protein content in sample weigh enough sample in a 50 mL disposable centrifuge tube on an analytical balance to make a 1 mg/mL sample solution for analysis. Dissolve the sample in equivalent volume of mobile phase make a 1 mg/mL solution. Tightly cap the tubes and mix the samples (in solution). Leave the sample solution for 30 minutes at room temperature. Gently mix the sample solution again for 1 minute and centrifuge at 4000 RPM for 10 minutes.

### D) HPLC Analysis of the Samples

Transfer 1.0 mL of all the standards and sample solutions into individual HPLC vials. Inject the molecular weight standards (one injection each) and each sample in duplicate. Analyze all the standards and sample solutions using the following HPLC conditions:

| | |
|---|---|
| Column | PolySep GFC P-4000 (7.8 × 300 mm) |
| Column Temperature | 25° C. |
| Detector | Refractive Index Detector (Temperature @ 35° |
| | C.) |
| Injection Volume | 25.0 μL |
| Mobile Phase | 0.1M Sodium Chloride solution in 0.0125M |
| | sodium phosphate buffer |
| Flow Rate | 1.0 mL/min |
| Run Time | 20.0 min |

### E) Data Analysis and Calculations—Calculation of Average Molecular Weight Using Cirrus Software

Upload the chromatography data files of the standards and the analytical samples into Cirrus SEC data collection and molecular weight analysis software. Calculate the weight average molecular weight (M_{w}), number average molecular weight (Mₙ), peak average molecular weight (Mₚ), and polydispersity for each injection of the sample.

Spider Silk Fragments

Spider silks are natural polymers that consist of three domains: a repetitive middle core domain that dominates the protein chain, and non-repetitive N-terminal and C-terminal domains. The large core domain is organized in a block copolymer-like arrangement, in which two basic sequences, crystalline [poly(A) or poly(GA)] and less crystalline (GGX or GPGXX) polypeptides alternate. Dragline silk is the protein complex composed of major ampullate dragline silk protein 1 (MaSp1) and major ampullate dragline silk protein 2 (MaSp2). Both silks are approximately 3500 amino acid long. MaSp1 can be found in the fibre core and the periphery, whereas MaSp2 forms clusters in certain core areas. The large central domains of MaSp1 and MaSp2 are organized in block copolymer-like arrangements, in which two basic sequences, crystalline [poly(A) or poly(GA)] and less crystalline (GGX or GPGXX) polypeptides alternate in core domain. Specific secondary structures have been assigned to poly(A)/(GA), GGX and GPGXX motifs including β-sheet, α-helix and β-spiral respectively. The primary sequence, composition and secondary structural elements of the repetitive core domain are responsible for mechanical properties of spider silks; whereas, non-repetitive N- and C-terminal domains are essential for the storage of liquid silk dope in a lumen and fibre formation in a spinning duct.

The main difference between MaSp1 and MaSp2 is the presence of proline (P) residues accounting for 15% of the total amino acid content in MaSp2, whereas MaSp1 is proline-free. By calculating the number of proline residues in N. clavipes dragline silk, it is possible to estimate the presence of the two proteins in fibres; 81% MaSp1 and 19% MaSp2. Different spiders have different ratios of MaSp1 and MaSp2. For example, a dragline silk fibre from the orb weaver Argiope aurantia contains 41% MaSp1 and 59% MaSp2. Such changes in the ratios of major ampullate silks can dictate the performance of the silk fibre.

At least seven different types of silk proteins are known for one orb-weaver species of spider. Silks differ in primary sequence, physical properties and functions. For example, dragline silks used to build frames, radii and lifelines are known for outstanding mechanical properties including strength, toughness and elasticity. On an equal weight basis, spider silk has a higher toughness than steel and Kevlar. Flageliform silk found in capture spirals has extensibility of up to 500%. Minor ampullate silk, which is found in auxiliary spirals of the orb-web and in prey wrapping, possesses high toughness and strength almost similar to major ampullate silks, but does not supercontract in water.

Spider silks are known for their high tensile strength and toughness. The recombinant silk proteins also confer advantageous properties to cosmetic or dermatological compositions, in particular to be able to improve the hydrating or softening action, good film forming property and low surface density. Diverse and unique biomechanical properties together with biocompatibility and a slow rate of degradation make spider silks excellent candidates as biomaterials for tissue engineering, guided tissue repair and drug delivery, for cosmetic products (e.g. nail and hair strengthener, skin care products), and industrial materials (e.g. nanowires, nanofibers, surface coatings).

In an embodiment, a silk protein may include a polypeptide derived from natural spider silk proteins. The polypeptide is not limited particularly as long as it is derived from natural spider silk proteins, and examples of the polypeptide include natural spider silk proteins and recombinant spider silk proteins such as variants, analogs, derivatives or the like of the natural spider silk proteins. In terms of excellent tenacity, the polypeptide may be derived from major dragline silk proteins produced in major ampullate glands of spiders. Examples of the major dragline silk proteins include major ampullate spidroin MaSp1 and MaSp2 from Nephila clavipes, and ADF3 and ADF4 from Araneus diadematus, etc. Examples of the polypeptide derived from major dragline silk proteins include variants, analogs, derivatives or the like of the major dragline silk proteins. Further, the polypeptide may be derived from flagelliform silk proteins produced in flagelliform glands of spiders. Examples of the flagelliform silk proteins include flagelliform silk proteins derived from Nephila clavipes, etc.

Examples of the polypeptide derived from major dragline silk proteins include a polypeptide containing two or more units of an amino acid sequence represented by the formula 1: REP1-REP2 (1), preferably a polypeptide containing five or more units thereof, and more preferably a polypeptide containing ten or more units thereof. Alternatively, the polypeptide derived from major dragline silk proteins may be a polypeptide that contains units of the amino acid sequence represented by the formula 1: REP1-REP2 (1) and that has, at a C-terminal, an amino acid sequence represented by any of SEQ ID NOS: 1 to 3 of U.S. Pat. No. 9,051,453 or an amino acid sequence having a homology of 90% or more with the amino acid sequence represented by any of SEQ ID NOS: 1 to 3 of U.S. Pat. No. 9,051,453. In the polypeptide derived from major dragline silk proteins, units of the amino acid sequence represented by the formula 1: REP1-REP2 (1) may be the same or may be different from each other. In the case of producing a recombinant protein using a microbe such as Escherichia coli as a host, the molecular weight of the polypeptide derived from major dragline silk proteins is 500 kDa or less, or 300 kDa or less, or 200 kDa or less, in terms of productivity.

In the formula (1), the REP1 indicates polyalanine. In the REP1, the number of alanine residues arranged in succession is preferably 2 or more, more preferably 3 or more, further preferably 4 or more, and particularly preferably 5 or more. Further, in the REP1, the number of alanine residues arranged in succession is preferably 20 or less, more preferably 16 or less, further preferably 12 or less, and particularly preferably 10 or less. In the formula (1), the REP2 is an amino acid sequence composed of 10 to 200 amino acid residues. The total number of glycine, serine, glutamine and alanine residues contained in the amino acid sequence is 40% or more, preferably 60% or more, and more preferably 70% or more with respect to the total number of amino acid residues contained therein.

In the major dragline silk, the REP1 corresponds to a crystal region in a fiber where a crystal β sheet is formed, and the REP2 corresponds to an amorphous region in a fiber where most of the parts lack regular configurations and that has more flexibility. Further, the [REP1-REP2] corresponds to a repetitious region (repetitive sequence) composed of the crystal region and the amorphous region, which is a characteristic sequence of dragline silk proteins.

Recombinant Silk Fragments

In some embodiments, the recombinant silk protein refers to recombinant spider silk polypeptides, recombinant insect silk polypeptides, or recombinant mussel silk polypeptides. In some embodiments, the recombinant silk protein fragment disclosed herein include recombinant spider silk polypeptides of Araneidae or Araneoids, or recombinant insect silk polypeptides of Bombyx mori. In some embodiments, the recombinant silk protein fragment disclosed herein include recombinant spider silk polypeptides of Araneidae or Araneoids. In some embodiments, the recombinant silk protein fragment disclosed herein include block copolymer having repetitive units derived from natural spider silk polypeptides of Araneidae or Araneoids. In some embodiments, the recombinant silk protein fragment disclosed herein include block copolymer having synthetic repetitive units derived from spider silk polypeptides of Araneidae or Araneoids and non-repetitive units derived from natural repetitive units of spider silk polypeptides of Araneidae or Araneoids.

Recent advances in genetic engineering have provided a route to produce various types of recombinant silk proteins. Recombinant DNA technology has been used to provide a more practical source of silk proteins. As used herein “recombinant silk protein” refers to synthetic proteins produced heterologously in prokaryotic or eukaryotic expression systems using genetic engineering methods.

Various methods for synthesizing recombinant silk peptides are known and have been described by Ausubel et al., Current Protocols in Molecular Biology § 8 (John Wiley & Sons 1987, (1990)), incorporated herein by reference. A gram-negative, rod-shaped bacterium E. coli is a well-established host for industrial scale production of proteins. Therefore, the majority of recombinant silks have been produced in E. coli. E. coli which is easy to manipulate, has a short generation time, is relatively low cost and can be scaled up for larger amounts protein production.

The recombinant silk proteins can be produced by transformed prokaryotic or eukaryotic systems containing the cDNA coding for a silk protein, for a fragment of this protein or for an analog of such a protein. The recombinant DNA approach enables the production of recombinant silks with programmed sequences, secondary structures, architectures and precise molecular weight. There are four main steps in the process: (i) design and assembly of synthetic silk-like genes into genetic ‘cassettes’, (ii) insertion of this segment into a DNA recombinant vector, (iii) transformation of this recombinant DNA molecule into a host cell and (iv) expression and purification of the selected clones.

The term “recombinant vectors”, as used herein, includes any vectors known to the skilled person including plasmid vectors, cosmid vectors, phage vectors such as lambda phage, viral vectors such as adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, or plant) or in in vitro expression systems. Cloning vectors are generally used to engineer and amplify a certain desired DNA fragment and may lack functional sequences needed for expression of the desired DNA fragments.

The prokaryotic systems include Gram-negative bacteria or Gram-positive bacteria. The prokaryotic expression vectors can include an origin of replication which can be recognized by the host organism, a homologous or heterologous promoter which is functional in the said host, the DNA sequence coding for the spider silk protein, for a fragment of this protein or for an analogous protein. Nonlimiting examples of prokaryotic expression organisms are Escherichia coli, Bacillus subtilis, Bacillus megaterium, Corynebacterium glutamicum, Anabaena, Caulobacter, Gluconobacter, Rhodobacter, Pseudomonas, Para coccus, Bacillus (e.g. Bacillus subtilis) Brevibacterium, Corynebacterium, Rhizobium (Sinorhizobium), Flavobacterium, Klebsiella, Enterobacter, Lactobacillus, Lactococcus, Methylobacterium, Propionibacterium, Staphylococcus or Streptomyces cells.

The eukaryotic systems include yeasts and insect, mammalian or plant cells. In this case, the expression vectors can include a yeast plasmid origin of replication or an autonomous replication sequence, a promoter, a DNA sequence coding for a spider silk protein, for a fragment or for an analogous protein, a polyadenylation sequence, a transcription termination site and, lastly, a selection gene. Nonlimiting examples of eukaryotic expression organisms include yeasts, such as Saccharomyces cerevisiae, Pichia pastoris, basidiosporogenous, ascosporogenous, filamentous fungi, such as Aspergillus niger, Aspergillus oryzae, Aspergillus nidulans, Trichoderma reesei, Acremonium chrysogenum, Candida, Hansenula, Kluyveromyces, Saccharomyces (e.g. Saccharomyces cerevisiae), Schizosaccharomyces, Pichia (e.g. Pichia pastoris) or Yarrowia cells etc., mammalian cells, such as HeLa cells, COS cells, CHO cells etc., insect cells, such as Sf9 cells, MEL cells, etc., “insect host cells” such as Spodoptera frugiperda or Trichoplusia ni cells. SF9 cells, SF-21 cells or High-Five cells, wherein SF-9 and SF-21 are ovarian cells from Spodoptera frugiperda, and High-Five cells are egg cells from Trichoplusia ni., “plant host cells”, such as tobacco, potato or pea cells.

A variety of heterologous host systems have been explored to produce different types of recombinant silks. Recombinant partial spidroins as well as engineered silks have been cloned and expressed in bacteria (Escherichia coli), yeast (Pichia pastoris), insects (silkworm larvae), plants (tobacco, soybean, potato, Arabidopsis), mammalian cell lines (BHT/hamster) and transgenic animals (mice, goats). Most of the silk proteins are produced with an N- or C-terminal His-tags to make purification simple and produce enough amounts of the protein.

In some embodiments, the host suitable for expressing the recombinant spider silk protein using heterogeneous system may include transgenic animals and plants. In some embodiments, the host suitable for expressing the recombinant spider silk protein using heterogeneous system comprises bacteria, yeasts, mammalian cell lines. In some embodiments, the host suitable for expressing the recombinant spider silk protein using heterogeneous system comprises E. coli. In some embodiments, the host suitable for expressing the recombinant spider silk protein using heterogeneous system comprises transgenic B. mori silkworm generated using genome editing technologies (e.g. CRISPR).

The recombinant silk protein in this disclosure comprises synthetic proteins which are based on repeat units of natural silk proteins. Besides the synthetic repetitive silk protein sequences, these can additionally comprise one or more natural nonrepetitive silk protein sequences.

In some embodiments, “recombinant silk protein” refers to recombinant silkworm silk protein or fragments thereof. The recombinant production of silk fibroin and silk sericin has been reported. A variety of hosts are used for the production including E. coli, Saccharomyces cerevisiae, Pseudomonas sp., Rhodopseudomonas sp., Bacillus sp., and Strepomyces. See EP 0230702, which is incorporate by reference herein by its entirety.

Provided herein also include design and biological-synthesis of silk fibroin protein-like multiblock polymer comprising GAGAGX hexapeptide (X is A, Y, V or S) derived from the repetitive domain of B. mori silk heavy chain (H chain)

In some embodiments, this disclosure provides silk protein-like multiblock polymers derived from the repetitive domain of B. mori silk heavy chain (H chain) comprising the GAGAGS hexapeptide repeating units. The GAGAGS hexapeptide is the core unit of H-chain and plays an important role in the formation of crystalline domains. The silk protein-like multiblock polymers containing the GAGAGS hexapeptide repeating units spontaneously aggregate into β-sheet structures, similar to natural silk fibroin protein, where in the silk protein-like multiblock polymers having any weight average molecular weight described herein.

In some embodiments, this disclosure provides silk-peptide like multiblock copolymers composed of the GAGAGS hexapeptide repetitive fragment derived from H chain of B. mori silk heavy chain and mammalian elastin VPGVG motif produced by E. coli. In some embodiments, this disclosure provides fusion silk fibroin proteins composed of the GAGAGS hexapeptide repetitive fragment derived from H chain of B. mori silk heavy chain and GVGVP produced by E. coli, where in the silk protein-like multiblock polymers having any weight average molecular weight described herein.

In some embodiments, this disclosure provides B. mori silkworm recombinant proteins composed of the (GAGAGS)₁₆ repetitive fragment. In some embodiments, this disclosure provides recombinant proteins composed of the (GAGAGS)₁₆ repetitive fragment and the non-repetitive (GAGAGS)₁₆-F-COOH, (GAGAGS)₁₆-F-F-COOH, (GAGAGS)₁₆-F-F-F-COOH, (GAGAGS)₁₆-F-F-F-F-COOH, (GAGAGS)₁₆-F-F-F-F-F-F-F-F-COOH, (GAGAGS)₁₆-F-F-F-F-F-F-F-F-F-F-F-F-COOH produced by E. coli, where F has the following amino acid sequence SGFGPVANGGSGEASSESDFGSSGFGPVANASSGEASSESDFAG, and where in the silk protein-like multiblock polymers having any weight average molecular weight described herein.

In some embodiments, “recombinant silk protein” refers to recombinant spider silk protein or fragments thereof. The productions of recombinant spider silk proteins based on a partial cDNA clone have been reported. The recombinant spider silk proteins produced as such comprise a portion of the repetitive sequence derived from a dragline spider silk protein, Spidroin 1, from the spider Nephila clavipes. see Xu et al. (Proc. Natl. Acad. Sci. U.S.A., 87:7120-7124 (1990). cDNA clone encoding a portion of the repeating sequence of a second fibroin protein, Spidroin 2, from dragline silk of Nephila clavipes and the recombinant synthesis thereof is described in J. Biol. Chem., 1992, volume 267, pp. 19320-19324. The recombinant synthesis of spider silk proteins including protein fragments and variants of Nephila clavipes from transformed E. coli is described in U.S. Pat. Nos. 5,728,810 and 5,989,894. cDNA clones encoding minor ampullate spider silk proteins and the expression thereof is described in U.S. Pat. Nos. 5,733,771 and 5,756,677. cDNA clone encoding the flagelliform silk protein from an orb-web spinning spider is described in U.S. Pat. No. 5,994,099. U.S. Pat. No. 6,268,169 describes the recombinant synthesis of spider silk like proteins derived from the repeating peptide sequence found in the natural spider dragline of Nephila clavipes by E. coli, Bacillus subtilis, and Pichia pastoris recombinant expression systems. WO 03/020916 describes the cDNA clone encoding and recombinant production of spider spider silk proteins having repeative sequences derived from the major ampullate glands of Nephila madagascariensis, Nephila senegalensis, Tetragnatha kauaiensis, Tetragnatha versicolor, Argiope aurantia, Argiope trifasciata, Gasteracantha mammosa, and Latrodectus geometricus, the flagelliform glands of Argiope trifasciata, the ampullate glands of Dolomedes tenebrosus, two sets of silk glands from Plectreurys tristis, and the silk glands of the mygalomorph Euagrus chisoseus. Each of the above reference is incorporated herein by reference in its entirety.

In some embodiments, the recombinant spider silk protein is a hybrid protein of a spider silk protein and an insect silk protein, a spider silk protein and collagen, a spider silk protein and resilin, or a spider silk protein and keratin. The spider silk repetitive unit comprises or consists of an amino acid sequence of a region that comprises or consists of at least one peptide motif that repetitively occurs within a naturally occurring major ampullate gland polypeptide, such as a dragline spider silk polypeptide, a minor ampullate gland polypeptide, a flagelliform polypeptide, an aggregate spider silk polypeptide, an aciniform spider silk polypeptide or a pyriform spider silk polypeptide.

In some embodiments, the recombinant spider silk protein in this disclosure comprises synthetic spider silk proteins derived from repetitive units of natural spider silk proteins, consensus sequence, and optionally one or more natural non-repetitive spider silk protein sequences. The repeated units of natural spider silk polypeptide may include dragline spider silk polypeptides or flagelliform spider silk polypeptides of Araneidae or Araneoids.

As used herein, the spider silk “repetitive unit” comprises or consists of at least one peptide motif that repetitively occurs within a naturally occurring major ampullate gland polypeptide, such as a dragline spider silk polypeptide, a minor ampullate gland polypeptide, a flagelliform polypeptide, an aggregate spider silk polypeptide, an aciniform spider silk polypeptide or a pyriform spider silk polypeptide. A “repetitive unit” refers to a region which corresponds in amino acid sequence to a region that comprises or consists of at least one peptide motif (e.g. AAAAAA) or GPGQQ) that repetitively occurs within a naturally occurring silk polypeptide (e.g. MaSpI, ADF-3, ADF-4, or Flag) (i.e. identical amino acid sequence) or to an amino acid sequence substantially similar thereto (i.e. variational amino acid sequence). A “repetitive unit” having an amino acid sequence which is “substantially similar” to a corresponding amino acid sequence within a naturally occurring silk polypeptide (i.e. wild-type repetitive unit) is also similar with respect to its properties, e.g. a silk protein comprising the “substantially similar repetitive unit” is still insoluble and retains its insolubility. A “repetitive unit” having an amino acid sequence which is “identical” to the amino acid sequence of a naturally occurring silk polypeptide, for example, can be a portion of a silk polypeptide corresponding to one or more peptide motifs of MaSpI, MaSpII, ADF-3 and/or ADF-4. A “repetitive unit” having an amino acid sequence which is “substantially similar” to the amino acid sequence of a naturally occurring silk polypeptide, for example, can be a portion of a silk polypeptide corresponding to one or more peptide motifs of MaSpI, MaSpII, ADF-3 and/or ADF-4, but having one or more amino acid substitution at specific amino acid positions.

As used herein, the term “consensus peptide sequence” refers to an amino acid sequence which contains amino acids which frequently occur in a certain position (e.g. “G”) and wherein, other amino acids which are not further determined are replaced by the place holder “X”. In some embodiments, the consensus sequence is at least one of (i) GPGXX, wherein X is an amino acid selected from A, S, G, Y, P and Q; (ii) GGX, wherein X is an amino acid selected from Y, P, R, S, A, T, N and Q, preferably Y, P and Q; (iii) Aₓ, wherein x is an integer from 5 to 10.

The consensus peptide sequences GPGXX and GGX, i.e. glycine rich motifs, provide flexibility to the silk polypeptide and thus, to the thread formed from the silk protein containing said motifs. In detail, the iterated GPGXX motif forms turn spiral structures, which imparts elasticity to the silk polypeptide. Major ampullate and flagelliform silks both have a GPGXX motif. The iterated GGX motif is associated with a helical structure having three amino acids per turn and is found in most spider silks. The GGX motif may provide additional elastic properties to the silk. The iterated polyalanine Ax (peptide) motif forms a crystalline β-sheet structure that provides strength to the silk polypeptide, as described for example in WO 03/057727.

In some embodiments, the recombinant spider silk protein in this disclosure comprises two identical repetitive units each comprising at least one, preferably one, amino acid sequence selected from the group consisting of: GGRPSDTYG and GGRPSSSYG derived from Resilin. Resilin is an elastomeric protein found in most arthropods that provides low stiffness and high strength.

As used herein, “non-repetitive units” refers to an amino acid sequence which is “substantially similar” to a corresponding non-repetitive (carboxy terminal) amino acid sequence within a naturally occurring dragline polypeptide (i.e. wild-type non-repetitive (carboxy terminal) unit), preferably within ADF-3 (SEQ ID NO:1), ADF-4 (SEQ ID NO:2), NR3 (SEQ ID NO:41), NR4 (SEQ ID NO:42), ADF-4 of the spider Araneus diadematus as described in U.S. Pat. No. 8,367,803, C16 peptide (spider silk protein eADF4, molecular weight of 47.7 kDa, AMSilk) comprising the 16 repeats of the sequence GSSAAAAAAAASGPGGYGPENQGPSGPGGYGPGGP, an amino acid sequence adapted from the natural sequence of ADF4 from A. diadematus. Non-repetitive ADF-4 and variants thereof display efficient assembly behavior.

Among the synthetic spider silk proteins, the recombinant silk protein in this disclosure comprises in some embodiments the C16-protein having the polypeptide sequence SEQ ID NO: 1 as described in U.S. Pat. No. 8,288,512. Besides the polypeptide sequence shown in SEQ ID NO:1, particularly functional equivalents, functional derivatives and salts of this sequence are also included.

As used herein, “functional equivalents” refers to mutant which, in at least one sequence position of the abovementioned amino acid sequences, have an amino acid other than that specifically mentioned.

In some embodiments, the recombinant spider silk protein in this disclosure comprises, in an effective amount, at least one natural or recombinant silk protein including spider silk protein, corresponding to Spidroin major 1 described by Xu et al., PNAS, USA, 87, 7120, (1990), Spidroin major 2 described by Hinman and Lewis, J. Biol. Chem., 267, 19320, (1922), recombinant spider silk protein as described in U.S. Patent Application No. 2016/0222174 and U.S. Pat. Nos. 9,051,453, 9,617,315, 9,689,089, 8,173,772, 8,642,734, 8,367,803 8,097,583, 8,030,024, 7,754,851, 7,148,039, 7,060,260, or alternatively the minor Spidroins described in patent application WO 95/25165. Each of the above-cited references is incorporated herein by reference in its entirety. Additional recombinant spider silk proteins suitable for the recombinant RSPF of this disclosure include ADF3 and ADF4 from the “Major Ampullate” gland of Araneus diadematus.

Recombinant silk is also described in other patents and patent applications, incorporated by reference herein: US 2004590196, U.S. Pat. No. 7,754,851, US 2007654470, U.S. Pat. No. 7,951,908, US 2010785960, U.S. Pat. No. 8,034,897, US 20090263430, US 2008226854, US 20090123967, US 2005712095, US 2007991037, US 20090162896, US 200885266, U.S. Pat. No. 8,372,436, US 2007989907, US 2009267596, US 2010319542, US 2009265344, US 2012684607, US 2004583227, U.S. Pat. No. 8,030,024, US 2006643569, U.S. Pat. No. 7,868,146, US 2007991916, U.S. Pat. No. 8,097,583, US 2006643200, U.S. Pat. Nos. 8,729,238, 8,877,903, US 20190062557, US 20160280960, US 20110201783, US 2008991916, US 2011986662, US 2012697729, US 20150328363, U.S. Pat. No. 9,034,816, US 20130172478, U.S. Pat. No. 9,217,017, US 20170202995, U.S. Pat. No. 8,721,991, US 2008227498, U.S. Pat. Nos. 9,233,067, 8,288,512, US 2008161364, U.S. Pat. No. 7,148,039, U.S. Ser. No. 19/992,47806, US 2001861597, US 2004887100, U.S. Pat. Nos. 9,481,719, 8,765,688, US 200880705, US 2010809102, U.S. Pat. No. 8,367,803, US 2010664902, U.S. Pat. No. 7,569,660, U.S. Ser. No. 19/991,38833, US 2000591632, US 20120065126, US 20100278882, US 2008161352, US 20100015070, US 2009513709, US 20090194317, US 2004559286, US 200589551, US 2008187824, US 20050266242, US 20050227322, and US 20044418.

Recombinant silk is also described in other patents and patent applications, incorporated by reference herein: US 20190062557, US 20150284565, US 20130225476, US 20130172478, US 20130136779, US 20130109762, US 20120252294, US 20110230911, US 20110201783, US 20100298877, U.S. Pat. Nos. 10,478,520, 10,253,213, 10,072,152, 9,233,067, 9,217,017, 9,034,816, 8,877,903, 8,729,238, 8,721,991, 8,097,583, 8,034,897, 8,030,024, 7,951,908, 7,868,146, and 7,754,851.

In some embodiments, the recombinant spider silk protein in this disclosure comprises or consists of 2 to 80 repetitive units, each independently selected from GPGXX, GGX and Aₓ as defined herein.

In some embodiments, the recombinant spider silk protein in this disclosure comprises or consists of repetitive units each independently selected from selected from the group consisting of GPGAS, GPGSG, GPGGY, GPGGP, GPGGA, GPGQQ, GPGGG, GPGQG, GPGGS, GGY, GGP, GGA, GGR, GGS, GGT, GGN, GGQ, AAAAA, AAAAAA, AAAAAAA, AAAAAAAA, AAAAAAAAA, AAAAAAAAAA, GGRPSDTYG and GGRPSSSYG, (i) GPYGPGASAAAAAAGGYGPGSGQQ, (ii) GSSAAAAAAAASGPGGYGPENQGPSGPGGYGPGGP, (iii) GPGQQGPGQQGPGQQGPGQQ: (iv) GPGGAGGPYGPGGAGGPYGPGGAGGPY, (v) GGTTIIEDLDITIDGADGPITISEELTI, (vi) PGSSAAAAAAAASGPGQGQGQGQGQGGRPSDTYG, (vii) SAAAAAAAAGPGGGNGGRPSDTYGAPGGGNGGRPSSSYG, (viii) GGAGGAGGAGGSGGAGGS (SEQ ID NO: 27), (ix) GPGGAGPGGYGPGGSGPGGYGPGGSGPGGY, (x) GPYGPGASAAAAAAGGYGPGCGQQ, (xi) GPYGPGASAAAAAAGGYGPGKGQQ, (xii) GSSAAAAAAAASGPGGYGPENQGPCGPGGYGPGGP, (xiii) GSSAAAAAAAASGPGGYGPKNQGPSGPGGYGPGGP, (xiv) GSSAAAAAAAASGPGGYGPKNQGPSGPGGYGPGGP, or variants thereof as described in U.S. Pat. No. 8,877,903, for example, a synthetic spider peptide having sequential order of GPGAS, GGY, GPGSG in the peptide chain, or sequential order of AAAAAAAA, GPGGY, GPGGP in the peptide chain, sequential order of AAAAAAAA, GPGQG, GGR in the peptide chain.

In some embodiments, this disclosure provides silk protein-like multiblock peptides that imitate the repeating units of amino acids derived from natural spider silk proteins such as Spidroin major 1 domain, Spidroin major 2 domain or Spidroin minor 1 domain and the profile of variation between the repeating units without modifying their three-dimensional conformation, wherein these silk protein-like multiblock peptides comprise a repeating unit of amino acids corresponding to one of the sequences (I), (II), (III) and/or (IV) below.

[(XGG)_{w}(XGA)(GXG)ₓ(AGA)_{y}(G)_{z}AG]ₚ Formula (I) in which: X corresponds to tyrosine or to glutamine, w is an integer equal to 2 or 3, x is an integer from 1 to 3, y is an integer from 5 to 7, z is an integer equal to 1 or 2, and p is an integer and having any weight average molecular weight described herein, and/or

[(GPG₂YGPGQ₂)ₐ(X′)₂S(A)_{b}]ₚ Formula (II) in which: X′ corresponds to the amino acid sequence GPS or GPG, a is equal to 2 or 3, b is an integer from 7 to 10, and p is an integer and having any weight average molecular weight described herein, and/or

[(GR)(GA)ₗ(A)ₘ(GGX)ₙ(GA)ₗ(A)ₘ]ₚ Formula (III) and/or [(GGX)ₙ(GA)ₘ(A)ₗ]ₚ Formula (IV) in which: X″ corresponds to tyrosine, glutamine or alanine, 1 is an integer from 1 to 6, m is an integer from 0 to 4, n is an integer from 1 to 4, and p is an integer.

In some embodiments, the recombinant spider silk protein or an analog of a spider silk protein comprising an amino acid repeating unit of sequence (V):

[(Xaa Gly Gly)_{w}(Xaa Gly Ala)(Gly Xaa Gly)ₓ(Ala Gly Ala)_{y}(Gly)_{z}Ala Gly]ₚ Formula (V), wherein Xaa is tyrosine or glutamine, w is an integer equal to 2 or 3, x is an integer from 1 to 3, y is an integer from 5 to 7, z is an integer equal to 1 or 2, and p is an integer.

In some embodiments, the recombinant spider silk protein in this disclosure is selected from the group consisting of ADF-3 or variants thereof, ADF-4 or variants thereof, MaSpI (SEQ ID NO: 43) or variants thereof, MaSpII (SEQ ID NO: 44) or variants thereof as described in U.S. Pat. No. 8,367,803.

In some embodiments, this disclosure provides water soluble recombinant spider silk proteins produced in mammalian cells. The solubility of the spider silk proteins produced in mammalian cells was attributed to the presence of the COOH-terminus in these proteins, which makes them more hydrophilic. These COOH-terminal amino acids are absent in spider silk proteins expressed in microbial hosts.

In some embodiments, the recombinant spider silk protein in this disclosure comprises water soluble recombinant spider silk protein C16 modified with an amino or carboxyl terminal selected from the amino acid sequences consisting of: GCGGGGGG, GKGGGGGG, GCGGSGGGGSGGGG, GKGGGGGGSGGGG, and GCGGGGGGSGGGG. In some embodiments, the recombinant spider silk protein in this disclosure comprises C₁₆NR4, C₃₂NR4, C16, C32, NR4C₁₆NR4, NR4C₃₂NR4, NR3C₁₆NR3, or NR3C₃₂NR3 such that the molecular weight of the protein ranges as described herein.

In some embodiments, the recombinant spider silk protein in this disclosure comprises recombinant spider silk protein having a synthetic repetitive peptide segments and an amino acid sequence adapted from the natural sequence of ADF4 from A. diadematus as described in U.S. Pat. No. 8,877,903. In some embodiments, the RSPF in this disclosure comprises the recombinant spider silk proteins having repeating peptide units derived from natural spider silk proteins such as Spidroin major 1 domain, Spidroin major 2 domain or Spidroin minor 1 domain, wherein the repeating peptide sequence is GSSAAAAAAAASGPGQGQGQGQGQGGRPSDTYG or SAAAAAAAAGPGGGNGGRPSDTYGAPGGGNGGRPSSSYG, as described in U.S. Pat. No. 8,367,803.

In some embodiments, this disclosure provides recombinant spider proteins composed of the GPGGAGPGGYGPGGSGPGGYGPGGSGPGGY repetitive fragment and having a molecular weight as described herein.

As used herein, the term “recombinant silk” refers to recombinant spider and/or silkworm silk protein or fragments thereof. In an embodiment, the spider silk protein is selected from the group consisting of swathing silk (Achniform gland silk), egg sac silk (Cylindriform gland silk), egg case silk (Tubuliform silk), non-sticky dragline silk (Ampullate gland silk), attaching thread silk (Pyriform gland silk), sticky silk core fibers (Flagelliform gland silk), and sticky silk outer fibers (Aggregate gland silk). For example, recombinant spider silk protein, as described herein, includes the proteins described in U.S. Patent Application No. 2016/0222174 and U.S. Pat. Nos. 9,051,453, 9,617,315, 9,689,089, 8,173,772, and 8,642,734.

Some organisms make multiple silk fibers with unique sequences, structural elements, and mechanical properties. For example, orb weaving spiders have six unique types of glands that produce different silk polypeptide sequences that are polymerized into fibers tailored to fit an environmental or lifecycle niche. The fibers are named for the gland they originate from and the polypeptides are labeled with the gland abbreviation (e.g. “Ma”) and “Sp” for spidroin (short for spider fibroin). In orb weavers, these types include Major Ampullate (MaSp, also called dragline), Minor Ampullate (MiSp), Flagelliform (Flag), Aciniform (AcSp), Tubuliform (TuSp), and Pyriform (PySp). This combination of polypeptide sequences across fiber types, domains, and variation amongst different genus and species of organisms leads to a vast array of potential properties that can be harnessed by commercial production of the recombinant fibers. To date, the vast majority of the work with recombinant silks has focused on the Major Ampullate Spidroins (MaSp).

Aciniform (AcSp) silks tend to have high toughness, a result of moderately high strength coupled with moderately high extensibility. AcSp silks are characterized by large block (“ensemble repeat”) sizes that often incorporate motifs of poly serine and GPX. Tubuliform (TuSp or Cylindrical) silks tend to have large diameters, with modest strength and high extensibility. TuSp silks are characterized by their poly serine and poly threonine content, and short tracts of poly alanine. Major Ampullate (MaSp) silks tend to have high strength and modest extensibility. MaSp silks can be one of two subtypes: MaSpI and MaSp2. MaSp1 silks are generally less extensible than MaSp2 silks, and are characterized by poly alanine, GX, and GGX motifs. MaSp2 silks are characterized by poly alanine, GGX, and GPX motifs. Minor Ampullate (MiSp) silks tend to have modest strength and modest extensibility. MiSp silks are characterized by GGX, GA, and poly A motifs, and often contain spacer elements of approximately 100 amino acids. Flagelliform (Flag) silks tend to have very high extensibility and modest strength. Flag silks are usually characterized by GPG, GGX, and short spacer motifs.

Silk polypeptides are characteristically composed of a repeat domain (REP) flanked by non-repetitive regions (e.g., C-terminal and N-terminal domains). In an embodiment, both the C-terminal and N-terminal domains are between 75-350 amino acids in length. The repeat domain exhibits a hierarchical architecture. The repeat domain comprises a series of blocks (also called repeat units). The blocks are repeated, sometimes perfectly and sometimes imperfectly (making up a quasi-repeat domain), throughout the silk repeat domain. The length and composition of blocks varies among different silk types and across different species. Table 1 of U.S. Published Application No. 2016/0222174, the entirety of which is incorporated herein, lists examples of block sequences from selected species and silk types, with further examples presented in Rising, A. et al., Spider silk proteins: recent advances in recombinant production, structure-function relationships and biomedical applications, Cell Mol. Life Sci., 68:2, pg 169-184 (2011); and Gatesy, J. et al., Extreme diversity, conservation, and convergence of spider silk fibroin sequences, Science, 291:5513, pg. 2603-2605 (2001). In some cases, blocks may be arranged in a regular pattern, forming larger macro-repeats that appear multiple times (usually 2-8) in the repeat domain of the silk sequence. Repeated blocks inside a repeat domain or macro-repeat, and repeated macro-repeats within the repeat domain, may be separated by spacing elements.

The construction of certain spider silk block copolymer polypeptides from the blocks and/or macro-repeat domains, according to certain embodiments of the disclosure, is illustrated in U.S. Published Patent Application No. 2016/0222174.

The recombinant block copolymer polypeptides based on spider silk sequences produced by gene expression in a recombinant prokaryotic or eukaryotic system can be purified according to methods known in the art. In a preferred embodiment, a commercially available expression/secretion system can be used, whereby the recombinant polypeptide is expressed and thereafter secreted from the host cell, to be easily purified from the surrounding medium. If expression/secretion vectors are not used, an alternative approach involves purifying the recombinant block copolymer polypeptide from cell lysates (remains of cells following disruption of cellular integrity) derived from prokaryotic or eukaryotic cells in which a polypeptide was expressed. Methods for generation of such cell lysates are known to those of skill in the art. In some embodiments, recombinant block copolymer polypeptides are isolated from cell culture supernatant.

Recombinant block copolymer polypeptide may be purified by affinity separation, such as by immunological interaction with antibodies that bind specifically to the recombinant polypeptide or nickel columns for isolation of recombinant polypeptides tagged with 6-8 histidine residues at their N-terminus or C-terminus Alternative tags may comprise the FLAG epitope or the hemagglutinin epitope. Such methods are commonly used by skilled practitioners.

A solution of such polypeptides (i.e., recombinant silk protein) may then be prepared and used as described herein.

In another embodiment, recombinant silk protein may be prepared according to the methods described in U.S. Pat. No. 8,642,734, the entirety of which is incorporated herein, and used as described herein.

In an embodiment, a recombinant spider silk protein is provided. The spider silk protein typically consists of from 170 to 760 amino acid residues, such as from 170 to 600 amino acid residues, preferably from 280 to 600 amino acid residues, such as from 300 to 400 amino acid residues, more preferably from 340 to 380 amino acid residues. The small size is advantageous because longer spider silk proteins tend to form amorphous aggregates, which require use of harsh solvents for solubilization and polymerization. The recombinant spider silk protein may contain more than 760 residues, in particular in cases where the spider silk protein contains more than two fragments derived from the N-terminal part of a spider silk protein, The spider silk protein comprises an N-terminal fragment consisting of at least one fragment (NT) derived from the corresponding part of a spider silk protein, and a repetitive fragment (REP) derived from the corresponding internal fragment of a spider silk protein. Optionally, the spider silk protein comprises a C-terminal fragment (CT) derived from the corresponding fragment of a spider silk protein. The spider silk protein comprises typically a single fragment (NT) derived from the N-terminal part of a spider silk protein, but in preferred embodiments, the N-terminal fragment include at least two, such as two fragments (NT) derived from the N-terminal part of a spider silk protein. Thus, the spidroin can schematically be represented by the formula NTₘ-REP, and alternatively NTₘ-REP-CT, where m is an integer that is 1 or higher, such as 2 or higher, preferably in the ranges of 1-2, 1-4, 1-6, 2-4 or 2-6. Preferred spidroins can schematically be represented by the formulas NT2-REP or NT-REP, and alternatively NT2-REP-CT or NT-REP-CT. The protein fragments are covalently coupled, typically via a peptide bond. In one embodiment, the spider silk protein consists of the NT fragment(s) coupled to the REP fragment, which REP fragment is optionally coupled to the CT fragment.

In one embodiment, the first step of the method of producing polymers of an isolated spider silk protein involves expression of a polynucleic acid molecule which encodes the spider silk protein in a suitable host, such as Escherichia coli. The thus obtained protein is isolated using standard procedures. Optionally, lipopolysaccharides and other pyrogens are actively removed at this stage.

In the second step of the method of producing polymers of an isolated spider silk protein, a solution of the spider silk protein in a liquid medium is provided. By the terms “soluble” and “in solution” is meant that the protein is not visibly aggregated and does not precipitate from the solvent at 60,000×g. The liquid medium can be any suitable medium, such as an aqueous medium, preferably a physiological medium, typically a buffered aqueous medium, such as a 10-50 mM Tris-HCl buffer or phosphate buffer. The liquid medium has a pH of 6.4 or higher and/or an ion composition that prevents polymerization of the spider silk protein. That is, the liquid medium has either a pH of 6.4 or higher or an ion composition that prevents polymerization of the spider silk protein, or both.

Ion compositions that prevent polymerization of the spider silk protein can readily be prepared by the skilled person utilizing the methods disclosed herein. A preferred ion composition that prevents polymerization of the spider silk protein has an ionic strength of more than 300 mM. Specific examples of ion compositions that prevent polymerization of the spider silk protein include above 300 mM NaCl, 100 mM phosphate and combinations of these ions having desired preventive effect on the polymerization of the spider silk protein, e.g. a combination of 10 mM phosphate and 300 mM NaCl.

The presence of an NT fragment improves the stability of the solution and prevents polymer formation under these conditions. This can be advantageous when immediate polymerization may be undesirable, e.g. during protein purification, in preparation of large batches, or when other conditions need to be optimized. It is preferred that the pH of the liquid medium is adjusted to 6.7 or higher, such as 7.0 or higher, or even 8.0 or higher, such as up to 10.5, to achieve high solubility of the spider silk protein. It can also be advantageous that the pH of the liquid medium is adjusted to the range of 6.4-6.8, which provides sufficient solubility of the spider silk protein but facilitates subsequent pH adjustment to 6.3 or lower.

In the third step, the properties of the liquid medium are adjusted to a pH of 6.3 or lower and ion composition that allows polymerization. That is, if the liquid medium wherein the spider silk protein is dissolved has a pH of 6.4 or higher, the pH is decreased to 6.3 or lower. The skilled person is well aware of various ways of achieving this, typically involving addition of a strong or weak acid. If the liquid medium wherein the spider silk protein is dissolved has an ion composition that prevents polymerization, the ion composition is changed so as to allow polymerization. The skilled person is well aware of various ways of achieving this, e.g. dilution, dialysis or gel filtration. If required, this step involves both decreasing the pH of the liquid medium to 6.3 or lower and changing the ion composition so as to allow polymerization. It is preferred that the pH of the liquid medium is adjusted to 6.2 or lower, such as 6.0 or lower. In particular, it may be advantageous from a practical point of view to limit the pH drop from 6.4 or 6.4-6.8 in the preceding step to 6.3 or 6.0-6.3, e.g. 6.2 in this step. In a preferred embodiment, the pH of the liquid medium of this step is 3 or higher, such as 4.2 or higher. The resulting pH range, e.g. 4.2-6.3 promotes rapid polymerization,

In the fourth step, the spider silk protein is allowed to polymerize in the liquid medium having pH of 6.3 or lower and an ion composition that allows polymerization of the spider silk protein. Although the presence of the NT fragment improves solubility of the spider silk protein at a pH of 6.4 or higher and/or an ion composition that prevents polymerization of the spider silk protein, it accelerates polymer formation at a pH of 6.3 or lower when the ion composition allows polymerization of the spider silk protein. The resulting polymers are preferably solid and macroscopic, and they are formed in the liquid medium having a pH of 6.3 or lower and an ion composition that allows polymerization of the spider silk protein. In a preferred embodiment, the pH of the liquid medium of this step is 3 or higher, such as 4.2 or higher. The resulting pH range, e.g. 4.2-6.3 promotes rapid polymerization, Resulting polymer may be provided at the molecular weights described herein and prepared as a solution form that may be used as necessary for article coatings.

Ion compositions that allow polymerization of the spider silk protein can readily be prepared by the skilled person utilizing the methods disclosed herein. A preferred ion composition that allows polymerization of the spider silk protein has an ionic strength of less than 300 mM. Specific examples of ion compositions that allow polymerization of the spider silk protein include 150 mM NaCl, 10 mM phosphate, 20 mM phosphate and combinations of these ions lacking preventive effect on the polymerization of the spider silk protein, e.g. a combination of 10 mM phosphate or 20 mM phosphate and 150 mM NaCl. It is preferred that the ionic strength of this liquid medium is adjusted to the range of 1-250 mM.

Without desiring to be limited to any specific theory, it is envisaged that the NT fragments have oppositely charged poles, and that environmental changes in pH affects the charge balance on the surface of the protein followed by polymerization, whereas salt inhibits the same event.

At neutral pH, the energetic cost of burying the excess negative charge of the acidic pole may be expected to prevent polymerization. However, as the dimer approaches its isoelectric point at lower pH, attractive electrostatic forces will eventually become dominant, explaining the observed salt and pH-dependent polymerization behavior of NT and NT-containing minispidroins. It is proposed that, in some embodiments, pH-induced NT polymerization, and increased efficiency of fiber assembly of NT-minispidroins, are due to surface electrostatic potential changes, and that clustering of acidic residues at one pole of NT shifts its charge balance such that the polymerization transition occurs at pH values of 6.3 or lower.

In a fifth step, the resulting, preferably solid spider silk protein polymers are isolated from said liquid medium. Optionally, this step involves actively removing lipopolysaccharides and other pyrogens from the spidroin polymers.

Without desiring to be limited to any specific theory, it has been observed that formation of spidroin polymers progresses via formation of water-soluble spidroin dimers. The present disclosure thus also provides a method of producing dimers of an isolated spider silk protein, wherein the first two method steps are as described above. The spider silk proteins are present as dimers in a liquid medium at a pH of 6.4 or higher and/or an ion composition that prevents polymerization of said spider silk protein. The third step involves isolating the dimers obtained in the second step, and optionally removal of lipopolysaccharides and other pyrogens. In a preferred embodiment, the spider silk protein polymer of the disclosure consists of polymerized protein dimers. The present disclosure thus provides a novel use of a spider silk protein, preferably those disclosed herein, for producing dimers of the spider silk protein.

According to another aspect, the disclosure provides a polymer of a spider silk protein as disclosed herein. In an embodiment, the polymer of this protein is obtainable by any one of the methods therefor according to the disclosure. Thus, the disclosure provides various uses of recombinant spider silk protein, preferably those disclosed herein, for producing polymers of the spider silk protein as recombinant silk based coatings. According to one embodiment, the present disclosure provides a novel use of a dimer of a spider silk protein, preferably those disclosed herein, for producing polymers of the isolated spider silk protein as recombinant silk based coatings. In these uses, it is preferred that the polymers are produced in a liquid medium having a pH of 6.3 or lower and an ion composition that allows polymerization of said spider silk protein. In an embodiment, the pH of the liquid medium is 3 or higher, such as 4.2 or higher. The resulting pH range, e.g. 4.2-6.3 promotes rapid polymerization,

Using the method(s) of the present disclosure, it is possible to control the polymerization process, and this allows for optimization of parameters for obtaining silk polymers with desirable properties and shapes.

In an embodiment, the recombinant silk proteins described herein, include those described in U.S. Pat. No. 8,642,734, the entirety of which is incorporated by reference.

In another embodiment, the recombinant silk proteins described herein may be prepared according to the methods described in U.S. Pat. No. 9,051,453, the entirety of which is incorporated herein by reference.

An amino acid sequence represented by SEQ ID NO: 1 of U.S. Pat. No. 9,051,453 is identical to an amino acid sequence that is composed of 50 amino acid residues of an amino acid sequence of ADF3 at the C-terminal (NCBI Accession No.: AAC47010, GI: 1263287). An amino acid sequence represented by SEQ ID NO: 2 of U.S. Pat. No. 9,051,453 is identical to an amino acid sequence represented by SEQ ID NO: 1 of U.S. Pat. No. 9,051,453 from which 20 residues have been removed from the C-terminal. An amino acid sequence represented by SEQ ID NO: 3 of U.S. Pat. No. 9,051,453 is identical to an amino acid sequence represented by SEQ ID NO: 1 from which 29 residues have been removed from the C-terminal.

An example of the polypeptide that contains units of the amino acid sequence represented by the formula 1: REP1-REP2 (1) and that has, at a C-terminal, an amino acid sequence represented by any of SEQ ID NOS: 1 to 3 or an amino acid sequence having a homology of 90% or more with the amino acid sequence represented by any of SEQ ID NOS: 1 to 3 of U.S. Pat. No. 9,051,453 is a polypeptide having an amino acid sequence represented by SEQ ID NO: 8 of U.S. Pat. No. 9,051,453. The polypeptide having the amino acid sequence represented by SEQ ID NO: 8 of U.S. Pat. No. 9,051,453 is obtained by the following mutation: in an amino acid sequence of ADF3 (NCBI Accession No.: AAC47010, GI: 1263287) to the N-terminal of which has been added an amino acid sequence (SEQ ID NO: 5 of U.S. Pat. No. 9,051,453) composed of a start codon, His 10 tags and an HRV3C Protease (Human rhinovirus 3C Protease) recognition site, 1^{st} to 13^{th} repetitive regions are about doubled and the translation ends at the 1154^{th} amino acid residue. In the polypeptide having the amino acid sequence represented by SEQ ID NO: 8 of U.S. Pat. No. 9,051,453, the C-terminal sequence is identical to the amino acid sequence represented by SEQ ID NO: 3.

Further, the polypeptide that contains units of the amino acid sequence represented by the formula 1: REP1-REP2 (1) and that has, at a C-terminal, an amino acid sequence represented by any of SEQ ID NOS: 1 to 3 of U.S. Pat. No. 9,051,453 or an amino acid sequence having a homology of 90% or more with the amino acid sequence represented by any of SEQ ID NOS: 1 to 3 of U.S. Pat. No. 9,051,453 may be a protein that has an amino acid sequence represented by SEQ ID NO: 8 of U.S. Pat. No. 9,051,453 in which one or a plurality of amino acids have been substituted, deleted, inserted and/or added and that has a repetitious region composed of a crystal region and an amorphous region.

Further, an example of the polypeptide containing two or more units of the amino acid sequence represented by the formula 1: REP1-REP2 (1) is a recombinant protein derived from ADF4 having an amino acid sequence represented by SEQ ID NO: 15 of U.S. Pat. No. 9,051,453. The amino acid sequence represented by SEQ ID NO: 15 of U.S. Pat. No. 9,051,453 is an amino acid sequence obtained by adding the amino acid sequence (SEQ ID NO: 5 of U.S. Pat. No. 9,051,453) composed of a start codon, His 10 tags and an HRV3C Protease (Human rhinovirus 3C Protease) recognition site, to the N-terminal of a partial amino acid sequence of ADF4 obtained from the NCBI database (NCBI Accession No.: AAC47011, GI: 1263289). Further, the polypeptide containing two or more units of the amino acid sequence represented by the formula 1: REP1-REP2 (1) may be a polypeptide that has an amino acid sequence represented by SEQ ID NO: 15 of U.S. Pat. No. 9,051,453 in which one or a plurality of amino acids have been substituted, deleted, inserted and/or added and that has a repetitious region composed of a crystal region and an amorphous region. Further, an example of the polypeptide containing two or more units of the amino acid sequence represented by the formula 1: REP1-REP2 (1) is a recombinant protein derived from MaSp2 that has an amino acid sequence represented by SEQ ID NO: 17 of U.S. Pat. No. 9,051,453. The amino acid sequence represented by SEQ ID NO: 17 of U.S. Pat. No. 9,051,453 is an amino acid sequence obtained by adding the amino acid sequence (SEQ ID NO: 5 of U.S. Pat. No. 9,051,453) composed of a start codon, His 10 tags and an HRV3C Protease (Human rhinovirus 3C Protease) recognition site, to the N-terminal of a partial sequence of MaSp2 obtained from the NCBI web database (NCBI Accession No.: AAT75313, GI: 50363147). Furthermore, the polypeptide containing two or more units of the amino acid sequence represented by the formula 1: REP1-REP2 (1) may be a polypeptide that has an amino acid sequence represented by SEQ ID NO: 17 of U.S. Pat. No. 9,051,453 in which one or a plurality of amino acids have been substituted, deleted, inserted and/or added and that has a repetitious region composed of a crystal region and an amorphous region.

Examples of the polypeptide derived from flagelliform silk proteins include a polypeptide containing 10 or more units of an amino acid sequence represented by the formula 2: REP3 (2), preferably a polypeptide containing 20 or more units thereof, and more preferably a polypeptide containing 30 or more units thereof. In the case of producing a recombinant protein using a microbe such as Escherichia coli as a host, the molecular weight of the polypeptide derived from flagelliform silk proteins is preferably 500 kDa or less, more preferably 300 kDa or less, and further preferably 200 kDa or less, in terms of productivity.

In the formula (2), the REP 3 indicates an amino acid sequence composed of Gly-Pro-Gly-Gly-X, where X indicates an amino acid selected from the group consisting of Ala, Ser, Tyr and Val.

A major characteristic of the spider silk is that the flagelliform silk does not have a crystal region, but has a repetitious region composed of an amorphous region. Since the major dragline silk and the like have a repetitious region composed of a crystal region and an amorphous region, they are expected to have both high stress and stretchability. Meanwhile, as to the flagelliform silk, although the stress is inferior to that of the major dragline silk, the stretchability is high. The reason for this is considered to be that most of the flagelliform silk is composed of amorphous regions.

An example of the polypeptide containing 10 or more units of the amino acid sequence represented by the formula 2: REP3 (2) is a recombinant protein derived from flagelliform silk proteins having an amino acid sequence represented by SEQ ID NO: 19 of U.S. Pat. No. 9,051,453. The amino acid sequence represented by SEQ ID NO: 19 of U.S. Pat. No. 9,051,453 is an amino acid sequence obtained by combining a partial sequence of flagelliform silk protein of Nephila clavipes obtained from the NCBI database (NCBI Accession No.: AAF36090, GI: 7106224), specifically, an amino acid sequence thereof from the 1220^{th} residue to the 1659^{th} residue from the N-terminal that corresponds to repetitive sections and motifs (referred to as a PR1 sequence), with a partial sequence of flagelliform silk protein of Nephila clavipes obtained from the NCBI database (NCBI Accession No.: AAC38847, GI: 2833649), specifically, a C-terminal amino acid sequence thereof from the 816^{th} residue to the 907^{th} residue from the C-terminal, and thereafter adding the amino acid sequence (SEQ ID NO: 5 of U.S. Pat. No. 9,051,453) composed of a start codon, His 10 tags and an HRV3C Protease recognition site, to the N-terminal of the combined sequence. Further, the polypeptide containing 10 or more units of the amino acid sequence represented by the formula 2: REP3 (2) may be a polypeptide that has an amino acid sequence represented by SEQ ID NO: 19 of U.S. Pat. No. 9,051,453 in which one or a plurality of amino acids have been substituted, deleted, inserted and/or added and that has a repetitious region composed of an amorphous region.

The polypeptide can be produced using a host that has been transformed by an expression vector containing a gene encoding a polypeptide. A method for producing a gene is not limited particularly, and it may be produced by amplifying a gene encoding a natural spider silk protein from a cell derived from spiders by a polymerase chain reaction (PCR), etc., and cloning it, or may be synthesized chemically. Also, a method for chemically synthesizing a gene is not limited particularly, and it can be synthesized as follows, for example: based on information of amino acid sequences of natural spider silk proteins obtained from the NCBI web database, etc., oligonucleotides that have been synthesized automatically with AKTA oligopilot plus 10/100 (GE Healthcare Japan Corporation) are linked by PCR, etc. At this time, in order to facilitate the purification and observation of protein, it is possible to synthesize a gene that encodes a protein having an amino acid sequence of the above-described amino acid sequence to the N-terminal of which has been added an amino acid sequence composed of a start codon and His 10 tags.

Examples of the expression vector include a plasmid, a phage, a virus, and the like that can express protein based on a DNA sequence. The plasmid-type expression vector is not limited particularly as long as it allows a target gene to be expressed in a host cell and it can amplify itself. For example, in the case of using Escherichia coli Rosetta (DE3) as a host, a pET22b(+) plasmid vector, a pCold plasmid vector, and the like can be used. Among these, in terms of productivity of protein, it is preferable to use the pET22b(+) plasmid vector. Examples of the host include animal cells, plant cells, microbes, etc.

The polypeptide used in the present disclosure is preferably a polypeptide derived from ADF3, which is one of two principal dragline silk proteins of Araneus diadematus. This polypeptide has advantages of basically having high strength-elongation and toughness and of being synthesized easily.

Accordingly, the recombinant silk protein (e.g., the recombinant spider silk-based protein) used in accordance with the embodiments, articles, and/or methods described herein, may include one or more recombinant silk proteins described above or recited in U.S. Pat. Nos. 8,173,772, 8,278,416, 8,618,255, 8,642,734, 8,691,581, 8,729,235, 9,115,204, 9,157,070, 9,309,299, 9,644,012, 9,708,376, 9,051,453, 9,617,315, 9,968,682, 9,689,089, 9,732,125, 9,856,308, 9,926,348, 10,065,997, 10,316,069, and 10,329,332; and U.S. Patent Publication Nos. 2009/0226969, 2011/0281273, 2012/0041177, 2013/0065278, 2013/0115698, 2013/0316376, 2014/0058066, 2014/0079674, 2014/0245923, 2015/0087046, 2015/0119554, 2015/0141618, 2015/0291673, 2015/0291674, 2015/0239587, 2015/0344542, 2015/0361144, 2015/0374833, 2015/0376247, 2016/0024464, 2017/0066804, 2017/0066805, 2015/0293076, 2016/0222174, 2017/0283474, 2017/0088675, 2019/0135880, 2015/0329587, 2019/0040109, 2019/0135881, 2019/0177363, 2019/0225646, 2019/0233481, 2019/0031842, 2018/0355120, 2019/0186050, 2019/0002644, 2020/0031887, 2018/0273590, 20191/094403, 2019/0031843, 2018/0251501, 2017/0066805, 2018/0127553, 2019/0329526, 2020/0031886, 2018/0080147, 2019/0352349, 2020/0043085, 2019/0144819, 2019/0228449, 2019/0340666, 2020/0000091, 2019/0194710, 2019/0151505, 2018/0265555, 2019/0352330, 2019/0248847, and 2019/0378191, the entirety of which are incorporated herein by reference.

Silk Fibroin-Like Protein Fragments

The recombinant silk protein in this disclosure comprises synthetic proteins which are based on repeat units of natural silk proteins. Besides the synthetic repetitive silk protein sequences, these can additionally comprise one or more natural nonrepetitive silk protein sequences. As used herein, “silk fibroin-like protein fragments” refer to protein fragments having a molecular weight and polydispersity as defined herein, and a certain degree of homology to a protein selected from native silk protein, fibroin heavy chain, fibroin light chain, or any protein comprising one or more GAGAGS hexa amino acid repeating units. In some embodiments, a degree of homology is selected from about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, or less than 75%.

As described herein, a protein such as native silk protein, fibroin heavy chain, fibroin light chain, or any protein comprising one or more GAGAGS hexa amino acid repeating units includes between about 9% and about 45% glycine, or about 9% glycine, or about 10% glycine, about 43% glycine, about 44% glycine, about 45% glycine, or about 46% glycine. As described herein, a protein such as native silk protein, fibroin heavy chain, fibroin light chain, or any protein comprising one or more GAGAGS hexa amino acid repeating units includes between about 13% and about 30% alanine, or about 13% alanine, or about 28% alanine, or about 29% alanine, or about 30% alanine, or about 31% alanine. As described herein, a protein such as native silk protein, fibroin heavy chain, fibroin light chain, or any protein comprising one or more GAGAGS hexa amino acid repeating units includes between 9% and about 12% serine, or about 9% serine, or about 10% serine, or about 11% serine, or about 12% serine.

In some embodiments, a silk fibroin-like protein described herein includes about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, or about 55% glycine. In some embodiments, a silk fibroin-like protein described herein includes about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, or about 39% alanine. In some embodiments, a silk fibroin-like protein described herein includes about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, or about 22% serine. In some embodiments, a silk fibroin-like protein described herein may include independently any amino acid known to be included in natural fibroin. In some embodiments, a silk fibroin-like protein described herein may exclude independently any amino acid known to be included in natural fibroin. In some embodiments, on average 2 out of 6 amino acids, 3 out of 6 amino acids, or 4 out of 6 amino acids in a silk fibroin-like protein described herein is glycine. In some embodiments, on average 1 out of 6 amino acids, 2 out of 6 amino acids, or 3 out of 6 amino acids in a silk fibroin-like protein described herein is alanine. In some embodiments, on average none out of 6 amino acids, 1 out of 6 amino acids, or 2 out of 6 amino acids in a silk fibroin-like protein described herein is serine.

Other Properties of SPF

Compositions of the present disclosure are “biocompatible” or otherwise exhibit “biocompatibility” meaning that the compositions are compatible with living tissue or a living system by not being toxic, injurious, or physiologically reactive and not causing immunological rejection or an inflammatory response. Such biocompatibility can be evidenced by participants topically applying compositions of the present disclosure on their skin for an extended period of time. In an embodiment, the extended period of time is about 3 days. In an embodiment, the extended period of time is about 7 days. In an embodiment, the extended period of time is about 14 days. In an embodiment, the extended period of time is about 21 days. In an embodiment, the extended period of time is about 30 days. In an embodiment, the extended period of time is selected from the group consisting of about 1 month, about 2 months, about 3 months, about 4 months, about 5 months, about 6 months, about 7 months, about 8 months, about 9 months, about 10 months, about 11 months, about 12 months, and indefinitely. For example, in some embodiments, the coatings described herein are biocompatible coatings.

In some embodiments, compositions described herein, which may be biocompatible compositions (e.g., biocompatible coatings that include silk), may be evaluated and comply with International Standard ISO 10993-1, titled the “Biological evaluation of medical devices—Part 1: Evaluation and testing within a risk management process.” In some embodiments, compositions described herein, which may be biocompatible compositions, may be evaluated under ISO 106993-1 for one or more of cytotoxicity, sensitization, hemocompatibility, pyrogenicity, implantation, genotoxicity, carcinogenicity, reproductive and developmental toxicity, and degradation.

Compositions of the present disclosure are “hypoallergenic” meaning that they are relatively unlikely to cause an allergic reaction. Such hypoallergenicity can be evidenced by participants topically applying compositions of the present disclosure on their skin for an extended period of time. In an embodiment, the extended period of time is about 3 days. In an embodiment, the extended period of time is about 7 days. In an embodiment, the extended period of time is about 14 days. In an embodiment, the extended period of time is about 21 days. In an embodiment, the extended period of time is about 30 days. In an embodiment, the extended period of time is selected from the group consisting of about 1 month, about 2 months, about 3 months, about 4 months, about 5 months, about 6 months, about 7 months, about 8 months, about 9 months, about 10 months, about 11 months, about 12 months, and indefinitely.

In an embodiment, the stability of a composition of the present disclosure is about 1 day. In an embodiment, the stability of a composition of the present disclosure is about 2 days. In an embodiment, the stability of a composition of the present disclosure is about 3 days. In an embodiment, the stability of a composition of the present disclosure is about 4 days. In an embodiment, the stability of a composition of the present disclosure is about 5 days. In an embodiment, the stability of a composition of the present disclosure is about 6 days. In an embodiment, the stability of a composition of the present disclosure is about 7 days. In an embodiment, the stability of a composition of the present disclosure is about 8 days. In an embodiment, the stability of a composition of the present disclosure is about 9 days. In an embodiment, the stability of a composition of the present disclosure is about 10 days.

In an embodiment, the stability of a composition of the present disclosure is about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days.

In an embodiment, the stability of a composition of the present disclosure is 10 days to 6 months. In an embodiment, the stability of a composition of the present disclosure is 6 months to 12 months. In an embodiment, the stability of a composition of the present disclosure is 12 months to 18 months. In an embodiment, the stability of a composition of the present disclosure is 18 months to 24 months. In an embodiment, the stability of a composition of the present disclosure is 24 months to 30 months. In an embodiment, the stability of a composition of the present disclosure is 30 months to 36 months. In an embodiment, the stability of a composition of the present disclosure is 36 months to 48 months. In an embodiment, the stability of a composition of the present disclosure is 48 months to 60 months.

In an embodiment, a SPF composition of the present disclosure is not soluble in an aqueous solution due to the crystallinity of the protein. In an embodiment, a SPF composition of the present disclosure is soluble in an aqueous solution. In an embodiment, the SPF of a composition of the present disclosure include a crystalline portion of about two-thirds and an amorphous region of about one-third. In an embodiment, the SPF of a composition of the present disclosure include a crystalline portion of about one-half and an amorphous region of about one-half. In an embodiment, the SPF of a composition of the present disclosure include a 99% crystalline portion and a 1% amorphous region. In an embodiment, the SPF of a composition of the present disclosure include a 95% crystalline portion and a 5% amorphous region. In an embodiment, the SPF of a composition of the present disclosure include a 90% crystalline portion and a 10% amorphous region. In an embodiment, the SPF of a composition of the present disclosure include a 85% crystalline portion and a 15% amorphous region. In an embodiment, the SPF of a composition of the present disclosure include a 80% crystalline portion and a 20% amorphous region. In an embodiment, the SPF of a composition of the present disclosure include a 75% crystalline portion and a 25% amorphous region. In an embodiment, the SPF of a composition of the present disclosure include a 70% crystalline portion and a 30% amorphous region. In an embodiment, the SPF of a composition of the present disclosure include a 65% crystalline portion and a 35% amorphous region. In an embodiment, the SPF of a composition of the present disclosure include a 60% crystalline portion and a 40% amorphous region. In an embodiment, the SPF of a composition of the present disclosure include a 50% crystalline portion and a 50% amorphous region. In an embodiment, the SPF of a composition of the present disclosure include a 40% crystalline portion and a 60% amorphous region. In an embodiment, the SPF of a composition of the present disclosure include a 35% crystalline portion and a 65% amorphous region. In an embodiment, the SPF of a composition of the present disclosure include a 30% crystalline portion and a 70% amorphous region. In an embodiment, the SPF of a composition of the present disclosure include a 25% crystalline portion and a 75% amorphous region. In an embodiment, the SPF of a composition of the present disclosure include a 20% crystalline portion and a 80% amorphous region. In an embodiment, the SPF of a composition of the present disclosure include a 15% crystalline portion and a 85% amorphous region. In an embodiment, the SPF of a composition of the present disclosure include a 10% crystalline portion and a 90% amorphous region. In an embodiment, the SPF of a composition of the present disclosure include a 5% crystalline portion and a 90% amorphous region. In an embodiment, the SPF of a composition of the present disclosure include a 1% crystalline portion and a 99% amorphous region.

As used herein, the term “substantially free of inorganic residuals” means that the composition exhibits residuals of 0.1% (w/w) or less. In an embodiment, substantially free of inorganic residuals refers to a composition that exhibits residuals of 0.05% (w/w) or less. In an embodiment, substantially free of inorganic residuals refers to a composition that exhibits residuals of 0.01% (w/w) or less. In an embodiment, the amount of inorganic residuals is between 0 ppm (“non-detectable” or “ND”) and 1000 ppm. In an embodiment, the amount of inorganic residuals is ND to about 500 ppm. In an embodiment, the amount of inorganic residuals is ND to about 400 ppm. In an embodiment, the amount of inorganic residuals is ND to about 300 ppm. In an embodiment, the amount of inorganic residuals is ND to about 200 ppm. In an embodiment, the amount of inorganic residuals is ND to about 100 ppm. In an embodiment, the amount of inorganic residuals is between 10 ppm and 1000 ppm.

As used herein, the term “substantially free of organic residuals” means that the composition exhibits residuals of 0.1% (w/w) or less, in an embodiment, substantially free of organic residuals refers to a composition that exhibits residuals of 0.05% (w/w) or less. In an embodiment, substantially free of organic residuals refers to a composition that exhibits residuals of 0.01% (w/w) or less. In an embodiment, the amount of organic residuals is between 0 ppm (“non-detectable” or “ND”) and 1000 ppm. In an embodiment, the amount of organic residuals is ND to about 500 ppm. In an embodiment, the amount of organic residuals is ND to about 400 ppm. In an embodiment, the amount of organic residuals is ND to about 300 ppm. In an embodiment, the amount of organic residuals is ND to about 200 ppm. In an embodiment, the amount of organic residuals is ND to about 100 ppm. In an embodiment, the amount of organic residuals is between 10 ppm and 1000 ppm.

Compositions of the present disclosure exhibit “biocompatibility” meaning that the compositions are compatible with living tissue or a living system by not being toxic, injurious, or physiologically reactive and not causing immunological rejection. Such biocompatibility can be evidenced by participants topically applying compositions of the present disclosure on their skin for an extended period of time. In an embodiment, the extended period of time is about 3 days. In an embodiment, the extended period of time is about 7 days, in an embodiment, the extended period of time is about 14 days, in an embodiment, the extended period of time is about 21 days. In an embodiment, the extended period of time is about 30 days. In an embodiment, the extended period of time is selected from the group consisting of about I month, about 2 months, about 3 months, about 4 months, about 5 months, about 6 months, about 7 months, about 8 months, about 9 months, about 10 months, about 11 months, about 12 months, and indefinitely.

Compositions of the present disclosure are “hypoallergenic” meaning that they are relatively unlikely to cause an allergic reaction. Such hypoallergenicity can be evidenced by participants topically applying compositions of the present disclosure on their skin for an extended period of time. In an embodiment, the extended period of time is about 3 days. In an embodiment, the extended period of time is about 7 days. In an embodiment, the extended period of time is about 14 days. In an embodiment, the extended period of time is about 21 days. In an embodiment, the extended period of time is about 30 days. In an embodiment, the extended period of time is selected from the group consisting of about 1 month, about 2 months, about 3 months, about 4 months, about 5 months, about 6 months, about 7 months, about 8 months, about 9 months, about 10 months, about 11 months, about 12 months, and indefinitely.

Following are non-limiting examples of suitable ranges for various parameters in and for preparation of the silk solutions of the present disclosure. The silk solutions of the present disclosure may include one or more, but not necessarily all, of these parameters and may be prepared using various combinations of ranges of such parameters.

In an embodiment, the percent SPF in the solution is less than 30.0 wt. %. In an embodiment, the percent SPF in the solution is less than 25.0 wt. %. In an embodiment, the percent SPF in the solution is less than 20.0 wt. %. In an embodiment, the percent SPF in the solution is less than 19.0 wt. %. In an embodiment, the percent SPF in the solution is less than 18.0 wt. %. In an embodiment, the percent SPF in the solution is less than 17.0 wt. %. In an embodiment, the percent SPF in the solution is less than 16.0 wt. %. In an embodiment, the percent SPF in the solution is less than 15.0 wt. %. In an embodiment, the percent SPF in the solution is less than 14.0 wt. %. In an embodiment, the percent SPF in the solution is less than 13.0 wt. %. In an embodiment, the percent SPF in the solution is less than 12.0 wt. %. In an embodiment, the percent SPF in the solution is less than 11.0 wt. %. In an embodiment, the percent SPF in the solution is less than 10.0 wt. %. In an embodiment, the percent SPF in the solution is less than 9.0 wt. %. In an embodiment, the percent SPF in the solution is less than 8.0 wt. %. In an embodiment, the percent SPF in the solution is less than 7.0 wt. %. In an embodiment, the percent SPF in the solution is less than 6.0 wt. %. In an embodiment, the percent SPF in the solution is less than 5.0 wt. %. In an embodiment, the percent SPF in the solution is less than 4.0 wt. %. In an embodiment, the percent SPF in the solution is less than 3.0 wt. %. In an embodiment, the percent SPF in the solution is less than 2.0 wt. %. In an embodiment, the percent SPF in the solution is less than 1.0 wt. %. In an embodiment, the percent SPF in the solution is less than 0.9 wt. %. In an embodiment, the percent SPF in the solution is less than 0.8 wt. %. In an embodiment, the percent SPF in the solution is less than 0.7 wt. %. In an embodiment, the percent SPF in the solution is less than 0.6 wt. %. In an embodiment, the percent SPF in the solution is less than 0.5 wt. %. In an embodiment, the percent SPF in the solution is less than 0.4 wt. %. In an embodiment, the percent SPF in the solution is less than 0.3 wt. %. In an embodiment, the percent SPF in the solution is less than 0.2 wt. %. In an embodiment, the percent SPF in the solution is less than 0.1 wt. %.

In an embodiment, the percent SPF in the solution is greater than 0.1 wt. %. In an embodiment, the percent SPF in the solution is greater than 0.2 wt. %. In an embodiment, the percent SPF in the solution is greater than 0.3 wt. %. In an embodiment, the percent SPF in the solution is greater than 0.4 wt. %. In an embodiment, the percent SPF in the solution is greater than 0.5 wt. %. In an embodiment, the percent SPF in the solution is greater than 0.6 wt. %. In an embodiment, the percent SPF in the solution is greater than 0.7 wt. %. In an embodiment, the percent SPF in the solution is greater than 0.8 wt. %. In an embodiment, the percent SPF in the solution is greater than 0.9 wt. %. In an embodiment, the percent SPF in the solution is greater than 1.0 wt. %. In an embodiment, the percent SPF in the solution is greater than 2.0 wt. %. In an embodiment, the percent SPF in the solution is greater than 3.0 wt. %. In an embodiment, the percent SPF in the solution is greater than 4.0 wt. %. In an embodiment, the percent SPF in the solution is greater than 5.0 wt. %. In an embodiment, the percent SPF in the solution is greater than 6.0 wt. %. In an embodiment, the percent SPF in the solution is greater than 7.0 wt. %. In an embodiment, the percent SPF in the solution is greater than 8.0 wt. %. In an embodiment, the percent SPF in the solution is greater than 9.0 wt. %. In an embodiment, the percent SPF in the solution is greater than 10.0 wt. %. In an embodiment, the percent SPF in the solution is greater than 11.0 wt. %. In an embodiment, the percent SPF in the solution is greater than 12.0 wt. %. In an embodiment, the percent SPF in the solution is greater than 13.0 wt. %. In an embodiment, the percent SPF in the solution is greater than 14.0 wt. %. In an embodiment, the percent SPF in the solution is greater than 15.0 wt. %. In an embodiment, the percent SPF in the solution is greater than 16.0 wt. %. In an embodiment, the percent SPF in the solution is greater than 17.0 wt. %. In an embodiment, the percent SPF in the solution is greater than 18.0 wt. %. In an embodiment, the percent SPF in the solution is greater than 19.0 wt. %. In an embodiment, the percent SPF in the solution is greater than 20.0 wt. %. In an embodiment, the percent SPF in the solution is greater than 25.0 wt. %.

In an embodiment, the percent SPF in the solution ranges from about 0.1 wt. % to about 30.0 wt. %. In an embodiment, the percent SPF in the solution ranges from about 0.1 wt. % to about 25.0 wt. %. In an embodiment, the percent SPF in the solution ranges from about 0.1 wt. % to about 20.0 wt. %. In an embodiment, the percent SPF in the solution ranges from about 0.1 wt. % to about 15.0 wt. %. In an embodiment, the percent SPF in the solution ranges from about 0.1 wt. % to about 10.0 wt. %. In an embodiment, the percent SPF in the solution ranges from about 0.1 wt. % to about 9.0 wt. %. In an embodiment, the percent SPF in the solution ranges from about 0.1 wt. % to about 8.0 wt. %. In an embodiment, the percent SPF in the solution ranges from about 0.1 wt. % to about 7.0 wt. %. In an embodiment, the percent SPF in the solution ranges from about 0.1 wt. % to about 6.5 wt. %. In an embodiment, the percent SPF in the solution ranges from about 0.1 wt. % to about 6.0 wt. %. In an embodiment, the percent SPF in the solution ranges from about 0.1 wt. % to about 5.5 wt. %. In an embodiment, the percent SPF in the solution ranges from about 0.1 wt. % to about 5.0 wt. %. In an embodiment, the percent SPF in the solution ranges from about 0.1 wt. % to about 4.5 wt. %. In an embodiment, the percent SPF in the solution ranges from about 0.1 wt. % to about 4.0 wt. %. In an embodiment, the percent SPF in the solution ranges from about 0.1 wt. % to about 3.5 wt. %. In an embodiment, the percent SPF in the solution ranges from about 0.1 wt. % to about 3.0 wt. %. In an embodiment, the percent SPF in the solution ranges from about 0.1 wt. % to about 2.5 wt. %. In an embodiment, the percent SPF in the solution ranges from about 0.1 wt. % to about 2.0 wt. %. In an embodiment, the percent SPF in the solution ranges from about 0.1 wt. % to about 2.4 wt. %. In an embodiment, the percent SPF in the solution ranges from about 0.5 wt. % to about 5.0 wt. %. In an embodiment, the percent SPF in the solution ranges from about 0.5 wt. % to about 4.5 wt. %. In an embodiment, the percent SPF in the solution ranges from about 0.5 wt. % to about 4.0 wt. %. In an embodiment, the percent SPF in the solution ranges from about 0.5 wt. % to about 3.5 wt. %. In an embodiment, the percent SPF in the solution ranges from about 0.5 wt. % to about 3.0 wt. %. In an embodiment, the percent SPF in the solution ranges from about 0.5 wt. % to about 2.5 wt. %. In an embodiment, the percent SPF in the solution ranges from about 1.0 wt. % to about 4.0 wt. %. In an embodiment, the percent SPF in the solution ranges from about 1.0 wt. % to about 3.5 wt. %. In an embodiment, the percent SPF in the solution ranges from about 1.0 wt. % to about 3.0 wt. %. In an embodiment, the percent SPF in the solution ranges from about 1.0 wt. % to about 2.5 wt. %. In an embodiment, the percent SPF in the solution ranges from about 1.0 wt. % to about 2.4 wt. %. In an embodiment, the percent SPF in the solution ranges from about 1.0 wt. % to about 2.0 wt. %.

In an embodiment, the percent SPF in the solution ranges from about 20.0 wt. % to about 30.0 wt. %. In an embodiment, the percent SPF in the solution ranges from about 0.1 wt. % to about 10.0 wt. %. In an embodiment, the percent SPF in the solution ranges from about 1.0 wt. % to about 10.0 wt. %. In an embodiment, the percent SPF in the solution ranges from about 2 wt. % to about 10.0 wt. %. In an embodiment, the percent SPF in the solution ranges from about 0.1 wt. % to about 6.0 wt. %. In an embodiment, the percent SPF in the solution ranges from about 6.0 wt. % to about 10.0 wt. %. In an embodiment, the percent SPF in the solution ranges from about 6.0 wt. % to about 8.0 wt. %. In an embodiment, the percent SPF in the solution ranges from about 6.0 wt. % to about 9.0 wt. %. In an embodiment, the percent SPF in the solution ranges from about 10.0 wt. % to about 20.0 wt. %. In an embodiment, the percent SPF in the solution ranges from about 11.0 wt. % to about 19.0 wt. %. In an embodiment, the percent SPF in the solution ranges from about 12.0 wt. % to about 18.0 wt. %. In an embodiment, the percent SPF in the solution ranges from about 13.0 wt. % to about 17.0 wt. %. In an embodiment, the percent SPF in the solution ranges from about 14.0 wt. % to about 16.0 wt. %. In an embodiment, the percent SPF in the solution is about 1.0 wt. %. In an embodiment, the percent SPF in the solution is about 1.5 wt. %. In an embodiment, the percent SPF in the solution is about 2.0 wt. %. In an embodiment, the percent SPF in the solution is about 2.4 wt. %. In an embodiment, the percent SPF in the solution is 3.0 wt. %. In an embodiment, the percent SPF in the solution is 3.5 wt. %. In an embodiment, the percent SPF in the solution is about 4.0 wt. %. In an embodiment, the percent SPF in the solution is about 4.5 wt. %. In an embodiment, the percent SPF in the solution is about 5.0 wt. %. In an embodiment, the percent SPF in the solution is about 5.5 wt. %. In an embodiment the percent SPF in the solution is about 6.0 wt. %. In an embodiment, the percent SPF in the solution is about 6.5 wt. %. In an embodiment, the percent SPF in the solution is about 7.0 wt. %. In an embodiment, the percent SPF in the solution is about 7.5 wt. %. In an embodiment, the percent SPF in the solution is about 8.0 wt. %. In an embodiment, the percent SPF in the solution is about 8.5 wt. %. In an embodiment, the percent SPF in the solution is about 9.0 wt. %. In an embodiment, the percent SPF in the solution is about 9.5 wt. %. In an embodiment, the percent SPF in the solution is about 10.0 wt. %.

In an embodiment, the percent sericin in the solution is non-detectable to 25.0 wt. %. In an embodiment, the percent sericin in the solution is non-detectable to 5.0 wt. %. In an embodiment, the percent sericin in the solution is 1.0 wt. %. In an embodiment, the percent sericin in the solution is 2.0 wt. %. In an embodiment, the percent sericin in the solution is 3.0 wt. %. In an embodiment, the percent sericin in the solution is 4.0 wt. %. In an embodiment, the percent sericin in the solution is 5.0 wt. %. In an embodiment, the percent sericin in the solution is 10.0 wt. %. In an embodiment, the percent sericin in the solution is 25.0 wt. %.

In some embodiments, the silk fibroin protein fragments of the present disclosure are shelf stable (they will not slowly or spontaneously gel when stored in an aqueous solution and there is no aggregation of fragments and therefore no increase in molecular weight over time), from 10 days to 3 years depending on storage conditions, percent SPF, and number of shipments and shipment conditions. Additionally, pH may be altered to extend shelf life and/or support shipping conditions by preventing premature folding and aggregation of the silk. In an embodiment, the stability of the LiBr-silk fragment solution is 0 to 1 year. In an embodiment, the stability of the LiBr-silk fragment solution is 0 to 2 years. In an embodiment, the stability of the LiBr-silk fragment solution is 0 to 3 years. In an embodiment, the stability of the LiBr-silk fragment solution is 0 to 4 years. In an embodiment, the stability of the LiBr-silk fragment solution is 0 to 5 years. In an embodiment, the stability of the LiBr-silk fragment solution is 1 to 2 years. In an embodiment, the stability of the LiBr-silk fragment solution is 1 to 3 years. In an embodiment, the stability of the LiBr-silk fragment solution is 1 to 4 years. In an embodiment, the stability of the LiBr-silk fragment solution is 1 to 5 years. In an embodiment, the stability of the LiBr-silk fragment solution is 2 to 3 years. In an embodiment, the stability of the LiBr-silk fragment solution is 2 to 4 years. In an embodiment, the stability of the LiBr-silk fragment solution is 2 to 5 years. In an embodiment, the stability of the LiBr-silk fragment solution is 3 to 4 years. In an embodiment, the stability of the LiBr-silk fragment solution is 3 to 5 years. In an embodiment, the stability of the LiBr-silk fragment solution is 4 to 5 years.

In an embodiment, the stability of a composition of the present disclosure is 10 days to 6 months. In an embodiment, the stability of a composition of the present disclosure is 6 months to 12 months. In an embodiment, the stability of a composition of the present disclosure is 12 months to 18 months. In an embodiment, the stability of a composition of the present disclosure is 18 months to 24 months. In an embodiment, the stability of a composition of the present disclosure is 24 months to 30 months. In an embodiment, the stability of a composition of the present disclosure is 30 months to 36 months. In an embodiment, the stability of a composition of the present disclosure is 36 months to 48 months. In an embodiment, the stability of a composition of the present disclosure is 48 months to 60 months.

In an embodiment, a composition of the present disclosure having SPF has non-detectable levels of LiBr residuals. In an embodiment, the amount of the LiBr residuals in a composition of the present disclosure is between 10 ppm and 1000 ppm. In an embodiment, the amount of the LiBr residuals in a composition of the present disclosure is between 10 ppm and 300 ppm. In an embodiment, the amount of the LiBr residuals in a composition of the present disclosure is less than 25 ppm. In an embodiment, the amount of the Li Br residuals in a composition of the present disclosure is less than 50 ppm. In an embodiment, the amount of the LiBr residuals in a composition of the present disclosure is less than 75 ppm. In an embodiment, the amount of the LiBr residuals in a composition of the present disclosure is less than 100 ppm. In an embodiment, the amount of the LiBr residuals in a composition of the present disclosure is less than 200 ppm. In an embodiment, the amount of the LiBr residuals in a composition of the present disclosure is less than 300 ppm. In an embodiment, the amount of the LiBr residuals in a composition of the present disclosure is less than 400 ppm. In an embodiment, the amount of the LiBr residuals in a composition of the present disclosure is less than 500 ppm. In an embodiment, the amount of the LiBr residuals in a composition of the present disclosure is less than 600 ppm. In an embodiment, the amount of the LiBr residuals in a composition of the present disclosure is less than 700 ppm. In an embodiment, the amount of the LiBr residuals in a composition of the present disclosure is less than 800 ppm. In an embodiment, the amount of the LiBr residuals in a composition of the present disclosure is less than 900 ppm. In an embodiment, the amount of the LiBr residuals in a composition of the present disclosure is less than 1000 ppm. In an embodiment, the amount of the LiBr residuals in a composition of the present disclosure is non-detectable to 500 ppm. In an embodiment, the amount of the LiBr residuals in a composition of the present disclosure is non-detectable to 450 ppm. In an embodiment, the amount of the LiBr residue in a composition of the present disclosure is non-detectable to 400 ppm. In an embodiment, the amount of the LiBr residuals in a composition of the present disclosure is non-detectable to 350 ppm. In an embodiment, the amount of the LiBr residuals in a composition of the present disclosure is non-detectable to 300 ppm. In an embodiment, the amount of the LiBr residuals in a composition of the present disclosure is non-detectable to 250 ppm. In an embodiment, the amount of the LiBr residuals in a composition of the present disclosure is non-detectable to 200 ppm. In an embodiment, the amount of the LiBr residuals in a composition of the present disclosure is non-detectable to 150 ppm. In an embodiment, the amount of the LiBr residuals in a composition of the present disclosure is non-detectable to 100 ppm. In an embodiment, the amount of the LiBr residuals in a composition of the present disclosure is 100 ppm to 200 ppm. In an embodiment, the amount of the LiBr residuals in a composition of the present disclosure is 200 ppm to 300 ppm. In an embodiment, the amount of the LiBr residuals in a composition of the present disclosure is 300 ppm to 400 ppm. In an embodiment, the amount of the LiBr residuals in a composition of the present disclosure is 400 ppm to 500 ppm.

In an embodiment, a composition of the present disclosure having SPF, has non-detectable levels of Na₂CO₃ residuals. In an embodiment, the amount of the Na₂CO₃ residuals in a composition of the present disclosure is less than 100 ppm. In an embodiment, the amount of the Na₂CO₃ residuals in a composition of the present disclosure is less than 200 ppm. In an embodiment, the amount of the Na₂CO₃ residuals in a composition of the present disclosure is less than 300 ppm. In an embodiment, the amount of the Na₂CO₃ residuals in a composition of the present disclosure is less than 400 ppm. In an embodiment, the amount of the Na₂CO₃ residuals in a composition of the present disclosure is less than 500 ppm. In an embodiment, the amount of the Na₂CO₃ residuals in a composition of the present disclosure is less than 600 ppm. In an embodiment, the amount of the Na₂CO₃ residuals in a composition of the present disclosure is less than 700 ppm. In an embodiment, the amount of the Na₂CO₃ residuals in a composition of the present disclosure is less than 800 ppm. In an embodiment, the amount of the Na₂CO₃ residuals in a composition of the present disclosure is less than 900 ppm. In an embodiment, the amount of the Na₂CO₃ residuals in a composition of the present disclosure is less than 1000 ppm. In an embodiment, the amount of the Na₂CO₃ residuals in a composition of the present disclosure is non-detectable to 500 ppm. In an embodiment, the amount of the Na₂CO₃ residuals in a composition of the present disclosure is non-detectable to 450 ppm. In an embodiment, the amount of the Na₂CO₃ residuals in a composition of the present disclosure is non-detectable to 400 ppm. In an embodiment, the amount of the Na₂CO₃ residuals in a composition of the present disclosure is non-detectable to 350 ppm. In an embodiment, the amount of the Na₂CO₃ residuals in a composition of the present disclosure is non-detectable to 300 ppm. In an embodiment, the amount of the Na₂CO₃ residuals in a composition of the present disclosure is non-detectable to 250 ppm. In an embodiment, the amount of the Na₂CO₃ residuals in a composition of the present disclosure is non-detectable to 200 ppm. In an embodiment, the amount of the Na₂CO₃ residuals in a composition of the present disclosure is non-detectable to 150 ppm. In an embodiment, the amount of the Na₂CO₃ residuals in a composition of the present disclosure is non-detectable to 100 ppm. In an embodiment, the amount of the Na₂CO₃ residuals in a composition of the present disclosure is 100 ppm to 200 ppm. In an embodiment, the amount of the Na₂CO₃ residuals in a composition of the present disclosure is 200 ppm to 300 ppm. In an embodiment, the amount of the Na₂CO₃ residuals in a composition of the present disclosure is 300 ppm to 400 ppm. In an embodiment, the amount of the Na₂CO₃ residuals in a composition of the present disclosure is 400 ppm to 500 ppm.

A unique feature of the SPF compositions of the present disclosure are shelf stability (they will not slowly or spontaneously gel when stored in an aqueous solution and there is no aggregation of fragments and therefore no increase in molecular weight over time), from 10 days to 3 years depending on storage conditions, percent silk, and number of shipments and shipment conditions. Additionally pH may be altered to extend shelf-life and/or support shipping conditions by preventing premature folding and aggregation of the silk. In an embodiment, a SPF solution composition of the present disclosure has a shelf stability for up to 2 weeks at room temperature (RT). In an embodiment, a SPF solution composition of the present disclosure has a shelf stability for up to 4 weeks at RT. In an embodiment, a SPF solution composition of the present disclosure has a shelf stability for up to 6 weeks at RT. In an embodiment, a SPF solution composition of the present disclosure has a shelf stability for up to 8 weeks at RT. In an embodiment, a SPF solution composition of the present disclosure has a shelf stability for up to 10 weeks at RT. In an embodiment, a SPF solution composition of the present disclosure has a shelf stability for up to 12 weeks at RT. In an embodiment, a SPF solution composition of the present disclosure has a shelf stability ranging from about 4 weeks to about 52 weeks at RT.

Table 18 below shows shelf stability test results for embodiments of SPF compositions of the present disclosure.

TABLE 18
Shelf Stability of SPF Compositions of the Present Disclosure

| % Silk | Temperature | Time to Gelation |
|---|---|---|
| 2 | RT | 4 weeks |
| 2 | 4° C. | >9 weeks |
| 4 | RT | 4 weeks |
| 4 | 4° C. | >9 weeks |
| 6 | RT | 2 weeks |
| 6 | 4° C. | >9 weeks |

In some embodiments, the water solubility of the silk film derived from silk fibroin protein fragments as described herein can be modified by solvent annealing (water annealing or methanol annealing), chemical crosslinking, enzyme crosslinking and heat treatment.

In some embodiments, the process of annealing may involve inducing beta-sheet formation in the silk fibroin protein fragment solutions used as a coating material. Techniques of annealing (e.g., increase crystallinity) or otherwise promoting “molecular packing” of silk fibroin-protein based fragments have been described. In some embodiments, the amorphous silk film is annealed to introduce beta-sheet in the presence of a solvent selected from the group of water or organic solvent. In some embodiments, the amorphous silk film is annealed to introduce beta-sheet in the presence of water (water annealing process). In some embodiments, the amorphous silk fibroin protein fragment film is annealed to introduce beta-sheet in the presence of methanol. In some embodiments, annealing (e.g., the beta sheet formation) is induced by addition of an organic solvent. Suitable organic solvents include, but are not limited to methanol, ethanol, acetone, isopropanol, or combination thereof.

In some embodiments, annealing is carried out by so-called “water-annealing” or “water vapor annealing” in which water vapor is used as an intermediate plasticizing agent or catalyst to promote the packing of beta-sheets. In some embodiments, the process of water annealing may be performed under vacuum. Suitable such methods have been described in Jin H-J et al. (2005), Water-stable Silk Films with Reduced Beta-Sheet Content, Advanced Functional Materials, 15: 1241-1247; Xiao H. et al. (2011), Regulation of Silk Material Structure by Temperature-Controlled Water Vapor Annealing, Biomacromolecules, 12(5): 1686-1696.

The important feature of the water annealing process is to drive the formation of crystalline beta-sheet in the silk fibroin protein fragment peptide chain to allow the silk fibroin self-assembling into a continuous film. In some embodiments, the crystallinity of the silk fibroin protein fragment film is controlled by controlling the temperature of water vapor and duration of the annealing. In some embodiments, the annealing is performed at a temperature ranging from about 65° C. to about 110° C. In some embodiments, the temperature of the water is maintained at about 80° C. In some embodiments, annealing is performed at a temperature selected from the group of about 65° C., about 70° C., about 75° C., about 80° C., about 85° C., about 90° C., about 95° C., about 100° C., about 105° C., and about 110° C.

In some embodiments, the annealing process lasts a period of time selected from the group of about 1 minute to about 40 minutes, about 1 minute to about 50 minutes, about 1 minute to about 60 minutes, about 1 minute to about 70 minutes, about 1 minute to about 80 minutes, about 1 minute to about 90 minutes, about 1 minute to about 100 minutes, about 1 minute to about 110 minutes, about 1 minute to about 120 minutes, about 1 minute to about 130 minutes, about 5 minutes to about 40 minutes, about 5 minutes to about 50 minutes, about 5 minutes to about 60 minutes, about 5 minutes to about 70 minutes, about 5 minutes to about 80 minutes, about 5 minutes to about 90 minutes, about 5 minutes to about 100 minutes, about 5 minutes to about 110 minutes, about 5 minutes to about 120 minutes, about 5 minutes to about 130 minutes, about 10 minutes to about 40 minutes, about 10 minutes to about 50 minutes, about 10 minutes to about 60 minutes, about 10 minutes to about 70 minutes, about 10 minutes to about 80 minutes, about 10 minutes to about 90 minutes, about 10 minutes to about 100 minutes, about 10 minutes to about 110 minutes, about 10 minutes to about 120 minutes, about 10 minutes to about 130 minutes, about 15 minutes to about 40 minutes, about 15 minutes to about 50 minutes, about 15 minutes to about 60 minutes, about 15 minutes to about 70 minutes, about 15 minutes to about 80 minutes, about 15 minutes to about 90 minutes, about 15 minutes to about 100 minutes, about 15 minutes to about 110 minutes, about 15 minutes to about 120 minutes, about 15 minutes to about 130 minutes, about 20 minutes to about 40 minutes, about 20 minutes to about 50 minutes, about 20 minutes to about 60 minutes, about 20 minutes to about 70 minutes, about 20 minutes to about 80 minutes, about 20 minutes to about 90 minutes, about 20 minutes to about 100 minutes, about 20 minutes to about 110 minutes, about 20 minutes to about 120 minutes, about 20 minutes to about 130 minutes, about 25 minutes to about 40 minutes, about 25 minutes to about 50 minutes, about 25 minutes to about 60 minutes, about 25 minutes to about 70 minutes, about 25 minutes to about 80 minutes, about 25 minutes to about 90 minutes, about 25 minutes to about 100 minutes, about 25 minutes to about 110 minutes, about 25 minutes to about 120 minutes, about 25 minutes to about 130 minutes, about 30 minutes to about 40 minutes, about 30 minutes to about 50 minutes, about 30 minutes to about 60 minutes, about 30 minutes to about 70 minutes, about 30 minutes to about 80 minutes, about 30 minutes to about 90 minutes, about 30 minutes to about 100 minutes, about 30 minutes to about 110 minutes, about 30 minutes to about 120 minutes, about 30 minutes to about 130 minutes, about 35 minutes to about 40 minutes, about 35 minutes to about 50 minutes, about 35 minutes to about 60 minutes, about 35 minutes to about 70 minutes, about 35 minutes to about 80 minutes, about 35 minutes to about 90 minutes, about 35 minutes to about 100 minutes, about 35 minutes to about 110 minutes, about 35 minutes to about 120 minutes, about 35 minutes to about 130 minutes, about 40 minutes to about 50 minutes, about 40 minutes to about 60 minutes, about 40 minutes to about 70 minutes, about 40 minutes to about 80 minutes, about 40 minutes to about 90 minutes, about 40 minutes to about 100 minutes, about 40 minutes to about 110 minutes, about 40 minutes to about 120 minutes, about 40 minutes to about 130 minutes, about 45 minutes to about 50 minutes, about 45 minutes to about 60 minutes, about 45 minutes to about 70 minutes, about 45 minutes to about 80 minutes, about 45 minutes to about 90 minutes, about 45 minutes to about 100 minutes, about 45 minutes to about 110 minutes, about 45 minutes to about 120 minutes, and about 45 minutes to about 130 minutes. In some embodiments, the annealing process lasts a period of time ranging from about 1 minute to about 60 minutes. In some embodiments, the annealing process lasts a period of time ranging from about 45 minutes to about 60 minutes. The longer water annealing post-processing corresponded an increased crystallinity of silk fibroin protein fragments.

In some embodiments, the annealed silk fibroin protein fragment film is immersing the wet silk fibroin protein fragment film in 100% methanol for 60 minutes at room temperature. The methanol annealing changed the composition of silk fibroin protein fragment film from predominantly amorphous random coil to crystalline antiparallel beta-sheet structure.

In some embodiments, the SPF as described herein can be used to prepare SPF microparticles by precipitation with methanol. Alternative flash drying, fluid-bed drying, spray drying or vacuum drying can be applied to remove water from the silk solution. The SPF powder can then be stored and handled without refrigeration or other special handling procedures. In some embodiments, the SPF powders comprise low molecular weight silk fibroin protein fragments. In some embodiments, the SPF powders comprise mid-molecular weight silk fibroin protein fragments. In some embodiments, the SPF powders comprise a mixture of low molecular weight silk fibroin protein fragments and mid-molecular weight silk fibroin protein fragment.

In an embodiment, the water solubility of pure silk fibroin protein fragments of the present disclosure is 50 to 100%. In an embodiment, the water solubility of pure silk fibroin protein fragments of the present disclosure is 60 to 100%. In an embodiment, the water solubility of pure silk fibroin protein fragments of the present disclosure is 70 to 100%. In an embodiment, the water solubility of pure silk fibroin protein fragments of the present disclosure is 80 to 100%. In an embodiment, the water solubility is 90 to 100%. In an embodiment, the silk fibroin fragments of the present disclosure are non-soluble in aqueous solutions.

In an embodiment, the solubility of pure silk fibroin protein fragments of the present disclosure in organic solutions is 50 to 100%. In an embodiment, the solubility of pure silk fibroin protein fragments of the present disclosure in organic solutions is 60 to 100%. In an embodiment, the solubility of pure silk fibroin protein fragments of the present disclosure in organic solutions is 70 to 100%. In an embodiment, the solubility of pure silk fibroin protein fragments of the present disclosure in organic solutions is 80 to 100%. In an embodiment, the solubility of pure silk fibroin protein fragments of the present disclosure in organic solutions is 90 to 100%. In an embodiment, the silk fibroin fragments of the present disclosure are non-soluble in organic solutions.

In some embodiments, the silk fibroin protein fragments comprise cationic quaternized amino acid residue (cationic quaternized silk fibroin) with fatty alkyl groups, wherein the silk fibroin protein fragments having any weight average molecular weight and polydispersity described herein. In some embodiments, the fatty alkyl group for quaternization of amine groups of the silk fibroin protein fragment is selected from the group of cocodimonium hydroxypropyl, hydroxypropyltrimonium, lauryidimonium hydroxypropyl, steardimonium hydroxypropyl, quaternium-79, and combinations thereof.

In some embodiments, lyophilized silk powder can be resuspended in water, hexafluoroisopropanol (HFIP), or organic solution following storage to create silk solutions of varying concentrations, including higher concentration solutions than those produced initially. In another embodiment, the silk fibroin-based protein fragments are dried using a rototherm evaporator or other methods known in the art for creating a dry protein form containing less than 10% water by mass. In an embodiment, the solubility of silk fibroin-based protein fragments of the present disclosure in organic solutions ranges from about 50.0% to about 100%. In an embodiment, the solubility of silk fibroin-based protein fragments of the present disclosure in organic solutions ranges from about 60.0% to about 100%. In an embodiment, the solubility of silk fibroin-based protein fragments of the present disclosure in organic solutions ranges from about 70.0% to about 100%. In an embodiment, the solubility of silk fibroin-based protein fragments of the present disclosure in organic solutions ranges from about 80.0% to about 100%. In an embodiment, the solubility of silk fibroin-based protein fragments of the present disclosure in organic solutions ranges from about 90.0% to about 100%. In an embodiment, the silk fibroin-based fragments of the present disclosure are non-soluble in organic solutions.

In some embodiments, SPF, e.g., without limitation silk fibroin protein fragments useful for applications in hair care products also include an aqueous gel of the SPF, e.g., without limitation silk fibroin protein fragments. The gelation of silk solutions may be induced by sonication, vortex, heating, solvent treatment (e.g. methanol, ethanol), electrogelation, ultrasonication, chemicals (e.g. vitamin C), or the like.

Silk peptide is an extract from natural silk fibroin hydrolysate. Silk peptide exhibits pearl luster and silky feel when incorporated into personal care products. The structure of silk peptide is similar to human hair and skin tissue. The silk peptides are serine rich polypeptides having 10 or more amino acid residues and weight average molecular weights as described herein. In some embodiments, the silk peptide extract can be easily absorbed by hair, for example human hair, provide nutrients for hair, and promote the metabolism of hair.

In some embodiments, SPF, e.g., without limitation silk fibroin protein fragment solutions useful for applications in hair care products also include low molecular weight silk fibroin peptides (weight average molecular weight of about 200 Da to 5 kDa). The low molecular weight silk fibroin peptides derived from silk fibroin protein hydrolysate can complement the natural moisturizing factors in the free amino acids to improve the hair scalp moisture content. In some embodiments, the low molecular weight silk fibroin peptides can penetrate deep into the hair follicle to repair, replenish water, nourish hair, improve the moisture balance, and prevent dandruff generation. In some embodiments, SPF, e.g., without limitation silk fibroin protein fragment solutions useful for applications in hair care products also include silk fibroin protein amino acids derived from the hydrolyzed silk fibroin.

In some embodiments, the SPF, e.g., without limitation silk fibroin protein fragments as described herein can act as detergents for cleansing, wetting agents for better spreadability, emulsifiers to create stable mixtures of oil and water, conditioning agents to improve the appearance of hair. In some embodiments, the SPF, e.g., without limitation silk fibroin protein fragment solution exhibits enhanced emulsification power as compared with colloidal silk fibroin protein. In some embodiments, the hair care composition incorporated with SPF, e.g., without limitation silk fibroin protein fragment solution exhibits enhanced beneficial effects of the self-assembly and coating properties of the silk fibroin peptides in view of those of the full length silk fibroin protein with functional folding structure.

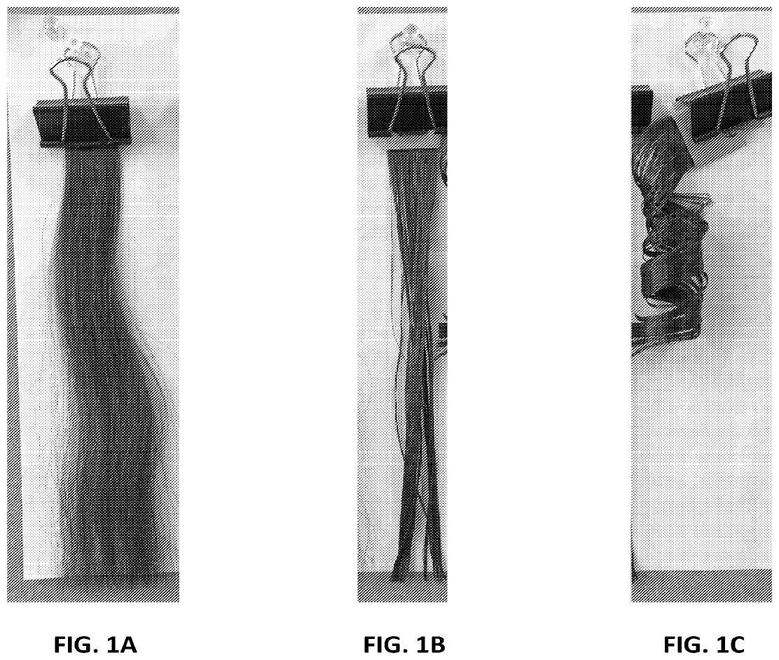

In an embodiment, this disclosure provides the use of SPF, e.g., without limitation silk fibroin protein fragments for straightening hair, in particular, and preferably, the naturally wavy hair (FIG. 1). In an embodiment, this disclosure provides the use of SPF, e.g., without limitation silk fibroin protein fragments for curling hair (FIG. 1C). In an embodiment, this disclosure provides the use of SPF, e.g., without limitation silk fibroin protein fragments for hair frizz control. In an embodiment, this disclosure provides the use of SPF, e.g., without limitation silk fibroin protein fragments for hair conditioning. In an embodiment, this disclosure provides the use of SPF, e.g., without limitation silk fibroin protein fragments as lathering agent for a shampoo composition.

Keratin interacts with SPF, e.g., without limitation silk fibroin protein fragments resulting in smooth, soft hair and an upgraded hair straightening quality, leaving hair smooth and silky.

### A Chemical Modifier or a Physical Modifier

In some embodiments, the chemical modifier is chemically linked to one or more of a silk fibroin side group and a silk fibroin terminal group. In some embodiments, the silk fibroin side group and the silk fibroin terminal group are independently selected from an amine group, an amide group, a carboxyl group, a hydroxyl group, a thiol group, and a sulfhydryl group. In some embodiments, the chemical modifier is chemically linked to one or more functional groups on the substrate. In some embodiments, the functional group on the substrate is selected from an amine group, an amide group, a carboxyl group, a hydroxyl group, a thiol group, and a sulfhydryl group. In some embodiments, the chemical modifier includes one or more of a chemically linked functional group, or functional group residue, and a linker. In some embodiments, the chemical modifier includes one or more of —CR^{a}₂—, —CR^{a}═CR^{a}—, —C≡C—, -alkyl-, -alkenyl-, -alkynyl-, -aryl-, -heteroaryl-, —O—, —S—, —OC(O)—, —N(R^{a})—, —N═N—, ═N—, —C(O)—, —C(O)O—, —OC(O)N(R^{a})—, —C(O)N(R^{a})—, —N(R^{a})C(O)O—, —N(R^{a})C(O)—, —N(R^{a})C(O)N(R^{a})—, —N(R^{a})C(NR^{a})N(R^{a})—, —N(R^{a})S(O)ₜ—, —S(O)ₜO—, —S(O)ₜN(R^{a})—, —S(O)ₜN(R^{a})C(O)—, —OP(O)(OR^{a})O—, wherein t is 1 or 2, and wherein at each independent occurrence R^{a} is selected from hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

Structure

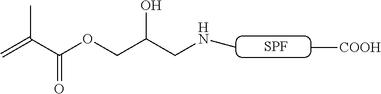

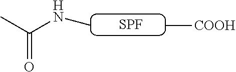

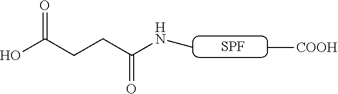

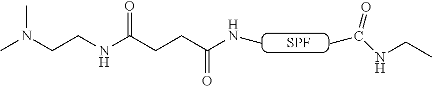

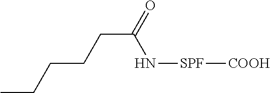

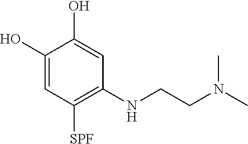

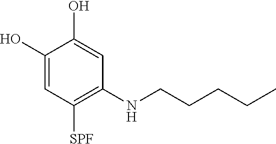

Chemical Structure

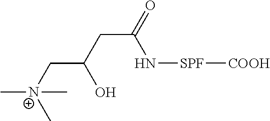

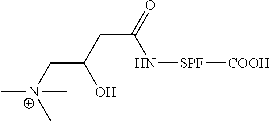

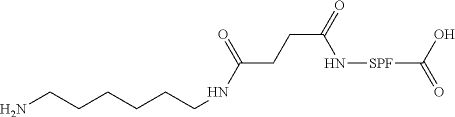

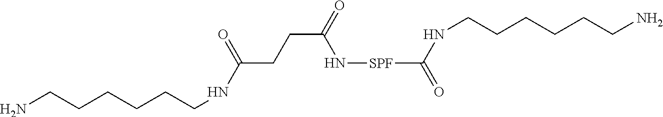

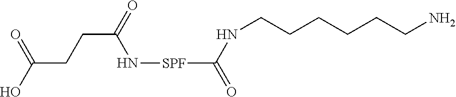

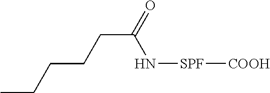

In some embodiments, the chemical modifier is chemically linked to one or more of a silk fibroin side group and a silk fibroin terminal group. In some embodiments, the silk fibroin side group and the silk fibroin terminal group are independently selected from an amine group, an amide group, a carboxyl group, a hydroxyl group, a thiol group, and a sulfhydryl group. In some embodiments, the chemical modifier is chemically linked to one or more functional groups on the substrate. In some embodiments, the functional group on the substrate is selected from an amine group, an amide group, a carboxyl group, a hydroxyl group, a thiol group, and a sulfhydryl group. In some embodiments, the chemical modifier includes one or more of a chemically linked functional group, or functional group residue, and a linker. In some embodiments, the chemical modifier includes one or more of —CR^{a}₂—, —CR^{a}═CR^{a}—, —C≡C—, -alkyl-, -alkenyl-, -alkynyl-, -aryl-, -heteroaryl-, —O—, —S—, —OC(O)—, —N(R^{a})—, —N═N—, ═N—, —C(O)—, —C(O)O—, —OC(O)N(R^{a})—, —C(O)N(R^{a})—, —N(R^{a})C(O)O—, —N(R^{a})C(O)—, —N(R^{a})C(O)N(R^{a})—, —N(R^{a})C(NR^{a})N(R^{a})—, —N(R^{a})S(O)ₜ—, —S(O)ₜO—, —S(O)ₜN(R^{a})—, —S(O)ₜN(R^{a})C(O)—, —OP(O)(OR^{a})O—, wherein t is 1 or 2, and wherein at each independent occurrence R^{a} is selected from hydrogen, alkyl, alkenyl, fluoroalkyl, carbocyclyl, carbocyclylalkyl, aryl, aralkyl, heterocycloalkyl, heterocycloalkylalkyl, heteroaryl, or heteroarylalkyl.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocyclyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acylsulfonamide” refers a —S(O)₂—N(R^{a})—C(═O)— radical, where R^{a} is hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Unless stated otherwise specifically in the specification, an acylsulfonamide group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{a}, and NR^{a}R^{a} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “aryloxy” refers to the group —O-aryl.

The term “substituted aryloxy” refers to aryloxy wherein the aryl substituent is substituted (i.e., —O-(substituted aryl)). Unless stated otherwise specifically in the specification, the aryl moiety of an aryloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Nitro” refers to the —NO₂ radical.

“Oxa” refers to the —O— radical.

“Oxo” refers to the ═O radical.

“Isomers” are different compounds that have the same molecular formula.

“Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. “Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)— (optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

### Hair Conditioning Agents

When washing hair with conventional shampoo compositions, the natural oils are removed together with the dirt and unwanted oils. When too much of the natural oil is removed, for example by especially frequent washing, the hair becomes less easy to comb or style, and subject to static build-up causing “flyaway”. Hair conditioning agents have been incorporated into the hair care composition to restore the condition of the hair. It is believed that the conditioning agent provides improved conditioning benefits to the hair, particularly clean hair feel and wet rinse feel.

### A. Silk Conditioning Agent

In some embodiments, one or more of the SPF, e.g., without limitation silk fibroin protein based fragments, the silk fibroin amino acids and silk peptides are incorporated as functional additives to the hair care composition to impart hair conditioning benefits, for example, adding water soluble SPF, e.g., without limitation silk fibroin protein derived peptide having 2-50 amino acid units to a detersive surfactant for shampoo, silk fibroin hydrolysate as humectant, silk fibroin protein hydrolysate with an average molecular weight of 1000 Da as hair conditioning agent for a shampoo composition, amino acids derived from the silk fibroin protein hydrolysate as hair nutrients.

In some embodiments, the silk fibroin amino acids are from commercially available hydrolyzed silk (CAS Number: 96690-41-4). The amino acid composition derived from the silk fibroin protein of Bombyx mori consists mainly of Gly (43%), Ala (30%), and Ser (12%).

In some embodiments, the silk conditioning agent is low molecular weight silk peptides have a weight average molecular weight ranging from about 200 Da to about 4 kDa. In some embodiments, the silk conditioning agent is the silk fibroin-based protein fragments have a weight average molecular weight ranging from about 200 Da to about 1 kDa. In some embodiments, the silk conditioning agent is the silk fibroin-based protein fragments have a weight average molecular weight of about 1 kDa.

In some embodiments, the silk conditioning agent is the silk fibroin-based protein fragments having a weight average molecular weight ranging from about 5 kDa to about 17 kDa, wherein the silk fibroin-based protein fragments have a polydispersity ranging from about 1.5 to about 3.0.

In some embodiments, the silk conditioning agent comprises the silk fibroin-protein based fragments covalently modified with at least one functional groups. In some embodiments, the functional group comprises cationic functional group, anionic functional group, hexamine, 2-hydroxy-γ-amino acid, succinic acid, C6-C36 fatty acid to provide functionalized silk fibroin-based protein fragments. In some embodiments, the functionalized silk fibroin-protein based fragments comprise Low-MW silk. In some embodiments, the functionalized silk fibroin-protein based fragments comprise Mid-MW silk.

In some embodiments, the at least one functional group attached to silk fibroin-protein based fragments via a spacer. In some embodiments, the spacer links the silk fibroin-based protein fragments and the functional group via an amide bond formed by N-hydroxysuccinimide/carbodiimide (NHS/EDC) coupling chemistry.

In some embodiments, the spacer is selected from the group of polyethylene glycol having 2-50 repeating units, ε-maleimidocaproic acid, para-aminobenzyloxy carbamate, and combinations thereof. In some embodiments, the spacer comprises polyamino acid having 2-30 amino acid residues. In some embodiments, the spacer comprises a linear polylysine, or polyglutamine. In some embodiments, the spacer may include succinic acid, hexamine.

In some embodiments, the functionalized silk fibroin-protein based fragments have Formula (1)

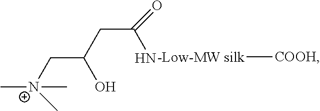

wherein the Low-MW silk has a weight average molecular weight ranging from about 5 kDa to about 30 kDa, and a polydispersity ranging from 1.0 to about 5.0. In some embodiments, the functionalized silk fibroin-protein based fragments comprise the Low-MW silk having a weight average molecular weight ranging from about 5 kDa to about 30 kDa and a polydispersity ranging from about 1.5 to about 3.0. In some embodiments, the functionalized silk fibroin-protein based fragments comprise the Low-MW silk having a weight average molecular weight ranging from about 5 kDa to about 17 kDa and a polydispersity ranging from about 1.5 to about 3.0.

In some embodiments, the functionalized silk fibroin-protein based fragments have Formula (2)

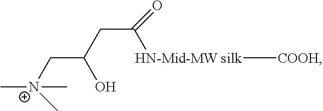

wherein the Mid-MW silk has a weight average molecular weight ranging from about 25 kDa to about 60 kDa, and a polydispersity ranging from 1.0 to about 5.0. In some embodiments, the functionalized silk fibroin-protein based fragments comprise Mid-MW silk having a weight average molecular weight ranging from about 25 kDa to 60 kDa, and a polydispersity ranging from about 1.5 to about 3.0. In some embodiments, the functionalized silk fibroin-protein based fragments comprise Mid-MW silk having a weight average molecular weight ranging from about 39 kDa to about 54 kDa, and a polydispersity ranging from about 1.5 to about 3.0.

In some embodiments, the functionalized silk fibroin-protein based fragments is a compound selected from Formulae (3), (4), or (5):

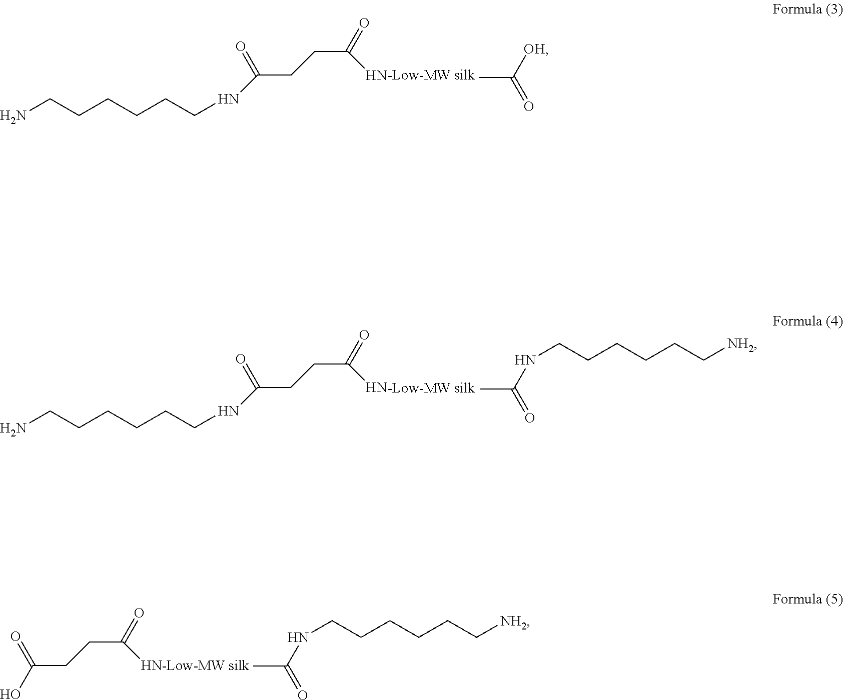

wherein the Low-MW silk has a weight average molecular weight ranging from about 5 kDa to about 25 kDa, and a polydispersity ranging from 1.0 to about 5.0. In some embodiments, the functionalized silk fibroin-protein based fragments comprise the Low-MW silk having a weight average molecular weight ranging from about 5 kDa to about 30 kDa and a polydispersity ranging from about 1.5 to about 3.0. In some embodiments, the functionalized silk fibroin-protein based fragments comprise the Low-MW silk having a weight average molecular weight ranging from about 5 kDa to about 17 kDa and a polydispersity ranging from about 1.5 to about 3.0.

In some embodiments, the functionalized silk fibroin-protein based fragments is a compound selected from Formulae (6), (7), or (8):

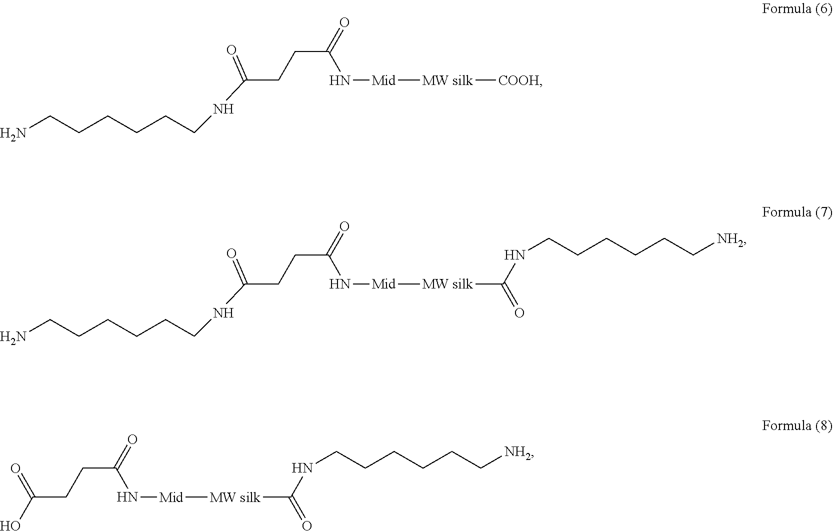

wherein the Mid-MW silk has a weight average molecular weight ranging from about 25 kDa to about 60 kDa, and a polydispersity ranging from 1.0 to about 5.0. In some embodiments, the functionalized silk fibroin-protein based fragments comprise Mid-MW silk having a weight average molecular weight ranging from about 25 kDa to 60 kDa, and a polydispersity ranging from about 1.5 to about 3.0. In some embodiments, the functionalized silk fibroin-protein based fragments comprise Mid-MW silk having a weight average molecular weight ranging from about 39 kDa to about 54 kDa, and a polydispersity ranging from about 1.5 to about 3.0.

In some embodiments, the functionalized silk fibroin-protein based fragments has Formula (9)

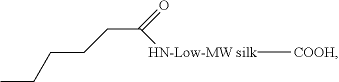

wherein the Low-MW silk has a weight average molecular weight ranging from about 5 kDa to about 30 kDa, and a polydispersity ranging from 1.0 to about 5.0. In some embodiments, the functionalized silk fibroin-protein based fragments comprise the Low-MW silk having a weight average molecular weight ranging from about 5 kDa to about 25 kDa and a polydispersity ranging from about 1.5 to about 3.0. In some embodiments, the functionalized silk fibroin-protein based fragments comprise the Low-MW silk having a weight average molecular weight ranging from about 5 kDa to about 17 kDa and a polydispersity ranging from about 1.5 to about 3.0.

In some embodiments, the functionalized silk fibroin-protein based fragments has Formula (10)

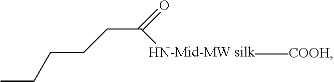

wherein the Mid-MW silk has a weight average molecular weight ranging from about 25 kDa to about 60 kDa, and a polydispersity ranging from 1.0 to about 5.0. In some embodiments, the functionalized silk fibroin-protein based fragments comprise Mid-MW silk having a weight average molecular weight ranging from about 25 kDa to 60 kDa, and a polydispersity ranging from about 1.5 to about 3.0. In some embodiments, the functionalized silk fibroin-protein based fragments comprise Mid-MW silk having a weight average molecular weight ranging from about 39 kDa to about 54 kDa, and a polydispersity ranging from about 1.5 to about 3.0.

In some embodiments, the functionalized silk fibroin-protein based fragments may find use as natural hair ingredient to replace the conventional toxic chemical agent, e.g., as substitute for thioglycolates or formaldehyde in keratin treatment product (known as Brazilian Blowout, Cezanne, Goldwell Kerasilk).

In some embodiments, the amount of silk conditioning agent incorporated in the hair care composition ranges from about 0.2 wt. % to about 0.6 wt. % by the total weight of the hair care composition. In some embodiments, the use amount of silk conditioning agent incorporated in the hair care composition is selected from: about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, about 0.5 wt. %, about 0.51 wt. %, about 0.52 wt. %, about 0.53 wt. %, about 0.54 wt. %, about 0.55 wt. %, about 0.56 wt. %, about 0.57 wt. %, about 0.58 wt. %, about 0.59 wt. %, about 0.6 wt. % by the total weight of the hair care composition.

### B. Organic Conditioning Oils

In some embodiments, the hair care composition comprises at least one organic conditioning oil in addition to silk conditioning agent. Without wishing to be bound by any particular theory, it is believed that these organic conditioning oils provide the hair care composition with improved conditioning performance when used in combination with the silk conditioning agent. The conditioning oils may add shine and luster to the hair. Additionally, they may enhance dry combing and dry hair feel.

The organic conditioning oils suitable for use as the conditioning agent herein are preferably low viscosity, water insoluble liquids selected from the group consisting of hydrocarbon oils, polyolefins, fatty esters, and mixtures thereof. In some embodiments, the organic conditioning oil is selected from hydrocarbon oils and fatty esters, and combination thereof.

In some embodiments, the hair conditioning agent comprises hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils preferably are from about C₁₂ to about C₁₉. Branched chain hydrocarbon oils, including hydrocarbon polymers, typically will contain more than 19 carbon atoms.

In some embodiments, the hydrocarbon oil is selected from the group consisting of liquid paraffin, liquid isoparaffin, squalene, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, permethyl-substituted isomers, e.g., the permethyl-substituted isomers of hexadecane and eicosane (e.g., 2,2,4,4,6,6,8,8-dimethyl-10-methylundecane and 2,2,4,4,6,6-dimethyl-8-methylnonane), copolymer of isobutylene and butane, poly-α-olefins (e.g., polymer of ethylene, propylene, 1-butene, 1-pentene, 1-hexene, 1-octene, 1-decene, 1-dodecene, 1-tetradecene), and combination thereof.

In some embodiments, the organic oil conditioning agent comprises fatty ester oil selected from the group consisting of isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, oleyl adipate, isopropyl myristate, glycol stearate, and isopropyl laurate, isocetyl stearoyl stearate, diisopropyl adipate, tristearyl citrate, triolein, tristearin glyceryl dilaurate, C₈-C₁₀ triester of trimethylolpropane, tetraester of 3,3 diethanol-1,5 pentadiol, C₈-C₁₀ diester of adipic acid, ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, sorbitan fatty acid esters, and polyoxyethylene sorbitan fatty acid esters (e.g. polyoxyethylene (20) sorbitan monooleate, polysorbate 80, Tween 80@), and combination thereof.

In some embodiments, the organic conditioning oils have a viscosity ranging from about 1 centipoise to about 200 centipoise, as measured at 40° C. In some embodiments, the organic conditioning oils have a viscosity ranging from about 1 centipoise to about 100 centipoise, as measured at 40° C. In some embodiments, the organic conditioning oils have a viscosity ranging from about 2 centipoise to about 50 centipoise, as measured at 40° C.

In some embodiments, the organic conditioning oil is presented in the hair care composition at a weight percent ranging from about 0.05 wt. % to about 10.0 wt. % by the total weight of the hair care composition. In some embodiments, organic conditioning oil is presented in the hair care composition at a weight percent ranging from about 0.2 wt. % to about 5.0 wt. % by the total weight of the hair care composition. In some embodiments, organic conditioning oil is presented in the hair care composition at a weight percent ranging from about 0.5 wt. % to about 3.0 wt. % by the total weight of the hair care composition.

### C. Silicone Conditioning Agents

In some embodiments, the hair care composition further comprises one or more silicone conditioning agents. In some embodiments, the silicone conditioning agents are hydrophobic and have a HLB of about 8 or less, preferably 5 or less. In some embodiments, the silicone conditioning agents are non-volatile, and are liquid at 25° C. In some embodiments, the silicone conditioning agent have a viscosity selected from: about 200 mm²·s⁻¹, from about 200 mm²·s⁻¹ to about 300,000 mm²·s⁻¹, from about 200 mm²·s⁻¹ to about 50,000 mm²·s⁻¹ at 25° C., in view of providing conditioning benefits.

The two most common types of hair conditioning silicone oils are referred to in the International Cosmetic Ingredient Dictionary (CTFA) as “dimethicone” and “dimethiconol”. Dimethicone is defined as a mixture of fully methylated linear siloxane polymers end blocked with trimethylsiloxy units. Dimethiconol is a dimethyl silicone polymer terminated with hydroxyl groups. Such hair conditioning silicone oils are relatively non-volatile liquids.

In some embodiments, the silicone hair conditioning agent is selected from the group consisting of either cyclic or linear polydimethylsiloxanes, phenyl and alkyl phenyl silicones, dimethyl polysiloxane, methylphenyl polysiloxane, amino-modified silicone, alcohol-modified silicone, aliphatic alcohol-modified silicone, polyether-modified silicone, epoxy-modified silicone, fluorine-modified silicone, and cyclic silicone or alkyl-modified silicone. In some embodiments, the hair care composition comprises dimethyl polysiloxane (degree of polymerization: 500 or above), polyether-modified silicone, amino-modified silicone, cyclic silicone, and combination thereof. The silicone hair conditioning agent are capable of imparting a good texture to the hair.

In some embodiments, the hair care composition comprises a polyalkylsiloxane selected from the group consisting of polydimethyl siloxanes with terminal trimethyl silyl groups or terminal dimethyl silanol groups (dimethiconol), and polyalkyl (C1-C25) siloxanes. In some embodiments, the hair care composition comprises a polyalkylarylsiloxane selected from the group consisting of linear or branched polydimethyl methyl phenyl siloxanes, linear or branched polydimethyl diphenyl siloxanes, and combination thereof.

In some embodiments, the hair care composition comprises a polydiorganosiloxane gum having a number-average molecular weight ranging from about 200,000 Da to 1,000,000 Da, used alone or mixed with a solvent. In some embodiments, the hair care composition comprises a silicone gum selected from the group consisting of polymethyl siloxane, polydimethyl siloxane/methyl vinyl siloxane gums, polydimethyl siloxane/diphenyl siloxane, polydimethyl siloxane/phenyl methyl siloxane and polydimethyl siloxane/diphenyl siloxane/methyl vinyl siloxanes, and combination thereof.

In some embodiments, the hair care composition comprises a silicone resins selected from silicones with a dimethyl/trimethyl siloxane structure, and resins of the trimethyl siloxysilicate type.

In some embodiments, the hair care composition comprises from about 0.1 wt. % to 10.0 wt. % by the total weight of a volatile silicone as the hair conditioning agent. Volatile silicones are well known in the art and are commercially available and include, e.g. linear and cyclic silicone compounds. Volatile silicone oils are preferably linear or cyclic polydimethylsiloxanes containing from about three to about nine silicon atoms. In some embodiments, the volatile silicone oil is octamethylcydotetrasiloxane (D4), or decamethylcydopentasiloxane (D5).

In some embodiments, the silicone hair conditioning agent comprises an aminosilicone. In some embodiments, an aminosilicone has the Formula (I):

(R¹)ₐX₃₋ₐ—Si—(—OSiX₂)ₙ—(—OSiX_{b}(R¹)_{2-b})ₘ—O—SiX₃₋ₐ(R¹)ₐ Formula (I)

wherein in Formula (I) X is hydrogen, phenyl, hydroxy, or C₁-C₈ alkyl, preferably methyl; a is an integer having a value from 1 to 3, preferably 1; b is 0, 1 or 2, preferably 1; n is a number from 1 to 2,000, preferably from 100 to 1,800, more preferably from 300 to 800, still more preferably 500-600; m is 0; R¹ is a monovalent radical conforming to the general formula C_{q}H_{2q}L, wherein q is an integer having a value from 2 to 8 and L is selected from the following groups: —N(R²)CH₂—CH₂—N(R²)₂; —N(R²)₂; —N(R²)₃A-; —N(R²)CH₂—CH₂—NR²H₂A-; wherein R² is hydrogen, phenyl, benzyl, or a saturated hydrocarbon radical, preferably an alkyl radical from about C1 to about C20; A- is a halide ion. In some embodiments, L is —N(CH₃)₂ or —NH₂. In some embodiments, L is —NH₂.

One highly preferred amino silicones are those corresponding to formula (I) wherein m=0, a=1, q=3, X=methyl, n is preferably from about 1400 to about 1700, more preferably around 1600; and L is —N(CH₃)₂ or —NH₂, more preferably —NH₂. Another further highly preferred amino silicones are those corresponding to formula (I) wherein m=0, a=1, q=3, X=methyl, n is preferably from about 400 to about 800, more preferably about 500 to around 600; and is —N(CH₃)₂ or —NH₂, more preferably —NH₂.

In some embodiments, the hair care composition comprises aminosilicones having one or both ends of the silicone chain terminated by nitrogen containing group. Such terminal aminosilicones provide improved friction reduction compared to graft aminosilicones.

In some embodiments, the hair care composition comprises aminosilicones having an amine content of less than about 0.12 mmol/g. In some embodiments, the hair care composition comprises aminosilicones having an amine content of less than about 0.1 mmol/g. In some embodiments, the hair care composition comprises aminosilicones having an amine content of less than about 0.08 mmol/g. In some embodiments, the hair care composition comprises aminosilicones having an amine content of less than about 0.06 mmol/g, in view of friction reduction benefit.

In some embodiments, the silicone hair conditioning agent is selected from the group consisting of dimethicone/sodium PG-propyldimethicone thiosulfate copolymer (Abil® S 201 from Goldschmidt); trimethylsilyl amodimethicone (DC Q2-8220 from Dow Corning); amodimethicone, cetrimonium chloride, and Trideceth-12 (DC 949 Dow Corning); cyclomethicone and trimethylsiloxysilicate (DC 749 rom Dow Corning); cetyl dimethicone (DC2502 from Dow Corning); amino functionalized silicone microemulsions (BC97/004 and BC 99/088 from Basildon Chemicals); amino functionalized silicone microemulsions (GE SME253 and SM2115-D2 and SM2658 and SF1708 General Electric); siliconized meadowfoam seed oil, and combination thereof.

In some embodiments, the silicone hair conditioning agent is selected from the group consisting of cyclomethicone, dimethicone, phenyldimethicone, dimethicone copolyol, amodimethicone, and trimethylsilylamodimethicone.

In some embodiments, the silicone hair conditioning agent is selected from the group consisting of cyclomethicone, dimethicone, and amodimethicone.

In some embodiments, the total amount of silicone hair conditioning agent in the hair care composition ranges from about 0.01 wt. % to 20.0 wt. % by the total weight of the hair care composition. In some embodiments, the total amount of silicone hair conditioning agent in the hair care composition ranges from about 0.01 wt. % to 10.0 wt. % by the total weight of the hair care composition. In some embodiments, the total amount of silicone hair conditioning agent in the hair care composition ranges from 0.05 wt. % to 10.0 wt. % by the total weight of the hair care composition. The total amount of silicone hair conditioning agent in the hair care composition ranges from about 0.1 wt. % to 5.0 wt. % by the total weight of the hair care composition. The total amount of silicone hair conditioning agent in the hair care composition ranges from about 0.5 wt. % to 3.0 wt. % by the total weight of the hair care composition. In some embodiments, The total amount of silicone hair conditioning agent in the hair care composition is selected from: about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.25 wt. %, about 1.50 wt. %, about 1.75 wt. %, about 2.0 wt. %, about 2.25 wt. %, about 2.5 wt. %, about 2.75 wt. %, about 3.0 wt. %, about 3.25 wt. %, about 3.5 wt. %, about 3.75 wt. %, about 4.0 wt. %, about 4.25 wt. %, about 4.5 wt. %, about 4.75 wt. %, about 5.0 wt. %, about 5.25 wt. %, about 5.5 wt. %, about 5.75 wt. %, about 6.0 wt. %, about 6.25 wt. %, about 7.5 wt. %, about 7.75 wt. %, about 8.0 wt. %, about 8.25 wt. %, about 8.5 wt. %, about 8.75 wt. %, about 9.0 wt. %, about 9.25 wt. %, about 9.5 wt. %, about 9.75 wt. %, about 10.0 wt. % by the total weight of the hair care composition, in view of conditioning benefits and/or product clarity.

### D. Cationic Hair Conditioning Agent

In some embodiments, the hair care composition may further comprise a cationic conditioning agent to provide hair grooming. The cationic conditioning agents, in particular cationic surfactants, is used singly or in admixture. The cationic hair conditioning agents may include one or more cationic quaternary nitrogen groups, and one or more hydrophobic long chain aliphatic or silicone polymer. The long chain hydrophobic groups, which are derived from long chain fatty acids, or are silicone polymers, can provide hair conditioning functions.

Cationic surfactants useful in the hair care compositions of the disclosure contain amino or quaternary ammonium hydrophilic moieties, which are positively charged when dissolved in the aqueous composition.

In some embodiments, the cationic hair conditioning agents may include cationic silicone polymers. The silicone cationic polymers may include diquaternary polyammonium polydimethylsiloxanes having from about 5 to about 50 dimethylsiloxy repeating units, preferably 25-35 dimethylsiloxy repeating units, and a weight average molecular weight ranging from about 1 kDa to about 4 kDa. Diquaternary dimethylpolysiloxanes have been assigned the CTFA generic name “Quaternium 80”. A specific grade of “Quaternium 80” hair conditioning agent useful herein is ABIL-Quat® 3272 by Goldschmidt AG, Essen, Germany. ABIL-Quat® 3272 silicone compounds have an average molecular weight of around 3000, a siloxane chain containing an average of 30 dimethylsiloxy units, and short chain alkyl groups containing 5 carbons. Another suitable “Quaternium 80” hair conditioning agent is ABIL-Quat® 3270 having an average molecular weight of about 1500, a siloxane chain containing an average of 10 dimethylsiloxy units, and short chain alkyl groups of 5 carbon length.

In some embodiments, the cationic silicone hair conditioning agents having diquaternary ammonium together with amido amine groups grafted to the polydimethylsiloxane chains.

In some embodiments, the weight amount of the cationic silicone hair conditioning agent ranges from about 0.1 wt. % to about 1.5 wt. % by the total weight of the hair care composition.

In some embodiments, the weight amount of the cationic silicone hair conditioning agent ranges from about 0.2 wt. % to about 1.0 wt. % by the total weight of the hair care composition. In some embodiments, the weight amount of the cationic silicone hair conditioning agent is selected from: about 0.1 wt. %, about 0.15 wt. %, about 0.25 wt. %, about 0.3 wt. %, about 0.35 wt. 00 about 0.40 wt. %, about 0.45 wt. %, about 0.50 wt. %, about 0.55 wt. %, about 0.6 wt. %, about 0.65 wt. %, about 0.70 wt. %, about 0.75 wt. %, about 1.0 wt. %, about 1.05 wt. %, about 1.10 wt. %, about 1.15 wt. %, about 1.20 wt. %, about 1.25 wt. %, about 1.30 wt. %, about 1.25 wt. %, about 1.30 wt. %, about 1.35 wt. %, about 1.40 wt. %, about 1.45 wt. %, and about 1.50 wt. % by the total weight of the hair care composition. In some embodiments, the weight amount of the cationic silicone hair conditioning agent is about 0.25 wt. % by the total weight of the hair care composition. In some embodiments, the weight amount of the cationic silicone hair conditioning agent is about 1.0 wt. % by the total weight of the hair care composition.

Cationic hair conditioning agents useful herein may include quaternary ammonium salts or the salts of fatty amines, such as cetyl ammonium chloride. In some embodiments, the cationic quaternary ammonium salt surfactant has Formula (I) [NR¹R²R³R⁴)⁺X⁻, wherein R¹, R², R³, and R⁴ are independently selected from (a) an aliphatic group of from 1 to 22 carbon atoms, or (b) an aromatic, alkoxy, polyoxyalkylene, alkylamido, hydroxyalkyl, aryl or alkylaryl group having up to 22 carbon atoms; and X is a negatively charge counterion selected from halide, (e.g. chloride, bromide), acetate, citrate, lactate, glycolate, phosphate nitrate, sulphate, or alkylsulphate. The most preferred cationic surfactants for conditioner compositions of the present disclosure are monoalkyl quaternary ammonium compounds in which the alkyl chain length is C₈ to C₁₄.

In some embodiments, the monoalkyl cationic quaternary ammonium salt surfactant conditioning agent may include: (i) lauryl trimethylammonium chloride (available commercially as Arquad® C35 ex-Akzo); cocodimethyl benzyl ammonium chloride (available commercially as Arquad® DMCB-80 ex-Akzo).

In some embodiments, the cationic conditioning agent may be selected from the group consisting of octyltrimethylammonium chloride, dodecyltrimethylammonium chloride, hexadecyltrimethylammonium chloride, cetyltrimethylammonium chloride, octyldimethylbenzylammonium chloride, decyldimethylbenzylammonium chloride, stearyldi-methylbenzylammonium chloride, didodecyldimethylammonium chloride, dioctadecyldimethylammonium chloride, tallow trimethylammonium chloride, cocotrimethylammonium chloride, cocotrimethylammonium bromides, cocotrimethylammonium hydroxides, cetylpyridinium chloride, diallyly quaternary ammonium salt/acrylamide copolymers, quaternium-5, polyquaternium-1, polyquaternium-2, polyquaternium-5, polyquaternium-6, polyquaternium-7, polyquaternium-8, polyquaternium-9, polyquaternium-11, polyquaternium-12, polyquaternium-13, polyquaternium-14, polyquaternium-15, polyquaternium-16, polyquaternium-17, quaternium-18, polyquaternium-19, polyquaternium-20, polyquaternium-22, polyquaternium-27, polyquaternium-28, polyquaternium-29, polyquaternium-30, polyquaternium-31, and combination thereof.

In some embodiments, the cationic surfactant conditioning agent is present in a weight percent ranging from 0.01 wt. % to about 10.0 wt. %, preferably, from about 0.05 wt. % to about 5.0 wt. %, and most preferably from about 0.1 wt. % to about 2.0 wt. %.

In some embodiments, the hair conditioning agent is presented in the hair care composition at a weight percent ranging from about 0.01 wt. % to about 10.0 wt. % by the total weight of the silk hair care composition. In some embodiments, the hair conditioning agent is presented in the hair care composition at a weight percent ranging from about 0.05 wt. % to about 5.0 wt. % by the total weight of the silk hair care composition. In some embodiments, the hair conditioning agent is presented in the hair care composition at a weight percent ranging from about 0.1 wt. % to about 2.0 wt. % by the total weight of the silk hair care composition. In some embodiments, the hair conditioning agent is presented in the hair care composition at a weight percent selected from: about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.25 wt. %, about 1.50 wt. %, about 1.75 wt. %, about 2.0 wt. %, about 2.25 wt. %, about 2.5 wt. %, about 2.75 wt. %, about 3.0 wt. %, about 3.25 wt. %, about 3.5 wt. %, about 3.75 wt. %, about 4.0 wt. %, about 4.25 wt. %, about 4.5 wt. %, about 4.75 wt. %, about 5.0 wt. %, about 5.25 wt. %, about 5.5 wt. %, about 5.75 wt. %, about 6.0 wt. %, about 6.25 wt. %, about 7.5 wt. %, about 7.75 wt. %, about 8.0 wt. %, about 8.25 wt. %, about 8.5 wt. %, about 8.75 wt. %, about 9.0 wt. %, about 9.25 wt. %, about 9.5 wt. %, about 9.75 wt. %, about 10.0 wt. % by the total weight of the hair care composition.

In some embodiments, the hair care composition comprises conditioning agents selected from the group consisting of SPF, e.g., without limitation silk fibroin protein based fragments, silk peptides, silk amino acids, silicones (e.g. silicone oils, cationic silicones, silicone gums, high refractive silicones, and silicone resins), organic conditioning oils (e.g. hydrocarbon oils, polyolefins, and fatty esters), and combinations thereof. Each of the SPF, e.g., without limitation silk fibroin protein based fragments, silk peptides, silk amino acids alone or in combination are considered as silk conditioning agent.

In some embodiments, the hair care composition comprises conditioning agents selected from the group consisting of glycerol, propylene glycol, erythritol, sodium PC A, hyaluronic acid, sorbitol, fructose, fatty acids (e.g., stearic acid and oleic acid), fatty alcohols, sorbitol, fructose, a polyquaternium polymer, a cationic surfactant, proteins, amino acids (e.g. silk fibroin amino acids), silk solution, oils, mineral oil, silicones, fatty acid esters, glycerin, cetrimonium chloride, fatty alcohols, dimethicone, and combination thereof.

In some embodiments, the hair care composition comprises silk fibroin amino acid, silk solution, or combination thereof as conditioning agents. In some embodiments, the hair care composition comprises hydrocarbon oil conditioning agent, silk fibroin amino acid, silk solution, or combination thereof as conditioning agents. In some embodiments, the hair care composition comprises silicone conditioning agent, silk fibroin amino acid, silk solution, or combination thereof as conditioning agents. In some embodiments, the hair care composition comprises cationic quaternary ammonium salt surfactant, silk fibroin amino acid, silk solution, or combination thereof as conditioning agents. In some embodiments, the hair care composition comprises a polyquaternium surfactant, silk fibroin amino acid, silk solution, or combination thereof as conditioning agents.

### Hair Styling Material

### A. Pressure Sensitive Adhesive

In some embodiments, the silk hair care composition comprises a pressure sensitive adhesive (PSA) as styling polymer. “Pressure sensitive adhesive” materials are permanently tacky at room temperature and able to develop measurable adhesion to a surface simply upon contact or by the application of a light pressure. Generally, PSAs do not require heat. No chemical reaction takes place between the adhesive and the adherent, no curing of the adhesive is necessary and no solvent is required to be lost during the adhesion process.

In some embodiments, the pressure sensitive adhesive is selected from acrylic PSAs, silicone PSAs. The acrylic sensitive pressure adhesive is preferably in the form of an emulsion. Suitable water-born acrylic sensitive pressure adhesives include Roderm® MD-5800. Small molecule additives such as tackifiers may be included, essentially to adjust the T_{g} and optimize dissipative properties. In some embodiments, the pressure sensitive adhesive is present at a weight percent ranging from 0.01 wt. % to 10.0 wt. % by the total weight of the hair care composition. In some embodiments, the pressure sensitive adhesive is present at a weight percent ranging from 0.1 wt. % to 5.0 wt. % by the total weight of the hair care composition. In some embodiments, the pressure sensitive adhesive is present at a weight percent ranging from 0.5 wt. % to 3.5 wt. % by the total weight of the hair care composition.

### B. Film Forming Agent

In some embodiments, the hair care composition comprises a film-forming substance to form a protective film on the hair that protects the hair from damages caused by various environmental factors including grooming and styling, shampooing, ultraviolet light, and the reactive chemicals commonly used in permanent wave agents, hair coloring products, bleaches, and hair straighteners. Moreover, these film-forming substances improve the elasticity of the hair. In some embodiments, the SPF, e.g., without limitation silk fibroin protein fragments may be functioning as film forming agent for the hair care compositions. SPF, e.g., without limitation silk fibroin protein fragments have protein structure similar to the hair. The SPF, e.g., without limitation silk fibroin protein fragments having a weight average molecular weight ranging from about 1,000 Da to about 390,000 Da easily form resilient and transparent film on the hair. The SPF, e.g., without limitation silk fibroin proteins are ideally suited for film-forming and coating applications due to their ability to self-assemble in solution. The self-assembly property of silk proteins is due to the formation of anti-parallel beta-pleated sheets via hydrogen bonding and electrostatic interactions.

In some embodiments, the SPF, e.g., without limitation silk fibroin protein fragment has a weight average molecular weight ranging from 1 kDa to 18 kDa forms a film coating on the hair that has soft-holding strength. In some embodiments, the SPF, e.g., without limitation silk fibroin protein fragment has a weight average molecular weight ranging from 1 kDa to 10 kDa forms a film coating on the hair that has soft-holding strength. In some embodiments, the SPF, e.g., without limitation silk fibroin protein fragment has a weight average molecular weight ranging from 1 kDa to 5 kDa forms a film coating on the hair that has soft-holding strength. In some embodiments, the SPF, e.g., without limitation silk fibroin protein fragment has a weight average molecular weight ranging from 5 kDa to 10 kDa forms a film coating on the hair that has soft-holding strength. In some embodiments, the SPF, e.g., without limitation silk fibroin protein fragment has a weight average molecular weight ranging from 5 kDa to 18 kDa forms a film coating on the hair that has soft-holding strength.

In some embodiments, the SPF, e.g., without limitation silk fibroin protein fragment has a weight average molecular weight ranging from 10 kDa to 18 kDa forms a film coating on the hair that has soft-holding strength. In some embodiments, the weight amount of the SPF, e.g., without limitation silk fibroin protein film forming agent in the hair care product exhibiting soft-holding strength ranges from about 1.0 wt. % to about 3.0 wt. % by the total weight of the hair care composition. In some embodiments, the weight amount of the SPF, e.g., without limitation silk fibroin protein film forming agent in the hair care product is selected from: about 1.0%, about 1.1%, about 1.2% about 1.3%, about 1.4% about 1.5%, about 1.6% about 1.70% about 1.8%, about 1.9% about 2.0% about 2.1%, about 2.2% about 2.3%, about 2.40% about 2.5%, about 2.6% about 2.7% about 2.8% about 2.9% and about 3.0% (w/v) by the total weight of the hair care composition.

In some embodiments, the SPF, e.g., without limitation silk fibroin protein fragment has a weight average molecular weight ranging from 19 kDa to 300 kDa forms a film coating on the hair that has high-holding strength. In some embodiments, the SPF, e.g., without limitation silk fibroin protein fragment has a weight average molecular weight ranging from 19 kDa to 90 kDa forms a film coating on the hair that has high-holding strength. In some embodiments, the SPF, e.g., without limitation silk fibroin protein fragment has a weight average molecular weight ranging from 20 kDa to 39 kDa forms a film coating on the hair that has high-holding strength. In some embodiments, the SPF, e.g., without limitation silk fibroin protein fragment has a weight average molecular weight ranging from 40 kDa to 90 kDa forms a film coating on the hair that has high-holding strength. The SPF, e.g., without limitation silk fibroin protein film forming agent exhibited excellent curl retention (See FIG. 1C). In some embodiments, the weight amount of the SPF, e.g., without limitation silk fibroin protein film forming agent in the hair care product exhibiting high-holding strength ranges from about 3.0 wt. % to about 6.0 wt. % by the total weight of the hair care composition. In some embodiments, the weight amount of the SPF, e.g., without limitation silk fibroin protein film forming agent in the hair care product high-holding is selected from: about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, and about 6.0 wt. % by the total weight of the hair care composition. In some embodiments, the weight amount of the SPF, e.g., without limitation silk fibroin protein film forming agent in the hair care product exhibiting high-holding strength is about 6.0 wt. %.

In some embodiments, additional film forming agent is added to the hair care composition. In some embodiments, the additional film forming agent is selected from the group consisting of polyacrylic acids, polyacrylates, polyacylamides, silicones, polyquaterium compounds, elastomeric materials, latexes, polyurethanes, polyethylenes, polystyrenes, nylon, polysaccharides, proteins, polysiloxanes (e.g. polyether modified silicone), long chain alkyl quaternary ammoniums, polyvinylpyrrolidone (PVP), PVPK30, quaternary ammonium derivatives of cellulose ethers, copolymers of hydroxyethylcellulose and dimethyldiallylammonium halide, quaternary ammonium derivatives of copolymers of vinylpyrrolidone and dimethylaminoethylmethacrylate, copolymers of acrylamide and dimethyldiallylammonium halide, quaternary ammonium derivatives of copolymers of acrylamide and dimethylaminoethylmethacrylate, shellac, polyvinylpyrrolidone-ethyl methacrylate-methacrylic acid terpolymer, vinyl acetate-crotonic acid copolymer, vinyl acetate-crotonic acid-vinyl neodeconate terpolymer, poly(vinylpyrrolidone)-ethyl methacrylate methacrylic acid copolymer, vinyl methyl ether-maleic anhydride copolymer, octylacrylamide-acrylate-butylaminoethyl-methacrylate copolymer, and poly(vinylpyrrolidone-dimethylaminoethylmethacrylate) copolymer and derivatives, polyquaternium-46, chitosan, microcrystalline chitosan, quaternary ammonium derivative of chitosan, quaternary cellulose derivatives; vinylpyrrolidone-vinyl acetate copolymers, polyvinylpyrrolidone-ethyl methacrylate-methacrylic acid terpolymer, vinyl acetate-crotonic acid copolymer, vinyl acetate-crotonic acid-vinyl neodeconate terpolymer, poly(vinylpyrrolidone)-ethyl methacrylate methacrylic acid copolymer, vinyl methyl ether-maleic anhydride copolymer, octylacrylamide-acrylate-butylaminoethyl-methacrylate copolymer, and poly(vinylpyrrolidone-dimethylaminoethyl-methacrylate) copolymer, shellac, collagen, keratin, and elastin.

In some embodiments, the film forming agent is selected from the group consisting of SPF, e.g., without limitation silk fibroin protein fragments, PVP-PVP-VA, polyquaternium-46, and combination thereof. In some embodiments, the film forming agent comprises SPF, e.g., without limitation silk fibroin protein fragments and polyquaternium-46. In some embodiments, the additional film forming agent is selected from the group consisting of copolymers of acrylamide and dimethyldiallylammonium halide, copolymers of hydroxyethylcellulose and dimethyldiallylammonium halide, and combination thereof. The hair care compositions containing filming forming polymers having quaternary ammonium groups provide more effective and more durable films when applied to hair.

In some embodiments, the additional film forming agent comprises amino-modified silicone resin selected from the group consisting of polydimethylsiloxane containing aminoethylaminopropyl, N-(aminoethylaminomethyl) phenyl, N-(2-aminoethyl)-3-aminopropyl, and bis(2-hydroxyethyl)-3-aminopropyl groups), DC 929 grade silicone fluid (Dow Corning), and combination thereof.

Without wishing to be bound by any particular theory, it is believed that silicones have at least two properties particularly advantageous in hair holding applications. Certain silicone materials form films which are hydrophobic and produce solutions of low viscosity. The amino-modified silicon resins have been found to provide higher humidity resistance than organic film forming materials at lower use levels. In contrast to the organic resins, the viscosity of silicone solution is low even at high use amount. Unlike the organic film formers, the silicone film forming agent does not require neutralization to provide water compatibility. Additionally, the silicone resins permit variations in hair holding strength from high-holding to soft holding through structure modifications on the film forming components. This is in contrast to the organic polymers which are high-holding when are not neutralized and can only provide soft hold through neutralization required for achieving soft holding. Furthermore, the silicone resins offer additional formulation benefits including compatibility with ethanol and hydrocarbon propellants, good sheen, low buildup, lack of tackiness, non-irritability, and reduced flaking.

In some embodiments, the additional film forming agent comprises “silicone gum” which is polydiorganosiloxanes having a weight average molecular weight of from 200,000 Da to 1,000,000 Da. In some embodiments, the polydiorganosiloxane is selected from the group consisting of polydimethyl siloxane copolymer, polydimethyl siloxane/diphenyl/methylvinylsiloxane copolymer, polydimethylsiloxane/methylvinylsiloxane copolymer, and mixtures thereof.

In some embodiments, the additional film forming agent is incorporated into the hair care composition with a solvent. In some embodiments, the solvent may be a hydrocarbon, an alcohol, or a blend of alcohol and water. Other solvents which may be employed include volatile silicones including linear and cyclic siloxanes, hydrocarbons, and aqueous emulsion systems. In some embodiments, the solvent for the silicone gum is a hydrocarbon oil selected from the group consisting of liquefied petroleum gas, propane, isobutane, and combination thereof. In some embodiments, the solvent for the silicone gum is dimethylether.

### Additional Hair Care Active Agents

### A. Plant Extracts

In some embodiments, the hair care composition optionally comprises plant extract that enhances the beneficial effects of SPF, e.g., without limitation silk fibroin protein fragments. In some embodiments, the plant extract is selected from the group consisting of extracts from rice, oat, almond, Camellia Sinensis (green tea) extract, Butyrospermum parkii (shea butter), coconut, papaya, mango, peach, lemon, wheat, rosemary, apricot, algae, grapefruit, sandalwood, lime, orange, Acacia concinna, Butea parviflora, Butea superb, Butea frondosa, Campanulata (fire tulip), Adansonia Digitata (Baobab), Phoenix Dactylifera (date), Hibiscus Sabdariffa (hibiscus), Aframomum melegueta (African pepper), Khaya Senegalensis (mahogany wood), Tamarindus indica (tamarind, or curcumin), Cyperus Papyrus (papyrus), Ageratum spp., birch, burdock, horsetail, lavender, marjoram, nettle, tail cat, thyme, oak bark, echinacea, stinging nettle, witch hazel, hops, henna, chamomile, whitethorn, lime-tree blossom, almond, pine needles, horse chestnut, juniper, kiwi, melon, mallow, cuckoo flower, wild thyme, yarrow, melissa, rest harrow, coltsfoot, marshmallow, rice meristem, moringa, ginseng and ginger root, aloe vera, aloe barbadensis leaf extract, Lavandula angustifolia (lavender) flower extract, Sambucus nigra (elderberry) fruit extract, Phoenix dactylifera (date) seed extract, Avandula stoechas (spanish lavender) extract, Spiraea ulmaria (meadowsweet) leave extract, Chamomilla recutita (chamomile) leaf extract, and Symphytum officinale (comfrey) leaf extract and combination thereof. The extracts of these plants are obtained from seeds, roots, stem, leaves, flowers, bark, fruits, and/or whole plant.

In some embodiments, the plant extract is presented in the hair composition at a weight percent ranging from about 0.001 wt. % to about 10.0 wt. % by the total weight of the hair care composition. In some embodiments, the plant extract is presented in the hair composition at a weight percent ranging from about 0.005 wt. % to about 5.0 wt. % by the total weight of the hair care composition. In some embodiments, the plant extract is presented in the hair composition at a weight percent ranging from about 0.01 wt. % to about 2.0 wt. % by the total weight of the hair care composition. In some embodiments, the plant extract is presented in the hair composition at a weight percent ranging from 0.0045 wt. % to 0.0055 wt. % by the total weight of the hair care composition.

B. UV filter

In some embodiments, the hair care composition optionally comprises a UV filter that absorbs ultraviolet light of wavelengths between 290 to 329 nm. In some embodiments, the hair care composition water UV filter selected from the group consisting of para-aminobenzoic acid, ethyl para-aminobenzoate, amyl para-aminobenzoate, octyl para-aminobenzoate, ethylene glycol salicylate, phenyl salicylate, octyl salicylate, benzyl salicylate, butylphenyl salicylate, homomenthyl salicylate, benzyl cinnamate, 2-ethoxyethyl para-methoxycinnamate, octyl para-methoxycinnamate, glyceryl mono(2-ethylhexanoate) dipara-methoxycinnamate, isopropyl para-methoxycinnamate, diisopropyl-diisopropylcinnamic acid ester mixtures, urocanic acid, ethyl urocanate, hydroxymethoxybenzophenone, hydroxymethoxybenzophenonesulfonic acid and salts thereof, dihydroxymethoxybenzophenone, sodium dihydroxymethoxybenzophenonedisulfonate, dihydroxybenzophenone, tetrahydroxybenzophenone, 4-tert-butyl-4′-methoxydibenzoylmethane, 2,4,6-trianilino-p-(carbo-2′-ethylhexyl-1′-oxy)-1,3,5-triazine, and 2-(2-hydroxy-5-methylphenyl)benzotriazole. In some embodiments, the water soluble ultraviolet absorbent selected from the group consisting of 2-ethylhexyl-p-methoxycinnamate, 4-tert-butyl-4′-methoxydibenzoylmethane, octocrylene, 2,4-bis-[{4-(2-ethylhexyloxy)-2-hydroxy}-phenyl]-6-(4-methoxyphenyl)-1,3,5-triazine, methylene bis-benzotriazolyl tetramethylbutylphenol, 2,4,6-tris-[4-(2-ethylhexyloxycarbonyl)anilino]-1,3,5-triazine, diethylamino hydroxybenzoyl hexyl benzoate, oxybenzone, 2,2′-dihydroxy-4,4′-dimethoxy benzophenone, and combination thereof.

In some embodiments, the UV filter is selected from the group consisting of butyl methoxydibenzoylmethane, ethylhexyl methoxycinnamate, ethylhexyl salicylate, octocrylene, ethylhexyl methoxycinnamate, isoamyl-p-methoxycinnamate, ethylhexyltriazone, diethylhexyl butamido triazone, methylene bis-benzotriazolyl tetramethylbutylphenol, disodium phenyl dibenzimidazole tetrasulfonate, bis-ethylhexyloxyphenol methoxyphenyl triazine, benzophenone-3, and combination thereof.

In some embodiments, the hair care composition comprises an inorganic pigment as UV filters selected from TiO₂, SiO₂, Fe₂O₃, ZrO₂, MnO, Al₂O₃, and combination thereof.

In some embodiments, the UV filter is presented in the hair composition at a weight percent ranging from about 0.001 wt. % to about 20.0 wt. % by the total weight of the hair care composition. In some embodiments, the UV filter is presented in the hair composition at a weight percent ranging from about 0.01 wt. % to about 10.0 wt. % by the total weight of the hair care composition. In some embodiments, the UV filter is presented in the hair composition at a weight percent ranging from about 0.05 wt. % to about 8.0 wt. % by the total weight of the hair care composition.

### C. Emollients

In some embodiments, the hair care composition optionally comprises an emollient selected from the group consisting of a hydrocarbon oil, a hydrocarbon wax, a silicone oil, an acetoglyceride ester, an ethoxylated glyceride, an alkyl ester of a fatty acid, an alkenyl ester of a fatty acid, a fatty acid, a fatty alcohol, a fatty alcohol ether, an ether-ester, lanolin, a lanolin derivative, a polyhydric alcohol, a polyether derivative, a polyhydric ester, a wax ester, a beeswax derivative, a vegetable wax, a natural or essential oil, a phospholipid, a sterol, an amide, and combination thereof.

In some embodiments, the emollients incorporated in the hair care compositions comprise ne or more of (1) hydrocarbon oils and waxes, e.g., mineral oil, petrolatum, paraffin, ozokerite, microcrystalline wax, polyethylene, squalene, and perhydrosqualene; (2) silicone oils, e.g., dimethyl polysiloxanes, methylphenyl polysiloxanes, water-soluble and alcohol-soluble silicone glycol copolymers; (3) acetoglyceride esters, e.g., acetylated monoglycerides; (4) ethoxylated glycerides, e.g., ethoxylated glyceryl monostearate; (5) alkyl esters of fatty acids having 10 to 20 carbon atoms, e.g., hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, methyl, isopropyl, butyl esters of fatty acids; (6) alkenyl esters of fatty acids having 10 to 20 carbon atoms, e.g., oleyl myristate, oleyl stearate, and oleyl oleate; (7) fatty acids having 10 to 20 carbon atoms, e.g., pelargonic, lauric, myristic, palmitic, stearic, isostearic, hydroxystearic, oleic, linoleic, ricinoleic, arachidic, behenic, and erucic acids; (8) fatty alcohols having 10 to 20 carbon atoms, e.g., lauryl, myristyl, cetyl, hexadecyl, stearyl, isostearyl, hydroxystearyl, oleyl, ricinoleyl, behenyl, erucyl alcohols, and 2-octyl dodecanol; (9) fatty alcohols ethers, e.g., ethoxylated fatty alcohols of 10 to 20 carbon atoms, lauryl, cetyl, stearyl, isostearyl, oelyl, and cholesterol alcohols having attached thereto from 1 to 50 ethylene oxide groups or 1 to 50 propylene oxide groups; (10) ether-esters, e.g. fatty acid esters of ethoxylated fatty alcohols; (11) lanolin and its derivatives, e.g., lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, ethoxylated sorbitol lanolin, and liquid and semisolid lanolin absorption bases; (12) polyhydric alcohols and polyether derivatives, e.g., propylene glycol, dipropylene glycol, polypropylene glycols 2000 and 4000, polyoxyethylene glycols, polyoxypropylene polyoxyethylene glycols, glycerol, sorbitol, ethoxylated sorbitol, hydroxypropyl sorbitol, polyethylene glycols 200-6000, methoxy polyethylene glycols 350, 550, 750, 2000 and 5000, poly[ethylene oxide]homopolymers (weight average molecular weight of 100,000-5,000,000 Da), polyalkylene glycols and derivatives, hexylene glycol (2-methyl-2,4-pentanediol), 1, 3-butylene glycol, 1,2,6-hexanetriol, ethohexadiol USP (2-ethyl-1,3-hexanediol), C15-C18 vicinal glycol, and polyoxypropylene derivatives of trimethylolpropane; (13) polyhydric alcohol esters, e.g., ethylene glycol mono- and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters, sucrose cocoate, sucrose dilaurate, sucrose distearate, sucrose hexaerucate, sucrose laurate, sucrose myristate, sucrose oleate, sucrose palmitate, sucrose pentaerucate, sucrose polybehenate, sucrose polycottonseedate, sucrose polylaurate, sucrose polylinoleate, sucrose polyoleate, sucrose polypalmate, sucrose polysoyate, sucrose polystearate, sucrose ricinoleate, sucrose stearate, sucrose tetraisostearate, sucrose tribehenate, sucrose tristearat; (14) wax esters, e.g., beeswax, spermaceti, myristyl myristate, and stearyl stearate; (15) beeswax derivatives, e.g., polyoxyethylene sorbitol beeswax which are reaction products of beeswax with ethoxylated sorbitol of varying ethylene oxide content; (16) vegetable waxes, e.g., carnauba and candelilla waxes; (17) natural or essential oils, e.g., citrus oil, non-citrus fruit oil, nut oils, oils having flavors, perfume or scents, canola oil, corn oil, neem oil, olive oil, cottonseed oil, coconut oil, fractionated coconut oil, palm oil, nut oils, safflower oil, sesame oil, soybean oil, peanut oil, almond oil, cashew oil, hazelnut oil, macadamia oil, pecan oil, pine nut oil, pistachio oil, walnut oil, grapefruit seed oil, lemon oil, orange oil, sweet orange oil, tangerine oil, lime oil, mandarin oil, omega 3 oil, flaxseed oil (linseed oil), apricot oil, avocado oil, carrot oil, cocoa butter oil, coconut oil, fractionated coconut oil, hemp oil, papaya seed oil, rice bran oil, shea butter oil, tea tree seed oil, and wheat germ oil, lavender oil, rosemary oil, tung oil, jojoba oil, poppy seed oil, shea butter, castor oil, mango oil, rose hip oil, tall oil chamomile oil, cinnamon oil, citronella oil, eucalyptus oil, fennel seed oil, jasmine oil, juniper berry oil, raspberry seed oil, lavender oil, primrose oil, lemon grass oil, nutmeg oil, patchouli oil, peppermint oil, pine oil, rose oil, rose hip oil, rosemary oil, eucalyptus oil, tea tree oil, rosewood oil, sandalwood oil, sassafras oil, spearmint oil, Ricinus communis (castor) seed oil, wintergreen oil; (18) phospholipids, e.g., lecithin and derivatives; (19) sterols, e.g., cholesterol and cholesterol fatty acid esters; and (20) fatty acid amides, ethoxylated fatty acid amides, and solid fatty acid alkanolamides, (21) lanolin, Therbroma cacao (cocoa) seed butter, petrolatum, Euphorbia cerifera (candelilla) wax, honey, geraniol, menthol, camphor, cetyl esters, mineral oil, salicylic acid, phenol, palmitoyl isoleucine,

### D. Moisturizers

In some embodiments, the hair care composition optionally comprises a moisturizer selected from the group consisting of water-soluble, low molecular weight moisturizers, fat-soluble, low molecular weight moisturizers, water-soluble, high molecular weight moisturizers and fat-soluble, high molecular weight moisturizers, humectant, and combination thereof.

In some embodiments, the moisturizer comprises a humectant. As used herein, the term “humectant” refer to a hygroscopic substance used to keep things moist. A humectant attracts and retains the moisture in the air nearby via absorption, drawing the water vapor into or beneath the organism's or object's surface.

In some embodiments, the hair care composition optionally comprises a water-soluble silk fibroin peptide as humectant. The amino peptides derived from the SPF, e.g., without limitation silk fibroin protein fragments can be easily absorbed by hair fiber. In some embodiments, a water-soluble silk fibroin peptide may be added to the hair care composition to give an enhanced after use feeling. In some embodiments, amino acids derived from the SPF, e.g., without limitation silk fibroin protein fragments may be added to the hair care composition as a conditioning agent (e.g. to exert excellent condition effects such as moist feel, softness, smoothness, gloss).

In some embodiments, the hair care composition may comprise one or more additional humectant selected from the group consisting of honey, aloe vera, aloe vera leaf juice, aloe vera leaf extract, sorbitol, urea, lactic acid, sodium lactate, pyrrolidone carboxylic acid, trehalose, maltitol, alpha-hydroxy acids, sodium pyroglutamate, pyrolidonecarboxylate, N-acetyl-ethanolamine, sodium lactate, isopropanol, polyalkylene glycols (e.g., ethylene glycol, propylene glycol, hexylene glycol, 1,3-butylene glycol, dipropylene glycol, triethylene glycol), 1,3-propanediol, diethylene glycol monoethyl ether, glyceryl coconate, hydroxystearate, myristate, oleate, sodium hyaluronate, hyaluronic acid, chondroitin sulfuric acid, phospholipids, collagen, elastin, ceramides, lecithin sorbitol, PEG-4, and combination thereof.

In some embodiments, the hair care composition optionally comprises polyhydric alcohols as moisturizer selected from the group consisting of ethylene glycol, propylene glycol, 1,3 butylene glycol, glycerin, sorbitol, polyethylene glycol, glutamine, mannitol, pyrrolidone-sodium carboxylate, (polymerization degree n=2 or more), polypropylene glycol (polymerization degree n=2 or more), polyglycerin (polymerization degree n=2 or more), lactic acid, lactate, and combination thereof.

In some embodiments, the hair care composition optionally comprises fat-soluble, low molecular weight moisturizers selected from the group consisting of cholesterol and cholesterol ester. In some embodiments, the hair care composition optionally comprises water-soluble, high molecular weight moisturizers selected from the group consisting of carboxyvinyl polymers, polyaspartate, tragacanth, xanthane gum, methyl cellulose, hydroxymethyl cellulose, hydroxyethyl cellulose, hydroxypropyl cellulose, carboxymethyl cellulose, water-soluble chitin, chitosan and dextrin. In some embodiments, the hair care composition optionally comprises fat-soluble, high molecular weight moisturizers selected from the group consisting of polyvinylpyrrolidone-eicosene copolymers, polyvinylpyrrolidone-hexadecene copolymers, nitrocellulose, dextrin fatty acid ester and high molecular silicone.

Additional suitable moisturizers include polymeric moisturizers that are water soluble and/or water swellable in nature. In some embodiments, hyaluronic acid, or chitosan is combined with moisturizers to enhance their properties.

In some embodiments, the hair care composition contains moisturizer at about 0.1 wt. % to about 30.0 wt. % by the total weight of the hair care composition. In some embodiments, the hair care composition contains moisturizer at about 0.5 wt. % to about 25.0 wt. % by the total weight of the hair care composition. In some embodiments, the hair care composition contains moisturizer at about 1.0 wt. % to about 20.0 wt. % by the total weight of the hair care composition.

### E. Coloring Agent

In some embodiments, the hair care composition optionally comprises hair coloring agent selected from natural pigments and dyes, synthetic pigments and dyes, lakes, and combination thereof.

In some embodiments, the hair care composition optionally comprises pigments and dyes selected from grape skin pigment, carmine dye, pigment orange rouge, anthocyanins, carminic acid, betacyanins, amaranthin, flavonoids, Verbena hybrida haematochrome, berberine-based pigment, hinokitiol, betel nut pigment, quercetin, rutin, logwood pigment, henna tannin and catechin, curcumin, cactus flavin, rosewood pigment, bixin or decreasing annatto, saffron extract, buckwheat extract, crocin, genipin, henna (Lawsonia alba), camomile (Matricaria chamomila or Anthemis nobilis), indigo, gardenia pigment, gardenia red, pigment, gardenia enzyme-treated pigment, lac pigment, cochineal pigment, brazilin pigment, annatto pigment, turmeric pigment, logwood pigment, and walnut hull extract, and combination thereof. In some embodiments, the hair care composition optionally comprises synthetic pigments and dyes, lakes selected from D&C pigment, FD&C pigment, HC Blue 2, HC Yellow 4, HC Red 3, Disperse Violet 4, Disperse Black 9, HC Blue 7, HC Yellow 2, Disperse Blue 3, Disperse violet 1, Citrus Red No. 2 (CAS No. 6358-53-8), FD&C Yellow No. 6 (CAS No. 2783-94-0), FD&C Yellow No. 6 Lakes (CAS No. 15790-07-5), FD&C Red No. 40 (CAS No. 25956-17-6), FD&C Red No. 40 Lakes (CAS No. 68583-95-9), FD&C Yellow No. 5 (CAS No. 1934-21-0), FD&C Yellow No. 5 Lakes (CAS No. 12225-21-7), Acid Red 18 (CAS No. 2611-82-7), Orange B (CAS No. 15139-76-1), FD&C Green No. 3 (CAS No. 2352-45-9), FD&C Blue No. 1 (CAS No. 3844-45-9), FD&C Blue No. 1 Lakes (CAS No. 68921-42-6), FD&C Red No. 3 (CAS No. 16423-68-0), FD&C Red No. 3 Lakes (CAS No. 12227-78-0), FD&C Blue No. 2 (CAS No. 860-22-0), FD&C Blue No. 2 Aluminum Lake (CAS No. 16521-38-3), Arianor dyes basic brown 17, C.I. (color index)—no. 12,251; basic red 76, CI.—12,245; basic brown 16, CI.—12,250; basic yellow 57, CI. —12,719 and basic blue 99, CI.—56,059 and further direct action dyes such as acid yellow 1, C.I. —10,316 (D&C yellow no. 7); acid yellow 9, C.I.—13,015; basic violet C.I.—45,170; disperse yellow 3, C.I.—11,855; basic yellow 57, CI.—12,719; disperse yellow 1, CI.—10,345; basic violet 1, CI.—42,535, basic violet 3, C.I.—42,555; greenish blue, C.I.—42090 (FD&C Blue no. 1); yellowish red, C.I.—14700 (FD&C red no. 4); yellow, CI.19140 (FD&C yellow no 5); yellowish orange, CI.15985 (FD&C yellow no. 6); bluish green, C.I.42053 (FD&C green no. 3); yellowish red, CI.16035 (FD&C red no. 40); bluish green, CI.61570 (D&C green no. 3); orange, C.I.45370 (D&C orange no. 5); red, CI.15850 (D&C red no. 6); bluish red, CI.15850 (D&C red no. 7); slight bluish red, CI.45380 (D&C red no. 22); bluish red, CI.45410 (D&C red no. 28); bluish red, CI.73360 (D&C red no. 30); reddish purple, CI.17200 (D&C red no. 33); dirty blue red, CI.15880 (D&C red no. 34); bright yellow red, CI.12085 (D&C red no. 36); bright orange, CI.15510 (D&C orange no. 4); greenish yellow, CI.47005 (D&C yellow no. 10); bluish green, CI.59040 (D&C green no. 8); bluish violet, CI.60730 (Ext. D&C violet no. 2); greenish yellow, CI.10316 (Ext. D&C yellow no. 7), Acridine Orange C.I. 46005, metal oxides, carbon black, phthalocyanine pigments, quinacridone pigments, azo pigments and xanthene pigment, nitroaryl amines, aminoanthraquinones, anthraquinone dyes, naphthoquinone dyes, metal oxide coated mica, and combination thereof.

In some embodiments, this disclosure provides a hair coloring composition comprising the silk fibroin fragments, the coloring agent, and the dermatologically acceptable carrier as disclosed herein. In some embodiments, this disclosure provides a method of preventing color loss from dyed hair fibers of a subject comprising contacting the hair of the subject with the hair coloring composition as described herein.

Even though the hair coloring compositions are formulated to give hair long lasting and richly hued colors, mechanical and environmental factors can accelerate color changes and fading due to the loss of coloring agents. In a hair coloring process, the hair fibers are dyed through the deposition of the coloring agents to the hair cortex and cuticle regions. As time passes, the coloring agents affixed inside the hair shaft start to diffuse towards the surface of the hair fiber, and come into more direct contact with water during washing. Thus the primary cause of color fading is due to the coloring agent diffusion and hair's contact with water. Washing both increases the rate of diffusion of the color molecules, as well as eventually washes the color molecules from the hair.

The silk hair coloring composition disclosed herein provides the benefits of preventing color loss from dyed hair as well as enhanced color delivery efficiency due to the film forming property of the silk fibroin fragments. It is believed that the silk fibroin fragments form a film coatings on the hair fibers which slow the loss of the coloring agents resulting from: (a) filling up the channels in the hair cuticle, thus blocking the path for diffusion of the coloring agents to the hair surface, and ii) by stably affixing to the coloring agents thus slowing the rate of diffusion or the color molecules to the fiber surface.

In some embodiments, the hair care composition contains a single coloring agent at about 0.001 wt. % to about 6.0 wt. % by the total weight of the hair care composition. In some embodiments, the hair care composition contains each coloring agent at about 0.01 wt. % to about 2.0 wt. % by the total weight of the hair care composition.

In some embodiments, the hair care composition contains combined level of coloring agents at about 0.01 wt. % to about 15.0 wt. % by the total weight of the hair care composition. In some embodiments, the hair care composition contains combined level of coloring agents at about 0.1 wt. % to about 10.0 wt. % by the total weight of the hair care composition. In some embodiments, the hair care composition contains combined level of coloring agents at about 0.5 wt. % to about 5.0 wt. % by the total weight of the hair care composition.

### F. Anti-Dandruff Agent

In some embodiments, the hair care composition optionally comprises an anti-dandruff agent. In some embodiments, the anti-dandruff agent comprises a heavy metal salt of a particulate metal pyrithione, selenium disulfide, clotrimazole, D-xylose, particulate sulfur, or a mixture thereof. In some embodiments, the metal ions for the particulate metal pyrithione salt are selected from the group consisting of zinc, tin, cadmium, magnesium, aluminum and zirconium. In some embodiments, the particulate metal pyrithione is zinc pyrithione, or a zinc salt of 1-hydroxy-2-pyridinethione. In some embodiments, the anti-dandruff agent is D-xylose.

In some embodiments, the amount of the anti-dandruff agent presented in the hair care composition ranges from about 0.1 wt. % to about 4.0 wt. %, by the total weight of the hair care composition. In some embodiments, the amount of the anti-dandruff agent presented in the hair care composition ranges from about 0.1 wt. % to about 3.0 wt. %, by the total weight of the hair care composition. In some embodiments, the amount of the anti-dandruff agent presented in the hair care composition ranges from about 0.3 wt. % to about 2.0 wt. %, by the total weight of the hair care composition. In some embodiments, the amount of the anti-dandruff agent presented in the hair care composition is selected from: about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. % by the total weight of the hair care composition.

### G. Particles

In some embodiments, the silk hair care composition optionally comprise a particle, wherein the particle may include polymeric particle, mica, silica, mud, and clay. The particles in the silk hair care composition provide the benefits of smoothness, reduced friction, slippery feel whilst leaving the hair feeling clean, light and airy, and improved texture when spread on the hands and/or hair.

In some embodiments, the silk hair composition contains a polymeric particle formed of a polymer selected from the group consisting of an anionic and/or nonionic and/or zwitterionic polymer. In some embodiments, the silk hair composition contains a polymeric particle formed of a polymer selected from the group consisting of polystyrene, polyvinylacetate, polydivinylbenzene, polymethylmethacrylate, poly-n-butylacrylate, poly-n-butylmethacrylate, poly-2-ethylhexylmethyacrylate, 6,12-nylon, poyurethanes, epoxy resins, styrene/vinyl acetate copolymers, styrene/trimethylaminoethyl methacrylate chlroide copolymers, and combinations thereof.

In some embodiments, the silk hair composition contains a cationically polymeric particle formed of a hydrophobic polymer selected from the group consisting of polyethylene homopolymers, ethylene-acrylic acid copolymer, polyamide polymer having a molecular weight in the range of from about 6,000 Da to about 12,000 Da, polyethylene-vinyl acetate copolymer, silicone-synthetic wax copolymer, silicone-natural wax copolymer, candelilla-silicone copolymer, ozokerite-silicone copolymer, synthetic paraffin wax-silicone copolymer, and combinations thereof.

In some embodiments, the silk hair composition contains swollen polymer particles for depositing discrete particles onto the hair surface that enhance hair body and aid in the creation of a hair style. In some embodiments, the swollen polymer particles are selected from the group consisting of particulate silicone polymers and surface-alkylated spherical silicon particles. In some embodiments, the silicone polymers forming the swollen polymer particles are selected from the group consisting of polydiorganosiloxanes, polymonoorganosiloxanes, and cross-linked polydimethyl siloxanes, crosslinked polymonomethyl siloxanes optionally having end groups including hydroxyl or methyl, and crosslinked polydimethyl siloxane (DC 2-9040 silicone fluid by Dow Corning). The polydisorganosiloxanes are preferably derived from suitable combinations of R₃SiO_{0.5} repeating units and R₂SiO repeating units. The polymonoorganosiloxanes are derived from R₁SiO_{1.5}. Each R independently represents an alkyl, alkenyl (e.g. vinyl), alkaryl, aralkyl, or aryl (e.g. phenyl) group. In some embodiments, R is a methyl group.

In some embodiments, the polymeric particles are nanoparticles having a median particle size of less than 1000 nm. In some embodiments, the polymeric particles have a median particle size of about 5 nm to about 600 nm. In some embodiments, the polymeric particles have a median particle size of about 10 nm to about 500 nm. In some embodiments, the polymeric particles have a median particle size of about 10 nm to about 400 nm. In some embodiments, the polymeric particles have a median particle size of about 20 nm to about 300 nm. In some embodiments, the polymeric particles have a median particle size of about 50 nm to about 600 nm.

In some embodiments, the silk hair composition contains clay particles forming a dispersion or a suspension in the dermatologically acceptable carrier as disclosed herein. Throughout this specification, the term “clay” is intended to mean fine-grained earthy materials that become plastic when mixed with water. The clay may be a natural, synthetic or chemically modified clay. Clays include hydrous aluminum silicates which contain impurities, e.g. potassium, sodium, magnesium, or iron in small amounts.

In one embodiment, the clay is a material containing from 38.8% to 98.2% of SiO₂ and from 0.3% to 38.0% of Al₂O₃, and further contains one or more of metal oxides selected from Fe₂O₃, CaO, MgO, TiO₂, ZrO₂, Na₂O and K₂O. In some embodiments, the clay has a layered structure comprising hydrous sheets of octahedrally coordinated aluminum, magnesium or iron, or of tetrahedrally coordinated silicon.

In one embodiment, the clay is selected from the group consisting of kaolin, talc, 2:1 phyllosilicates, 1:1 phyllosilicates, smectite, bentonite, montmorillonites (also known as bentonites), hectorites, volchonskoites, nontronites, saponites, beidelites, sauconites, and mixtures thereof. In one embodiment, the clay is kaolin or bentonite. In some embodiments, the clay is a synthetic hectorite. In another embodiment, the clay is a bentonite.

In some embodiments, the clays have a cation exchange capacity of from about 0.7 meq/100 g to about 150 meq/100 g. In some embodiments, the clays have a cation exchange capacity of from about 30 meq/100 g to about 100 meq/100 g.

In some embodiments, the silk hair care composition optionally comprise a composite particle having an anionically charged clay electrostatically complexed with the cationically charged hair conditioning agents as disclosed herein.

Commercially available synthetic hectorites include those products sold under the trade names Laponite® RD, Laponite® RDS, Laponite® XLG, Laponite® XLS, Laponite® D, Laponite® DF, Laponite® DS, Laponite® S, and Laponite® JS (Southern Clay products, Texas, USA). Commercially available bentonites include those products sold under the trade names Gelwhite® GP, Gelwhite® H, Gelwhite® L, Mineral Colloid® BP, Mineral Colloid® MO, Gelwhite® MAS 100 (sc), Gelwhite® MAS 101, Gelwhite® MAS 102, Gelwhite® MAS 103, Bentolite® WH, Bentolite® L10, Bentolite® H, Bentolite® L, Permont® SX10A, Permont® SC20, and Permont® HN24 (Southern Clay Products, Texas, USA); Bentone® EW and Bentone® MA (Dow Corning); and Bentonite® USP BL 670 and Bentolite® H4430 (Whitaker, Clarke & Daniels).

In order to achieve good deposition onto hair and a stable formulation, the particles have a median particle size ranging from about 1 μm to about 100 μm. In some embodiments, the particles have a median particle size ranging from about 2 μm to about 50 μm. In some embodiments, the particles have a median particle size ranging from about 2 μm to about 20 μm. In some embodiments, the particles have a median particle size ranging from about 4 μm to about 10 μm. In some embodiments, the particles have a median particle size selected from: about 1 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 2.1 μm, about 2.2 μm, about 2.3 μm, about 2.4 μm, about 2.5 μm, about 2.6 μm, about 2.7 μm, about 2.8 μm, about 2.9 μm, about 3.0 μm, about 3.1 μm, about 3.2 μm, about 3.3 μm, about 3.4 μm, about 3.5 μm, about 3.6 μm, about 3.7 μm, about 3.8 μm, about 3.9 μm, about 4.0 μm, about 4.1 μm, about 4.2 μm, about 4.3 μm, about 4.4 μm, about 4.5 μm, about 4.6 μm, about 4.7 μm, about 4.8 μm, about 4.9 μm, about 5.0 μm, about 5.1 μm, about 5.2 μm, about 5.3 μm, about 5.4 μm, about 5.5 μm, about 5.6 μm, about 5.7 μm, about 5.8 μm, about 5.9 μm, about 6.0 μm, about 6.1 μm, about 6.2 μm, about 6.3 μm, about 6.4 μm, about 6.5 μm, about 6.6 μm, about 6.7 μm, about 6.8 μm, about 6.9 μm, about 7.0 μm, about 7.1 μm, about 7.2 μm, about 7.3 μm, about 7.4 μm, about 7.5 μm, about 7.6 μm, about 7.7 μm, about 7.8 μm, about 7.9 μm, about 8.0 μm, about 8.1 μm, about 8.2 μm, about 8.3 μm, about 8.4 μm, about 8.5 μm, about 8.6 μm, about 8.7 μm, about 8.8 μm, about 8.9 μm, about 9.0 μm, about 9.1 μm, about 9.2 μm, about 9.3 μm, about 9.4 μm, about 9.5 μm, about 9.6 μm, about 9.7 μm, about 9.8 μm, about 9.9 μm, and about 10.0 μm.

In some embodiments, the weight ratio of the cationically charged hair conditioning agent to the clay is from 0.05:1 to 20:1. In some embodiments, the weight ratio of the cationically charged hair conditioning agent to the clay is from 0.1:1 to 10:1. In some embodiments, the weight ratio of the cationically charged hair conditioning agent to the clay is from 0.2:1 to 5:1. In some embodiments, the weight ratio of the cationically charged hair conditioning agent to the clay is selected from 0.05:1, 0.1:1, 0.2:1, 0.5:1, 0.75:1, 1:1, 1.5:1, 2:1, 2.5:1, 3:1, 3.5:1, 4.0:1, 4.5:1, 5.0:1, 5.5:1, 6.0:1, 6.5:1, 7.0:1, 7.5:1, 8.0:1, 8.5:1, 9.0:1, 9.5:1, 10.0:1, 10.5:1, 11.0:1, 11.5:1, 12.0:1, 12.5:1, 13.0:1, 13.5:1, 14.0:1, 14.5:1, 15.0:1, 15.5:1, 16.0:1, 16.5:1, 17.0:1, 17.5:1, 18.0:1, 18.5:1, 19.0:1, 19.5:1, ND 20.0:1.

In some embodiments, the particle is present in the silk hair care composition at a weight percent ranging from about 0.01 wt. % to about 10.0 wt. % by the total weight of the silk hair care composition. In some embodiments, the particle is present in the silk hair care composition at a weight percent ranging from about 0.1 wt. % to about 10.0 wt. % by the total weight of the silk hair care composition. In some embodiments, the particle is present in the silk hair care composition at a weight percent ranging from about 0.1 wt. % to about 2.0 wt. % by the total weight of the silk hair care composition. In some embodiments, the particle is present in the silk hair care composition at a weight percent ranging from about 1.0 wt. % to about 9.0 wt. % by the total weight of the silk hair care composition. In some embodiments, the particle is present in the silk hair care composition at a weight percent ranging from about 1.0 wt. % to about 5.0 wt. % by the total weight of the silk hair care composition. In some embodiments, the particle is present in the silk hair care composition at a weight percent selected from: about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6.0 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7.0 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8.0 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9.0 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9 wt. %, and about 10.0 wt. % by the total weight of the silk hair care composition.

In some embodiments, the silk hair care composition optionally comprise a colloidal stabilizer to maintain particle dispersive stability, particularly of larger sized particles. Suitable colloidal stabilizer is selected from the group consisting of propylene oxide-ethylene oxide copolymers or ethyleneoxide-propylenoxide graphted polyethylenimines, polyoxyethylene (20-80 units POE) isooctylphenyl ether, fatty alcohol ethoxylates, polyethoxylated polyterephthalate block co-polymers containing polyvinylpyrrolidone, copolymers containing vinylpyrolidone repeating units, and combinations thereof.

### 3) Dermatologically Acceptable Carrier

### I. Emulsion Carrier

In some embodiments, the hair care composition comprises an emulsion as the dermatologically acceptable carrier. In some embodiments, the dermatologically acceptable carrier exists as a conventional emulsion. In some embodiments, the dermatologically acceptable carrier exits as a microemulsion. In some embodiments, the dermatologically acceptable carrier exits as a water-in-oil emulsion. In some embodiments, the dermatologically acceptable carrier exits as an oil-in-water emulsion. In some embodiments, the dermatologically acceptable carrier exits as a nano-emulsion. In some embodiments, the dermatologically acceptable carrier exits as a water-in-silicone oil emulsion. In some embodiments, the dermatologically acceptable carrier exits as a silicone oil-in-water emulsion.

As used herein, the conventional emulsions have one continuous phase and one disperse phase, which is present as very small spheres stabilized by coating with surfactants. Depending on the nature of the continuous phase, the emulsions are described as oil-in-water or water-in-oil. These emulsions are kinetically stable in the ideal case, i.e. they are retained even for a prolonged period, but not indefinitely. During temperature fluctuations in particular, they may have a tendency toward phase separation as a result of sedimentation, creaming, thickening or flocculation.

As used herein, the microemulsions are thermodynamically stable, isotropic, fluid, optically clear single liquid phase containing a ternary system having three ingredients of an oily component, an aqueous component and a surfactant. Microemulsions arise when a surfactant, or more frequently a mixture of a surfactant and a cosurfactant, reduces the oil/water interfacial tension to extremely low values, often in the range 10³ to 10⁹, preferably 10⁴ to 10⁶ N/m, such that the two insoluble phases remain dispersed by themselves in a homogeneous manner as a result of the thermal agitation. Microemulsions often have bicontinuous structures with equilibrium regions, so-called subphases in the order of magnitude from 100 to 1000 Angstroms. The microemulsion refers to either one state of an O/W (oil-in-water) type microemulsion in which oil is solubilized by micelles, or a bicontinuous microemulsion in which the number of associations of surfactant molecules are rendered infinite so that both the aqueous phase and oil phase have a continuous structure.

For properties, the microemulsion appears transparent or translucent and may exist as a solution in a monophasic state in which all the formulated ingredients and components are uniformly dissolved therein.

Regardless of manufacturing processes, microemulsions may take the same state if they have the same formulation components and prepared at the same temperature. Therefore, the above-described three ingredients (oil, water and surfactant) and the remaining ingredients may be added and mixed in any orders as appropriate and may be agitated using mechanical forces at any power to consequently yield a microemulsion having substantially the same state (in appearance, viscosity, feeling of use, etc.).

Bicontinuous microemulsions comprise two phases, a water phase and an oil phase, in the form of extended adjoining and intertwined domains at whose interface stabilizing interface-active surfactants are concentrated in a monomolecular layer. Bicontinuous micro emulsions form very readily, usually spontaneously due to the very low interfacial tension, when the individual components, water, oil and a suitable emulsifier system, are mixed. Since the domains have only very small extensions in the order of magnitude of nanometers in at least one dimension, the microemulsions appear visually transparent and are thermodynamically, i.e. indefinitely, stable in a certain temperature range depending on the emulsifier system used.

As used herein, the term nanoemulsions refer to emulsions presenting transparent or translucent appearances due to their nano particle sizes, e.g. less than 1000 nm.

### A. Emulsifier System

Emulsifiers (e.g., surfactants) are substances which reduce the interfacial tension between liquid phases which are not miscible with one another, a polar phase, often water and a nonpolar, organic phase, and thus increase their mutual solubility. Surfactants have a characteristic structure feature of at least one hydrophilic and one hydrophobic structural unit. This structure feature is also referred to as amphiphilic.

Anionic, cationic, amphoteric and nonionic surfactants have conventionally been used as emulsifiers for production of emulsified cosmetic materials by emulsification of water and oily substances. However, since synthetic surfactants have been implicated in the destruction of skin surface tissue and constituting a cause of liver damage when entering the body, numerous naturally-derived protein-based emulsifiers including natural protein based emulsifiers have been employed because of their high safety.

Although emulsified cosmetic materials obtained using protein-based emulsifiers generally have a soft, moist feel during use, it is often the case finished products impart a crumbling feel and lack spreadability. The important factors for emulsifiers used in cosmetic products include not only safety and emulsifying power, but also feel during use. The disclosure provides the use of SPF, e.g., without limitation silk fibroin protein fragments as emulsifier (thereafter silk emulsifier) to stabilize the emulsion carrier for the hair care composition disclosed herein.

### (i) Silk Emulsifier

In an embodiment, the hair care composition comprises an emulsion as carrier having a silk emulsifier in the emulsifier system.

Silk fibroin is an amphiphilic polymer with large hydrophobic domains occupying the major component of the polymer, which has a high molecular weight. The hydrophobic regions are interrupted by small hydrophilic spacers, and the N- and C-termini of the chains are also highly hydrophilic. The hydrophobic domains of the H-chain contain a repetitive hexapeptide sequence of Gly-Ala-Gly-Ala-Gly-Ser and repeats of Gly-Ala/Ser/Tyr dipeptides, which can form stable anti-parallel-sheet crystallites. The amino acid sequence of the L-chain is non-repetitive, so the L-chain is more hydrophilic and relatively elastic. The hydrophilic (Tyr, Ser) and hydrophobic (Gly, Ala) chain segments in silk fibroin molecules are arranged alternatively such that allows self-assembling of silk fibroin molecules.

In some embodiments, the emulsifier system comprises a silk emulsifier and a small molecule having high HLB value. The composition of hydrophobic repeating groups is one penta-peptide -Gly-Ala-Gly-Ala-Gly- for each hydrophilic -Ser-, the hydrophilic-hydrophobic balance (HLB) for the silk fibroin protein can be modified to a range from 7.95-16.74 in a hydrophilic environment created by the addition of a hydrophilic molecule having high HLB value (i.e. >10). This range of HLB value of the SPF, e.g., without limitation silk fibroin protein fragments allows the preparation of a wide range of emulsions from O/W type emulsions to W/O type emulsions. In some embodiments, the hydrophilic molecule having high HLB value is selected from the group consisting of glycerol HLB 11.28, butantetraol HLB 12.7, xylitol HLB 14.13, D-sorbitol HLB 15.55, inositol HLB 16.74, polysaccharide including hyaluronic acid, hyaluronate, carrageenan, pullulan, alginic acid, alginate, microbial exopolysaccharides, glucosamine, chondroitin sulfate, glycosaminoglycans, glucomannan, and combination thereof. In some embodiments, the emulsifier system comprises the silk emulsifier and glycerol.

In some embodiments, the silk emulsifier and hydrophilic molecule having high HLB value are incorporated in the emulsion carrier at a weight ratio of silk emulsifier to the hydrophilic molecule of 1:1 to 1:10. In some embodiments, the silk emulsifier and hydrophilic molecule having high HLB value are incorporated in the emulsion carrier at a weight ratio of silk emulsifier to the hydrophilic molecule selected from: 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5.0, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:8, 1:9 and 1:10. In some embodiments, the silk emulsifier and hydrophilic molecule having high HLB value are incorporated in the emulsion carrier at a weight ratio of silk emulsifier to the hydrophilic molecule of 1:1. In some embodiments, the emulsifier system comprises the silk emulsifier and glycerol at a weight ratio of silk emulsifier to glycerol of 1:1 to 1:3. In some embodiments, the emulsifier system comprises the silk emulsifier and glycerol at a weight ratio of silk emulsifier to glycerol selected from: 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3.0.

In an embodiment, this disclosure provides an aqueous solution of SPF, e.g., without limitation silk fibroin protein fragments or the aqueous gel of SPF, e.g., without limitation silk fibroin protein based fragments as described above as emulsifier (hereafter as silk emulsifier) for the emulsion carrier. The aqueous solution of SPF, e.g., without limitation silk fibroin protein fragments or the aqueous gel of SPF, e.g., without limitation silk fibroin protein fragments as described above may be admixed with an oily component to achieve uniform emulsification between the water in the aqueous solution or aqueous gel of the SPF, e.g., without limitation silk fibroin protein fragments and the oily component.

In some embodiments, the SPF, e.g., without limitation silk fibroin protein fragments used as emulsifier has a weight average molecular weight of greater than about 5 kDa. In some embodiments, the silk fibroin protein used as emulsifier has a weight average molecular weight ranging from about 5 kDa to about 350 kDa. In some embodiments, the silk fibroin protein used as emulsifier has a weight average molecular weight ranging from about 20 kDa to about 80 kDa. In some embodiments, the silk fibroin protein used as emulsifier has a weight average molecular weight ranging from about 40 kDa to about 60 kDa. In other embodiments, any silk fibroin fragments described herein can be used as emulsifiers.

In some embodiments, the amount of the silk emulsifier presented in the emulsion carrier ranges from about 0.1 wt. % to about 15.0 wt. % by the total weight of the emulsion carrier. In some embodiments, the amount of the silk emulsifier presented in the emulsion carrier ranges from about 0.75 wt. % to about 10.0 wt. % by the total weight of the emulsion carrier. In some embodiments, the amount of the silk emulsifier presented in the emulsion carrier is selected from the group consisting of about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.25 wt. %, about 1.50 wt. %, about 1.75 wt. %, about 2.0 wt. %, about 2.25 wt. %, about 2.5 wt. %, about 2.75 wt. %, about 3.0 wt. %, about 3.25 wt. %, about 3.5 wt. %, about 3.75 wt. %, about 4.0 wt. %, about 4.25 wt. %, about 4.5 wt. %, about 4.75 wt. %, about 5.0 wt. %, about 5.25 wt. %, about 5.5 wt. %, about 5.75 wt. %, about 6.0 wt. %, about 6.25 wt. %, about 7.5 wt. %, about 7.75 wt. %, about 8.0 wt. %, about 8.25 wt. %, about 8.5 wt. %, about 8.75 wt. %, about 9.0 wt. %, about 9.25 wt. %, about 9.5 wt. %, about 9.75 wt. %, about 10.0 wt. %, about 10.25 wt. %, about 10.5 wt. %, about 10.75 wt. %, about 11.0 wt. %, about 11.25 wt. %, about 11.5 wt. %, about 11.75 wt. %, about 12.0 wt. %, about 12.25 wt. %, about 12.50 wt. %, about 12.75 wt. %, about 13.0 wt. %, about 13.25 wt. %, about 13.50 wt. 00 about 13.75 wt. %, about 14.0 wt. %, about 14.25 wt. %, about 14.50 wt. %, about 14.75 wt. 00 and about 15.0 wt. %.

Silk protein in the aqueous solution tends to fibrillate more readily by shear of vibration or stirring if it has a higher molecular weight. The fibrillated protein consists of water-insoluble masses causes reduction of pleasant feel during use of the cosmetic materials.

In some embodiments, the SPF, e.g., without limitation silk fibroin protein fragments are blended with hydrophilic substance with high HLB value to enhance the hydrophilic environment and such hydrophilic substance includes glycerol, butantetraol, xylitol, D-sorbitol, inositol polyethylene glycol, polyethylene oxide, polylactic acid, cellulose, chitin and polyvinyl alcohol to prevent silk fibroin solution from gelation. It is important to prevent fibroin transformation from random coils to β-sheet structure (fibrillate).

In some embodiments, a sucrose fatty ester based emulsifier having HLB value >10 is added to the silk fibroin protein as emulsion stabilizer to enhance silk fibroin protein emulsification efficiency.

In some embodiments, the emulsifying system for the hair care composition may include a sucrose fatty ester based emulsifier and an aqueous solution of silk fibroin protein or the aqueous gel of silk fibroin protein.

In some embodiments, an aqueous solution or an aqueous gel containing SPF, e.g., without limitation silk fibroin protein fragments may be used as co-emulsifier for the hair care composition, wherein the aqueous solution or gel of silk protein is obtained by dissolving unscoured, partially scoured or scoured spun silkworm fibers (cocoon filaments) with a neutral salt (e.g. lithium bromide).

In some embodiments, the sucrose fatty ester is sucrose palmitate and sucrose laurate ester.

In some embodiments, silk proteins may be employed as surfactants for the hair care composition with enhanced emulsifying efficiency. In some embodiments, phospholipids (e.g. lecithin) may be used to complex with SPF, e.g., without limitation silk fibroin protein fragments derived co-emulsifiers to increase their emulsifying power (efficiency of surfactant).

In some embodiments, the hair care composition containing microemulsion obtained using SPF, e.g., without limitation silk fibroin protein fragments-based emulsifier generally have good spreadability, a soft, and moist feel during use.

### (ii) Additional Surfactants as Emulsifiers

In some embodiments, the emulsion carrier for the silk hair care composition may further comprise one or more ionic surfactants as co-emulsifiers.

An ionic surfactant is a surfactant that is ionized to have an electric charge in an aqueous solution; depending on the type of the electric charge, it is classified into ampholytic surfactants, cationic surfactants, or anionic surfactants. When an anionic surfactant and an ampholytic surfactant, or an anionic surfactant and a cationic surfactant, are mixed in an aqueous solution, the interfacial tension against oil decreases.

An ampholytic surfactant has at least one cationic functional group and one anionic functional group, is cationic when the solution is acidic and anionic when the solution is alkaline, and assumes characteristics similar to a nonionic surfactant around the isoelectric point.

Ampholytic surfactants are classified, based on the type of the anionic group, into the carboxylic acid type, the sulfuric ester type, the sulfonic acid type, and the phosphoric ester type. For the present disclosure, the carboxylic acid type, the sulfuric ester type, and the sulfonic acid type are preferable. The carboxylic acid type is further classified into the amino acid type and the betaine type. Particularly preferable is the betaine type.

Specific examples include: imidazoline type ampholytic surfactants (for example, 2-undecyl-1-hydroxyethyl-1-carboxymethyl-4,5-dihydro-2-imidazolium sodium salt and 1-[2-(carboxymethoxy)ethyl]-1-(carboxymethyl)-4,5-dihydro-2-norcocoalkylimidazolium hydroxide disodium salt); and betaine type surfactants (for example, 2-heptadecyl-N-carboxymethyl-N-hydroxyethyl imidazolinium betaine, lauryldimethylarninoacetic acid betaine, alkyl betaine, amide betaine, and sulfobetaine).

Examples of the cationic surfactant include quaternary ammonium salts such as cetyltrimethylammonium chloride, stearyltrimethylammonium chloride, benenyltrimethylammonium chloride, behenyldimethylhydroxyethylammonium chloride, stearyldimethylbenzylammonium chloride, and cetyltrimethylammonium methylsulfate. Other examples include amide amine compounds such as stearic diethylaminoethylamide, stearic dimethylaminoethylamide, palmitic diethylaminoethylamide, palmitic dimethylaminoethylamide, myristic diethylaminoethylamide, myristic dimethylaminoethylamide, behenic diethylaminoethylamide, behenic dimethylaminoethylamide, stearic diethylaminopropylamide, stearic dimethylaminopropylamide, palmitic diethylaminopropylamide, palmitic dimethylaminopropylamide, myristic diethylaminopropylamide, myristic dimethylaminopropylamide, behenic diethylaminopropylamide, and behenic dimethylaminopropylamide.

In some embodiments, the emulsifier system for the silk hair care composition may further comprise one or more anionic surfactants. Anionic surfactants are classified into the carboxylate type such as fatty acid soaps, N-acyl glutamates, and alkyl ether acetates, the sulfonic acid type such as α-olefin sulfonates, alkane sulfonates, and alkylbenzene sulfonates, the sulfuric ester type such as higher alcohol sulfuric ester salts, and phosphoric ester salts. Preferable are the carboxylate type, the sulfonic acid type, and the sulfuric ester salt type; particularly preferable is the sulfuric ester salt type.

In some embodiments, the anionic surfactant for the hair care composition is selected from the group consisting of higher alkyl sulfuric acid ester salts (for example, sodium lauryl sulfate and potassium lauryl sulfate); alkyl ether sulfuric acid ester salts (e.g., POE-triethanolamine lauryl sulfate and sodium POE-lauryl sulfate); N-acyl sarcosinic acids (e.g., sodium lauroyl sarcosinate); higher fatty acid amide sulfonic acid salts (e.g., sodium N-myristoyl N-methyl taurate, Sodium N-cocoyl-N-methyl taurate, and Sodium jauroylmethyl taurate); phosphoric ester salts (e.g., sodium POE-oleyl ether phosphate and POE stearyl ether phosphoric acid); sulfosuccinates (e.g., sodium di-2-ethylhexylsulfosuccinate, sodium monolauroyl monoethanol amide polyoxyethylene sulfosuccinate, and sodium lauryl polypropylene glycol sulfosuccinate); alkyl benzene sulfonates (e.g., sodium linear dodecyl benzene sulfonate, triethanolamine linear dodecyl benzene sulfonate, and linear dodecyl benzene sulfonic acid); higher fatty acid ester sulfates (e.g., hydrogenated coconut oil aliphatic acid glyceryl sodium sulfate); N-acyl glutamates (e.g., mono sodium N-lauroylglutamate, disodium N-stearoylglutamate, and sodium N-myristoyl-L-glutamate); sulfated oils (e.g., turkey red oil); POE-alkyl ether carboxylic acid; POE-alkyl aryl ether carboxylate; α-olefin sulfonate; higher fatty acid ester sulfonates; sec-alcohol sulfates; higher fatty acid alkyl amide sulfates; sodium lauroyl monoethanolamine succinates; ditriethanolamine N-palmitoylaspartate; and sodium caseinate.

In some embodiments, the emulsifier system for the silk hair care composition may further comprise one or more nonionic surfactants as co-emulsifiers. The nonionic surfactant preferably has an HLB value of 8.9-14. It is generally known that the solubility into water and the solubility into oil balance when the HLB is 7. That is, a surfactant preferable for the present disclosure would have medium solubility in oil/water.

The nonionic surfactants may include: (1) polyethylene oxide extended sorbitan monoalkylates (e.g., polysorbates); (2) polyalkoxylated alkanols; (3) polyalkoxylated alkylphenols include polyethoxylated octyl or nonyl phenols having HLB values of at least about 14, which are commercially available under the trade designations ICONOL® and TRITON®; (4) polaxamers. Surfactants based on block copolymers of ethylene oxide (EO) and propylene oxide (PO) may also be effective. Both EO-PO-EO blocks and PO-EO-PO blocks are expected to work well as long as the HLB is at least about 14, and preferably at least about 16. Such surfactants are commercially available under the trade designations PLURONIC® and TETRONIC® from BASF; (5) polyalkoxylated esters: polyalkoxylated glycols such as ethylene glycol, propylene glycol, glycerol, and the like may be partially or completely esterified, i.e. one or more alcohols may be esterified, with a (C8 to C22) alkyl carboxylic acid. Such polyethoxylated esters having an HLB of at least about 14, and preferably at least about 16, may be suitable for use in compositions of the present disclosure; (6) alkyl polyglucosides. This includes glucopon 425, which has a (C8 to C16) alkyl chain length; (7) sucrose fatty acid ester having high HLB value (8-18): sucrose cocoate, sucrose dilaurate, sucrose distearate, sucrose hexaerucate, sucrose hexaoleate/hexapalmitate/hexstearate, sucrose hexapalmitate, sucrose laurate, sucrose myristate, sucrose oleate, sucrose palmitate, sucrose pentaerucate, sucrose polybehenate, sucrose polycottonseedate, sucrose polylaurate, sucrose polylinoleate, sucrose polyoleate, sucrose polypalmate, sucrose polysoyate, sucrose polystearate, sucrose ricinoleate, sucrose stearate, sucrose tetraisostearate, sucrose trilaurate.

In some embodiments, the emulsifier system comprises a lipophilic nonionic surfactants selected from the group consisting of sorbitan fatty acid esters (e.g., sorbitan mono oleate monooleate, sorbitan mono isostearate monoisostearate, sorbitan mono laurate monolaurate, sorbitan mono palmitate monopalmitate, sorbitan mono stearate monostearate, sorbitan sesquioleate, sorbitan trioleate, diglyceryl sorbitan penta-2-ethylhexylate, diglyceryl sorbitan tetra-2-ethylhexylate); glyceryl and polyglyceryl aliphatic acids (e.g., mono cottonseed oil fatty acid glycerine, glyceryl monoerucate, glyceryl sesquioleate, glyceryl monostearate, α,α′-glyceryl oleate pyroglutamate, monostearate glyceryl malic acid); propylene glycol fatty acid esters (e.g., propylene glycol monostearate); hydrogenated castor oil derivatives; glyceryl alkylethers, and combination thereof.

In some embodiments, the emulsifier system comprises a hydrophilic nonionic surfactants selected from the group consisting of POE-sorbitan fatty acid esters (e.g., POE-sorbitan monooleate, POE-sorbitan monostearate, POE-sorbitan monooleate, and POE-sorbitan tetraoleate); POE sorbitol fatty acid esters (e.g., POE sorbitol monolaurate, POE-sorbitol monooleate, POE-sorbitolpentaoleate, and POE-sorbitol monostearate); POE-glyceryl fatty acid esters (e.g., POE-monooleates such as POE-glyceryl monostearate, POE-glyceryl monoisostearate, and POE glycerin glyceryl triisostearate); POE-fatty acid esters (e.g, POE-distearate, POE-monodioleate, and ethylene glycol distearate); POE-alkylethers (e.g., POE-lauryl ether, POE-oleyl ether, POE-stearyl ether, POE-behenyl ether, POE 2-octyl dodecyl ether, and POE-cholestanol ether); pluaronics (e.g., pluaronic); POE-POP-alkylethers (e.g, POE-POP-cetyl ether, POE-POP₂-decyl tetradecyl ether, POE-POP-monobutyl ether, POE-POP-lanolin hydrate, and POE-POP glycerin glyceryl ether); tetra POE-tetra POP-ethylenediamino condensates (e.g., tetronic); POE-castor oil hydrogenated castor oil derivatives (e.g., POE-castor oil, POE-hydrogenated castor oil, POE-hydrogenated castor oil monoisostearate, POE-hydrogenated castor oil triisostearate, POE-hydrogenated castor oil monopyroglutamic monoisostearic diester, and POE-hydrogenated castor oil maleic acid); POE-beeswax-lanolin derivatives (e.g., POE-sorbitol beeswax); alkanol amides (e.g., palm oil fatty acid diethanol amide, laurate monoethanolamide, and fatty acid isopropanol amide); POE-propylene glycol fatty acid esters; POE-alkylamines; POE-fatty acid amides; sucrose fatty acid esters; alkyl ethoxydimethylamine oxides; and trioleyl phosphoric acid.

In some embodiments, the emulsifier system comprises mono-glycerol derivatives and/or diglycerol derivatives. Specific examples include: monoglycerol derivatives such as monoglycerol monooctanoate, monooctyl monoglyceryl ether, monoglycerol monononanoate, monononyl monoglyceryl ether, monoglycerol monodecanoate, monodecyl monoglyceryl ether, monoglycerol monoundecylenate, monoundecylenyl glyceryl ether, monoglycerol monododecanoate, monododecyl monoglyceryl ether, monoglycerol monotetradecanoate, monoglycerol monohexadecanoate, monoglycerol monooleate, and monoglycerol monoisostearate, as well as diglycerol derivatives such as diglycerol monooctanoate, monooctyl diglyceryl ether, diglycerol monononanoate, monononyl diglyceryl ether, diglycerol monodecanoate, monodecyl diglyceryl ether, diglycerol monoundecylenate, monoundecylenyl glyceryl ether, diglycerol monododecanoate, monododecyl diglyceryl ether, diglycerol monotetradecanoate, diglycerol monohexadecanoate, diglycerol monooleate, and diglycerol monoisostearate.

In some embodiments, the emulsifier system comprises the silk emulsifier and one or more of sucrose laurate, and sucrose palmitate. In some embodiments, the emulsifier system comprises the silk emulsifier and sucrose laurate. In some embodiments, the emulsifier system comprises the silk emulsifier and sucrose palmitate. In some embodiments, the emulsifier system comprises the silk emulsifier, sucrose laurate, and sucrose palmitate, wherein sucrose laurate, and sucrose palmitate in the emulsion carrier has a weight ratio of sucrose laurate to sucrose palmitate ranging from 1:1 to 1:3. In some embodiments, the emulsifier system comprises the silk emulsifier, sucrose laurate, and sucrose palmitate, wherein sucrose laurate, and sucrose palmitate in the emulsion carrier has a weight ratio of sucrose laurate to sucrose palmitate selected from: 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, and 1:3.0. In some embodiments, the emulsifier system comprises the silk emulsifier, sucrose laurate, and sucrose palmitate, wherein sucrose laurate, and sucrose palmitate in the emulsion carrier has a weight ratio of sucrose laurate to sucrose palmitate selected from: 1:1, 1:1.1, 1:1.2 and 1:1.3. In some embodiments, the emulsifier system comprises the silk emulsifier, sucrose laurate, and sucrose palmitate, wherein sucrose laurate, and sucrose palmitate in the emulsion carrier has a weight ratio of sucrose laurate to sucrose palmitate of 1:1.

In some embodiments, the emulsifier system comprises the silk emulsifier, glycerol, sucrose laurate, and sucrose palmitate, wherein sucrose laurate and sucrose palmitate in the emulsion carrier has a weight ratio of sucrose laurate to sucrose palmitate selected from: 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, and 1:3.0, wherein the silk emulsifier and the glycerol in the emulsion carrier has a weight ratio of silk emulsifier to glycerol selected from: 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, and 1:3.0.

In some embodiments, the emulsifier system comprises the silk emulsifier, glycerol, sucrose laurate, and sucrose palmitate, wherein sucrose laurate and sucrose palmitate in the emulsion carrier has a weight ratio of sucrose laurate to sucrose palmitate selected from: 1:1, 1:1.1, 1:1.2, and 1:1.3, wherein the silk emulsifier and the glycerol in the emulsion carrier has a weight ratio of silk emulsifier to glycerol selected from: 1:1, 1:2, and 1:3.0.

In some embodiments, the emulsifier system is incorporated in the emulsion carrier at a weight percent ranging from 0.1 wt. % to 5.0 wt. % by the total weight of the hair care composition. In some embodiments, the emulsifier system is incorporated in the emulsion carrier at a weight percent ranging from 0.1 wt. % to 3.0 wt. % by the total weight of the hair care composition. In some embodiments, the emulsifier system is incorporated in the emulsion carrier at a weight percent ranging from 0.1 wt. % to 2.0 wt. % by the total weight of the hair care composition.

### B. Oil Phase

In some embodiments, the emulsion carrier comprises an oil phase emulsified with the emulsifier system containing the silk emulsifier as described above. The fatty materials may be useful for forming the oil phase. The fatty material is selected from the group consisting of hydrocarbon oils, silicon oil, higher fatty acids, higher alcohols, synthetic ester oils, silicone oils, liquid oils/fats, solid oils/fats, waxes, and combination thereof.

In an embodiment, the fatty material optionally comprises a wax. The wax is selected from the group consisting of polyethylene wax, polypropylene wax, beeswax, candelilla wax, paraffin wax, ozokerite, microcrystalline waxes, carnauba wax, cotton wax, esparto wax, carnauba wax, bayberry wax, tree wax, whale wax, montan wax, bran wax, lanolin, kapok wax, lanolin acetate, liquid lanolin, sugar cane wax, lanolin fatty acid isopropyl ester, hexyl laurate, reduced lanolin, jojoba wax, hard lanolin, shellac wax, POE lanolin alcohol ether, POE lanolin alcohol acetate, POE cholesterol ether, lanolin fatty acid polyethylene glycol, POE hydrogenated lanolin alcohol ether, and combination thereof.

In an embodiment, the fatty material optionally comprises an ester oil. The ester oil is selected from the group consisting of cholesteryl isostearate, isopropyl palmitate, isopropyl myristate, neopentylglycol dicaprate, isopropyl isostearate, octadecyl myristate, cetyl 2-ethylhexanoate, cetearyl isononanoate, cetearyl octanoate, isononyl isononanoate, isotridecyl isononanoate, glyceryl tri-2-ethylhexanoate, glyceryl tri(caprylatelcaprate), diethylene glycol monoethyl ether oleate, dicaprylyl ether, caprylic acid/capric acid propylene glycol diester, and combination thereof.

In an embodiment, the fatty material optionally comprises a glyceride fatty ester. As used herein, the term “glyceride fatty esters” refers to the mono-, di-, and tri-esters formed between glycerol and long chain carboxylic acids such as C₆-C₃₀ carboxylic acids. The carboxylic acids may be saturated or unsaturated or contain hydrophilic groups such as hydroxyl. Preferred glyceride fatty esters are derived from carboxylic acids of carbon chain length ranging from C₁₀ to C₂₄, preferably C₁₀ to C₂₂ most preferably C₁₂ to C₂₀.

In an embodiment, the fatty material optionally comprises synthetic ester oils. In some embodiments, the synthetic ester oil is selected from the group consisting of isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di-2-ethylhexylate, dipentaerythritol fatty acid ester, N-alkyl glycol monoisostearate, neopentyl glycol dicaprate, diisostearyl malate, glyceryl di-2-heptylundecanoate, trimethylolpropane tri-2-ethylhexylate, trimethylolpropane triisostearate, pentaneerythritol tetra-2-ethylhexylate, glyceryl tri-2-ethylhexylate, trimethylolpropane triisostearate, cetyl 2-ethylhexanoate, 2-ethylhexyl palmitate, glyceryl trimyristate, tri-2-heptylundecanoic glyceride, castor oil fatty acid methyl ester, oleyl oleate, cetostearyl alcohol, acetoglyceride, 2-heptylundecyl palmitate, diisopropyl adipate, N-lauroyl-L-glutamic acid-2-octyldodecyl ester, di-2-heptylundecyl adipate, ethyl laurate, di-2-ethylhexyl cebatate. 2-hexyldecyl myristate, 2-hexyldecyl palmitate, 2-hexyldecyl adipate, diisopropyl cebatate, 2-ethylhexyl succinate, ethyl acetate, butyl acetate, amyl acetate and triethyl citrate, and combination thereof.

In an embodiment, the fatty material optionally comprises ether oil. In some embodiments, the ether oils are selected from the group consisting of alkyl-1,3-dimethylethyl ether, nonylphenyl ether, and combination thereof.

In an embodiment, the fatty material optionally comprises higher fatty acids. As used herein, the higher fatty acids have a carbon number ranging from 8 to 22. In some embodiments, the higher fatty acid is selected from the group consisting of lauric acid, myristic acid, palmitic acid, stearic acid, behenic acid, oleic acid, 12-hydroxystearic acid, undecylenic acid, tall oil, isostearic acid, linoleic acid, linolenic acid, eicosapentaenoic acid (EPA), docosahexaenoic acid (DHA), and combination thereof.

In an embodiment, the fatty material optionally comprises higher fatty alcohols. As used herein, the higher fatty alcohols have a carbon number ranging from 8 to 22. In some embodiments, the higher fatty acid is selected from the group consisting of straight chain alcohols (for example, lauryl alcohol, cetyl alcohol, stearyl alcohol, behenyl alcohol, myristyl alcohol, oleyl alcohol, and cetostearyl alcohol) and branched chain ethyl alcohols (for example, mono stearyl glyceryl ether (batyl alcohol), 2-decyltetradecynol, lanolin alcohol, cholesterol, phytosterol, hexyl dodecanol, isostearyl alcohol, and octyl dodecanol), and combination thereof.

In an embodiment, the fatty material optionally comprises one or more silicone oils. As used herein, the term “silicone oil” (also as silicone fluid) is used herein to designate water-insoluble silicone polymers which are applied to hair to improve its feel or appearance. Silicone oils can provide the hair with a silky, lubricious feel. They can also provide a lusterization effect. These results are obtained by coating hair strands with thin films of silicone oil. Since silicone oils are substantially water-insoluble, after application to the hair they tend to remain thereon despite rinsing with water. Silicone oil can therefore be applied in a shampoo, or in a hair conditioner applied after shampooing, and then followed by water-rinsing.

In some embodiments, the oil phase comprises a non-volatile silicone, which may be a polyalkyl siloxane, a polyalkylaryl siloxane, or mixtures thereof. Suitable polyalkyl siloxanes include polydimethyl siloxanes having a viscosity of from 5 to 100,000 centistokes at 25° C. These siloxanes are available commercially from the General Electric Company as the VISCASIL® series and from Dow Corning as the DC 200 series.

In some embodiments, the silicone oil is selected from the group consisting of linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1000 to 150 000 g/mol. The polymethylphenyl polysiloxanes having a viscosity of from 15 to 65 centistokes at 25° C. These siloxanes are available commercially from Dow Corning as DC-556 grade silicone fluid.

In some embodiments, the silicone oils is selected from the group consisting of methyl polysiloxane, decamethylcydopentasiloxane, octamethylcydotetrasiloxane, and combination thereof.

In some embodiments, the silicone oil comprises volatile silicon oil selected from the group consisting of cyclic siloxanes have four to eight membered rings. In some embodiments, the volatile silicone comprises cyclomethicone selected from the group consisting of dodecamethyl cyclohexasiloxane, decamethylcydopentasiloxane (D5), octamethylcydotetrasiloxane (D4), and combination thereof.

In some embodiments, the fatty phase comprises liquid oils/fats. In some embodiments, the liquid oils/fats are selected from the group consisting of avocado oil, tsubaki oil, turtle oil, macademia nut oil, corn oil, mink oil, olive oil, rape seed oil, egg yolk oil, sesame seed oil, persic oil, wheat germ oil, sasanqua oil, castor oil, linseed oil, safflower oil, cotton seed oil, perilla oil, soybean oil, peanut oil, tea seed oil, kaya oil, rice bran oil, chinese wood oil, Japanese wood oil, jojoba oil, germ oil, triglycerol, glyceryl trioctanoate and glyceryl triisopalmitate, and combination thereof.

In some embodiments, the fatty phase comprises solid fats/oils. In some embodiments, the solid oils/fats are selected from the group consisting of cacao butter, coconut oil, horse tallow, hardened coconut oil, palm oil, beef tallow, sheep tallow, hardened beef tallow, palm kernel oil, pork tallow, beef bone tallow, Japanese core wax, hardened oil, neatsfoot tallow, Japanese wax and hydrogenated castor oil, and combination thereof.

In some embodiments, the fatty phase comprises vegetable oils. In some embodiments, the vegetable oils are selected from the group consisting of buriti oil, soybean oil, olive oil, tea tree oil, rosemary oil, jojoba oil, coconut oil, sesame seed oil, sesame oil, palm oil, avocado oil, babassu oil, rice oil, almond oil, argon oil, sunflower oil, and combination thereof. In some embodiments, the vegetable oil is selected from the group consisting of coconut oil, sunflower oil and sesame oil. In some embodiments, the oily component is selected from cocoa butter, palm stearin, sunflower oil, soybean oil and coconut oil.

In some embodiments, the oil phase for the hair care composition comprises lipid material. In some embodiments, the lipid materials are selected from the group consisting of ceramides, phospholipids (e.g., soy lecithin, egg lecithin), glycolipids, and combination thereof.

In some embodiments, the oil phase for the hair care composition comprises hydrocarbon oil. As used herein, the hydrocarbon oils have average carbon chain length less than 20 carbon atoms. Suitable hydrocarbon oils include cyclic hydrocarbons, straight chain aliphatic hydrocarbons (saturated or unsaturated), and branched chain aliphatic hydrocarbons (saturated or unsaturated). Straight chain hydrocarbon oils will typically contain from about 6 to about 16 carbon atoms, preferably from about 8 up to about 14 carbon atoms. Branched chain hydrocarbon oils can and typically may contain higher numbers of carbon atoms, e.g. from about 6 up to about 20 carbon atoms, preferably from about 8 up to about 18 carbon atoms. Suitable hydrocarbon oils of the disclosure will generally have a viscosity at ambient temperature (25 to 30° C.) of from 0.0001 to 0.5 Pa·s, preferably from 0.001 to 0.05 Pa·s, more preferably from 0.001 to 0.02 Pa·s.

In some embodiments, the hydrogen carbon oils are selected from the group consisting of liquid petrolatum, squalane, pristane, paraffin, isoparaffin, ceresin, squalene, mineral oil, light mineral oil, blend of light mineral oil and heavy mineral oil, polyisobutene, hydrogenated polyisobutene, terpene oil and combination thereof.

In some embodiments, the hydrogen carbon oils light mineral oil. As used herein, mineral oils are clear oily liquids obtained from petroleum oil, from which waxes have been removed, and the more volatile fractions removed by distillation. The fraction distilling between 250° C. to 300° C. is termed mineral oil, and it consists of a mixture of hydrocarbons, in which the number of carbon atoms per hydrocarbon molecule generally ranges from C10 to C40. Mineral oil may be characterized in terms of its viscosity, where light mineral oil is relatively less viscous than heavy mineral oil, and these terms are defined more specifically in the U.S. Pharmacopoeia, 22nd revision, p. 899 (1990). A commercially available example of a suitable light mineral oil for use in the disclosure is Sirius® M40 (carbon chain length C0-C28 mainly C12-C20, viscosity 4.3×10 Pa·s), available from Silkolene. Other hydrocarbon oils that may be used in the disclosure include relatively lower molecular weight hydrocarbons including linear saturated hydrocarbons such a tetradecane, hexadecane, and octadecane, cyclic hydrocarbons such as dioctylcyclohexane (e.g. CETIOL® S from Henkel), branched chain hydrocarbons (e.g. ISOPAR® and ISOPAR® V from Exxon Corp.).

In some embodiments, the fatty material for the oil phase is selected from the group consisting of neopentyl glycol diheptanoate, propylene glycol dicaprylate, dioctyl adipate, coco-caprylate/caprate, diethylhexyl adipate, diisopropyl dimer dilinoleate, diisostearyl dimer dilinoleate, butyrospermumparkii (shea) butter, C12-C13 alkyl lactate, di-C12-C13 alkyl tartrate, tri-C12-C13 alkyl citrate, C12-C15 alkyl lactate, ppg dioctanoate, diethylene glycol dioctanoate, meadow foam oil, C12-15 alkyl oleate, tridecyl neopentanoate, cetearyl alcohol and polysorbate 60, C18-C26 triglycerides, cetearyl alcohol & cetearyl glucoside, acetylated lanolin, vp/eicosene copolymer, glyceryl hydroxystearate, C18-36 acid glycol ester, C18-36 triglycerides, glyceryl hydroxystearate and mixtures thereof. also suitable and preferred are cetyl alcohol & glyceryl stearate & PEG-75, stearate & ceteth-20 & steareth-20, lauryl glucoside & polyglyceryl-2 dipolyhydroxystearate, beheneth-25, polyamide-3 & pentaerythrityl tetra-di-t-butyl hydroxycinnamate, polyamide-4 and PEG-100 stearate, potassium cethylphosphate, stearic acid and hectorites.

In some embodiments, the fatty material for the oil phase is selected from the group consisting of liquid paraffin, liquid isoparaffin, neopentylglycol dicaprate, isopropyl isostearate, cetyl 2-ethylhesanoate, isononyl isononanoate, glyceryl tri(caprylatelcaprate), alky-1,3-dimethylbutyl ether, methyl polysiloxane having a molecular weight ranging from 100 to 500, decamethylcydopentasiloxane, octamethylcydotetrasiloxane, higher fatty acids having a carbon number ranging from 12 to 22, higher alcohols having a carbon number ranging from 12 to 22, ceramides, glycolipids, and terpene oil.

In some embodiments, the fatty material for the oil phase is selected from the group consisting of paraffin oil, glyceryl stearate, isopropyl myristate, diisopropyl adipate, cetylstearyl 2-ethylhexanoate, hydrogenated polyisobutene, Vaseline, caprylic/capric triglycerides, microcrystalline wax, lanolin and stearic acid, silicone oils and combination thereof.

In an embodiment, the fatty material for the oil phase is selected from the group consisting of vegetable oils including jojoba oil, olive oil, camella oil, avocado oil, cacao oil, sunflower oil, persic oil, palm oil, castor oil, buriti oil, medium chain triglycerides.

In an embodiment, the oily materials emulsiferable by the silk emulsifier is selected from the group consisting of a vegetable oil, isododecane, and isohexadecane, and one or more oily esters of fatty acids, wherein the vegetable oil is selected from jojoba oils and/or camellia oils, wherein said oily esters are selected from isononyl isononanoate and coco caprylate.

In some embodiments, the oil phase is present in the hair care composition at a weight percent ranging from 1.0 wt. % to about 95 wt. % by the total weight of the hair care composition. In some embodiments, the oil phase is present in the hair care composition at a weight percent ranging from 45.0 wt. % to about 95 wt. % by the total weight of the hair care composition. In some embodiments, the oil phase is present in the hair care composition at a weight percent ranging from 45.0 wt. % to about 65.0 wt. % by the total weight of the hair care composition. In some embodiments, the oil phase is present in the hair care composition at a weight percent ranging from 5.0 wt. % to about 45 wt. % by the total weight of the hair care composition. In some embodiments, the oil phase is present in the hair care composition at a weight percent ranging from 5.0 wt. % to about 35 wt. % by the total weight of the hair care composition. In some embodiments, the oil phase is present in the hair care composition at a weight percent ranging from 10.0 wt. % to about 25 wt. % by the total weight of the hair care composition.

In some embodiments, the oil phase is presented in the hair care composition in a weight percent ranging from about 50.0 wt. % to 95.0 weight % by the total weight of the emulsion carrier. In some embodiments, the oil phase is presented in the hair care composition in a weight percent ranging from about 5 wt. % to 45 weight % by the total weight of the emulsion carrier, because such a content allows the emulsion carrier to have a stability over a wider temperature range around the room temperatures and a good feeling.

### C. Aqueous Phase

In some embodiments, the aqueous phase for the emulsion carrier comprises water, an aqueous solution, a blend of alcohol and water, or a lyotropic liquid crystalline phase as aqueous carrier. Selection of the water contained in the hair care composition of the present disclosure is not limited in particular; specific examples include purified water, ion-exchanged water, and tap water. In some embodiments, the aqueous further comprise one or more small molecule polyhydric alcohols selected from the group consisting of ethanediol, propanediol, glycerol, butanediol, butantetraol, xylitol, sorbitol, inositol, ethylene glycol, polyethylene glycol. In some embodiments, the aqueous phase further comprise one or more low alcohol solvent including methanol, ethanol, and isopropanol.

The blend ratio of water and polyhydric alcohol is determined appropriately based on emulsion formulation types.

In some embodiments, the emulsion comprises from about 50 wt. % to about 98 wt. % of the aqueous phase by the total weight of the hair care composition. In some embodiments, the emulsion comprises from about 60 wt. % to about 90 wt. % of the aqueous phase by the total weight of the hair care composition. In some embodiments, the amount of the aqueous phase in the emulsion is selected from: about 50.0 wt. %, about 51.0 wt. %, about 52.0 wt. %, about 53.0 wt. %, about 54.0 wt. %, about 55.0 wt. %, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. %, about 69.0 wt. %, about 70.0 wt. %, about 71.0 wt. %, about 72.0 wt. %, about 73.0 wt. %, about 74.0 wt. %, about 75.0 wt. %, about 76.0 wt. %, about 77.0 wt. %, about 78.0 wt. %, about 79.0 wt. %, about 80.0 wt. %, about 81.0 wt. %, about 82.0 wt. %, about 83.0 wt. %, about 84.0 wt. %, about 85.0 wt. %, about 86.0 wt. %, about 87.0 wt. %, about 88.0 wt. %, about 89.0 wt. %, about 90.0 wt. %, about 91.0 wt. %, about 92.0 wt. %, about 93.0 wt. %, about 94.0 wt. %, about 95.0 wt. %, about 96.0 wt. %, about 97.0 wt. %, about 98.0 wt. %, by the total weight of the hair care composition.

In some embodiments, the silk containing emulsifier system is present in the aqueous phase.

### II. Shampoo Base Carrier

In some embodiments, the carrier for the hair care composition comprises a shampoo base, wherein the shampoo base carrier contains a cleansing surfactant component containing SPF, e.g., without limitation silk fibroin protein fragments, viscosity modifying/thickening agent, and an aqueous carrier. The shampoo base serves to produce foam. The shampoo base has a pH value about 5.

### A. Cleansing Surfactants

### (i) Silk Lathering Surfactant

In some embodiments, the hair care composition comprises a detersive surfactant component to provide cleansing performance. The detersive surfactant may be selected from anionic detersive surfactant, zwitterion or amphoteric detersive surfactant, or a combination thereof. Such surfactants should be physically and chemically compatible with the essential components described herein, or should not otherwise unduly impair product stability, aesthetics or performance. The anionic detersive surfactant is believed to provide cleaning and lather performance to the shampoo.

In some embodiments, the shampoo base comprising SPF, e.g., without limitation silk fibroin protein fragments as a lathering surfactant. The shampoo resulted thereof provides a lather when massaged into hair, washed out well, and “squeaky” feel during use.

In some embodiments, the shampoo base comprises about 2.0 wt. % to about 5.0 wt. % of silk fibroin-based protein fragments that are substantially devoid of sericin, wherein the silk fibroin-based protein fragments have a weight average molecular weight ranging from about 5 kDa to about 80 kDa, wherein the silk fibroin-based protein fragments have a polydispersity of between about 1.5 and about 3.0. In some embodiments, the shampoo base comprises about 2.0 wt. % to about 5.0 wt. % of any silk fibroin-based protein fragments described herein.

In some embodiments, the shampoo base comprises about 2.0 wt. % to about 5.0 wt. % of silk fibroin-based protein fragments that are substantially devoid of sericin, wherein the silk fibroin-based protein fragments have a weight average molecular weight ranging from about 5 kDa to about 17 kDa, wherein the silk fibroin-based protein fragments have a polydispersity of between about 1.5 and about 3.0.

In some embodiments, the shampoo base comprises about 2.0 wt. % to about 5.0 wt. % of silk fibroin-based protein fragments that are substantially devoid of sericin, wherein the silk fibroin-based protein fragments have a weight average molecular weight ranging from about 17 kDa to about 39 kDa, wherein the silk fibroin-based protein fragments have a polydispersity of between about 1.5 and about 3.0.

In some embodiments, the shampoo base comprises about 2.0 wt. % to about 5.0 wt. % of silk fibroin-based protein fragments that are substantially devoid of sericin, wherein the silk fibroin-based protein fragments have a weight average molecular weight ranging from about 39 kDa to about 80 kDa, wherein the silk fibroin-based protein fragments have a polydispersity of between about 1.5 and about 3.0.

### (ii) Additional Detersive Surfactant

In some embodiments, the shampoo base optionally comprise one or more additional detersive surfactant selected from soap, anionic surfactant, and amphiprotic surfactant.

In some embodiments, the shampoo base optionally comprises a fatty acid soap as cleansing agent. Soap as used herein refers to the salts of fatty acids of which the fatty carbon chain has 12 to 32 carbon atoms. In some embodiments, the shampoo base comprises C12-C14 fatty acid soap, C16-C18 fatty acid soap, or 80/20 blend of 80 C16-C18/20 C12-C14 fatty acid soap. The C16-C18 fatty acid soap can be obtained from tallow, and the C12-C14 fatty acid soap can be obtained from lauric, palm kernel, or coconut oils. In some embodiments, the fatty acid soaps are selected from the group consisting of sodium laurate and sodium palmitate. In some embodiments, small amount of fatty acid is added to the fatty acid soap cleansing agent to improve lather quality.

In some embodiments, the shampoo base optionally comprises sulfonates and sulfates as cleansing agent. The suitable sulfonates and sulfates are selected from the group consisting of alkyl sulfates, alkylether sulfates, alkyl sulfonates, alkylether sulfonates, alkylbenzene sulfonates, alkylbenzene ether sulfates, alkylsulfoacetates, secondary alkane sulfonates, secondary alkylsulfates, alkyl sulfosuccinates and combination thereof. The alkyl and acyl groups generally contain from 8 to 18, preferably from 10 to 16 carbon atoms and may be unsaturated. The alkyl ether sulphates, alkyl ether sulphosuccinates, alkyl ether phosphates and alkyl ether carboxylic acids and salts thereof may contain from 1 to 20 ethylene oxide or propylene oxide units per molecule.

In some embodiments, the anionic detersive surfactant is selected from the group consisting of sodium oleyl succinate, ammonium lauryl sulphosuccinate, sodium lauryl sulphate, sodium lauryl ether sulphate, sodium lauryl ether sulphosuccinate, ammonium lauryl sulphate, ammonium lauryl ether sulphate, sodium dodecylbenzene sulphonate, triethanolamine dodecylbenzene sulphonate, sodium cocoyl isethionate, sodium lauryl isethionate, lauryl ether carboxylic acid, sodium lauryl sulphate and sodium lauryl ether sulphate (EO)₁₋₃, sodium lauryl ether sulphate (EO)₁₋₃, sodium lauryl ether sulphate (EO), and sodium N-lauryl sarcosinate.

In some embodiments, the optional detersive surfactant for the shampoo base may include water-soluble salts of higher fatty acid monoglyceride monosulfates, such as the sodium salt of the monosulfated monoglyceride of hydrogenated coconut oil fatty acids, higher alkyl sulfates such as sodium lauryl sulfate, alkyl aryl sulfonates such as sodium dodecyl benzene sulfonate, higher alkyl sulfoacetates, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like.

In some embodiments, the shampoo base optionally comprises phosphates and phosphonates as detersive surfactant. The suitable phosphates and phosphonates are selected from the group consisting of alkyl phosphates, alkylether phosphates, aralkylphosphates, aralkylether phosphates, trilaureth-4-phosphate (a mixture of mono-, di- and tri-(alkyltetraglycolether)-o-phosphoric acid esters, HOSTAPHAT® 340KL from Clariant Corp.), PPG-5 ceteth 10 phosphate (CRODAPHOS® SG from Croda Inc., Parsipanny, N.J.).

In some embodiments, the shampoo base optionally comprises amine oxides as cleansing agent. The suitable amine oxide surfactants are selected from the group consisting of lauryldimethylamine oxide, laurylamidopropyldimethylamine oxide, and/or cetyl amine oxide. Typical anionic detergents include ammonium laurel sulfosuccinate, ammonium laurel sulfate, triethanolamine dodecalbenzene sulfonate, and ammonium laureth sulfate. The most preferred anionic detergents are the laurel sulfates, particularly monoethanolamine, triethanolamine and ammonium laurel sulfates and the corresponding laureth sulfates.

In some embodiments, small amounts of free fatty acid, e.g. about 0.01 wt. % to about 1.0 wt. % is added to the shampoo base to produce creamier and thicker lather. In order to provide a combination of quick lathering and length of lather, a combination of ammonium laurel sulfate and ammonium laureth sulfate is particularly preferred. Addition of cocomonoethanol amide to ammonium laurel sulfate to the shampoo base increase the lather. Behenyl alcohol may be combined with the cleansing surfactant to improve lather quality similar to the effects of adding small amounts of free fatty acid.

In some embodiments, the shampoo base optionally comprises sarcosinates and sarcosine derivatives as detersive surfactant. As used herein, sarcosinates are the derivatives of sarcosine and N-methyl glycine, acylated with a fatty acid chloride. In some embodiments, the sarcosinate is selected from sodium lauryl sarcosinate, lauryl sarcosine, cocoyl sarcosine, and combination thereof. In some embodiments, the sarcosinate is sodium lauryl sarcosinate.

The amount of the anionic surfactant component in the hair care composition should be sufficient to provide the desired cleaning and lather performance, and generally range from about 5.0 wt. % to about 50.0 wt. % by the total weight of the hair care composition. In some embodiments, the amount of the anionic surfactant component in the hair care composition ranges from about 8.0 wt. % to about 30.0 wt. % by the total weight of the hair care composition. In some embodiments, the amount of the anionic surfactant component in the hair care composition ranges from about 10.0 wt. % to about 25.0 wt. % by the total weight of the hair care composition. In some embodiments, the amount of the anionic surfactant component in the hair care composition ranges from about 12.0 wt. % to about 22.0 wt. % by the total weight of the hair care composition.

In some embodiments, the shampoo base comprises zwitterion or amphoteric detersive surfactants as detersive surfactant. Amphoteric detersive surfactants suitable for use in the hair composition include surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxyl, sulfonate, sulfate, phosphate, or phosphonate.

In some embodiments, the amphoteric detersive surfactants are selected from the group consisting of cocoampho acetate, cocoamphodiacetate, lauroamphoacetate, lauroamphodiacetate, cocobetaine and cocamidopropyl betaine, monoacetates (e.g. sodium lauroamphoacetate), diacetates (e.g. disodium lauroamphoacetate), amino- and alkylamino-propionates (e.g. lauraminopropionic acid), sultaines (also as sulfobetaines), cocamidopropylhydroxysultaine, and combination thereof.

In some embodiments, the amphoteric detersive surfactant is present in the hair care composition at a weight percent ranges from about 0.5 wt. % to about 20 wt. % by the total weight of the hair care composition. In some embodiments, the amphoteric detersive surfactant is present in the hair care composition at a weight percent ranges from about 1.0 wt. % to about 10.0 wt. % by the total weight of the hair care composition. Additional examples of suitable zwitterion or amphoteric surfactants can be found in U.S. Pat. Nos. 5,104,646 and 5,106,609.

In some embodiments, the cleansing surfactant may consist of a single surfactant, usually an anionic surfactant (to provide foam) such as sodium lauryl ether sulphate, or more commonly a mixture of sodium lauryl ether sulphate with a co-surfactant to provide mildness. The most preferred co-surfactant is cocoamidopropyl betaine.

In some embodiments, suitable detersive surfactant for shampoo base is selected from the group consisting of sodium lauryl sulfate, sodium laureth sulfate, disodium lauryl sulfosuccinate, cocoyl sarcosinate, cocoamphocarboxyglycinate and cocobetaine, and combination thereof. In some embodiments, suitable cleansing surfactant for shampoo base is non-sulfate surfactant selected from the group consisting of Cannabis sativa seed oil PEG-8 esters, sodium lauryl oat amino acid, sodium cocoyl glutamate, sodium cocoyl hydrolyzed, amaranth protein, disodium sulfosuccinate laurylglucoside cross-polymer, potassium olivoyl hydrolyzed oat protein, sodium cocoyl apple amino acids, sodium, sweetalmondamphoacetate, saponins, betaine, and combination thereof.

In some embodiments, the total weight amount of cleansing surfactant and co-surfactant in a shampoo base ranges from about 1.0 wt. % to about 50.0 wt. % by the total weight of the hair care composition. In some embodiments, the total weight amount of cleansing surfactant and co-surfactant in a shampoo base ranges from about 2.0 wt. % to about 40.0 wt. % by the total weight of the hair care composition. In some embodiments, the total weight amount of cleansing surfactant and co-surfactant in a shampoo base ranges from about 10.0 wt. % to about 25.0 wt. % by the total weight of the hair care composition. In some embodiments, the total weight amount of cleansing surfactant and co-surfactant in a shampoo base is selected from: about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, about 11.0 wt. %, about 12.0 wt. %, about 13.0 wt. %, about 14.0 wt. %, about 15.0 wt. %, about 16.0 wt. %, about 17.0 wt. %, about 18.0 wt. %, about 19.0 wt. %, about 20.0 wt. %, about 21.0 wt. %, about 22.0 wt. %, about 23.0 wt. %, about 24.0 wt. %, about 25.0 wt. %, about 26.0 wt. %, about 27.0 wt. %, about 28.0 wt. 00 about 29.0 wt. %, about 30.0 wt. %, about 31.0 wt. %, about 32.0 wt. %, about 33.0 wt. 00 about 34.0 wt. %, about 35.0 wt. %, about 36.0 wt. %, about 37.0 wt. %, about 38.0 wt. %, about 39.0 wt. %, about 40.0 wt. %, about 41.0 wt. %, about 42.0 wt. %, about 43.0 wt. %, about 44.0 wt. %, about 45.0 wt. %, about 46.0 wt. %, about 47.0 wt. %, about 48.0 wt. %, about 49.0 wt. %, about 50.0 wt. % by the total weight of the hair care composition.

### B. Thickener and Viscosity Modifying Agent

In some embodiments, the shampoo base comprises viscosity modifiers and/or thickeners.

In some embodiments, the thickener is selected from the group consisting of ethylene glycol monostearate, carbomer polymers, carboxyvinyl polymer, acrylic copolymers, methyl cellulose, copolymers of lactide and glycolide monomers, ethyl cellulose, hydroxyethyl cellulose, hydroxypropyl cellulose, carrageenan, hydrophobically modified hydroxy-ethyl-cellulose, laponite and water soluble salts of cellulose ethers such as sodium carboxymethylcellulose and sodium carboxymethyl hydroxyethyl cellulose, natural gums such as gum karaya, gum arabic, Guars, HP Guars, heteropolysaccharide gums (e.g., xanthan gum), and gum tragacanth.

In some embodiments, the thickener is selected from the group consisting of talc, fumed silica, polymeric polyether compound (e.g., polyethylene or polypropylene oxide (MW 300 to 1,000,000), capped with alkyl or acyl groups containing 1 to about 18 carbon atoms), ethylene glycol stearate, alkanolamides of fatty acids having from 16 to 22 carbon atoms, polyethylene glycol 3 distearate, polyacrylic acids (e.g., Carbopol® 420, Carbopol® 488 or Carbopol® 493), cross-linked polymers of acrylic acid, copolymers of acrylic acid with a hydrophobic monomer, copolymers of carboxylic acid-containing monomers and acrylic esters (e.g. Carbopol® 1342), cross-linked copolymers of acrylic acid and acrylate esters, polyacrylic acids cross-linked with polyfunctional agent (e.g., Carbopol® 910, Carbopol® 934, Carbopol® 940, Carbopol® 941 and Carbopol® 980, Ultrez® 10), and crystalline long chain acyl derivatives.

In some embodiments, the shampoo base comprises from about 0.1 wt. % to about 15.0 wt. % of thickener/viscosity modifying agent by the total weight of the hair care composition. In some embodiments, the shampoo base comprises from about 0.1 wt. % to about 10.0 wt. % of thickener/viscosity modifying agent by the total weight of the hair care composition. In some embodiments, the shampoo base comprises from about 0.5 wt. % to about 6.0 wt. % of thickener/viscosity modifying agent by the total weight of the hair care composition. In some embodiments, the shampoo base comprises from about 0.9 wt. % to about 4.0 wt. % of thickener/viscosity modifying agent by the total weight of the hair care composition. In some embodiments, the shampoo base comprises about 2.0 wt. % of thickener/viscosity modifying agent by the total weight of the hair care composition. In some embodiments, the amount of the thickener/viscosity modifying agent presented in the shampoo base is selected from the group consisting of about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.25 wt. %, about 1.50 wt. %, about 1.75 wt. %, about 2.0 wt. %, about 2.25 wt. %, about 2.5 wt. %, about 2.75 wt. %, about 3.0 wt. %, about 3.25 wt. %, about 3.5 wt. %, about 3.75 wt. %, about 4.0 wt. %, about 4.25 wt. %, about 4.5 wt. %, about 4.75 wt. %, about 5.0 wt. %, about 5.25 wt. %, about 5.5 wt. %, about 5.75 wt. %, about 6.0 wt. %, about 6.25 wt. %, about 7.5 wt. %, about 7.75 wt. %, about 8.0 wt. %, about 8.25 wt. %, about 8.5 wt. %, about 8.75 wt. %, about 9.0 wt. %, about 9.25 wt. %, about 9.5 wt. %, about 9.75 wt. %, about 10.0 wt. %, about 10.1 wt. %, about 10.2 wt. %, about 10.3 wt. %, about 10.4 wt. %, about 10.5 wt. %, about 10.6 wt. %, about 10.7 wt. %, about 10.8 wt. %, about 10.9 wt. %, about 11.0 wt. %, about 11.1 wt. %, about 11.2 wt. %, about 11.3 wt. %, about 11.4 wt. %, about 11.5 wt. %, about 11.6 wt. %, about 11.7 wt. %, about 11.8 wt. %, about 11.9 wt. %, about 12.0 wt. %, about 12.1 wt. %, about 12.2 wt. %, about 12.3 wt. %, about 12.4 wt. %, about 12.5 wt. %, about 12.6 wt. %, about 12.7 wt. %, about 12.8 wt. %, about 12.9 wt. %, about 13.0 wt. %, about 13.1 wt. %, about 13.2 wt. %, about 13.3 wt. %, about 13.4 wt. %, about 13.5 wt. %, about 13.6 wt. %, about 13.7 wt. %, about 13.8 wt. %, about 13.9 wt. %, about 14.0 wt. %, about 14.1 wt. %, about 14.2 wt. %, about 14.3 wt. %, about 14.4 wt. %, about 14.5 wt. %, about 14.6 wt. %, about 14.7 wt. %, about 14.8 wt. %, about 14.9 wt. %, about 15.0 wt. %, by the total weight of the hair care composition.

### C. Aqueous Carrier

In some embodiments, the shampoo base comprises water, an aqueous solution, an alcohol, a blend of alcohol and water, or a lyotropic liquid crystalline phase as aqueous carrier. Selection of the water contained in the hair care composition of the present disclosure is not limited in particular; specific examples include purified water, ion-exchanged water, and tap water. In some embodiments, the shampoo base further comprise one or more small molecule polyhydric alcohols selected from the group consisting of ethanediol, propanediol, glycerol, butanediol, butantetraol, xylitol, sorbitol, inositol, ethylene glycol, polyethylene glycol. In some embodiments, the shampoo base further comprise one or more low alcohol solvent including methanol, ethanol, and isopropanol.

In some embodiments, the shampoo base comprises from about 50 wt. % to about 98 wt. % of the aqueous carrier by the total weight of the hair care composition. In some embodiments, the shampoo base comprises from about 60 wt. % to about 90 wt. % of the aqueous carrier by the total weight of the hair care composition. In some embodiments, the amount of the aqueous carrier in the shampoo base is selected from: about 50.0 wt. %, about 51.0 wt. %, about 52.0 wt. %, about 53.0 wt. %, about 54.0 wt. %, about 55.0 wt. %, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. %, about 69.0 wt. %, about 70.0 wt. %, about 71.0 wt. %, about 72.0 wt. %, about 73.0 wt. %, about 74.0 wt. %, about 75.0 wt. %, about 76.0 wt. %, about 77.0 wt. %, about 78.0 wt. %, about 79.0 wt. %, about 80.0 wt. %, about 81.0 wt. %, about 82.0 wt. %, about 83.0 wt. %, about 84.0 wt. %, about 85.0 wt. %, about 86.0 wt. %, about 87.0 wt. %, about 88.0 wt. %, about 89.0 wt. %, about 90.0 wt. %, about 91.0 wt. %, about 92.0 wt. %, about 93.0 wt. %, about 94.0 wt. %, about 95.0 wt. %, about 96.0 wt. %, about 97.0 wt. %, about 98.0 wt. %, by the total weight of the hair care composition.

### III. Non-Aqueous Liquid Carrier

In some embodiments, the hair care composition comprises a non-aqueous liquid carrier. Non-aqueous liquid carrier as used herein means that the liquid carrier is substantially free of water. In the present disclosure, “the liquid carrier being substantially free of water” means that: the liquid carrier is free of water; or, if the liquid carrier contains water, the level of water is very low. In the present disclosure, the level of water, if included, 1% or less, preferably 0.5% or less, more preferably 0.3% or less, still more preferably 0.1% or less, even more preferably 0% by weight of the hair care composition.

In some embodiments, the non-aqueous liquid carrier comprises an oily material selected from the group consisting of mineral oil, hydrocarbon oils, hydrogenated polydecene, polyisobutene, isoparaffin, isododecane, isohexadecane, volatile silicone oil, non-volatile silicone oil, isohexadecane, squalene, squalene, ester oil and combination thereof. In some embodiments, the non-aqueous liquid carrier comprises an oily material selected from the group consisting of white mineral oils, squalane, hydrogenated polyisobutene, isohexadecane, and isododecane. In some embodiments, the non-aqueous liquid carrier comprises squalane and hydrogenated polyisobutene. In some embodiments, the non-aqueous liquid carrier comprises white mineral oils, isohexadecane, and isododecane.

In some embodiments, the non-aqueous liquid carrier comprises a volatile isoparaffin having from about 8 to about 20 carbon atoms. In some embodiments, the non-aqueous liquid carrier comprises a volatile isoparaffin having from about 8 to about 16 carbon atoms. In some embodiments, the non-aqueous liquid carrier comprises a volatile isoparaffin having from about 10 to about 16 carbon atoms. In some embodiments, the volatile isoparaffin is selected from the group consisting of trimer, tetramer, and pentamer of isobutene, and mixtures thereof. Commercially available isoparaffin hydrocarbons may have distributions of its polymerization degree, and may be mixtures of, for example, trimer, tetramer, and pentamer. What is meant by tetramer herein is that a commercially available isoparaffin hydrocarbons in which tetramer has the highest content, i.e., tetramer is included at a level of preferably 70% or more, more preferably 80% or more, still more preferably 85% or more.

In some embodiments, the volatile isoparaffin is a mixture of several grades of isoparaffins. In some embodiments, the volatile isoparaffin has a viscosity range selected from: about 0.5 mm²·s⁻¹ to about 50 mm²·s⁻¹, about 0.8 mm²·s⁻¹ to about 40 mm²·s⁻¹, about 1 mm²·s⁻¹ to about 30 mm²·s⁻¹, about 1 mm²·s⁻¹ to about 20 mm²·s⁻¹, and about 1 mm²·s⁻¹ to about 10 mm² s⁻¹, at 37.8° C. When using two or more isoparaffin hydrocarbon solvents, it is preferred that the mixture of isoparaffin hydrocarbon solvents have the above viscosity.

In some embodiments, the non-aqueous liquid carrier comprises ester oil. In some embodiments, the ester oils have an HLB of 3 or less, and as liquid at room temperature. In some embodiments, the ester oil is selected from the group consisting of methyl palmitate, methyl stearate, methyl oleate, methyl linoleate, and methyl laurate. In an embodiment, the ester oil methyl stearate.

In some embodiments, the ester oil is included in the non-aqueous liquid carrier at a weight percent selected from: about 0.1 wt. % to about 25 wt. %, about 0.5 wt. % to about 15 wt. %, about 1.0 wt. % to about 10 wt. %, about 1.0 wt. % to about 5.0 wt. % by the total weight of the hair care composition, in view of the balance between conditioned feel and product stability, and/or in view of prevent foaming.

In some embodiments, the non-aqueous liquid carrier comprises fatty esters selected from the group consisting of trimethyloylpropane tricaprylate/tricaprylate, C12-C15 alkyl benzoate, ethylhexyl stearate, ethylhexyl cocoate, decyl oleate, decyl cocoate, ethyl oleate, isopropyl myristate, ethylhexyl perlagonate, pentaerythrityl tetracaprylate/tetracaprate, PPG-3 benzyl ether myristate, propyiene glycol dicaprylate/dicaprate, ethylhexyl isostearate, ethylhexyl palmitate and natural oils such as Glycine soja, Helianthus annuus, Simmondsia chinensis, Carthamus tinctorius, Oenothera biennis and Rapae oleum, and combination thereof.

In some embodiments, the non-aqueous liquid carrier comprises glyceride fatty ester. In some embodiments, the suitable glyceride fatty esters for use in hair oils of the disclosure have a viscosity at ambient temperature (25 to 30° C.) of from 0.01 to 0.8 Pa·s, preferably from 0.015 to 0.6 Pa·s, more preferably from 0.02 to 0.065 Pa·s.

In an embodiment, the fatty material comprises a glyceride fatty ester. As used herein, the term “glyceride fatty esters” refers to the mono-, di-, and tri-esters formed between glycerol and long chain carboxylic acids such as C6-C30 carboxylic acids. The carboxylic acids may be saturated or unsaturated or contain hydrophilic groups such as hydroxyl. Preferred glyceride fatty esters are derived from carboxylic acids of carbon chain length ranging from C10 to C24, preferably C10 to C22, most preferably C12 to C 20, most preferably C12 to C 18. In some embodiments, glyceride fatty ester is a medium-chain triglyceride having C6-C12 fatty acid chain.

In some embodiments, glyceride fatty ester is sourced from varieties of vegetable and animal fats and oils, such as camellia oil, coconut oil, castor oil, safflower oil, sunflower oil, peanut oil, cottonseed oil, corn oil, olive oil, cod liver oil, almond oil, avocado oil, palm oil, sesame oil, lanolin and soybean oil. Synthetic oils include trimyristin, triolein and tristearin glyceryl dilaurate. Vegetable derived glyceride fatty esters include almond oil, castor oil, coconut oil, palm kernel oil, sesame oil, sunflower oil and soybean oil.

In some embodiments, the glyceride fatty ester is selected from coconut oil, sunflower oil, almond oil and mixtures thereof.

The non-aqueous liquid carrier is included at a level by weight of the hair care composition of, from about 50% to about 99.9%, from about 60% to about 99.8%, more preferably from about 65% to about 98% by the total weight of the hair care composition.

### IV. Aqueous Liquid Carrier Substantially Free of Non-Silk Surfactant

In some embodiments, the hair care product comprises an aqueous liquid carrier substantially free of non-silk surfactant. In some embodiments, the aqueous liquid carrier is selected from water, an aqueous solution, an alcohol, a blend of alcohol and water, or a lyotropic liquid crystalline phase. Selection of the water contained in the hair care composition of the present disclosure is not limited in particular; specific examples include purified water, ion-exchanged water, and tap water.

In some embodiments, the aqueous liquid carrier comprises one or more small molecule polyhydric alcohols selected from the group consisting of ethanediol, propanediol, glycerol, butanediol, butantetraol, xylitol, sorbitol, inositol, ethylene glycol, polyethylene glycol. In some embodiments, the aqueous liquid carrier comprises water and glycerol. In some embodiments, the aqueous liquid carrier comprises water and glycerol in a weight ratio of water to glycerol at 1:10. In some embodiments, the aqueous liquid carrier comprises water and glycerol in a weight ratio of water to glycerol selected from 1:10, 1:9, 1:8, 1:7, 1:6, 1:5, 1:4, 1:3, 1:2, and 1:1. In some embodiments, the aqueous liquid carrier comprises water and glycerol in a weight ratio of water to glycerol at 1:1. In some embodiments, the aqueous liquid carrier comprises water and glycerol in a weight ratio of water to glycerol at 1:10. In some embodiments, the aqueous liquid carrier comprises SPF, e.g., without limitation silk fibroin protein fragments and glycerol in a weight ratio of SPF, e.g., without limitation silk fibroin protein fragments to glycerol selected from 1:10, 1:9, 1:8, 1:7, 1:6, 1:5, 1:4, 1:3, 1:2, and 1:1. In some embodiments, the aqueous liquid carrier comprises SPF, e.g., without limitation silk fibroin protein fragments and glycerol in a weight ratio of SPF, e.g., without limitation silk fibroin protein fragments to glycerol at 1:1.

In some embodiments, the pH of the aqueous liquid phase is adjusted ranging from about 4.0 to about 9.0. In some embodiments, the pH of the aqueous liquid phase is adjusted ranging from about 4.5 to about 8.5. In some embodiments, the pH of the aqueous liquid phase is adjusted ranging from about 5.0 to about 7.0. The pH adjusting agent may include buffer (e.g. PBS buffer), alkali metal salt, acid, citric acid, succinic acid, phosphoric acid, sodium hydroxide, ammonium hydroxide, ethanolamine, sodium carbonate, and combination thereof.

In some embodiments, the hair care composition comprises from about 1.0 wt. % to about 99.0 wt. % of the aqueous liquid carrier by the total weight of the hair care composition. In some embodiments, the hair care composition comprises from about 5.0 wt. % to about 45.0 wt. % of the aqueous liquid carrier by the total weight of the hair care composition. In some embodiments, the hair care composition comprises from about 5.0 wt. % to about 35.0 wt. % of the aqueous liquid carrier by the total weight of the hair care composition. In some embodiments, the hair care composition comprises from about 10.0 wt. % to about 30.0 wt. % of the aqueous liquid carrier by the total weight of the hair care composition. In some embodiments, the hair care composition comprises from about 45.0 wt. % to about 95.0 wt. % of the aqueous liquid carrier by the total weight of the hair care composition. In some embodiments, the hair care composition comprises from about 60.0 wt. % to about 90.0 wt. % of the aqueous liquid carrier by the total weight of the hair care composition. In some embodiments, the hair care composition comprises from about 45.0 wt. % to about 75.0 wt. % of the aqueous liquid carrier by the total weight of the hair care composition. In some embodiments, the hair care composition comprises from about 60.0 wt. % to about 75.0 wt. % of the aqueous liquid carrier by the total weight of the hair care composition. In some embodiments, the amount of the aqueous liquid carrier in the hair care composition is selected from: about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, about 11.0 wt. %, about 12.0 wt. %, about 13.0 wt. %, about 14.0 wt. %, about 15.0 wt. %, about 16.0 wt. %, about 17.0 wt. %, about 18.0 wt. %, about 19.0 wt. %, about 20.0 wt. %, about 21.0 wt. %, about 22.0 wt. %, about 23.0 wt. %, about 24.0 wt. %, about 25.0 wt. %, about 26.0 wt. %, about 27.0 wt. %, about 28.0 wt. %, about 29.0 wt. %, about 30.0 wt. %, about 31.0 wt. %, about 32.0 wt. %, about 33.0 wt. %, about 34.0 wt. %, about 35.0 wt. %, about 36.0 wt. %, about 37.0 wt. %, about 38.0 wt. %, about 39.0 wt. %, about 40.0 wt. %, about 41.0 wt. %, about 42.0 wt. %, about 43.0 wt. %, about 44.0 wt. %, about 45.0 wt. %, about 46.0 wt. %, about 47.0 wt. %, about 48.0 wt. %, about 49.0 wt. %, about 50.0 wt. %, about 51.0 wt. %, about 52.0 wt. %, about 53.0 wt. %, about 54.0 wt. %, about 55.0 wt. %, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. %, about 69.0 wt. %, about 70.0 wt. %, about 71.0 wt. %, about 72.0 wt. %, about 73.0 wt. %, about 74.0 wt. %, about 75.0 wt. %, about 76.0 wt. %, about 77.0 wt. %, about 78.0 wt. %, about 79.0 wt. %, about 80.0 wt. %, about 81.0 wt. %, about 82.0 wt. %, about 83.0 wt. %, about 84.0 wt. %, about 85.0 wt. %, about 86.0 wt. %, about 87.0 wt. %, about 88.0 wt. 00 about 89.0 wt. %, about 90.0 wt. %, about 91.0 wt. %, about 92.0 wt. %, about 93.0 wt. 00 about 94.0 wt. %, about 95.0 wt. %, about 96.0 wt. %, about 97.0 wt. %, about 98.0 wt. %, by the total weight of the hair care composition.

### 4) Formulation Additives

In some embodiments, the hair care composition optionally comprises one or more formulation additives selected from the group consisting of anti-static agents (e.g., tricetyl methyl ammonium chloride), natural or synthetic fragrant essential oils, gelling agents, foam enhancing agents, chelating agents (e.g., EDTA), antioxidants (e.g, butylhydroxytoluene, BHT), propyl, octyl and dodecyl esters of gallic acid, butylated hydroxyanisole (BHA), pH adjusting agents (e.g. sodium citrate, citric acid, succinic acid, phosphoric acid, sodium hydroxide, and sodium carbonate), preservatives (e.g. DMDM hydantoin, ethyl paraben and butyl paraben), antimicrobials (e.g. triclosan or triclocarbon), organic solvents or diluents, pearlescent aids, vitamins, fragrances, ultraviolet light scattering agents, heat protection agents, scalp protecting agents, proteins or decomposed proteins such as soybean protein, gelatin, collagen, silk fibroin and elastin, various amino acids such as silk amino acids derived from hydrolysis of silk fibroin fiber, hair growth promoting agent, biotin, pantothenic acid, blood flow promoting agents, T-oryzanol, sodium dextran sulfate, nicotine derivatives, anti-seborrhea agents, sulfur, thiantol and combination thereof.

In some embodiments, the hair care composition optionally comprises trans-4-tert-butyl-cyclohexanol as the scalp protecting agent.

In some embodiments, the hair care composition optionally comprises heat protection agents selected from the group consisting of the SPF, e.g., without limitation silk fibroin protein fragments as disclosed herein, glycerin, propylene glycol, hydrolyzed wheat protein PG-propylsilanetriol, sodium polystyrene sulfonate, Quaternium-70 (Ceraphyl™ 70), polyquaternium-11 (Gafquat™ 755N), cationic polymer PVP/DMAPA acrylates copolymer (Styleze™ CC-IO, ISP), sodium PEG-40 maleate/styrene sulfonate copolymer, triquaternary polydimethyl siloxane (Abil™ T Quat 60, Evonik), and combinations thereof.

In some embodiments, the hair care composition optionally comprises a natural or synthetic fragrant essential oil. In some embodiments, the fragrant essential oil is selected from the group consisting of eucalyptus oil, lavandin oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil, almond oil, argan oil, avocado oil, cedar oil, wheat germ oil, bergamot oil, and combination thereof.

In some embodiments, the hair care composition optionally comprises vitamins selected from the group selected from the group consisting of vitamin A, vitamin B, vitamin E, vitamin D, vitamin K, riboflavin, pyridoxin, coenzyme thiamine pyrophosphate, flavin adenine dinucleotide, folic acid, pyridoxal phosphate, tetradrofolic acid, and combination thereof.

In some embodiments, the hair care composition contains vitamin and/or coenzymes at about 0.01 wt. % to about 8.0 wt. % by the total weight of the hair care composition. In some embodiments, the hair care composition contains vitamin and/or coenzymes at about 0.001 wt. % to about 10.0 wt. % by the total weight of the hair care composition. In some embodiments, the hair care composition contains vitamin and/or coenzymes at about 0.05 wt. % to about 5.0 wt. % by the total weight of the hair care composition.

In some embodiments, the hair care composition optionally comprises a preservative selected from the group consisting of triazoles, imidazoles, naphthalene derivatives, benzimidazoles, morphline derivatives, dithiocarbamates, benzisothiazoles, benzamides, boron compounds, formaldehyde donors, isothiazolones, thiocyanates, quaternary ammonium compounds, iodine derivates, phenol derivatives, micobicides, pyridines, dialkylthiocarbamates, nitriles, parabens, alkyl parabens, and salts thereof.

In some embodiments, the hair care composition optionally comprises a volatile organic liquid as a foam enhancing agent to provide foaming upon use or as a propellant. The volatile organic liquid is used to enhance the foam produced by primary surfactant and is a gas producing agent, which when exposed to air and temperature will provide instant, copious lather. The volatile organic liquid foam enhancing agent preferably boils in the range of 25° C. to 50° C. at an atmospheric pressure. In some embodiments, the volatile organic liquid foam enhancing agent is selected from the group consisting of saturated hydrocarbons (e.g., n-pentane, iso-pentane, n-butane, isobutene), C1-C6 alkyl ethers (e.g. dimethyl either, diethyl ether, methylethyl ether, diisopropyl ether), and combination thereof. The use amount of volatile organic liquids in the hair care compositions will depend upon the type of hair care product being formulated and the function to be served by the volatile organic liquid. In some embodiments, the volatile organic liquid is present in an amount ranging from about 1 wt. % to about 7 wt. % by the total weight of the hair care composition. In some embodiments, the volatile organic liquid is present in an amount ranging from about 3 wt. % to about 4 wt. % by the total weight of the hair care composition.

### 2. Product Forms

In some embodiments, the hair care composition is formulated in a form selected from the group consisting of aqueous solution, ethanolic solution, oil, gel, emulsion, suspension, mousses, liquid crystal, solid, gels, lotions, creams, aerosol sprays, paste, foam and tonics. In some embodiments, the hair care composition is in a form selected from the group consisting of a cream, spray, aerosol mousse or gel.

In some embodiments, the hair care composition is a hair care product selected from the group consisting of pre-shampoo products, shampoo products, hair rinses, hair conditioners, hair treatments, setting lotions, blow-styling lotions, hair sprays, hair-styling foams, hair-styling gels, styling gels, styling creams, styling mousses, styling foams, styling sprays, styling spritz, styling mists, styling glazes, styling fixes, sculpting lotions, sculpting gels, sculpting glazes, sculpting sprays, glossing gels, glossing spritz; shaping gels, forming mousses, modeling spritz, finishing spritz, fixing gels, setting lotions, permanent waving agents hair oil, hair liquids, hair tonics, hair creams, and hair cosmetics.

In some embodiments, the disclosure provides a hair wax product including a hair care composition including silk fibroin fragments as described herein, and a dermatologically acceptable carrier as described herein.

In some embodiments, this disclosure provides a hair care composition capable of foaming comprising SPF, e.g., without limitation silk fibroin protein fragments as disclosed herein, and less than 20.0 wt. % by the total weight of the hair care composition of a surfactant system containing (a) a sulfate-based surfactant, (b) a silicon-based surfactant, (c) synthetic surfactant.

In some embodiments, this disclosure provides a hair care composition comprising silk fragments, capable of coating hair cuticles and managing hair frizz.

In some embodiments, this disclosure provides a hair care composition capable of increasing hair sheen comprising SPF, e.g., without limitation silk fibroin protein fragments as disclosed herein.

In some embodiments, this disclosure provides a hair care composition capable of enhancing hair color comprising SPF, e.g., without limitation silk fibroin protein fragments as disclosed herein.

### I. Hair Spray

In some embodiments, the hair care composition is a hair styling product comprising silk fibroin protein fragment solution, a polyhydric material (polyhydrialcohol and/or polysaccharide) having a HLB value higher than 10, plant extract and an aqueous liquid carrier as described above, wherein the polyhydric alcohols include glycerol, wherein the silk fibroin protein fragment is incorporated as film forming agent.

In some embodiments, the hair styling product is a hair spray or a specialty type fixative. In some embodiments, the disclosure provides a heat protection spray including a hair care composition including silk fibroin fragments as described herein, and a dermatologically acceptable carrier as described herein. In some embodiments, the silk hair care composition is useful for preventing the damages caused by the heated styling appliances such as straightening irons, hair dryers, curling tongs and the like. The heat protection spray disclosed hair provides protection against the damage caused by heating hair to temperatures ranging from 60° C. to 300° C., preferably to temperatures ranging from 170° C. to 230° C.

The film forming agent provides hair holding properties and curl retention, little flaking or powder on combing, rapid curing or drying on hair, nonstickiness, and be easily removable by shampooing. Film forming agent is delivered by a solvent which is usually an alcohol or a mixture of an alcohol and water and the alcohol having a HLB value higher than 10.

In some embodiments, the hair styling product may be in the form of a hair spray. The hair spray typically includes the hair styling agent and a cosmetically acceptable carrier. In some embodiments, the carrier is water or a water and alcohol mixture. The spray formulation optionally includes an antioxidant, sunscreen agent, vitamin, protein, peptide, plant extract, humectant, oil, emollient, lubricant, thickener, hair conditioning agent, polymer, and/or surfactant. In some embodiments, the hair spray includes a preservative. In some embodiments, the hair spray includes a fragrance. In some embodiments, the hair spray includes a surfactant. In some embodiments, the hair spray contains water, fragrance, a preservative, and silk fibroin protein fragment solution. In some embodiments, the hair spray contains water, fragrance, a preservative, and silk fibroin protein fragment solution. In some embodiments, the hair spray contains water, a preservative, fragrance, the crosslinking agent, and an anti-static agent. In some embodiments, the hair spray contains water, a preservative, fragrance, silk fibroin protein fragment solution, and a hair conditioning agent. In some embodiments, the hair spray contains water, a preservative, fragrance, silk fibroin protein fragment solution, and a surfactant.

In some embodiments, the hair spray comprises silk fibroin-based protein fragments that are substantially devoid of sericin of about 1.0 wt. % to about 6.0 wt. % by the total weight of the hair spray, wherein the silk fibroin-based protein fragments have a weight average molecular weight ranging from about 5 k Da to about 300 kDa, wherein the silk fibroin-based protein fragments have a polydispersity of between about 1.5 and about 3.0. In some embodiments, the hair spray comprises silk fibroin-based protein fragments that are substantially devoid of sericin of about 1.0 wt. % to about 3.0 wt. % by the total weight of the hair spray, wherein the silk fibroin-based protein fragments have a weight average molecular weight ranging from about 5 kDa to about 300 kDa, wherein the silk fibroin-based protein fragments have a polydispersity of between about 1.5 and about 3.0. In some embodiments, the hair spray comprises silk fibroin-based protein fragments that are substantially devoid of sericin of about 3.0 wt. % to about 6.0 wt. % by the total weight of the hair spray, wherein the silk fibroin-based protein fragments have a weight average molecular weight ranging from about 5 kDa to about 300 kDa, wherein the silk fibroin-based protein fragments have a polydispersity of between about 1.5 and about 3.0.

In some embodiments, the hair spray comprises silk fibroin-based protein fragments that are substantially devoid of sericin of about 1.0 wt. % to about 3.0 wt. % by the total weight of the hair spray, wherein the silk fibroin-based protein fragments have a weight average molecular weight ranging from about 6 kDa to about 17 kDa, wherein the silk fibroin-based protein fragments have a polydispersity of between about 1.5 and about 3.0. In some embodiments, the hair spray comprises silk fibroin-based protein fragments that are substantially devoid of sericin of about 1.0 wt. % to about 3.0 wt. % by the total weight of the hair spray, wherein the silk fibroin-based protein fragments have a weight average molecular weight ranging from about 17 kDa to about 39 kDa, wherein the silk fibroin-based protein fragments have a polydispersity of between about 1.5 and about 3.0. In some embodiments, the hair spray comprises silk fibroin-based protein fragments that are substantially devoid of sericin of about 3.0 wt. % to about 6.0 wt. % by the total weight of the hair spray, wherein the silk fibroin-based protein fragments have a weight average molecular weight ranging from about 39 kDa to about 80 kDa, wherein the silk fibroin-based protein fragments have a polydispersity of between about 1.5 and about 3.0.

In some embodiments, the hair spray comprises silk fibroin-based protein fragments that are substantially devoid of sericin, wherein the silk fibroin-based protein fragments have a weight average molecular weight ranging from about 6 kDa to about 17 kDa, about 17 kDa to about 39 kDa, and/or about 39 kDa to about 80 kDa wherein the silk fibroin-based protein fragments have a polydispersity of between about 1.5, and wherein the silk fibroin based protein fragment is presented in an amount selected from: about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6.0 wt. % by the total weight of the hair spray.

Aerosol-form compositions of hair spray will include an aerosol propellant which serves to expel the other materials from the container, and forms the mousse character in mousse compositions. The aerosol propellant included in styling compositions of the present disclosure can be any liquefiable gas conventionally used for aerosol containers. Examples of suitable propellants include dimethyl ether and hydrocarbon propellants such as propane, n-butane and iso-butane. The propellants may be used singly or admixed. Water insoluble propellants, especially hydrocarbons, are preferred because they form emulsion droplets on agitation and can create suitable mousse foam densities when needed.

The amount of the propellant used is governed by normal factors well known in the aerosol art. For mousses the level of propellant is generally up to 35%, preferably from 2% to 30%, most preferably from 3% to 15% by weight based on total weight of the composition. If a propellant such as dimethyl ether includes a vapor pressure suppressant (e.g. trichloroethane or dichloromethane), for weight percentage calculations, the amount of suppressant is included as part of the propellant. For aerosol sprays the levels of propellant are usually higher; preferably from 30 to 98 wt. % of the total composition, more preferably 50 to 95 wt. %.

Preferred propellants are selected from propane, n-butane, isobutene, dimethyl ether and mixtures thereof. Preferably, the propellant comprises dimethyl ether and at least one of propane, n-butane and isobutene. The method of preparing aerosol hair styling mousse compositions according to the disclosure follows conventional aerosol filling procedures. The composition ingredients (not including the propellant) are charged into a suitable pressurizable container which is sealed and then charged with the propellant according to conventional techniques.

### II. Hair Styling Cream, Hair Styling Gel

In some embodiments, the hair styling product may also take a non-foaming product form, such as a hair styling cream or gel. In some embodiments, the hair styling product may be in the form of a cream. In some embodiments, the hair styling product may be in the form of a gel. In some embodiments, the hair cream/gel is for leave in use. In some embodiments, the hair cream/gel is transparent, or translucent.

In some embodiments, the hair styling cream/gel comprises silk fibroin protein fragment solution, a polyhydric material (polyhydrialcohol and/or polysaccharide) having a HLB value higher than 10, plant extract, and an aqueous liquid carrier as described above, wherein the polyhydric alcohols include glycerol, wherein the silk fibroin protein fragment is incorporated as film forming agent.

In some embodiments, the hair styling cream/gel comprises about 1.0 wt. % to about 6.0 wt. % by the total weight of the hair styling product of silk fibroin-based protein fragments that are substantially devoid of sericin, wherein the silk fibroin-based protein fragments have a weight average molecular weight ranging from about 5 k Da to about 300 kDa, wherein the silk fibroin-based protein fragments have a polydispersity of between about 1.5 and about 3.0. In some embodiments, the hair styling cream/gel comprises about 1.0 wt. % to about 3.0 wt. % by the total weight of the hair styling product of silk fibroin-based protein fragments that are substantially devoid of sericin, wherein the silk fibroin-based protein fragments have a weight average molecular weight ranging from about 5 kDa to about 300 kDa, wherein the silk fibroin-based protein fragments have a polydispersity of between about 1.5 and about 3.0. In some embodiments, the hair styling cream/gel comprises about 3.0 wt. % to about 6.0 wt. % by the total weight of the hair styling product of silk fibroin-based protein fragments that are substantially devoid of sericin, wherein the silk fibroin-based protein fragments have a weight average molecular weight ranging from about 5 kDa to about 300 kDa, wherein the silk fibroin-based protein fragments have a polydispersity of between about 1.5 and about 3.0.

In some embodiments, the hair styling cream/gel comprises about 1.0 wt. % to about 3.0 wt. % by the total weight of the hair styling cream/gel of silk fibroin-based protein fragments that are substantially devoid of sericin, wherein the silk fibroin-based protein fragments have a weight average molecular weight ranging from about 17 kDa to about 39 kDa, wherein the silk fibroin-based protein fragments have a polydispersity of between about 1.5 and about 3.0. In some embodiments, hair styling cream/gel comprises about 3.0 wt. % to about 6.0 wt. % by the total weight of the hair styling cream/gel of silk fibroin-based protein fragments that are substantially devoid of sericin, wherein the silk fibroin-based protein fragments have a weight average molecular weight ranging from about 39 kDa to about 80 kDa, wherein the silk fibroin-based protein fragments have a polydispersity of between about 1.5 and about 3.0.

In some embodiments, the hair styling cream/gel comprises silk fibroin-based protein fragments that are substantially devoid of sericin, wherein the silk fibroin-based protein fragments have a weight average molecular weight ranging from about 6 kDa to about 17 kDa, about 17 kDa to about 39 kDa, and/or about 39 kDa to about 80 kDa, wherein the silk fibroin-based protein fragments have a polydispersity of between about 1.5, and wherein the silk fibroin based protein fragment is presented in an amount selected from: about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6.0 wt. % by the total weight of the hair styling cream/gel.

In some embodiments, the hair styling cream/gel comprises silk fibroin-based protein fragments that are substantially devoid of sericin, wherein the silk fibroin-based protein fragments have a weight average molecular weight ranging from about 39 kDa to about 80 kDa, wherein the silk fibroin-based protein fragments have a polydispersity of between about 1.5, and wherein the silk fibroin based protein fragment is presented in an amount selected from: about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6.0 wt. % by the total weight of the hair styling cream/gel.

Additionally, the cream may include an oil, a hair conditioning agent, and/or a thickening agent. The cream may also include a fragrance, a plant extract, and/or a surfactant. The cream may be packaged in a tube, tub, bottle, or other suitable container.

Additionally, the cream may include polymers which are substantially insoluble in liquid, volatile organic hydrocarbons and are dimensionally stable on exposure to air. Suitably the molecular weight of the carboxyvinyl polymer is at least 750,000, preferably at least 1,250,000, most preferably at least 3,000,000. Preferred carboxyvinyl polymers are copolymers of acrylic acid cross-linked with allyl sucrose or allylpentaerythritol (e.g CARBOPOL® 934, 940, 941 and 980). Other materials that can also be used as structurants or thickeners include those that can impart a gel-like viscosity to the composition, e.g. methylcellulose, hydroxyethylcellulose, hydroxypropylmethylcellulose and carboxymethylcellulose, guar gum, sodium alginate, gum arabic, xanthan gum, polyvinyl alcohol, polyvinylpyrrolidone, hydroxypropyl guar gum, starch and starch derivatives. It is also possible to use inorganic thickeners such as talc, fumed silica, bentonite or laponite clays.

Such a cream or gel will include a structurant or thickener, typically at a level of from 0.1% to 10%, preferably 0.5% to 3% by weight based on total weight of the hair styling cream or gel. Examples of suitable structurants or thickeners are polymeric thickeners, e.g., carboxyvinyl polymers. A carboxyvinyl polymer is an interpolymer of a monomeric mixture comprising a monomeric olefinically unsaturated carboxylic acid, and from about 0.01% to about 10% by weight of the total monomers of a polyether of a polyhydric alcohol. Carboxyvinyl

### III. Shampoo

In some embodiments, the hair care composition is a shampoo product comprising silk fibroin protein fragment solution, a polyhydric material (polyhydrialcohol and/or polysaccharide) having HLB value >10, and a shampoo bass as described above, wherein the water soluble SPF, e.g., without limitation silk fibroin protein fragments function as a detersive surfactant for the shampoo. The SPF, e.g., without limitation silk fibroin protein fragments containing shampoo provides a lather when massaged into hair, washed out well, and “squeaky” feel during use.

In some embodiments, the shampoo comprises about 2.0 wt. % to about 5.0 wt. % of silk fibroin-based protein fragments that are substantially devoid of sericin, wherein the silk fibroin-based protein fragments have a weight average molecular weight ranging from about 5 kDa to about 80 kDa, wherein the silk fibroin-based protein fragments have a polydispersity of between about 1.5 and about 3.0.

In some embodiments, the shampoo comprises about 3.5 wt. % to about 5.0 wt. % of silk fibroin-based protein fragments that are substantially devoid of sericin, wherein the silk fibroin-based protein fragments have a weight average molecular weight ranging from about 5 kDa to about 80 kDa, wherein the silk fibroin-based protein fragments have a polydispersity of between about 1.5 and about 3.0.

In some embodiments, the shampoo comprises silk fibroin-based protein fragments that are substantially devoid of sericin, wherein the silk fibroin-based protein fragments have a weight average molecular weight ranging from about 5 kDa to about 80 kDa, wherein the silk fibroin-based protein fragments have a polydispersity of between about 1.5, and wherein the silk fibroin based protein fragment is presented in an amount selected from about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, and about 5.0 wt. % by the total weight of the shampoo composition.

In some embodiments, the shampoo comprises about 5.0 wt. % of silk fibroin-based protein fragments that are substantially devoid of sericin, wherein the silk fibroin-based protein fragments have a weight average molecular weight ranging from about 5 kDa to about 80 kDa, wherein the silk fibroin-based protein fragments have a polydispersity of between about 1.5 and about 3.0.

In some embodiments, the shampoo comprises about 2.0 wt. % to about 5.0 wt. % of silk fibroin-based protein fragments that are substantially devoid of sericin, wherein the silk fibroin-based protein fragments have a weight average molecular weight ranging from about 6 kDa to about 17 kDa, wherein the silk fibroin-based protein fragments have a polydispersity of between about 1.5 and about 3.0. In some embodiments, the shampoo comprises about 3.5 wt. % to about 5.0 wt. % of silk fibroin-based protein fragments that are substantially devoid of sericin, wherein the silk fibroin-based protein fragments have a weight average molecular weight ranging from about 6 kDa to about 17 kDa, wherein the silk fibroin-based protein fragments have a polydispersity of between about 1.5 and about 3.0. In some embodiments, the shampoo comprises silk fibroin-based protein fragments that are substantially devoid of sericin, wherein the silk fibroin-based protein fragments have a weight average molecular weight ranging from about 6 kDa to about 17 kDa, wherein the silk fibroin-based protein fragments have a polydispersity of between about 1.5, and wherein the silk fibroin based protein fragment is presented in an amount selected from about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, and about 5.0 wt. % by the total weight of the shampoo composition. In some embodiments, the shampoo comprises about 5.0 wt. % of silk fibroin-based protein fragments that are substantially devoid of sericin, wherein the silk fibroin-based protein fragments have a weight average molecular weight ranging from about 5 kDa to about 15 kDa, wherein the silk fibroin-based protein fragments have a polydispersity of between about 1.5 and about 3.0.

In some embodiments, the shampoo comprises about 2.0 wt. % to about 5.0 wt. % of silk fibroin-based protein fragments that are substantially devoid of sericin, wherein the silk fibroin-based protein fragments have a weight average molecular weight ranging from about 17 kDa to about 39 kDa, wherein the silk fibroin-based protein fragments have a polydispersity of between about 1.5 and about 3.0.

In some embodiments, the shampoo comprises about 3.5 wt. % to about 5.0 wt. % of silk fibroin-based protein fragments that are substantially devoid of sericin, wherein the silk fibroin-based protein fragments have a weight average molecular weight ranging from about 17 kDa to about 39 kDa, wherein the silk fibroin-based protein fragments have a polydispersity of between about 1.5 and about 3.0.

In some embodiments, the shampoo comprises silk fibroin-based protein fragments that are substantially devoid of sericin, wherein the silk fibroin-based protein fragments have a weight average molecular weight ranging from about 17 kDa to about 39 kDa, wherein the silk fibroin-based protein fragments have a polydispersity of between about 1.5, and wherein the silk fibroin based protein fragment is presented in an amount selected from about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, and about 5.0 wt. % by the total weight of the shampoo composition.

In some embodiments, the shampoo comprises about 5.0 wt. % of silk fibroin-based protein fragments that are substantially devoid of sericin, wherein the silk fibroin-based protein fragments have a weight average molecular weight ranging from about 17 kDa to about 39 kDa, wherein the silk fibroin-based protein fragments have a polydispersity of between about 1.5 and about 3.0.

In some embodiments, the shampoo further comprises about 0.01 wt. % to about 3.0 wt. % by the total weight of the shampoo of silk fibroin amino acids (Gly+Ala+Ser) and/or low molecular weight silk fibroin peptides (200 Da to 5 kDa) as described above as conditioning agent to nourish and conditioning the hair.

### IV Hair Conditioner

In some embodiments, the hair care composition is in the form of a hair conditioner comprising SPF, e.g., without limitation silk fibroin protein fragments, a polyhydric material (polyhydrialcohol and/or polysaccharide) having HLB value >10, an emulsion base, and hair conditioning active agents as described above, wherein the oil phase of the emulsion carrier containing the oily materials emulsifyable by the silk emulsifier selected from the group consisting of a vegetable oil, isododecane, and isohexadecane, and one or more oily esters of fatty acids, wherein the vegetable oil is selected from jojoba oils and/or camellia oils, wherein said oily esters are selected from isononyl isononanoate and coco caprylate.

In some embodiments, the hair conditioner is for leave-in or leave-on use. In some embodiments, the hair conditioner is transparent, or translucent. In some embodiments, the hair conditioner imparts a good texture to the hair without giving any tackiness or greasiness. In some embodiments, the hair conditioner is for rinse-off use. In some embodiments, the hair conditioner comprises silk fibroin peptides as described above as hair conditioning agent to impart enhanced feel during use.

In some embodiments, the hair conditioner comprises any silk fibroin peptides or any silk fibroin fragments described herein. In some embodiments, the hair conditioner comprises silk fibroin peptides that are substantially devoid of sericin of about 0.01 wt. % to about 3.0 wt. % by the total weight of the hair conditioner, wherein the silk fibroin peptides have a weight average molecular weight ranging from about 200 Da to about 5 kDa. In some embodiments, the hair conditioner comprises silk fibroin peptides that are substantially devoid of sericin of about 0.01 wt. % to about 0.06 wt. % by the total weight of the hair conditioner, wherein the silk fibroin peptides have a weight average molecular weight ranging from about 200 Da to about 5 kDa. In some embodiments, the hair conditioner comprises silk fibroin peptides that are substantially devoid of sericin of about 0.1 wt. % to about 2.0 wt. % by the total weight of the hair conditioner, wherein the silk fibroin peptides have a weight average molecular weight ranging from about 200 Da to about 5 kDa. In some embodiments, the hair conditioner comprises silk fibroin peptides that are substantially devoid of sericin of about 0.5 wt. % to about 2.0 wt. % by the total weight of the hair conditioner, wherein the silk fibroin peptides have a weight average molecular weight ranging from about 200 Da to about 5 kDa. In some embodiments, the hair conditioner comprises silk fibroin peptides that are substantially devoid of sericin of about 0.6 wt. % to about 1.5 wt. % by the total weight of the hair conditioner, wherein the silk fibroin peptides have a weight average molecular weight ranging from about 200 Da to about 5 kDa. In some embodiments, the hair conditioner comprises silk fibroin peptides that are substantially devoid of sericin of about 0.6 wt. % to about 0.75 wt. % by the total weight of the hair conditioner, wherein the silk fibroin peptides have a weight average molecular weight ranging from about 200 Da to about 5 kDa.

In some embodiments, the hair conditioner comprises silk fibroin peptides that are substantially devoid of sericin of about 0.01 wt. % to about 3.0 wt. % by the total weight of the hair conditioner, wherein the silk fibroin peptides have a weight average molecular weight of ranging from about 200 Da to about 1000 Da. In some embodiments, the hair conditioner comprises silk fibroin peptides that are substantially devoid of sericin of about 0.01 wt. % to about 0.6 wt. % by the total weight of the hair conditioner, wherein the silk fibroin peptides have a weight average molecular weight of ranging from about 200 Da to about 1000 Da. In some embodiments, the hair conditioner comprises silk fibroin peptides that are substantially devoid of sericin of about 0.1 wt. % to about 2.0 wt. % by the total weight of the hair conditioner, wherein the silk fibroin peptides have a weight average molecular weight of ranging from about 200 Da to about 1000 Da. In some embodiments, the hair conditioner comprises silk fibroin peptides that are substantially devoid of sericin of about 0.6 wt. % to about 1.5 wt. % by the total weight of the hair conditioner, wherein the silk fibroin peptides have a weight average molecular weight of ranging from about 200 Da to about 1000 Da.

In some embodiments, the hair conditioner comprises silk fibroin peptides that are substantially devoid of sericin of about 0.01 wt. % to about 3.0 wt. % by the total weight of the hair conditioner, wherein the silk fibroin peptides have a weight average molecular weight of about 1000 Da. In some embodiments, the hair conditioner comprises silk fibroin peptides that are substantially devoid of sericin of about 0.1 wt. % to about 2.0 wt. % by the total weight of the hair conditioner, wherein the silk fibroin peptides have a weight average molecular weight of about 1000 Da. In some embodiments, the hair conditioner comprises silk fibroin peptides that are substantially devoid of sericin of about 0.6 wt. % by the total weight of the hair conditioner, wherein the silk fibroin peptides have a weight average molecular weight of about 1000 Da. In some embodiments, the hair conditioner comprises silk fibroin peptides that are substantially devoid of sericin of about 0.6 wt. % to about 1.6 wt. % by the total weight of the hair conditioner, wherein the silk fibroin peptides have a weight average molecular weight of about 1000 Da.

In some embodiments, the hair conditioner comprises silk fibroin amino acids as described above as hair conditioning agent to deliver as humectant and to nourish hair. In some embodiments, silk fibroin protein amino acids are derived from the hydrolyzed silk fibroin. In some embodiments, the silk fibroin amino acids are from commercially available hydrolyzed silk (CAS Number: 96690-41-4). The amino acid composition of silk fibroin protein from Bombyx mori consists mainly of Gly (43%), Ala (30%), and Ser (12%).

In some embodiments, the silk fibroin amino acids are present in the hair conditioner at a weight percent ranging from about 0.001 wt. % to about 1.5 wt. % by the total weight of the hair conditioner. In some embodiments, the silk fibroin amino acids are present in the hair conditioner at a weight percent ranging from about 0.1 wt. % to about 1.0 wt. % by the total weight of the hair conditioner. In some embodiments, the silk fibroin amino acids are present in the hair conditioner at a weight percent ranging from about 0.2 wt. % to about 0.6 wt. % by the total weight of the hair conditioner.

In some embodiments, the hair conditioner comprises a mixture of silk fibroin protein fragment (5 kDa to 80 kDa), and/or silk amino acids (Gly+Ala+Ser), and/or low molecular weight silk fibroin peptide (200 Da to 5 kDa), wherein silk fibroin protein fragment, the silk fibroin peptide and the silk fibroin amino acids each independently is present in the hair conditioner at a weight percent ranging from about 0.1 wt. % to about 1.0 wt. % by the total weight of the hair conditioner.

In some embodiments, the hair conditioner may include cationic polymers derived from polysaccharides, for example cationic cellulose derivatives, cationic starch derivatives, cationic guar derivatives and cationic locust bean gum derivatives, synthetic cationic polymers, mixtures or combinations of these agents. The formulation may comprise other synthetic or natural polymers or polymers derived from biological preparation processes, which are functionalized, where appropriate, for example with cationic or neutral groups. These polymers may have a stabilizing or strengthening action on the compositions, and/or a conditioning action (deposition on the surface of the skin or the hair).

In some embodiments, the emulsion carrier is a water-in-oil emulsion, a water-in-oil emulsion, a microemulsion, a nanoemulsion, or a bicontinuous microemulsion. The hair conditioner optionally includes an antioxidant, sunscreen agent, vitamin, protein, peptide, plant extract, humectant, oil, emollient, lubricant, thickener, hair conditioning agent, polymer, and/or surfactant. In some embodiments, the hair conditioner includes a preservative. In some embodiments, the hair conditioner includes a fragrance. In some embodiments, the hair spray includes a surfactant. In some embodiments, the hair conditioner contains water, fragrance, a preservative.

The hair conditioning agent may be included in any suitable concentration. In some embodiments, the amount of the hair conditioning agent present in the conditioner ranges from about 0.01 wt. % to about 50 wt. % by the total weight of the hair conditioner composition. In some embodiments, the amount of the hair conditioning agent present in the conditioner ranges from about 0.1 wt. % to about 25.0 wt. % by the total weight of the hair conditioner composition. In some embodiments, the amount of the hair conditioning agent present in the conditioner ranges from about 1.0 wt. % to about 15.0 wt. % by the total weight of the hair conditioner composition.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### General Procedures

The compositions of this disclosure may be made by various methods known in the art. Such methods include those of the following examples, as well as the methods specifically exemplified below. As used herein in the working examples, “low molecular weight,” “low MW,” or “low-MW” silk fibroin fragments include fragments with a molecular weight between about 14 and about 30 kDa. As used herein in the working examples, “medium molecular weight,” “medium MW,” or “mid-MW” silk fibroin fragments include fragments with a molecular weight between about 39 and about 54 kDa.

### Example 1: Straightened Style Silk Hair Care Composition

FIGS. 1A-1C illustrate silk fibroin protein fragments aqueous solutions as hair styling resin for straightening and curling hairs, provide style and curl retention. FIG. 1A: untreated brown tress; FIG. 1B: Medium molecular weight Silk Fibroin Solution (6 wt %, 0.8 mL) was applied to an untreated brown tress; the tress was combed 10 times with a fine-tooth comb, and blow-dried on high heat for 120 seconds. A straightened style is achieved; FIG. 1C: Medium molecular weight Silk Fibroin Solution (6 wt %, 0.8 mL) was applied to a brown tress, the tress was combed 10 times with a fine-tooth comb and blow-dried on high heat for 120 seconds while wrapped around a conical tube (30 mm diameter). A curled style is achieved; typical resin concentration in high-hold styling products: 3-6 wt %; typical resin concentration in relaxed-hold styling products: 1-3 wt %.

### Example 2: Surfactant/Shampoo Active Silk Hair Care Composition

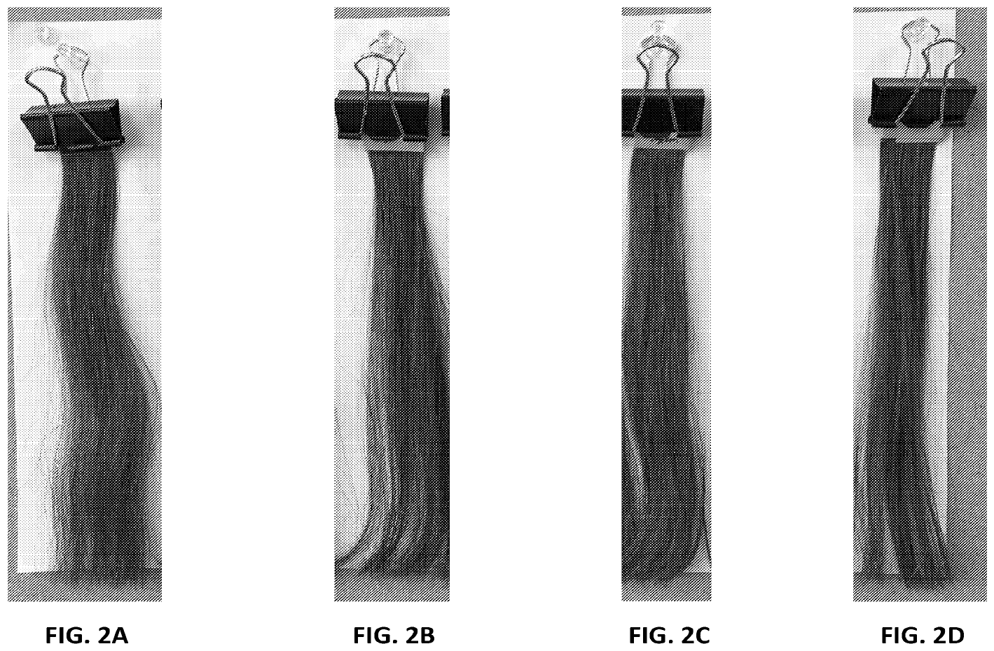

FIGS. 2A-2D illustrate silk fibroin protein fragments aqueous solution as a surfactant/shampoo active; silk provides a lather when massaged into hair, washes out well, and provides a clean, clarifying feeling during application. FIG. 2A: untreated brown tress; FIG. 2B water (0.3 mL) was applied to a brown tress to dampen it, and then Tween 20 (5 wt %, 0.6 mL) was applied to the tress; the tress was combed 10 times with a fine-tooth comb and then blow-dried on high heat for 120 seconds; FIG. 2C: water (0.3 mL) was applied to a brown tress to dampen it, and then medium molecular weight Silk Fibroin Solution (5% solids, 0.6 mL) was applied to the tress; the tress was combed 10 times with a fine-tooth comb and then blow-dried on high heat for 120 seconds; FIG. 2D: water (0.3 mL) was applied to a brown tress to dampen it, and Low-MW Silk Fibroin Solution (5% solids, 0.6 mL) was applied to the tress; the tress was combed 10 times with a fine-tooth comb and then blow-dried on high heat for 120 seconds. All surfactants tested provided some lather while washing. Low molecular weight Silk Fibroin Solution felt especially clarifying during massage, indicative of good surfactant performance. All samples blow-dried well, with no discernible residue left behind.

### Example 3: Surfactant Detergency of Silk Fibroin Solutions

Table 19 shows the oil removal efficacy of various surfactants, including Silk Fibroin Solution A and Silk Fibroin Solution B. The fibroin solutions were compared to Laureth-3, a commonly employed personal care surfactant. To obtain the data, tresses were labeled and masses recorded. N=2 tresses were prepared per treatment. For each surfactant of interest, a 1% solution of surfactant in tap water was made (1 L). To test, a tress was evenly coated with 0.45 mL olive oil. The tress was then dipped in the testing solution twenty times. The tress was then dipped in tap water (1 L) ten times. These steps were repeated for each surfactant of interest. The tresses were allowed to air dry for three days and massed again once dry to calculate the oil removal.

TABLE 19
Relative detergency of surfactants

| Surfactant | % Oil Removed | % silk |
|---|---|---|
| Laureth-3 | 6.6 | |
| Silk Fibroin Solution A | 7.8 | 1% Low-MW silk fibroin |
| Silk Fibroin Solution B | 6.3 | 1% Mid-MW silk fibroin |

### Example 4. Deposition of Silk Fibroin-Based Hair Smoothing Therapy on Hair

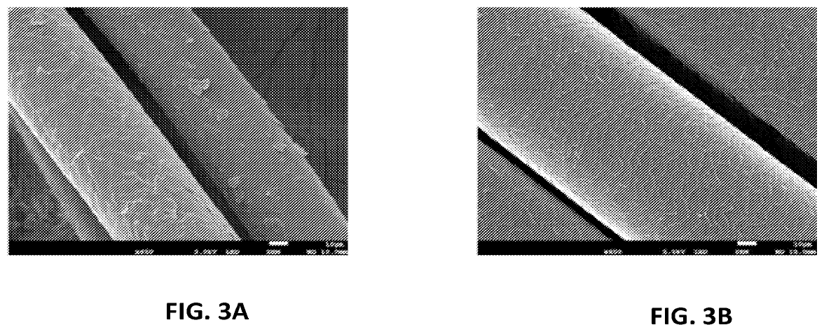

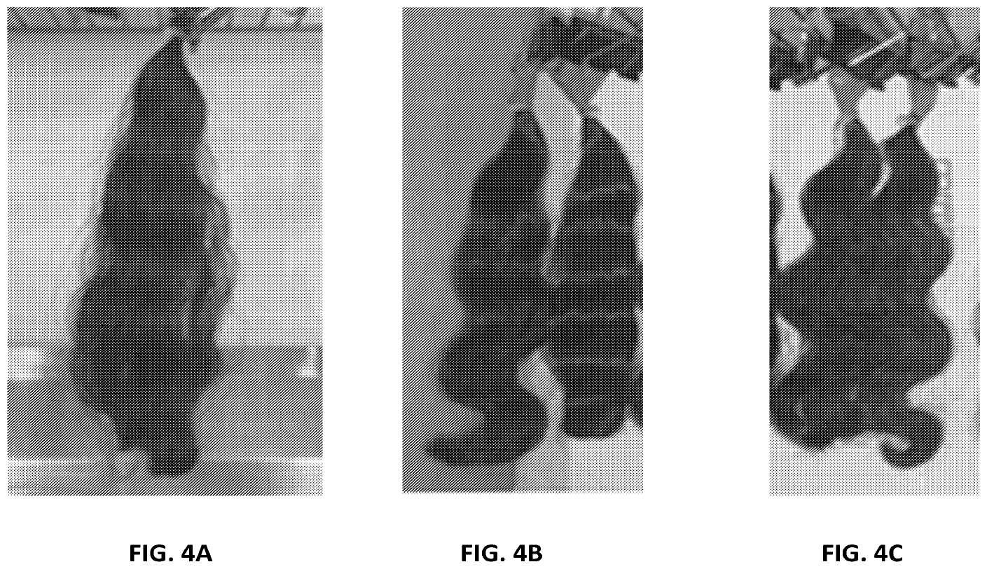

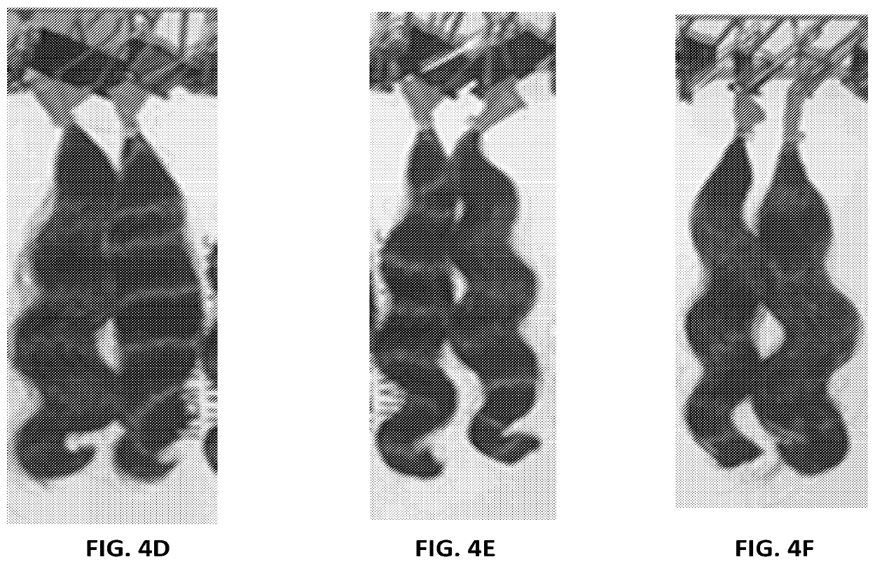

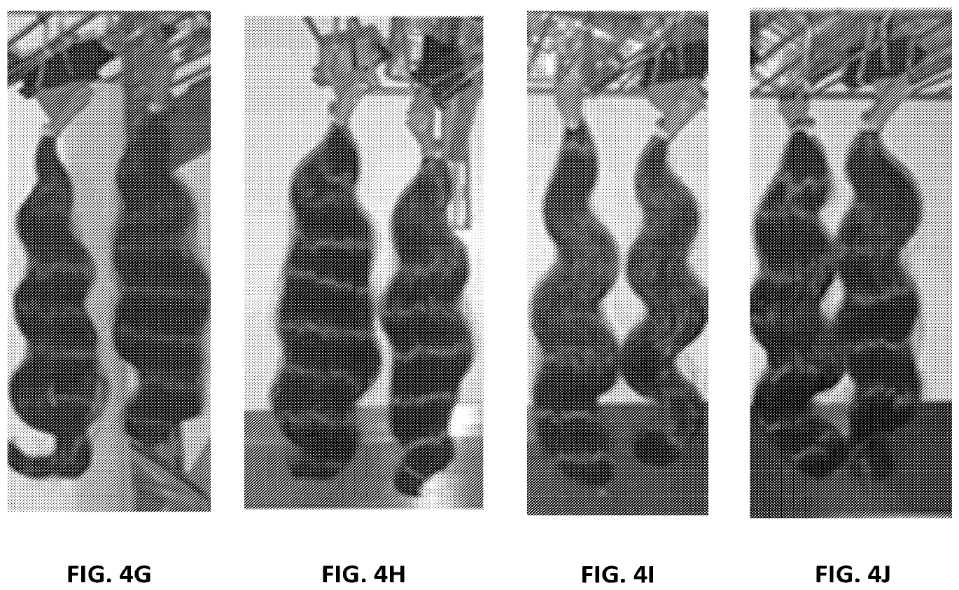

A bleached hair sample was rinsed with tap water, treated with 3% solution of Low MW Silk, rinsed again, and left to air dry overnight. A small sample of the treated hair as well as bleached untreated hair were sputter coated with a 10 nm (80:20) Pt:Pd coating and analyzed using a JEOL 7900F SEM. The side by side comparison of the SEM images for bleached untreated hair (FIG. 3A) and the SEM image for silk treated hair (FIG. 3B) demonstrated the smoothing effect to the keratin fiber by silk fibroin coatings.

### Example 5. Silk Fibroin-Based Hair Treatments and their Impacts on Combability and Gloss to the Hair

Bleached straight hair tresses were uniformly washed with 15% sodium lauryl sulfate and 70% isopropyl alcohol and dried. The tresses were rinsed for 10 seconds in tap water then 2 mL of solution (as described in Table 20) was applied to the hair tresses. The solution was again rinsed for 10 seconds in tap water while the tress was combed through 5 times. The combing was evaluated with wide side of comb. The combing was rated on a scale of 1 (very difficult to comb) to 5 (very easy to comb). The tresses were left to dry overnight and were evaluated again for ease of dry combing with tight side of comb with the same rating scale. The gloss of each tress was measured with the ETB-0833 Self-Calibration 200 60° 850 Glossmeter at the 60° setting before and after treatment with solution. The chemical structures for the functionalized silks applied in the testing are provided in Table 20. The testing results for combability and gloss imparted by the functionalized silk are summarized in Tables 21-22 below.

TABLE 20
Functionalized Silk used in the testing

| Functionalized Silk | Chemical Structure |
|---|---|
| (Low-MW-098-02- 01) | 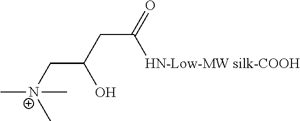 |
| (Mid-MW-098-02- 02) | 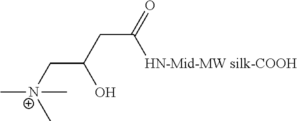 |
| Hexamine (098-10- 2) | 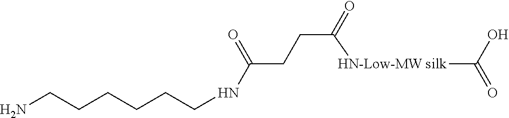 |
| | 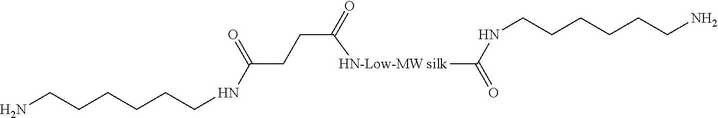 |
| | 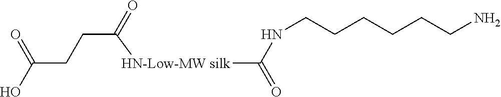 |
| (Mid-MW-098-08- 02) | 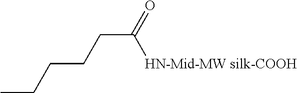 |

TABLE 21
Ratings of Silk Solutions Impact on Wet and Dry Combability
(1 = Worst, 5 = Best)

| | | Av. | Av. | Av. |
|---|---|---|---|---|
| | | Wet | Dry | Combined |
| | | Combing | Combing | Combing |
| | Solution | Rating | Rating | Rating |
| T001 | Water | 4.00 | 4.38 | 4.19 |
| T002 | Hydrolyzed Wheat Protein | 3.38 | 3.63 | 3.50 |
| | (2%) | | | |
| T003 | Low-MW Silk (2%) | 4.00 | 3.25 | 3.63 |
| T004 | Mid-MW Silk (2%) | 3.88 | 3.25 | 3.56 |
| T005 | Low-098-02-01 (2%) | 2.13 | 3.25 | 2.69 |
| T006 | Mid-098-02-02 (2%) | 2.50 | 2.75 | 2.63 |
| T007 | Hexamine (2%) | 3.50 | 4.00 | 3.75 |

TABLE 22
Change in Gloss with Application of Silk Solutions

| | | Av. | Av. | |
|---|---|---|---|---|
| | | Initial | Final | Av. % |
| | | Gloss | Gloss | Change in |
| | Solution | (GU) | (GU) | Gloss |
| T001 | Water | 2.17 | 2.53 | 17.08% |
| T002 | Hydrolyzed Wheat Protein (2%) | 2.20 | 2.68 | 22.01% |
| T003 | Low-MW Silk (2%) | 2.17 | 2.70 | 24.72% |
| T004 | Mid-MW Silk (2%) | 2.20 | 2.63 | 20.01% |
| T005 | Low-098-02-01 (2%) | 2.10 | 2.47 | 17.46% |
| T006 | Mid-098-02-02 (2%) | 2.32 | 2.48 | 8.07% |
| T007 | Hexamine (2%) | 2.12 | 2.42 | 14.37% |

### Example 6. Silk Fibroin-Based Hair Treatment and their Impacts on Smoothness, Frizz and Curl Definition to the Hair

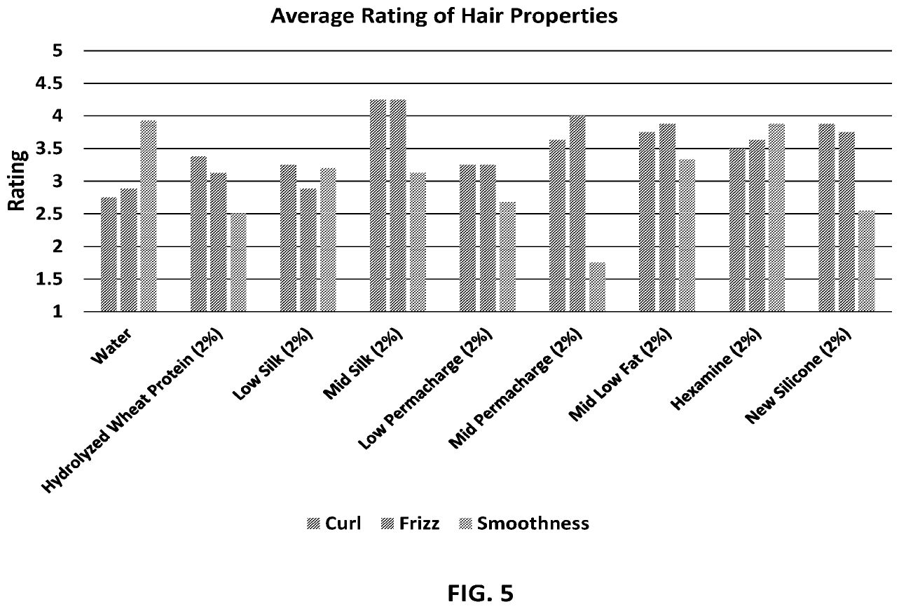

Curly hair tresses were uniformly washed with 15% sodium lauryl sulfate and dried. The tresses were rinsed for 20 seconds in tap water then 1 mL of hair treatment solution (as described in Table 23) was applied to the hair. The solution was gently massaged for 30 seconds. After application, the tress was again rinsed for 20 seconds in tap water. The tresses were left to dry overnight and were evaluated for curl definition, frizz, and smoothness on a scale of 1 (least desirable result) to 5 (most desirable result) based on the observations of 5 individuals. FIG. 5 is a summary graph of the visual ratings for each nourishing parameter. All of the analyzed silk types showed equal or greater performance to the industry standard hydrolyzed wheat protein.

TABLE 23
Ratings of Silk Solutions Impact on Curl Definition, Frizz,
and Smoothness (1 = Worst, 5 = Best)

| | Solution | Curl | Frizz | Smoothness |
|---|---|---|---|---|
| T001 | Water | 2.75 | 2.88 | 3.93 |
| T002 | Hydrolyzed Wheat Protein (2%) | 3.38 | 3.13 | 2.5 |
| T003 | Low-MW Silk (2%) | 3.25 | 2.88 | 3.2 |
| T004 | Mid-MW Silk (2%) | 4.25 | 4.25 | 3.13 |
| T005 | Low-098-02-01 (2%) | 3.25 | 3.25 | 2.68 |
| T006 | Mid-098-02-02 (2%) | 3.63 | 4.00 | 1.75 |
| T007 | Mid-098-08-02 (2%) | 3.75 | 3.88 | 3.33 |
| T008 | Hexamine 098-10-2 (2%) | 3.50 | 3.63 | 3.88 |
| T009 | New Silicone (2%) | 3.88 | 3.75 | 2.55 |

### Example 7. Silk Fibroin-Based Hair Treatment and their Impacts on Water Repellency to the Hair

Bleached hair tresses were uniformly cleaned with 15% Sodium Lauryl Sulfate and dried. The tresses were treated with 5 mL of solution (as described in Table 4) and then blow dried on a high heat. A disposable 3 mL pipet was used to distribute a single drop of tap water on a dried tress and the wetting time of the drop was recorded via stopwatch. Three drops were recorded for each tress, and their average wetting time recorded in Table 24.

TABLE 24
Summary of Average Wetting Time of Silk Solutions on Hair

| | | Av. Wetting |
|---|---|---|
| | | Time |
| Sample | Solution | (sec) |
| T001 | Water | 0.74 |
| T010 | Mid-MW Silk (6%) | 2.60 |
| T011 | Low-MW Silk (6%) | 1.49 |
| T012 | Mid-MW Silk (6%, pH ~4.5, citric acid) | 2.88 |
| T013 | Low-MW Silk (6%, pH ~4.5, citric acid) | 1.61 |

### Example 8. Silk Fibroin-Based Hair Treatment and their Impacts on Hair Styling and Texturizing

### Example 8a. Curl Retention

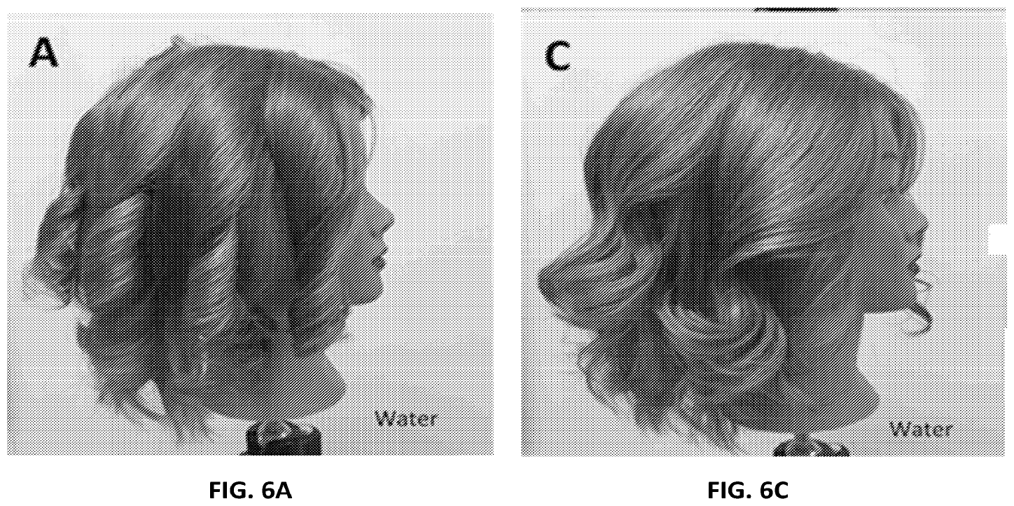

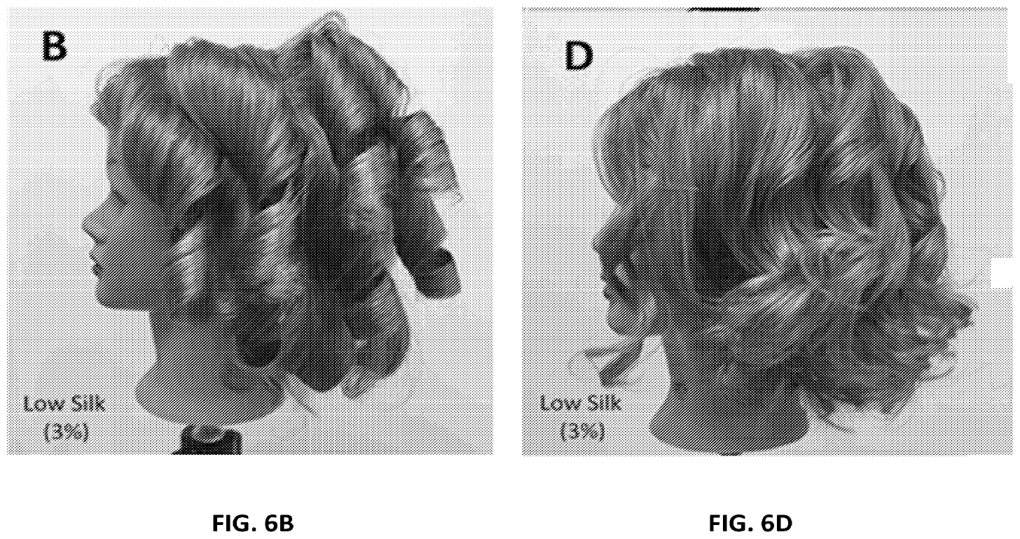

A mannequin head with human hair was evenly divided into left and right sections. The right side of the mannequin was treated with 22.6 g of 3% low silk and combed through. The wet hair was blow dried with finger combing. The hair was divided into 9 sections throughout the head. Each section was curled with a 1″ diameter curling iron at 350° F. for 10 seconds. The left side was treated with 22.6 g of tap water and blow dried and curled in the same fashion. The two sides of the head were qualitatively compared for curl retention after finger combing out the curls (results photographed in FIG. 6).

### Example 8b. Volume Retention

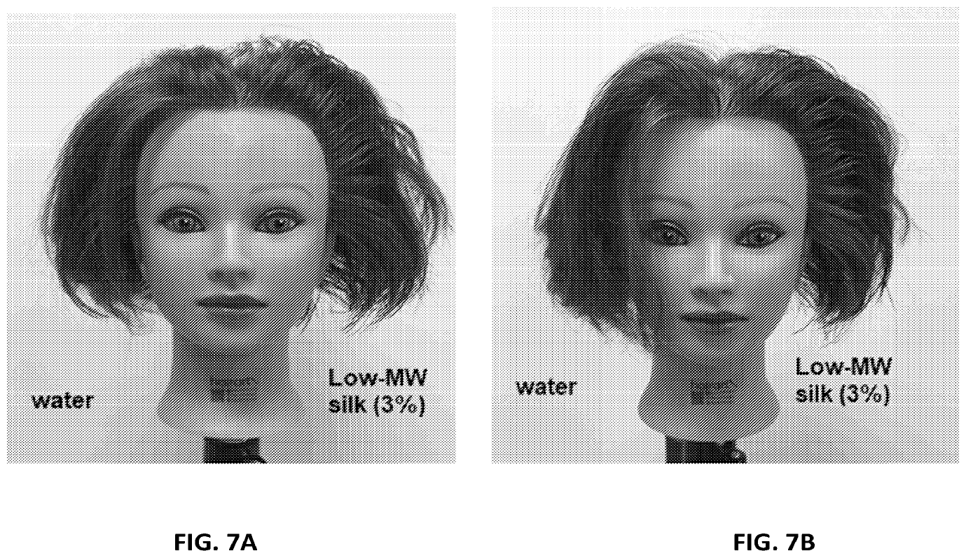

A mannequin head with human hair was evenly divided into left and right sections. The right side of the mannequin was treated with 17.5 g of 3% low silk and combed through. The wet hair was blow dried with a comb while pulling up the roots to create volume. The left side was treated with 17.5 g of water and blow dried in the same fashion, with a comb to pull the roots up to create volume. The two sides were qualitatively compared initially and then 24 hours later as seen in FIGS. 7A-B.

### Example 9. Silk Fibroin-Based Hair Treatment and their Impacts on Semi-Permanent and Permanent Hair Shaping

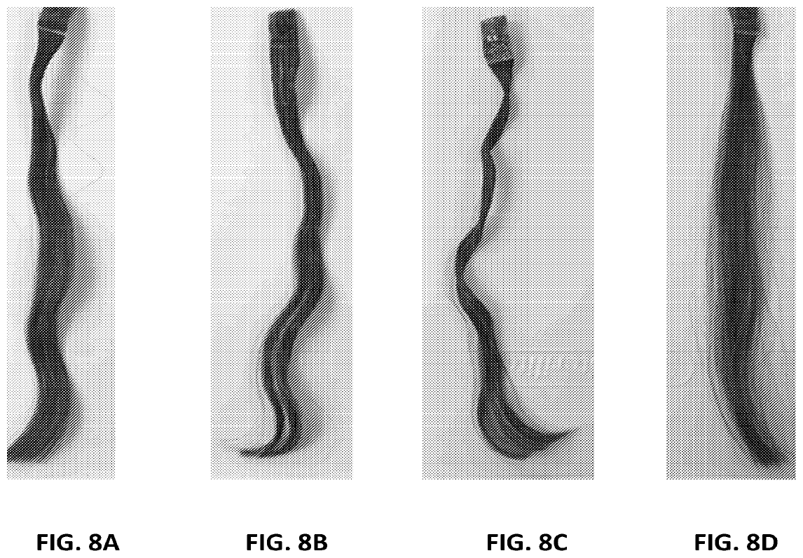

Solutions were prepared as per Table 25. Hair samples (1.25 cm wide) were placed in each of four weigh boats (labeled A-D). 4 mL of the appropriate solution was added to each boat. Solutions were worked into samples with a gloved finger until hair was saturated. Each sample was then wrapped around a roller and secured. Rollers were wrapped in aluminum foil and placed under a hooded hair dryer on “high” setting for 60 m minutes. Samples were then unwrapped, rinsed with lukewarm tap water and hung to dry. Results are depicted in FIGS. 8A-D.

TABLE 25
Reagent amounts used to prepare solutions for hair shaping.

| | | | | | | Suc- |
|---|---|---|---|---|---|---|
| | DI | Low MW | Mid MW | Sodium | Cys- | cinic |
| Sample | water | silk (6%) | silk (6%) | carbonate | teine | acid |
| 002-A | 40 mL | | | 2.54 g | 1.46 g | 0.71 g |
| 002-B | 20 mL | | 20 mL | 2.54 g | 1.46 g | 0.71 g |
| 002-C | 20 mL | 20 mL | | 2.54 g | 1.46 g | 0.71 g |
| 002-D | 40 mL | | | | | |

Silk has demonstrated various hair improvement qualities when used on its own, but it is reasonable to expect that silk in combination with other formulating ingredients would still be expected to demonstrate, and most likely enhance, these qualities. Examples of ingredients that could be used to improve combability include, but are not limited to, cationic surfactants and quaternary ammoniums such as hydroxypropyltrimonium chloride. Examples of ingredients that could be used to improve curl definition include, but are not limited to, fatty oils such as coconut oil, jojoba oil, and shea butter. Examples of ingredients that could be used to improve frizz and smoothness include, but are not limited to, hydrolyzed wheat protein, stearic acid, and pantothenic acid. Examples of ingredients that could be used to improve gloss and shine include, but are not limited to, glycerin and almond oil.

It is also reasonable to expect that variations on the formulations described in Table 25 would also be effective in semi-permanent to permanent hair shaping. The concept of using cysteine as a benign reducing agent can reasonably be expanded to include additional sulfhydryl-containing compounds such as (but not limited to)N-acetyl cysteine, cysteine methyl and ethyl esters, and salts thereof. Crosslinking agents used can be reasonably extended beyond succinic acid to include other bis- or tri-acids such as (but not limited to) glutaric, adipic, citric, malic, aspartic, glutamic, malonic and tartaric acids. Use of these active ingredients in the presence of additional formulating reagents (stabilizing, emulsifying, conditioning, etc.) is also a reasonable extension of this work.

### Example 10. Synthesis of Functionalized Silk

As used herein the symbols and conventions used in these processes, schemes and examples are consistent with those used in the contemporary scientific literature, for example, the Journal of the American Chemical Society or the Journal of Biological Chemistry.

Unless otherwise indicated, all temperatures are expressed in ° C. (degrees Centigrade). All reactions conducted under an inert atmosphere at room temperature unless otherwise noted. Reagents employed without synthetic details are commercially available or made according to literature procedures.

HPLC/Mass spectra were obtained on Dyonex series 3000 HPLC coupled with Q Exactive™ Hybrid Quadrupole-Orbitrap™ Mass Spectrometer. Detection is by MS, UV at 214 nM using either Atmospheric Chemical Ionization (APCI) or Electrospray Ionization (ESI) and an evaporative light-scattering detectior (ELSD). The data was acquired using Thermo Scientific™ Xcalibur™ Software. Data analysis was performed using PEAKS software.

Silk fibroin is secreted in the form of a 2.3 MDa protein complex which consists of six sets of heavy chain-light chain heterodimer and one molecule of fibrohexamerin (P25). Covalent modification of silk fibroin was confirmed for different subunits (heavy chain, light chain, and/or fibrohexamerin) based on m/z and ms2 fragmentation patterns.

Prior to HPLC/MS, the functionalized silk synthesized was subjected to protease digestion according to the procedures below. In general, the functionalized silk in each sample were denatured with 6M guanidine HCl and reduced with DTT at 60° C. for 30 minutes followed by alkylation with iodoacetamide at room temperature in the dark. The alkylation reaction was quenched by the addition of excess DTT and the reaction was allowed to proceed for another 30 minutes at room temperature. Chymotrypsin digestion was carried out at 37° C. overnight at a protein to protease ratio of 1:50.

Atenuated Total Reflection was conducted on lyophilized functionalized silk samples using Nicolet iS50 FTIR Spectrometer.

The functionalized silk prepared according to the experimental procedures described below are summarized in Table 26.

TABLE 26
Functionalized Silk

| Sample ID | Structure | Characterization |
|---|---|---|
| 077-027-1 | 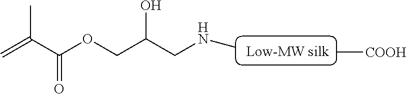 | MS, IEF^{a} |
| 077-024-2 | 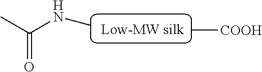 | MS, IEF |
| 077-028-2 | 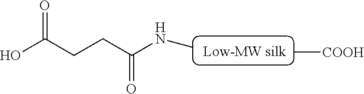 | MS, IEF |
| 077-030-1 | 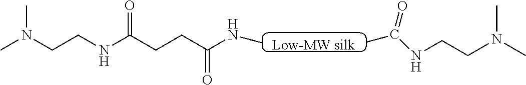 | MS, IEF |
| 098-08-02 | 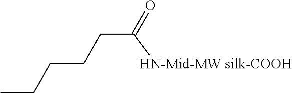 | FTIR |
| 098-29-02 | 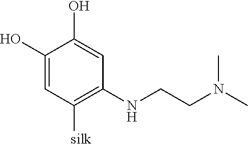 | SEC-RI, FTIR |
| 098-30-02 | 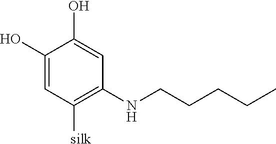 | SEC-RI, FTIR |

a. IEF stands for isoelectric focus.

### Example 10a. Sample 077-027-1

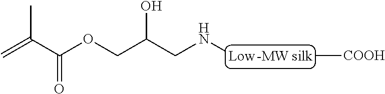

Low MW silk was placed on an ice bath and stirred at 300 rpm. The pH of the solution was adjusted to 9.5 and then glycidyl methacrylate was added in 3 portions, over 3 hours. After the addition, the ice bath was removed, and the mixture was allowed to warm up to room temperature (RT). The mixture was allowed to react at RT for 30 minutes. The reaction mixture was purified by dialysis against water using a 10 kDa MWCO dialysis tubing.

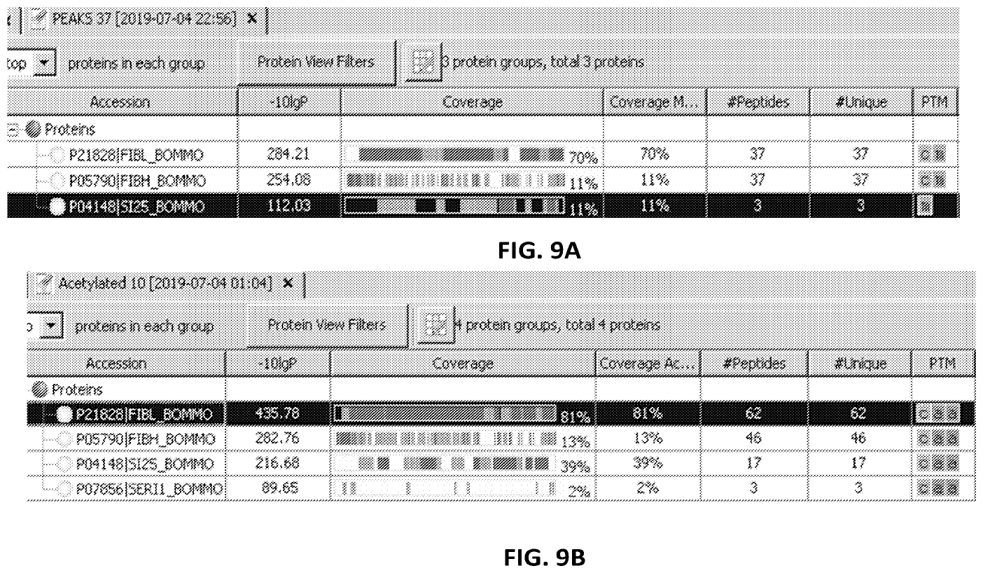

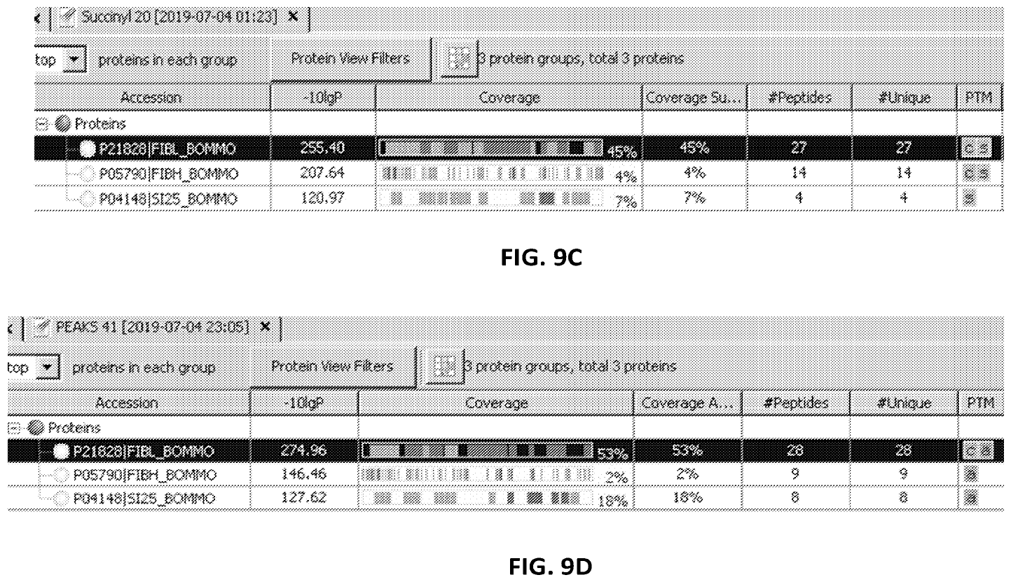

Covalent modification of Low-MW silk fibroin was confirmed for all three subunits (heavy chain, light chain, and fibrohexamerin) based on m/z and ms2 fragmentation patterns from the mass spectrum obtained in HPLC/MS analysis (See FIG. 9A and FIGS. 12A-B).

### Example 10b. Sample 077-024-2

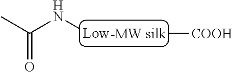

Low MW silk was placed on an ice bath and stirred at 300 rpm. Acetic anhydride was added in 3 portions, over 1 hour. After each portion the pH was adjusted to 8.5-9.5 with sodium hydroxide. After the last succinic acid addition, the ice bath was removed, and the reaction was allowed to warm up to room temperature. The mixture was allowed to react at RT for 30 minutes. The reaction mixture was purified by dialysis against water using a 10 kDa MWCO dialysis tubing.

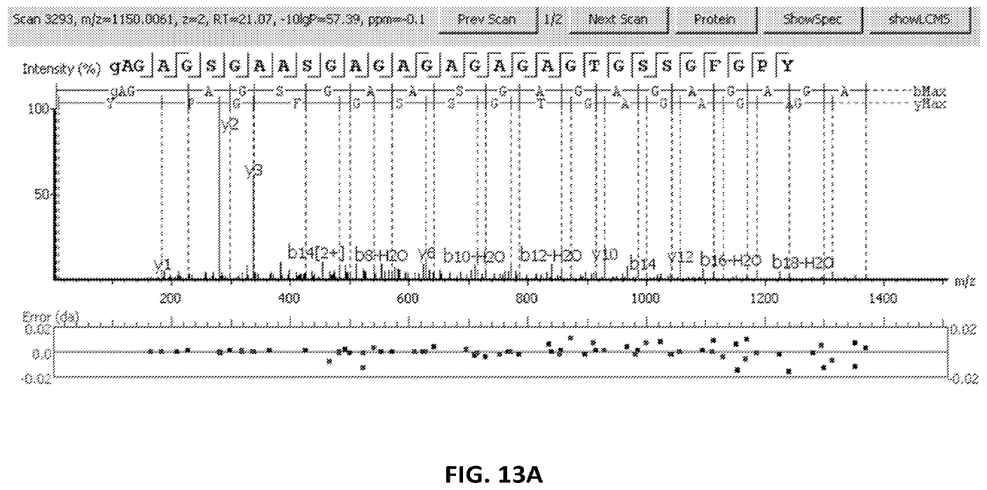

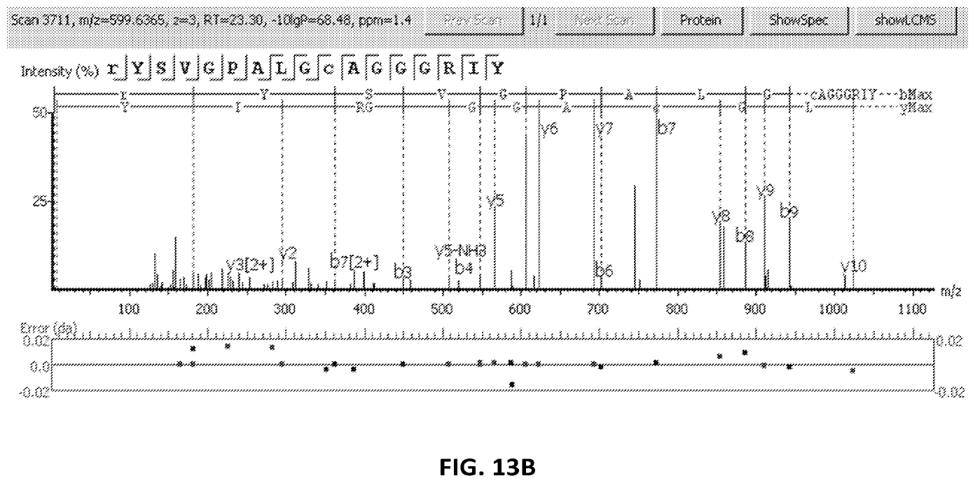

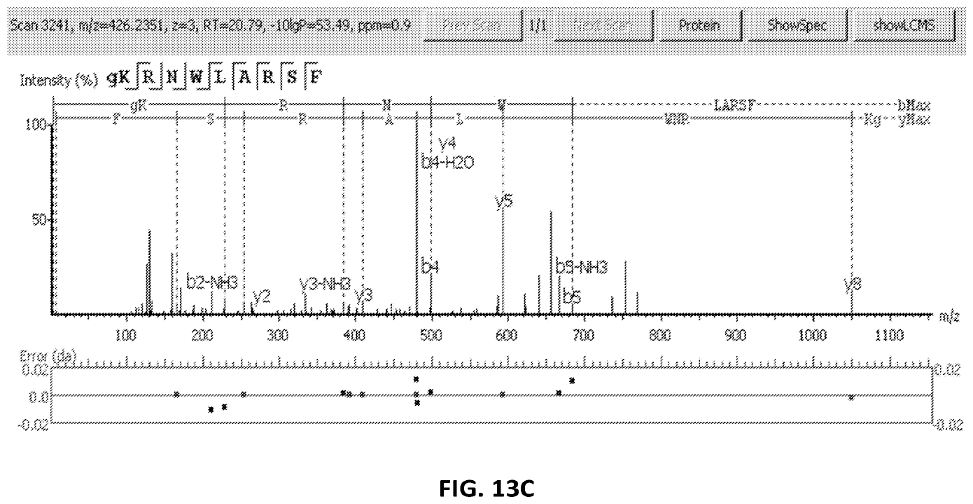

Covalent modification of Low-MW silk fibroin was confirmed for all three subunits (heavy chain, light chain, and fibrohexamerin) based on m/z and ms2 fragmentation patterns from the mass spectrum obtained in HPLC/MS analysis (See FIG. 9B and FIGS. 13A-C).

### Example 10c. Sample 077-028-2

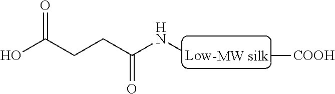

Low MW silk was placed on an ice bath and stirred at 300 rpm. Succinic anhydride was added in 3 portions, over 1 hour. After each portion the pH was adjusted to 8.5-9.5 with sodium hydroxide. After the last succinic acid addition, the ice bath was removed, and the reaction was allowed to warm up to room temperature. The mixture was allowed to react at RT for 30 minutes. The reaction mixture was purified by dialysis against water using a 10 kDa MWCO dialysis tubing.

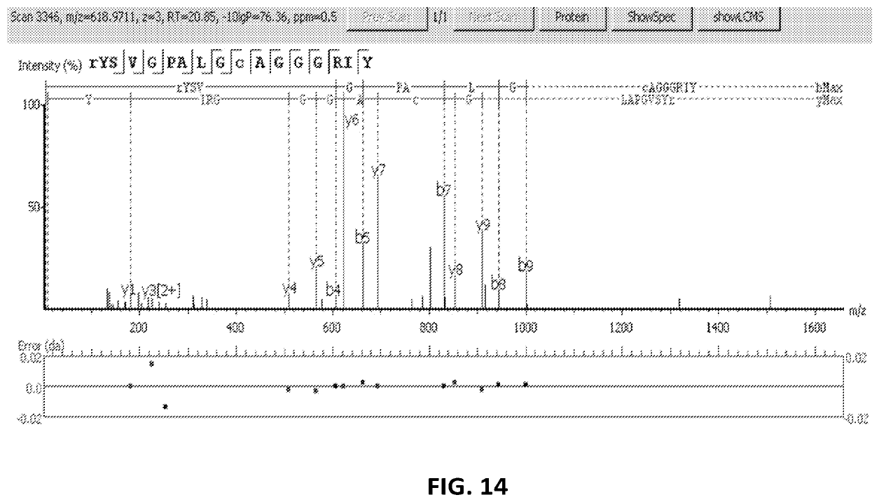

Covalent modification of Low-MW silk fibroin was confirmed for all three subunits (heavy chain, light chain, and fibrohexamerin) based on m/z and ms2 fragmentation patterns from the mass spectrum obtained in HPLC/MS analysis (See FIG. 9C and FIG. 14).

### Example 10d. Sample 077-30-1

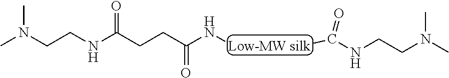

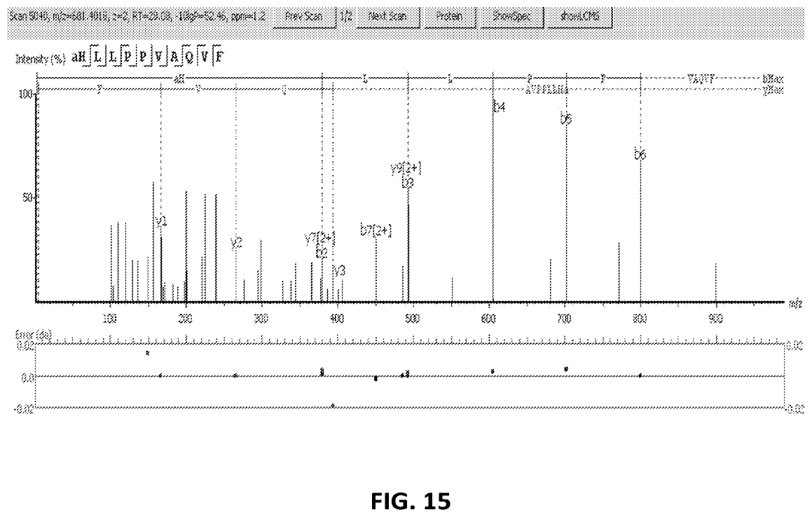

Covalent modification of Low-MW silk fibroin was confirmed for all three subunits (heavy chain, light chain, and fibrohexamerin) based on m/z and ms2 fragmentation patterns from the mass spectrum obtained in HPLC/MS analysis (See FIG. 9D and FIG. 15) Example 10e. Sample 098-08-02

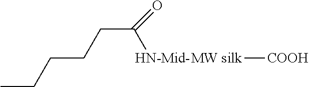

Mid MW silk was adjusted to pH 7.2 with phosphate buffer and heated to 37° C. Hexanal was then added, followed by hydrogen peroxide and the solution was allowed to react with stirring 24 hr. The solution was then cooled to room temperature and purified by dialysis against water using a 10 kDa MWCO dialysis tubing.

### Example 10f. Sample 098-29-02

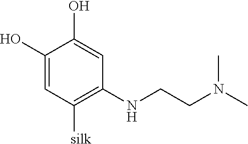

Mid MW silk was adjusted to pH 6.5 in phosphate buffer and heated to 35° C. Mushroom Tyrosinase was added and the solution was allowed to stir for 2 hr. The solution was then heated to 85° C. for 10 min to deactivate the tyrosinase enzyme, then the temperature was reduced to 60° C. and N,N-dimethylethylenediamine was added. The reaction mixture was allowed to react for 2 hr. The solution was then cooled to room temperature and purified by dialysis against water using a membrane with MWCO of 10 kDa.

### Example 10g. Sample 098-30-02

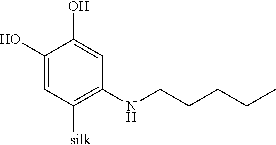

Mid MW silk was adjusted to pH 6.5 in phosphate buffer and heated to 35° C. Mushroom Tyrosinase was added and the solution was allowed to stir for 2 hr. The solution was then heated to 85° C. for 10 min to deactivate the tyrosinase enzyme, then the temperature was reduced to 60° C. and 1-aminopentane was added. The reaction mixture was allowed to react for 2 hr. The solution was then cooled to room temperature and purified by dialysis against water using a membrane with MWCO of 10 kDa.

### Example 11. Electrophoresis Gel

The molecular weight bands of the functionalized silk samples was obtained by gel electrophoresis using Novex precast 3-10 IEF gels. The gel electrophoresis experiments were run according to ThermoFisher Novex “Pre-Cast Gel Electrophoresis Guide” Version B, Jan. 27, 2003 IM-1002. In general, functionalized silk samples were diluted to 14.7 mg/ml protein concentration in 3-10 IEF sample buffer before loading and BioRad 4.45-9.6 IEF electrophoresis standards were used. The gels were focused at 100 constant volts for 1 hour, 200 constant volts for 1 hour, and 500 constant volts for 30 minutes. The gels were fixed in 12% TCA for ½ hour; stained in Coomassie Brilliant Blue R-250, stained in 10% acetic acid, and dried between cellophane sheets.

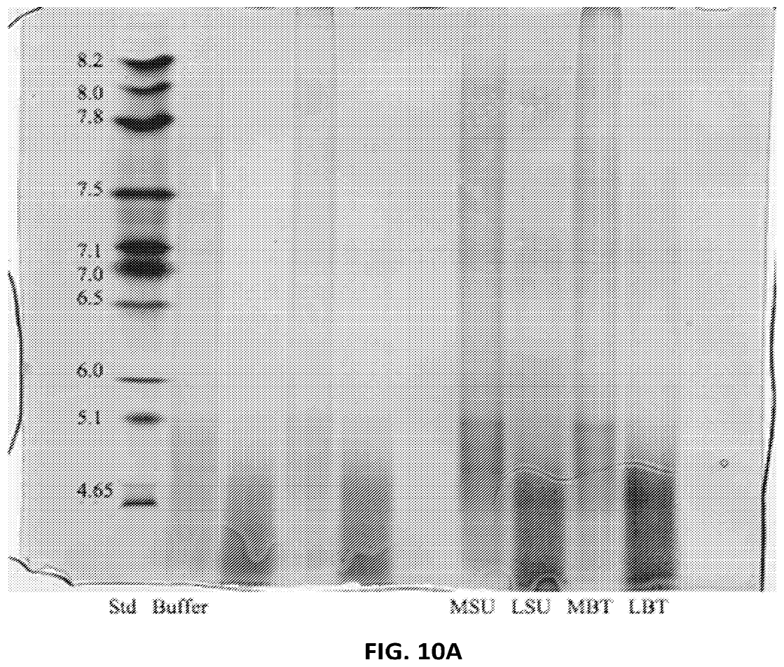

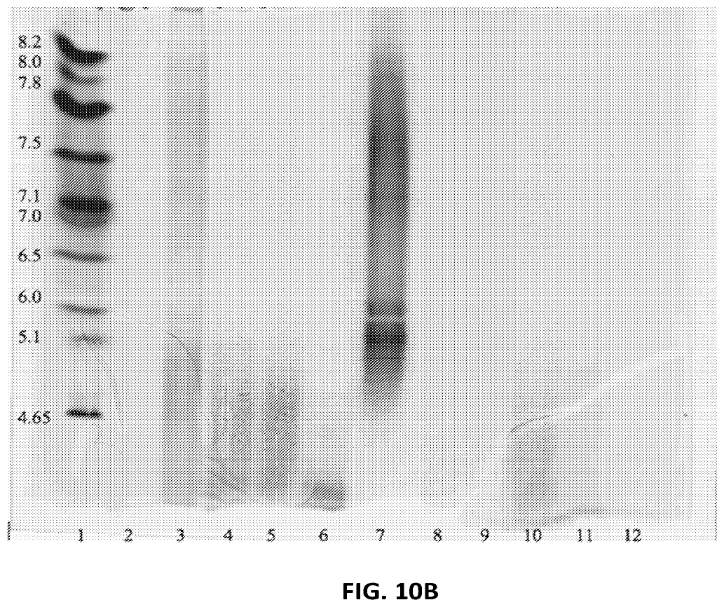

FIGS. 10A-B show the results for the electrophoresis gel experiments performed on the functionalized silk synthesized in Examples 10 above and the controls. FIG. 10A shows the electrophoresis gel from a few typical Activated Silks™, and FIG. 10B shows the electrophoresis gel for chemically modified Activated Silks™. Table 27 lists the Sample descriptions for each of Lanes 1-12 on FIG. 10B.

The mid-molecular weight Activated Silks™ have two isoelectric point ranges, one between pH 4-5 and a second one between pH 7-8. In contrast, low molecular weight Activated Silks™ have only one isoelectric point, in the range of pH 4-5. Upon chemical modification the isoelectric points of acetylated (sample 077-024-2) and methacrylated (sample 077-027-1) silks are unchanged. However, succinylation (sample 077-028-2) moves the isoelectric point to lower values (pH<4.65), while amination (sample 077-030-1) move the isoelectric point to a higher value (pH 5.1-6) and give rise to an additional isoelectric point (pH 7-8).

TABLE 27
Sample description for the gel electrophoresis
samples shown in FIG. 10B

| Lane | Sample | μg load | μl Load |
|---|---|---|---|
| 1 | BioRad IEF Stds | — | 6 |
| 2 | IEF Sample Buffer | — | 7.35 |
| 3 | 077-024-2 | 100 | 7.35 |
| 4 | 077-027-1 | 100 | 7.35 |
| 5 | 077-027-2 | 100 | 7.35 |
| 6 | 077-028-2 | 100 | 7.35 |
| 7 | 077-030-1 | 100 | 7.35 |
| 8 | MC-1 | 100 | 7.35 |
| 9 | S700-SP | 100 | 7.35 |
| 10 | DBr-7 | 100 | 7.35 |
| 11 | Ser-1 | 100 | 7.35 |
| 12 | — | — | — |

Molecular weight distribution for the functionalized silk samples was obtained by the size exclusion chromatography analysis. In general, sample solutions of the functionalized silk were analyzed on an Agilent 1100 HPLC equipped with a PolySep GFC P-4000 (7.8×300 mm) size exclusion column and a refractive index detector. The instrument was operated at a flow rate of 1 mL/min using a mobile phase containing 100 mM sodium chloride+12.5 mM sodium phosphate buffer (pH 7), for a sample run time of 20 minutes. The molecular weight distribution was calculated relative to Dextran standards using the Cirrus software package.

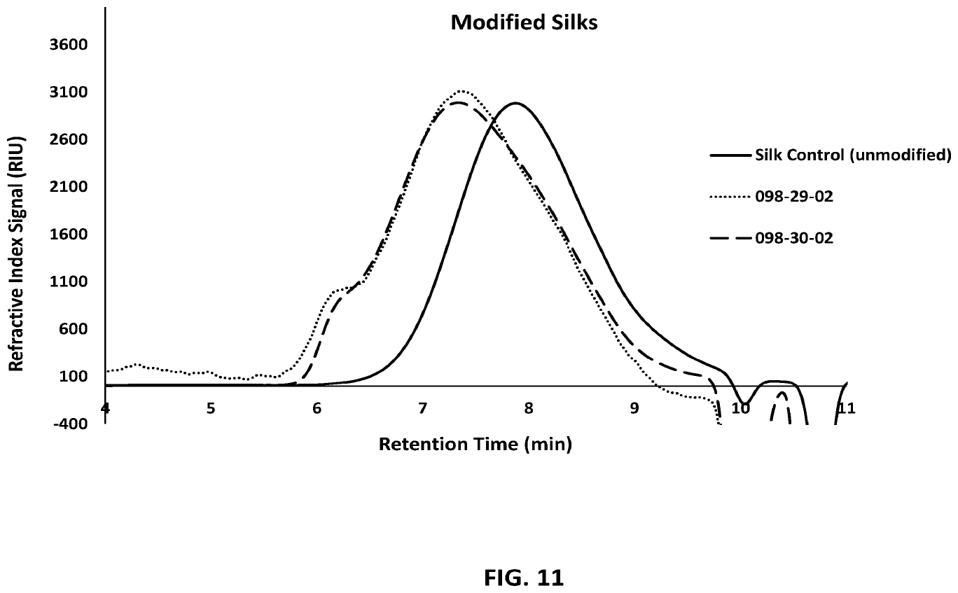

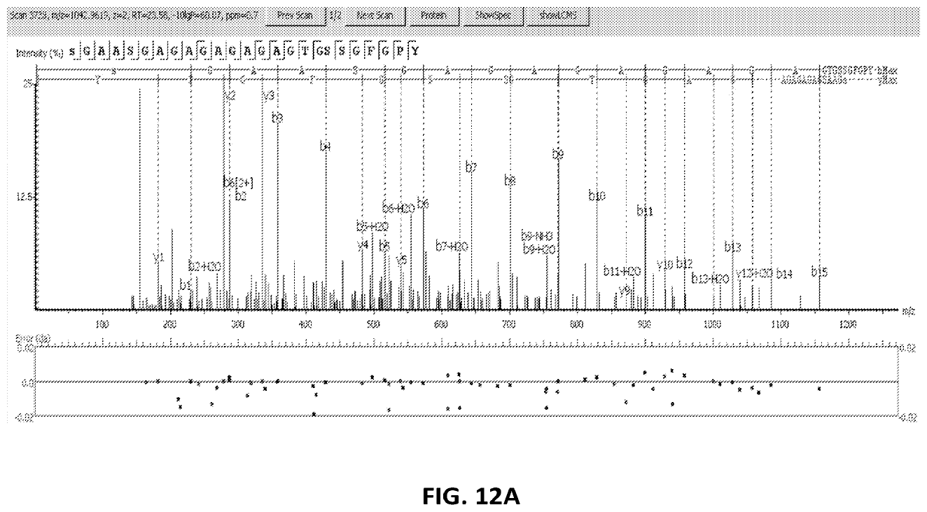

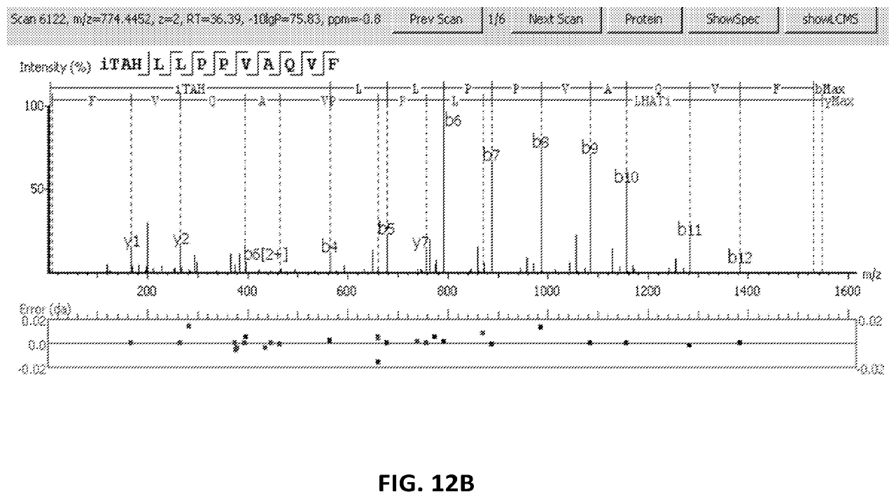

FIG. 11 shows the chromatograms of two modified mid molecular weight silks compared to a typical mid molecular silk. The two modified silks have higher molecular weight compared to the standard (evidenced by the shift towards early elution times).

1. A hair care composition comprising:
silk fibroin fragments having an average weight average molecular weight selected from between about 1 kDa and about 5 kDa, between about 5 kDa and about 10 kDa, between about 6 kDa and about 17 kDa, between about 10 kDa and about 15 kDa, between about 15 kDa and about 20 kDa, between about 17 kDa and about 39 kDa, between about 20 kDa and about 25 kDa, between about 25 kDa and about 30 kDa, between about 30 kDa and about 35 kDa, between about 35 kDa and about 40 kDa, between about 39 kDa and about 80 kDa, between about 40 kDa and about 45 kDa, between about 45 kDa and about 50 kDa, between about 60 kDa and about 100 kDa, and between about 80 kDa and about 144 kDa, and a polydispersity between 1 and about 5;
0 to 500 ppm lithium bromide;
0 to 500 ppm sodium carbonate; and
a dermatologically acceptable carrier.

2. The hair care composition of claim 1, wherein the silk fibroin fragments have a polydispersity between 1 and about 1.5, between about 1.5 and about 2.0, between about 1.5 and about 3.0, between about 2.0 and about 2.5, or between about 2.5 and about 3.0.

3-6. (canceled)

7. The hair care composition of claim 1, wherein the silk fibroin fragments are present in the hair care composition at about 0.01 wt. % to about 10.0 wt. % relative to the total weight of the hair care composition.

8. The hair care composition of claim 1, wherein the silk fibroin fragments are present in the hair care composition at about 0.01 wt. % to about 1.0 wt. %, at about 1.0 wt. % to about 2.0 wt. %, at about 2.0 wt. % to about 3.0 wt. %, at about 3.0 wt. % to about 4.0 wt. %, at about 4.0 wt. % to about 5.0 wt. %, or at about 5.0 wt. % to about 6.0 wt. % relative to the total weight of the hair care composition.

9-13. (canceled)

14. The hair care composition of claim 1, further comprising about 0.01% (w/w) to about 10% (w/w) sericin relative to the total weight of the hair care composition.

15. The hair care composition of claim 1, further comprising about 0.01% (w/w) to about 10% (w/w) sericin relative to the silk fibroin fragments.

16. The hair care composition of claim 1, wherein the silk fibroin fragments do not spontaneously or gradually gelate and do not visibly change in color or turbidity when in an aqueous solution for at least 10 days prior to formulation into the hair care composition.

17. The hair care composition of claim 1, wherein the dermatologically acceptable carrier comprises an oil phase.

18. The hair care composition of claim 1, wherein the dermatologically acceptable carrier comprises an aqueous phase.

19. The hair care composition of claim 1, further comprising an emulsifier.

20. The hair care composition of claim 1, wherein the dermatologically acceptable carrier comprises an oil-in-water emulsion or a water-in-oil emulsion.

21. The hair care composition of claim 1, further comprising a hydrocarbon oil, a fatty acid, a fatty oil, a fatty acid ester, a cationic quaternary ammonium salt, or any combination thereof.

22. The hair care composition of claim 1, further comprising a detersive, a detergent, or a combination thereof.

23. The hair care composition of claim 1, further comprising a hair styling polymer.

24. The hair care composition of claim 1, wherein the hair care composition is transparent, translucent, or opaque.

25. The hair care composition of claim 1, wherein the hair care composition is formulated as a hair pre-shampoo product, a hair shampoo product, a hair rinsing product, a hair conditioner product, a hair treatment product, a hair setting lotion product, a blow-styling lotion product, a hair spray product, a hair-styling foam product, a hair-styling gel product, a hair oil product, a hair liquid product, a hair tonic product, a hair cream product, a hair cosmetic product, a permanent waving agent product, a hair mousse product, a hair gel product, hair foam product, a hair paste product, a hair lotion product, or a hair wax product.

26-47. (canceled)

48. A hair care composition capable of foaming comprising the hair care composition of claim 1, and a surfactant system at less than 20.0 wt. % by the total weight of the hair care composition containing one or more of (a) a sulfate-based surfactant, (b) a silicon-based surfactant, and (c) a synthetic surfactant.

49. A hair dyeing composition comprising the hair care composition of claim 1, wherein the hair dyeing composition capable of coating hair cuticles and managing hair frizz.

50. A hair care composition capable of increasing hair sheen comprising the hair care composition of claim 1.

51. A hair care composition capable of enhancing hair color comprising the hair care composition of claim 1.

52-82. (canceled)
